(19) United States
(12) Patent Application Publication
(10) Pub. No.: US 2023/0052017 A1
(43) Pub. Date: Feb. 16, 2023

(54) New compounds for treatment of diseases related to DUX4 expression

(71) Applicant: Facio Intellectual Property B.V., Leiden (NL)

(72) Inventors: Pui Leng Loke, Abingdon, Oxfordshire (GB); Joris Herman De Maeyer, Mechelen (BE); Robert David Matthew Pace, Abingdon, Oxfordshire (GB); Gregory Foulkes, Abingdon, Oxfordshire (GB); Ainoa Rueda-Zubiaurre, Madrid (ES); Simon Fletcher Ellwood, Abingdon, Oxfordshire (GB); Adam James Davenport, Abingdon, Oxfordshire (GB); Anthony Paul Dickie, Abingdon, Oxfordshire (GB); Gerd Schnorrenberg, Biberach (DE)

(73) Assignee: Facio Intellectual Property B.V., Leiden (NL)

(21) Appl. No.: 17/779,139

(22) PCT Filed: Nov. 27, 2020
(86) PCT No.: PCT/EP2020/083796

§ 371 Date: May 24, 2022

(30) Foreign Application Priority Data
Nov. 29, 2019 (EP) 19212743.9

(51) Int. Cl. C07D 401/14; C07D 401/04; C07D 471/04; C07D 487/04; C07D 491/107; C07D 487/10; C07D 413/04; C07D 487/08; C07D 471/10; C07D 498/10; C07D 471/08

(52) U.S. Cl. CPC C07D 401/14; C07D 401/04; C07D 471/04; C07D 487/04; C07D 491/107; C07D 487/10; C07D 413/04; C07D 487/08; C07D 471/10; C07D 498/10; C07D 471/08

(57) ABSTRACT

The present invention relates to compounds for the treatment of diseases related to DUX4 expression, such as muscular dystrophies. It also relates to use of such compounds, or to methods of use of such compounds.

### FIELD OF THE INVENTION

The present invention relates to compounds for the treatment of diseases related to DUX4 expression, such as muscular dystrophies and cancer. It also relates to use of such compounds, or to methods of use of such compounds.

### BACKGROUND ART

Facioscapulohumeral muscular dystrophy (FSHD) is the most prevalent hereditary muscular dystrophy. Symptoms begin before the age of 20, with weakness and atrophy of the muscles around the eyes and mouth, shoulders, upper arms and lower legs. Later, weakness can spread to abdominal muscles and sometimes hip muscles with approximately 20% of patients eventually becoming wheelchair-bound. Patients currently rely on treatment of symptoms like pain and fatigue, involving the use of pain medication, cognitive therapy and physical exercise, sometimes supplemented with medical devices used to maintain the patient's mobility. Furthermore, increased scapular function may be obtained by surgical treatment of the scapula. At best, these interventions remain symptomatic in nature and do not affect disease progression, illustrating the need for a therapy that is able to modify disease progression.

Significant progress has been made in recent years in the understanding of the molecular basis of FSHD. This resulted in the identification and characterization of the fundamental genetic lesions causing FSHD, giving rise to the pathogenesis model in which gain-of-function of the Double Homeobox 4 (DUX4) retrogene in muscle cells underlies FSHD etiology (Lemmers et al., 2010, DOI: 10.1126/science.1189044; Sharma et al., 2016, DOI: 10.4172/2157-7412.1000303, Snider et al., 2010, DOI: 10.1371/journal.pgen.1001181; Tawil et al., 2014, DOI: 10.1186/2044-5040-4-12). DUX4 is a transcription factor that targets several genes and triggers pathology by initiating a transcription deregulation cascade that inhibits myogenesis and causes muscle atrophy, inflammation, and oxidative stress, ultimately resulting in progressive muscle cell dysfunction and death (Kowaljow et al., 2007, DOI: 10.1016/j.nmd.2007.04.002; Vanderplanck et al., 2011, doi: 10.1371/journal.pone.0026820; Geng et al., 2012, DOI: 10.1016/j.devcel.2011.11.013; Yao et al., 2014, DOI: 10.1093/hmg/ddu251; Wallace et al., 2011, DOI: 10.1002/ana.22275). DUX4 is normally abundantly expressed in germ cells of human testes, while being epigenetically repressed in somatic tissues. The DUX4 gene is located within a DNA tandem array (D4Z4) that is located in the subtelomeric region of chromosome 4q35.

FSHD is sometimes divided in two subtypes, namely FSHD1 and FSHD2. In the majority of patients (FSHD1), the disease is associated with large deletions within the D4Z4 array. Healthy, genetically unaffected individuals are defined as having between 10 and 100 D4Z4 repeat units on both 4q chromosome arms, whereas individuals with FSHD1 have between 1 and 10 D4Z4 repeat units on one 4q chromosome arm. The deletions of D4Z4 repeats that characterize FSHD remove a substantial portion of regulatory chromatin from this region, including several hundreds of histones and a significant amount of CpG-rich DNA. These elements are essential in the establishment of DNA methylation and heterochromatin and their loss significantly alters the epigenetic status of the D4Z4 array leading to derepression of the region. Patients carrying a smaller number of repeats (1-3 units) are on average more severely affected than those with a higher number of repeats (8-9) (Tawil et al., 1996, DOI: 10.1002/ana.410390610). The contraction of D4Z4 is by itself not pathogenic. Only when the contraction of D4Z4 occurs on a disease-permissive 4qA allele, containing a polymorphism that could affect the polyadenylation of the distal DUX4 transcript, the altered epigenetic context is associated with alternative splicing and increased expression of DUX4 in skeletal muscles of FSHD1 patients. In the much rarer form FSHD2, patients manifest similar symptoms, but genetically differ from FSHD1. These patients have longer D4Z4 repeats but exhibit similar derepression of the D4Z4 locus leading to DUX4 expression (Calandra et al., 2016; Jones et al., 2014; 2015). This loss of chromatin repression is caused by mutated forms of an epigenetic factor such as SMCHD1 or DNMT3B. Both forms of FSHD converge on undue DUX4 expression (Van den Boogaard et al., 2016, DOI: 10.1016/j.ajhg.2016.03.013).

In healthy individuals, DUX4 is expressed in the germline, but is epigenetically silenced in somatic tissues. In FSHD patients, burst-like DUX4 expression in only a small fraction of myofibers causes myocyte death ultimately leading to muscle weakness and wasting (Lemmers et al., 2010). In the simplest terms, DUX4-overexpression is a primary pathogenic insult underlying FSHD, and its repression is a promising therapeutic approach for FSHD. In support of this, short repeat sizes are generally associated with a severe FSHD phenotype. Moderate repeat contractions have a milder and more variable clinical severity. Patients with less than 10 D4Z4 repeat units (FSHD1) that also have a mutation in SMCHD1 (FSHD2) have a very severe clinical phenotype, illustrating that a combination of repeat size and activity of epigenetic modifiers, both contributing to derepression of DUX4, determines the eventual disease severity in FSHD.

Because of its causative role in FSHD, suppressing DUX4 is a primary therapeutic approach for halting disease progression. This approach could also be useful for treating other diseases, such as cancers including acute lymphoblastic leukemia (Yasuda et al., 2016, doi: 10.1038/ng.3535) and sarcomas (Oyama et al., 2017 DOI: 10.1038/s41598-017-04967-0; Bergerat et al., 2017, DOI: 10.1016/j.prp.2016.11.015), etc. It has recently been shown that DUX4 is also re-expressed in diverse solid cancers. Both cis-acting inherited genetic variation and somatically acquired mutations in trans-acting repressors contribute to DUX4 re-expression in cancer. DUX4-expressing cancers were characterized by reduced markers of anti-tumor cytolytic activity and lower major histocompatibility complex (MHC) class I gene expression. DUX4 expression blocks interferon-γ-mediated induction of MHC class I, implicating suppression of antigen presentation and a potential trole of DUX4 in immune evasion of the tumor. Clinical data in metastatic melanoma showed that DUX4 expression was associated with significantly reduced progression-free and overall survival in response to anti-CTLA-4. These data suggest that cancers can escape immune surveillance by reactivating DUX4 expression and that DUX4-mediated suppression of MHC class I-dependent antigen presentation is a clinically relevant biomarker for response to immune checkpoint blockade. This implies that repression of DUX4 is also a therapeutically relevant approach for several oncology indications and can be an adjuvant treatment to increase responsiveness to immune therapy in oncology (Chew et al., 2019, DOI: 10.1016/j.devcel.2019.06.011).

The mechanisms behind DUX4 expression are poorly understood and corresponding drug targets are poorly defined. As a result, there is no treatment for FSHD at present, and there is a need for compounds and compositions that can be used to suppress DUX4 expression.

### SUMMARY OF THE INVENTION

The invention relates to a compound of general formula (I):

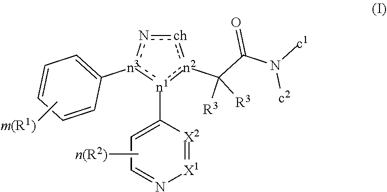

Wherein zero or one of n¹, n², and n³ are N, with the remainder of n¹, n², and n³ being C; ch is CH, C(halogen), C(OH), C(—C₁₋₄alkyl), C(—C₁₋₄haloalkyl), C(—C₃₋₆cycloalkyl), C(—C₃₋₆heterocycloalkyl), O, NH, N(—C₁₋₄alkyl), or N(—C₁₋₄haloalkyl); R¹ is H, halogen, nitrile, —C₁₋₄alkyl, —C₁₋₃alkyl-nitrile, —C₁₋₄haloalkyl, —C₁₋₃haloalkyl-nitrile, —O—C₁₋₄alkyl, —O—C₁₋₃alkyl-nitrile, —O—C₁₋₄haloalkyl, —O—C₁₋₃haloalkyl-nitrile, —S—C₁₋₄alkyl, —S—C₁₋₃alkyl-nitrile, —S—C₁₋₄haloalkyl, or —S—C₁₋₃haloalkyl-nitrile; m is 0, 1, 2, or 3; R² is H, halogen, nitrile, —C₁₋₄alkyl, —C₁₋₃alkyl-nitrile, —C₁₋₄haloalkyl, —C₁₋₃haloalkyl-nitrile, —O—C₁₋₄alkyl, —O—C₁₋₃alkyl-nitrile, —O—C₁₋₄haloalkyl, —O—C₁₋₃haloalkyl-nitrile, —S—C₁₋₄alkyl, —S—C₁₋₃alkyl-nitrile, —S—C₁₋₄haloalkyl, —S—C₁₋₃haloalkyl-nitrile, or R² together with Q forms a bridging moiety; n is 0, 1, or 2; R³ is in each instance independently selected from H, halogen, or C₁₋₄alkyl; X¹ is CH, C(R²), N, or C(Q); X² is CH, C(R²), or N; Q is H, halogen, C₁₋₆alkyl, —OH, —O—C₁₋₆alkyl, —O—C₁₋₆acyl, —NH₂, —NH—(C₁₋₆alkyl), —N(C₁₋₆alkyl)₂, —NH(C₁₋₈acyl), —N(C₁₋₈acyl)₂, —C₁₋₄alkyl-OH, —C₁₋₄alkyl-O—C₁₋₆alkyl, —C₁₋₄alkyl-O—C₁₋₆acyl, —C₁₋₄alkyl-NH₂, —C₁₋₄alkyl-NH—(C₁₋₆alkyl), —C₁₋₄alkyl-N(C₁₋₆alkyl)₂, —C₁₋₄alkyl-NH(C₁₋₈acyl), —C₁₋₄alkyl-N(C₁₋₈acyl)₂, —C₁₋₄alkyl-N—C(O)—NH—C₁₋₆alkyl, —C₁₋₄alkyl-N—C(O)—N(C₁₋₆alkyl)₂, —C₁₋₄alkyl-O—C(O)—NH—C₁₋₆alkyl, —C₁₋₄alkyl-O—C(O)—N(C₁₋₆alkyl)₂, —C₁₋₄alkyl-N—C(O)—O—C₁₋₆alkyl, or Q together with R² forms a bridging moiety selected from —NH—CH═CH—, —NH—(C₂₋₄alkyl)-, and —(C₁₋₃alkyl)-NH—(C₁₋₃alkyl)-; c¹ is H, C₁₋₆alkyl, (C₁₋₂alkyl)₀₋₁C₃₋₆cycloalkyl, or (C₁₋₂alkyl)₀₋₁C₄₋₆heterocycloalkyl, preferably c¹ is H and c² is C₄₋₈cycloalkyl, C₄₋₈heterocycloalkyl, C₄₋₈cycloalkyl-C₁₋₃alkyl, C₄₋₈heterocycloalkyl-C₁₋₃alkyl, C₁₋₃alkyl-C₄₋₈cycloalkyl, or C₁₋₃alkyl-C₄₋₈heterocycloalkyl, or c¹ and c² together form cyclic structure A; A is a C₄₋₁₂cycloalkyl that can be cyclic, bicyclic, and tricyclic, and which is optionally unsaturated, and which is optionally substituted with halogen, C₁₋₆alkyl, —O—C₁₋₄alkyl, hydroxyl, —NH₂, —NH(C₁₋₄alkyl), or —N(C₁₋₄alkyl)₂; wherein each instance of acyl, alkyl, cycloalkyl, or heterocycloalkyl individually is optionally unsaturated, and optionally substituted with halogen, oxy, hydroxyl, methyl, ethyl, propyl, methoxy, ethoxy, trifluoromethyl, or optionally interrupted by one or more heteroatoms; or a salt thereof.

In preferred embodiments, n² is N and n¹ is C and n³ is C; ch is CH, C(Cl), C(CH₃), C(isopropyl), C(CF₃), O, NH, or N(CH₃); R¹ is H, fluorine, chlorine, —CH₃, —CF₃, —O—CH₃, or nitrile; m is 0 or 1; R² is H, fluorine, chlorine, or forms a bridging moiety; n is 0; R³ is H or —CH₃; X¹ is C(Q); X² is CH; Q is H, F, —CH₃, —CH₂F, —CHF₂, —CF₃, —OCH₃, —OCH₂F, —OCHF₂, —OCF₃, —NH—C(O)—CH₃, —NH—C(O)— cyclopropyl, —NH—C(O)-phenyl, —NH—C(O)-halophenyl, —NH—C(O)-piperidinyl, —NH—C(O)-pyridinyl, —NH—C(O)-morpholinyl, —NH—C(O)-oxanyl, —NH₂, —NH(CH₃), —NH(cyclopentyl), —CH₂—NH—C(O)—CH₃, —CH₂—N(CH₃)₂, —CH₂—NH₂, —CH₂—NH—(CH₃), —CH₂—NH-(cyclopentyl), or together with R² forms —NH—CH═CH—; and/or c¹ is H and c² is pyridyl, —CH₂-pyridyl, piperidinyl, N-methylpiperidinyl, —CH₂-piperidinyl, —CH₂-(N-methylpiperidinyl), cyclopentyl, hydroxycyclopentyl, —CH₂-cyclopentyl, —CH₂-hydroxycyclopentyl, pyrrolidinyl, N-methylpyrrolidinyl, —CH₂-pyrrolidinyl, —CH₂-(N-methylpyrrolidinyl), or c¹ and c² together form cyclic structure A.

Preferably, Q is H, F, —NH—C(O)—CH₃, —NH—C(O)-cyclopropyl, —NH—C(O)-phenyl, —NH—C(O)— halophenyl, —NH₂, —NH(CH₃), —NH(cyclopentyl), —CH₂—NH—C(O)—CH₃, —CH₂—N(CH₃)₂, —CH₂—NH₂, —CH₂—NH—(CH₃), —CH₂—NH-(cyclopentyl), or together with R² forms —NH—CH═CH—; and/or wherein R³ is H; and/or wherein R¹ is H, fluorine, chlorine, —CH₃, —CF₃, or —O—CH₃. Preferably, A is optionally substituted and optionally unsaturated azetidinyl, pyrrolidinyl, imidazolidinyl, oxazolidinyl, piperidinyl, piperazinyl, morpholinyl, azacycloheptyl, diazacycloheptyl, or oxoazacycloheptyl; wherein each optional substitution can be a substitution with halogen, C₁₋₆alkyl, C₃₋₆cycloalkyl, C₃₋₆heterocycloalkyl, —O—C₁₋₄alkyl, hydroxyl, —NH₂, —NH(C₁₋₄alkyl), or —N(C₁₋₄alkyl)₂; preferably each optional substitution is independently selected from methyl, dimethylamine, methoxyl, propyl, hydroxyl, a bridging C₁₋₃alkyl moiety, spiro azetidinyl, spiro N-methylazetidinyl, spiro oxetanyl, oxetanyl, spiro piperidinyl, difluoropiperidinyl, spiro N-methylpiperidinyl, spiro cyclopropyl, fused pyrrolidinyl, or fused N-methylpyrrolidinyl.

The compound can also be of general formula (I-A):

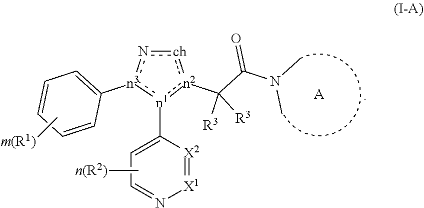

Preferably it is of general formula (II) or (II-A):

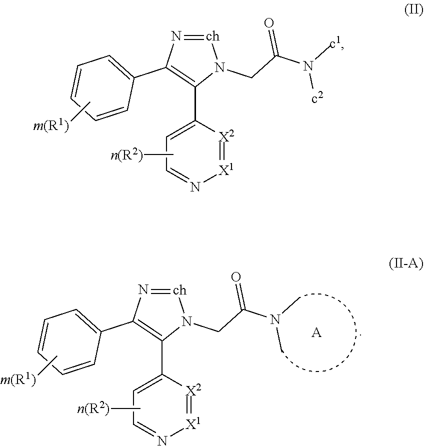

In preferred embodiments the compound is of general formula (III) or (III-A)

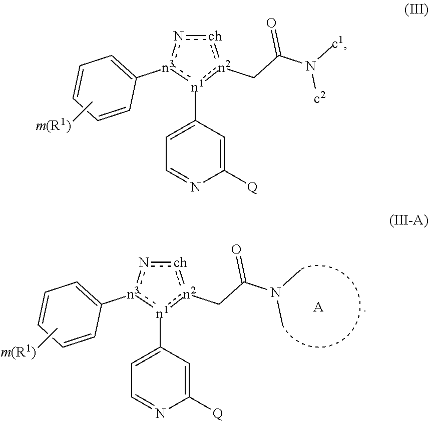

In preferred embodiments A comprises an amine, more preferably wherein A is selected from A1, A2, A4, A5, A7, A8, A10, A11, A12, A13, A16, A17, A18, A19, A20, A21, A22, A23, and A24. In other preferred embodiments, m is 1, and R¹ is para to the central ring, preferably R¹ is halogen, more preferably fluorine or chlorine, most preferably fluorine. Preferably the compound is selected from compounds 1-105 and 109-168 as listed in table 1. More preferably the compound is selected from compounds 2, 5, 10, 13, 14, 16, 18, 22, 28, 34, 40, 43, 45, 48, 49, 50, 51, 53, 55, 56, 57, 61, 63, 64, 90, 99, 3, 4, 6, 7, 8, 9, 11, 12, 15, 17, 19, 20, 21, 23, 24, 25, 26, 27, 29, 30, 31, 32, 33, 35, 36, 37, 38, 39, 41, 42, 44, 46, 47, 52, 58, 59, 62, 65, 81, 82, 83, 84, 85, 86, 87, 88, 89, 91, 92, 93, 94, 95, 96, 97, 98, 100, 101, 102, 103, and 105 as listed in table 1, more preferably from compounds 3, 4, 6, 7, 8, 9, 11, 12, 15, 17, 19, 20, 21, 23, 24, 25, 26, 27, 29, 30, 31, 32, 33, 35, 36, 37, 38, 39, 41, 42, 44, 46, 47, 52, 58, 59, 62, 65, 81, 82, 83, 84, 85, 86, 87, 88, 89, 91, 92, 93, 94, 95, 96, 97, 98, 100, 101, 102, 103, and 105 as listed in table 1.

The invention also provides a composition comprising at least one compound of general formula (I) as defined above, and a pharmaceutically acceptable excipient. The invention also provides the compound or composition as described above, for use as a medicament, wherein the medicament is preferably for use in the treatment of a disease or condition associated with DUX4 expression, and wherein the compound of general formula (I) reduces DUX4 expression, wherein more preferably said disease or condition associated with DUX4 expression is a muscular dystrophy or cancer, even more preferably wherein said disease or condition associated with DUX4 expression is a muscular dystrophy, most preferably facioscapulohumeral muscular dystrophy (FSHD). Also provided is an in vivo, in vitro, or ex vivo method for reducing DUX4 expression, the method comprising the step of contacting a cell with a compound of general formula (I) as defined above, or with a composition as defined above. The invention also provides a method for reducing DUX4 expression in a subject in need thereof, the method comprising the step of administering an effective amount of a compound of general formula (I) as defined above, or a composition as defined above.

### DESCRIPTION OF EMBODIMENTS

### Compound

The inventors have identified new compounds that function as DUX4 repressors. The invention provides a compound of general formula (I):

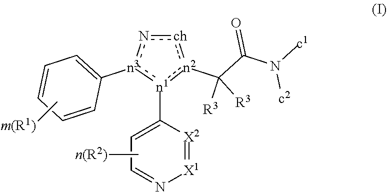

wherein
zero or one of n¹, n², and n³ are N, with the remainder of n¹, n², and n³ being C;
ch is CH, C(halogen), C(OH), C(—C₁₋₄alkyl), C(—C₁₋₄haloalkyl), C(—C₃₋₆cycloalkyl), C(—C₃₋₆heterocycloalkyl), O, NH, N(—C₁₋₄alkyl), or N(—C₁₋₄haloalkyl);
R¹ is H, halogen, nitrile, —C₁₋₄alkyl, —C₁₋₃alkyl-nitrile, —C₁₋₄haloalkyl, —C₁₋₃haloalkyl-nitrile, —O—C₁₋₄alkyl, —O—C₁₋₃alkyl-nitrile, —O—C₁₋₄haloalkyl, —O—C₁₋₃haloalkyl-nitrile, —S—C₁₋₄alkyl, —S—C₁₋₃alkyl-nitrile, —S—C₁₋₄haloalkyl, or —S—C₁₋₃haloalkyl-nitrile;
m is 0, 1, 2, or 3;
R² is H, halogen, nitrile, —C₁₋₄alkyl, —C₁₋₃alkyl-nitrile, —C₁₋₄haloalkyl, —C₁₋₃haloalkyl-nitrile, —O—C₁₋₄alkyl, —O—C₁₋₃alkyl-nitrile, —O—C₁₋₄haloalkyl, —O—C₁₋₃haloalkyl-nitrile, —S—C₁₋₄alkyl, —S—C₁₋₃alkyl-nitrile, —S—C₁₋₄haloalkyl, —S—C₁₋₃haloalkyl-nitrile, or R² together with Q forms a bridging moiety;
n is 0, 1, or 2;
R³ is in each instance independently selected from H, halogen, or C₁₋₄alkyl;
X¹ is CH, C(R²), N, or C(Q);
X² is CH, C(R²), or N;
Q is H, halogen, C₁₋₆alkyl, —OH, —O—C₁₋₆alkyl, —O—C₁₋₆acyl, —NH₂, —NH—(C₁₋₆alkyl), —N(C₁₋₆alkyl)₂, —NH(C₁₋₈acyl), —N(C₁₋₈acyl)₂, —C₁₋₄alkyl-OH, —C₁₋₄alkyl-O—C₁₋₆alkyl, —C₁₋₄alkyl-O—C₁₋₆acyl, —C₁₋₄alkyl-NH₂, —C₁₋₄alkyl-NH—(C₁₋₆alkyl), —C₁₋₄alkyl-N(C₁₋₆alkyl)₂, —C₁₋₄alkyl-NH(C₁₋₈acyl), —C₁₋₄alkyl-N(C₁₋₈acyl)₂, —C₁₋₄alkyl-N—C(O)—NH—C₁₋₆alkyl, —C₁₋₄alkyl-N—C(O)—N(C₁₋₆alkyl)₂, —C₁₋₄alkyl-O—C(O)—NH—C₁₋₆alkyl, —C₁₋₄alkyl-O—C(O)—N(C₁₋₆alkyl)₂, —C₁₋₄alkyl-N—C(O)—O—C₁₋₆alkyl, or Q together with R² forms a bridging moiety selected from —NH—CH═CH—, —NH—(C₂₋₄alkyl)-, and —(C₁₋₃alkyl)-NH—(C₁₋₃alkyl)-;
c¹ is H, C₁₋₆alkyl, (C₁₋₂alkyl)₀₋₁C₃₋₆cycloalkyl, or (C₁₋₂alkyl)₀₋₁C₄₋₆heterocycloalkyl, preferably c¹ is H and c² is C₄₋₈cycloalkyl, C₄₋₈heterocycloalkyl, C₄₋₈cycloalkyl-C₁-3alkyl, C₄₋₈heterocycloalkyl-C₁₋₃alkyl, C₁₋₃alkyl-C₄₋₈cycloalkyl, or C₁₋₃alkyl-C₄₋₈heterocycloalkyl, or c¹ and c² together form cyclic structure A;
A is a C₄₋₁₂cycloalkyl that can be cyclic, bicyclic, and tricyclic, and which is optionally unsaturated, and which is optionally substituted with halogen, C₁₋₆alkyl, —O—C₁₋₄alkyl, hydroxyl, —NH₂, —NH(C₁₋₄alkyl), or —N(C₁₋₄alkyl)₂;
wherein each instance of acyl, alkyl, cycloalkyl, or heterocycloalkyl individually is optionally unsaturated, and optionally substituted with halogen, oxy, hydroxyl, methyl, ethyl, propyl, methoxy, ethoxy, trifluoromethyl, or optionally interrupted by one or more heteroatoms;
or a salt thereof. Such a compound is referred to herein as a compound according to the invention. In preferred embodiments, the compound is a salt, more preferably an acid addition salt, most preferably a pharmaceutically acceptable acid addition salt.
   Preferably c¹ and c² together form cyclic structure A. In preferred embodiments a compound of general formula (I) is of general formula (I-A):

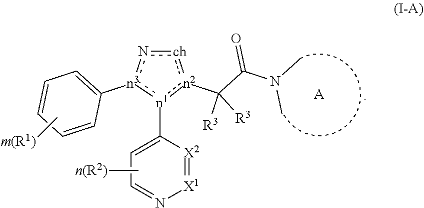

Central Ring of the Compound

Compounds according to the invention have a central five-membered ring that is aromatic and that comprises at least one nitrogen atom. This ring is referred to hereinafter as the central ring.

This ring has variables in ch, n¹, n², and n³.

ch is CH, C(halogen), C(OH), C(—C₁₋₄alkyl), C(—C₁₋₄haloalkyl), C(—C₃₋₆cycloalkyl), C(—C₃₋₆heterocycloalkyl), O, NH, N(—C₁₋₄alkyl), or N(—C₁₋₄haloalkyl); preferably it is CH, C(halogen), C(—C₁₋₄alkyl), C(—C₁₋₄haloalkyl), O, NH, N(—C₁₋₄alkyl), or N(—C₁₋₄haloalkyl); more preferably it is CH, C(Cl), C(CH₃), C(isopropyl), C(CF₃), O, NH, or N(CH₃); or more preferably it is C(OH), C(Br), C(cyclopropyl), C(4-methoxyphenyl), C(2-methoxypyridyn-5-yl), or C(1-methyl-1,2-diazacyclopenta-2,4-diene-4-yl); in some preferred embodiments it is CH, C(Cl), C(CH₃), C(isopropyl), C(CF₃), O, NH, N(CH₃), C(OH), C(Br), C(cyclopropyl), C(4-methoxyphenyl), C(2-methoxypyridyn-5-yl), or C(1-methyl-1,2-diazacyclopenta-2,4-diene-4-yl). For ch a preferred halogen is chlorine or fluorine. For haloalkyl in ch, a preferred halogen is fluorine. For C(halogen) in ch a preferred halogen is chlorine. Here, C(—C₁₋₄alkyl), C(—C₁₋₄haloalkyl), N(—C₁₋₄alkyl), and N(—C₁₋₄haloalkyl) are preferably C₁-3, more preferably C₁ or isopropyl, most preferably C₁. In preferred embodiments ch is C(Cl), C(CH₃), C(isopropyl), C(CF₃), O, NH, or N(CH₃).

In this context, C₃₋₆cycloalkyl and/or C₃₋₆heterocycloalkyl can be cyclic, bicyclic, and tricyclic, and which is optionally unsaturated, and which is optionally substituted with halogen, C₁₋₆alkyl, —O—C₁₋₄alkyl, hydroxyl, —NH₂, —NH(C₁₋₄alkyl), or —N(C₁₋₄alkyl)₂. In preferred embodiments there are no such optional substitutions. Multicyclic structures can be fused, bridged, or spiro. In preferred embodiments, C₃₋₆cycloalkyl and/or C₃₋₆heterocycloalkyl are not multicyclic. In a preferred embodiment, said C₃₋₆cycloalkyl is an optionally substituted cyclopropyl. In a preferred embodiment, said C₃₋₆heterocycloalkyl is an optionally substituted C₅₋₆ hetereocyloalkyl comprising one or two nitrogen atoms. In a more preferred embodiment, said C₃₋₆heterocycloalkyl is selected from the group consisting of optionally substituted pyridinyl and optionally substituted pyrazolyl, even more preferably from the group consisting of optionally substituted 4-pyridinyl and optionally substituted 4-pyrazolyl.

In preferred embodiments ch is C(halogen), C(—C₁₋₄alkyl), C(—C₁₋₄haloalkyl), O, NH, N(—C₁₋₄alkyl), or N(—C₁₋₄haloalkyl). In preferred embodiments ch is CH, C(—C₁₋₄alkyl), C(—C₁₋₄haloalkyl), O, NH, N(—C₁₋₄alkyl), or N(—C₁₋₄haloalkyl). In preferred embodiments ch is CH, C(halogen), C(—C₁₋₄haloalkyl), O, NH, N(—C₁₋₄alkyl), or N(—C₁₋₄haloalkyl). In preferred embodiments ch is CH, C(halogen), C(—C₁₋₄alkyl), O, NH, N(—C₁₋₄alkyl), or N(—C₁₋₄haloalkyl). In preferred embodiments ch is CH, C(halogen), C(—C₁₋₄alkyl), C(—C₁₋₄haloalkyl), NH, N(—C₁₋₄alkyl), or N(—C₁₋₄haloalkyl). In preferred embodiments ch is CH, C(halogen), C(—C₁₋₄alkyl), C(—C₁₋₄haloalkyl), O, N(—C₁₋₄alkyl), or N(—C₁₋₄haloalkyl). In preferred embodiments ch is CH, C(halogen), C(—C₁₋₄alkyl), C(—C₁₋₄haloalkyl), O, NH, or N(—C₁₋₄haloalkyl). In preferred embodiments ch is CH, C(halogen), C(—C₁₋₄alkyl), C(—C₁₋₄haloalkyl), O, NH, or N(—C₁₋₄alkyl). In preferred embodiments ch is CH, C(CH₃), C(isopropyl), C(CF₃), O, NH, or N(CH₃). In preferred embodiments ch is CH, C(Cl), C(isopropyl), O, NH, or N(CH₃). In preferred embodiments ch is CH, C(Cl), C(CH₃), C(isopropyl), C(CF₃), O, NH, or N(CH₃). In preferred embodiments ch is CH, C(Cl), C(CH₃), C(isopropyl), C(CF₃), NH, or N(CH₃). In preferred embodiments ch is CH, C(Cl), C(CH₃), C(isopropyl), C(CF₃), O, or N(CH₃). In preferred embodiments ch is CH, C(Cl), C(CH₃), C(isopropyl), C(CF₃), O, or NH.

In preferred embodiments, instances of alkyl or haloalkyl within ch are not unsaturated. In preferred embodiments, instances of alkyl or haloalkyl within ch are optionally unsaturated. In preferred embodiments, instances of alkyl or haloalkyl within ch are unsaturated. In preferred embodiments, instances of alkyl or haloalkyl within ch are not substituted with halogen, oxy, hydroxyl, methyl, ethyl, propyl, methoxy, ethoxy, trifluoromethyl, and not optionally interrupted by one or more heteroatoms. In preferred embodiments, instances of alkyl or haloalkyl within ch are optionally substituted with halogen, oxy, hydroxyl, methyl, ethyl, propyl, methoxy, ethoxy, trifluoromethyl, and not interrupted by one or more heteroatoms. In preferred embodiments, instances of alkyl or haloalkyl within ch are optionally substituted with halogen, oxy, hydroxyl, methyl, ethyl, propyl, methoxy, ethoxy, trifluoromethyl, and/or optionally interrupted by one or more heteroatoms, and/or optionally unsaturated.

n¹, n², and n³ can be N or C. In general formula (I) the chemical bonds are indicated as double bonds of which one bond is solid and the other bond is dashed. As a skilled person will understand, this is because the preferred location of the double bonds depends on the nature of n¹, n², and n³. In preferred embodiments each of n¹, n², and n³ is C. In other preferred embodiments, n¹ and n² are C, and n³ is N. In other preferred embodiments, n³ and n² are C, and n¹ is N. In the most preferred embodiments, n¹ and n³ are C, and n² is N.

In preferred embodiments the central ring of the compound is as shown below (reference name shown below the structures). CR1-CR10 are particularly preferred, CR10 is most preferred.

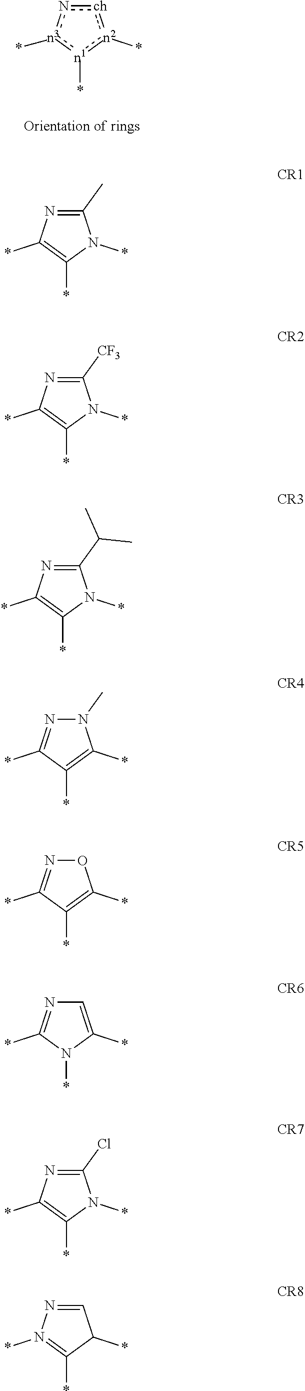

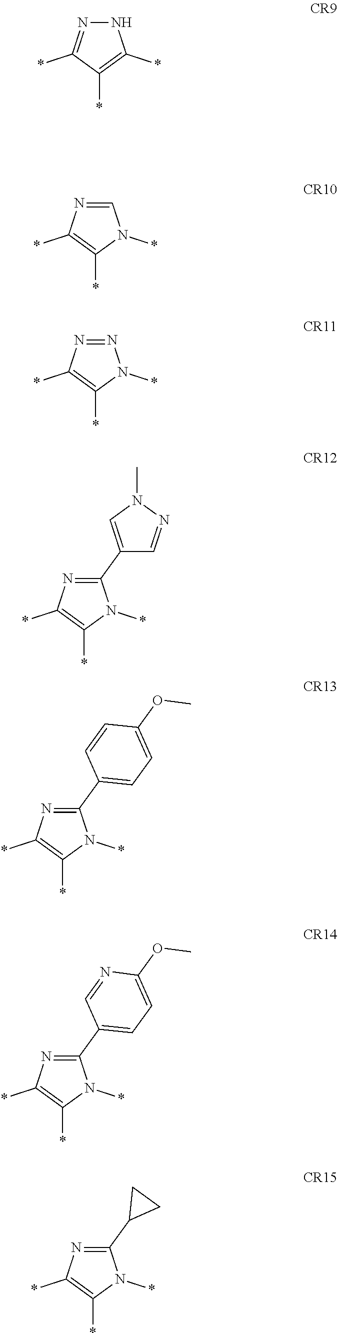

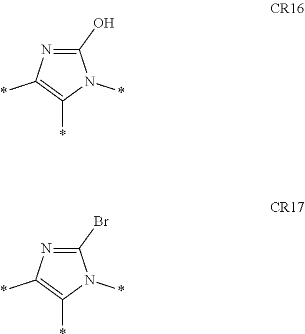 [SMILES: *c1nc(O)n(*)c1* ; *c1nc(Br)n(*)c1*]

Compounds of general formula (I) are preferably of generally formula (II):

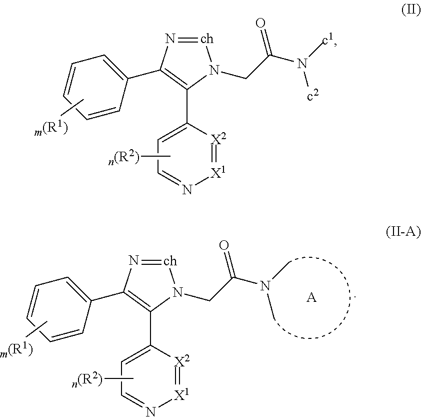

Phenylic Moiety of the Compound

Compounds according to the invention have a phenylic moiety that is attached to n³ of the central ring of the compound according to the invention. It is substituted with 0, 1, 2, or 3 instances of R¹. This moiety is herein referred to as the phenylic moiety. The amount of substitution by R¹ is denoted by m, which can be 0, 1, 2, or 3. In preferred embodiments, m is 0, 1, or 2. In preferred embodiments, m is 1, 2, or 3. In preferred embodiments, m is 1 or 2. In preferred embodiments, m is 0. In preferred embodiments, m is 1. In preferred embodiments, m is 2. In preferred embodiments, m is 3. Most preferably m is 0 or 1.

R¹ is a substituent that is H, halogen, nitrile, —C₁₋₄alkyl, —C₁₋₃alkyl-nitrile, —C₁₋₄haloalkyl, —C₁₋₃haloalkyl-nitrile, —O—C₁₋₄alkyl, —O—C₁₋₃alkyl-nitrile, —O—C₁₋₄haloalkyl, —O—C₁₋₃haloalkyl-nitrile, —S—C₁₋₄alkyl, —S—C₁₋₃alkyl-nitrile, —S—C₁₋₄haloalkyl, or —S—C₁₋₃haloalkyl-nitrile; preferably it is H, halogen, —C₁₋₄alkyl, —C₁₋₄haloalkyl, —O—C₁₋₄alkyl, —O—C₁₋₄haloalkyl, —S—C₁₋₄alkyl, or —S—C₁₋₄haloalkyl; in preferred embodiments R¹ is H, fluorine, chlorine, —CH₃, —CF₃, —O—CH₃, or nitrile; more preferably it is H, fluorine, chlorine, —CH₃, —CF₃, or —O—CH₃. Here, —C₁₋₄alkyl and —C₁₋₄haloalkyl are preferably —C₁₋₃alkyl or C₁₋₃haloalkyl, more preferably C₁ variants or isopropyl, most preferably C₁ variants.

In preferred embodiments, R¹ is halogen, —C₁₋₄alkyl, —C₁₋₄haloalkyl, —O—C₁₋₄alkyl, —O—C₁₋₄haloalkyl, —S—C₁₋₄alkyl, or —S—C₁₋₄haloalkyl. In preferred embodiments, R¹ is H, —C₁₋₄alkyl, —C₁₋₄haloalkyl, —O—C₁₋₄alkyl, —O—C₁₋₄haloalkyl, —S—C₁₋₄alkyl, or —S—C₁₋₄haloalkyl. In preferred embodiments, R¹ is H, halogen, —O—C₁₋₄alkyl, —O—C₁₋₄haloalkyl, —S—C₁₋₄alkyl, or —S—C₁₋₄haloalkyl. In preferred embodiments, R¹ is H, halogen, —C₁₋₄alkyl, —C₁₋₄haloalkyl, —S—C₁₋₄alkyl, or —S—C₁₋₄haloalkyl. In preferred embodiments, R¹ is H, halogen, —C₁₋₄alkyl, —C₁₋₄haloalkyl, —O—C₁₋₄alkyl, or —O—C₁₋₄haloalkyl.

When m is not 0, the phenylic moiety has at least one R¹. When R¹ is present, it is preferably meta or para to the central ring. In preferred embodiments it is ortho to the central ring. In preferred embodiments it is meta to the central ring. In preferred embodiments it is para to the central ring. In preferred embodiments it is ortho or meta to the central ring. In preferred embodiments it is ortho or para to the central ring. Most preferably when a single R¹ is present it is para to the central ring.

In preferred embodiments is provided the compound according to the invention, wherein m is 1, and wherein R¹ is para to the central ring, preferably wherein R¹ is halogen, more preferably fluorine or chlorine, most preferably fluorine.

In preferred embodiments the phenylic moiety of the compound is as shown below, with a reference name shown below each structure. Ph1-Ph9 and Ph11-Ph19 and Ph20-Ph21 are particularly preferred, Ph1-Ph9 and Ph11-Ph19 are preferred, Ph1-Ph9 are more preferred, Ph1-Ph8 are even more preferred, Ph5 and Ph8 are even more preferred, Ph8 is most preferred.

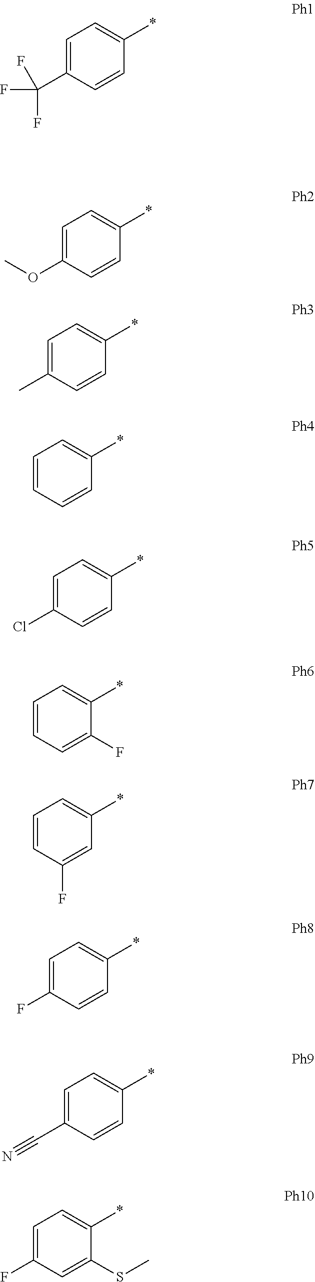

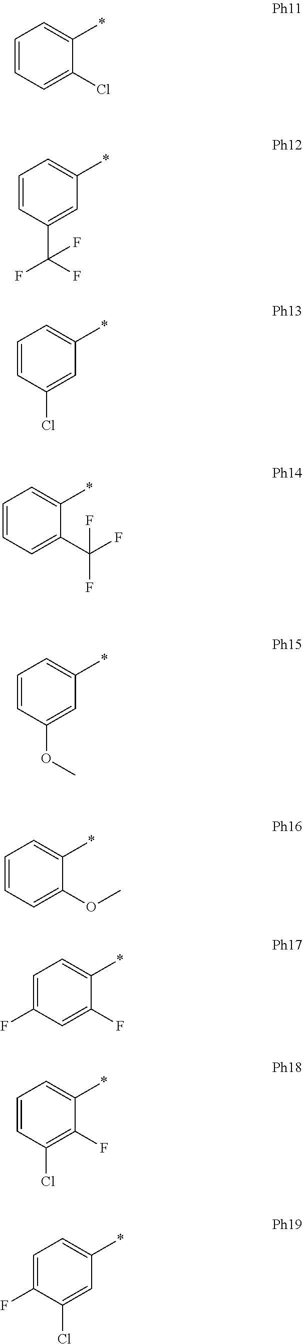

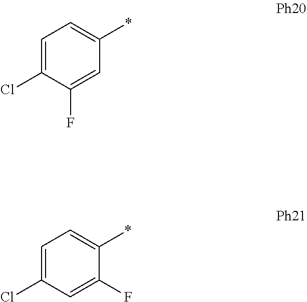 [SMILES: *c1ccc(Cl)c(F)c1 ; *c1ccc(Cl)cc1F]

In preferred embodiments, instances of alkyl or haloalkyl within R¹ are not unsaturated. In preferred embodiments, instances of alkyl or haloalkyl within R¹ are optionally unsaturated. In preferred embodiments, instances of alkyl or haloalkyl within R¹ are unsaturated. In preferred embodiments, instances of alkyl or haloalkyl within R¹ are not substituted with halogen, oxy, hydroxyl, methyl, ethyl, propyl, methoxy, ethoxy, trifluoromethyl, and not optionally interrupted by one or more heteroatoms. In preferred embodiments, instances of alkyl or haloalkyl within R¹ are optionally substituted with halogen, oxy, hydroxyl, methyl, ethyl, propyl, methoxy, ethoxy, trifluoromethyl, and not interrupted by one or more heteroatoms. In preferred embodiments, instances of alkyl or haloalkyl within R¹ are optionally substituted with halogen, oxy, hydroxyl, methyl, ethyl, propyl, methoxy, ethoxy, trifluoromethyl, and/or optionally interrupted by one or more heteroatoms, and/or optionally unsaturated.

Pyridinic Moiety of the Compound

Compounds according to the invention have a pyridinyl-like moiety that is attached to n¹ of the central ring of the compound according to the invention. It is substituted with 0, 1, or 2 instances of R². It is to be understood that this does not encompass R² when it is comprised in X¹ or X². This aromatic heterocycle is herein referred to as the pyridinic moiety. An amount of substitution by R² is denoted by n, which can be 0, 1, or 2. In preferred embodiments, n is 0 or 1. In preferred embodiments, n is 1 or 2. In preferred embodiments, n is 1. In preferred embodiments, n is 2. Most preferably n is 0. When n is 0, R² can still be present in X¹ or X².

When n is not 0, the pyridinic moiety has at least one R². When such an R² is present, it is ortho or meta to the central ring. In preferred embodiments it is ortho to the central ring. In preferred embodiments it is meta to the central ring.

R² is a substituent that is H, halogen, nitrile, —C₁₋₄alkyl, —C₁₋₃alkyl-nitrile, —C₁₋₄haloalkyl, —C₁₋₃haloalkyl-nitrile, —O—C₁₋₄alkyl, —O—C₁₋₃alkyl-nitrile, —O—C₁₋₄haloalkyl, —O—C₁₋₃haloalkyl-nitrile, —S—C₁₋₄alkyl, —S—C₁₋₃alkyl-nitrile, —S—C₁₋₄haloalkyl, —S—C₁₋₃haloalkyl-nitrile, or R² together with Q forms a bridging moiety; preferably it is H, halogen, —C₁₋₄alkyl, —C₁₋₄haloalkyl, —O—C₁₋₄alkyl, —O—C₁₋₄haloalkyl, —S—C₁₋₄alkyl, —S—C₁₋₄haloalkyl, or R² together with Q forms a bridging moiety; in preferred embodiments R² is H, fluorine, chlorine, or together with Q forms a bridging moiety; more preferably it is H, fluorine, or chlorine. Here, —C₁₋₄alkyl and —C₁₋₄haloalkyl are preferably —C₁₋₃alkyl or C₁₋₃haloalkyl, more preferably C₁ variants or isopropyl, most preferably C₁ variants.

In preferred embodiments, instances of alkyl or haloalkyl within R² are not unsaturated. In preferred embodiments, instances of alkyl or haloalkyl within R² are optionally unsaturated. In preferred embodiments, instances of alkyl or haloalkyl within R² are unsaturated. In preferred embodiments, instances of alkyl or haloalkyl within R² are not substituted with halogen, oxy, hydroxyl, methyl, ethyl, propyl, methoxy, ethoxy, trifluoromethyl, and not optionally interrupted by one or more heteroatoms. In preferred embodiments, instances of alkyl or haloalkyl within R² are optionally substituted with halogen, oxy, hydroxyl, methyl, ethyl, propyl, methoxy, ethoxy, trifluoromethyl, and not interrupted by one or more heteroatoms. In preferred embodiments, instances of alkyl or haloalkyl within R² are optionally substituted with halogen, oxy, hydroxyl, methyl, ethyl, propyl, methoxy, ethoxy, trifluoromethyl, and/or optionally interrupted by one or more heteroatoms, and/or optionally unsaturated.

X¹ is CH, C(R²), N, or C(Q); in preferred embodiment X¹ is CH, C(R²), or N; in preferred embodiment X¹ is CH, C(R²), or N; in preferred embodiment X¹ is CH, C(R²), or C(Q); in preferred embodiment X¹ is CH, N, or C(Q); in preferred embodiment X¹ is C(R²), N, or C(Q); in preferred embodiment X¹ is CH or C(R²); in preferred embodiment X¹ is CH or C(Q); in preferred embodiment X¹ is CH or N; in preferred embodiment X¹ is N or C(R²); in preferred embodiment X¹ is C(Q) or C(R²); in preferred embodiment X¹ is N or C(Q); in preferred embodiment X¹ is CH; in preferred embodiment X¹ is C(R²); in preferred embodiment X¹ is N; in the most highly preferred embodiment X¹ is C(Q).

X² is CH, C(R²), or N; in preferred embodiment X¹ is C(R²) or N; in preferred embodiment X¹ is CH or N; in preferred embodiment X¹ is CH or C(R²); in preferred embodiment X¹ is C(R²); in preferred embodiment X¹ is N; most preferably X² is CH. When X² is C(R²), the R² preferably forms a bridging moiety with Q.

Preferably, at most one of X¹ and X² is N. More preferably, when one of X¹ and X² is not CH, the other of X¹ and X² is CH.

Q is H, halogen, C₁₋₆alkyl, —OH, —O—C₁₋₆alkyl, —O—C₁₋₆acyl, —NH₂, —NH—(C₁₋₆alkyl), —N(C₁₋₆alkyl)₂, —NH(C₁₋₈acyl), —N(C₁₋₈acyl)₂, —C₁₋₄alkyl-OH, —C₁₋₄alkyl-O—C₁₋₆alkyl, —C₁₋₄alkyl-O—C₁₋₆acyl, —C₁₋₄alkyl-NH₂, —C₁₋₄alkyl-NH—(C₁₋₆alkyl), —C₁₋₄alkyl-N(C₁₋₆alkyl)₂, —C₁₋₄alkyl-NH(C₁₋₈acyl), —C₁₋₄alkyl-N(C₁₋₈acyl)₂, —C₁₋₄alkyl-N—C(O)—NH—C₁₋₆alkyl, —C₁₋₄alkyl-N—C(O)—N(C₁₋₆alkyl)₂, —C₁₋₄alkyl-O—C(O)—NH—C₁₋₆alkyl, —C₁₋₄alkyl-O—C(O)—N(C₁₋₆alkyl)₂, —C₁₋₄alkyl-N—C(O)—O—C₁₋₆alkyl, or Q together with R² forms a bridging moiety selected from —NH—CH═CH—, —NH—(C₂₋₄alkyl)-, and —(C₁₋₃alkyl)-NH—(C₁₋₃alkyl)-; preferably, Q is H, F, —CH₃, —CH₂F, —CHF₂, —CF₃, —OCH₃, —OCH₂F, —OCHF₂, —OCF₃, —NH—C(O)—CH₃, —NH—C(O)-cyclopropyl, —NH—C(O)-phenyl, —NH—C(O)-halophenyl, —NH—C(O)-piperidinyl, —NH—C(O)— pyridinyl, —NH—C(O)-morpholinyl, —NH—C(O)-oxanyl, —NH₂, —NH(CH₃), —NH(cyclopentyl), —CH₂—NH—C(O)—CH₃, —CH₂—N(CH₃)₂, —CH₂—NH₂, —CH₂—NH—(CH₃), —CH₂—NH-(cyclopentyl), or together with R² forms a bridging moiety that is preferably —NH—CH═CH—; more preferably, Q is H, F, —NH—C(O)—CH₃, —NH—C(O)-cyclopropyl, —NH—C(O)-phenyl, —NH—C(O)-halophenyl, —NH₂, —NH(CH₃), —NH(cyclopentyl), —CH₂—NH—C(O)—CH₃, —CH₂—N(CH₃)₂, —CH₂—NH₂, —CH₂—NH—(CH₃), —CH₂—NH-(cyclopentyl), or together with R² forms a bridging moiety that is preferably —NH—CH═CH—; eve more preferably, Q is H, F, —NH—C(O)—CH₃, —NH—C(O)-cyclopropyl, —NH—C(O)-phenyl, —NH—C(O)-halophenyl, —NH₂, —NH(CH₃), —NH(cyclopentyl), —CH₂—NH—C(O)—CH₃, —CH₂—NH-(cyclopentyl), or together with R² forms a bridging moiety that is preferably —NH—CH═CH—. Here, -alkyl and -acyl when terminal to a moiety are preferably —C₁₋₄alkyl or C₂₋₄acyl or C₃₋₆cycloalkyl or C₅₋₆aryl, more preferably C₃₋₆cycloalkyl or C₅-6aryl. Here, —C₁₋₄alkyl- when preceding a heteroatom is preferably C₁₋₂alkyl, more preferably —CH₂— or —CH₂CH₂—, most preferably —CH₂—. It is to be understood that for —N(C₁₋₆alkyl)₂, —N(C₁₋₈acyl)₂, —C₁₋₄alkyl-N(C₁₋₆alkyl)₂, —C₁₋₄alkyl-N(C₁₋₈acyl)₂, —C₁₋₄alkyl-N—C(O)—N(C₁₋₆alkyl)₂, and —C₁₋₄alkyl-O—C(O)—N(C₁₋₆alkyl)₂, the latter two alkyl or acyl moieties can, together with the N to which they are attached, form a heterocycle, preferably a C₄₋₆heterocycle or a C₅₋₆heteroaryl, most preferably a C₅₋₆heterocycle or a C₅₋₆heteroaryl, most preferably a C₅₋₆heterocycle.

A bridging moiety as formed by Q and R² is selected from —NH—CH═CH—, —NH—(C₂₋₄alkyl)-, and —(C₁₋₃alkyl)-NH—(C₁₋₃alkyl)-. Preferred examples are —NH—CH═CH—, —NH—CH₂—CH₂—, —NH—CH₂—, —N═CH—CH₂—CH₂—, —CH₂—CH₂—NH—CH₂—CH₂, and —CH₂—NH—CH₂.

In preferred embodiments, instances of alkyl or acyl within Q are not unsaturated. In preferred embodiments, instances of alkyl or acyl within Q are optionally unsaturated. In preferred embodiments, instances of alkyl or acyl within Q are unsaturated. In preferred embodiments, instances of alkyl or acyl within Q are not substituted with halogen, oxy, hydroxyl, methyl, ethyl, propyl, methoxy, ethoxy, trifluoromethyl, and not optionally interrupted by one or more heteroatoms. In preferred embodiments, instances of alkyl or acyl within Q are optionally substituted with halogen, oxy, hydroxyl, methyl, ethyl, propyl, methoxy, ethoxy, trifluoromethyl, and not interrupted by one or more heteroatoms. In preferred embodiments, instances of alkyl or acyl within Q are optionally substituted with halogen, oxy, hydroxyl, methyl, ethyl, propyl, methoxy, ethoxy, trifluoromethyl, and/or optionally interrupted by one or more heteroatoms, and/or optionally unsaturated.

In preferred embodiments the pyridinic moiety of the compound is as shown below, with a reference name shown below each structure. Py1-Py26 and Py29-Py33 are particularly preferred, Py1-Py26 are preferred, Py1-Py15 are even more preferred, Py1-Py12 are still more preferred, and Py1 is most preferred.

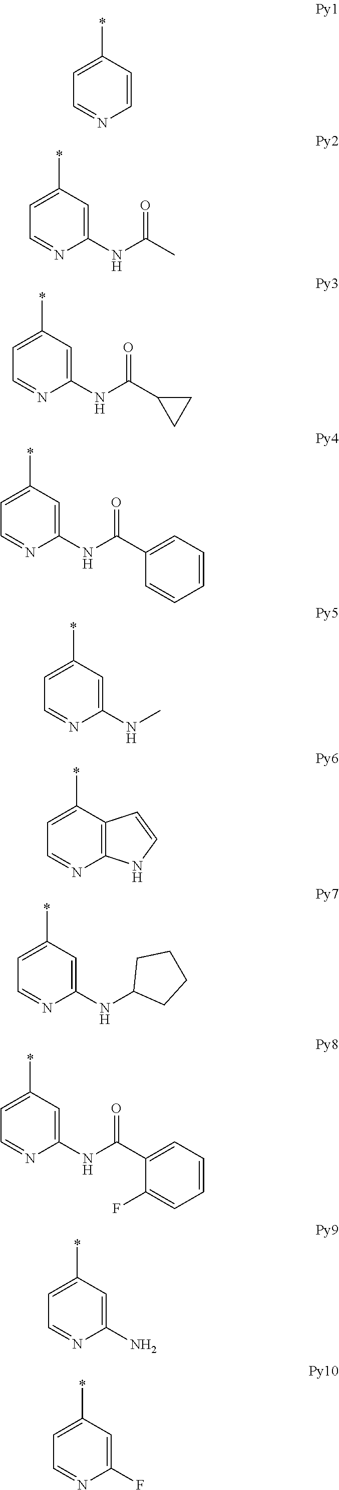

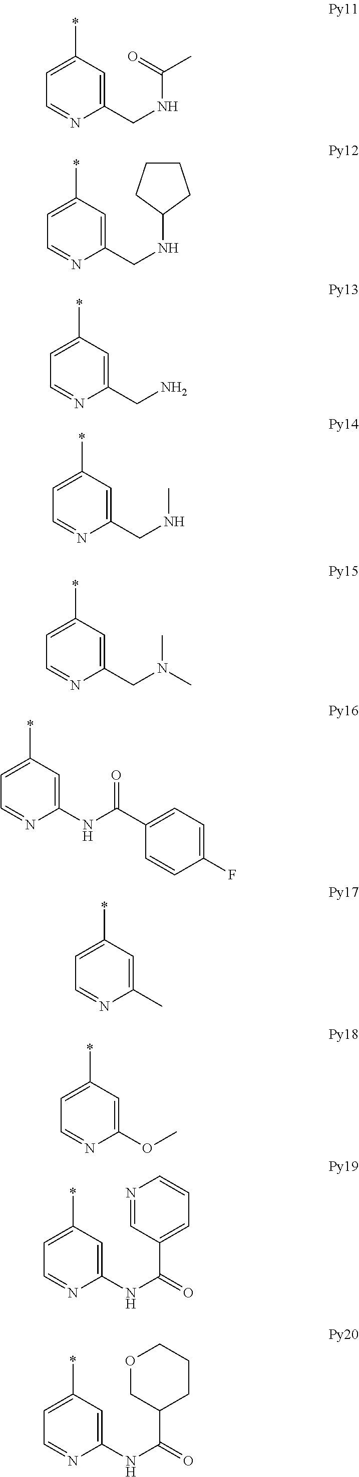

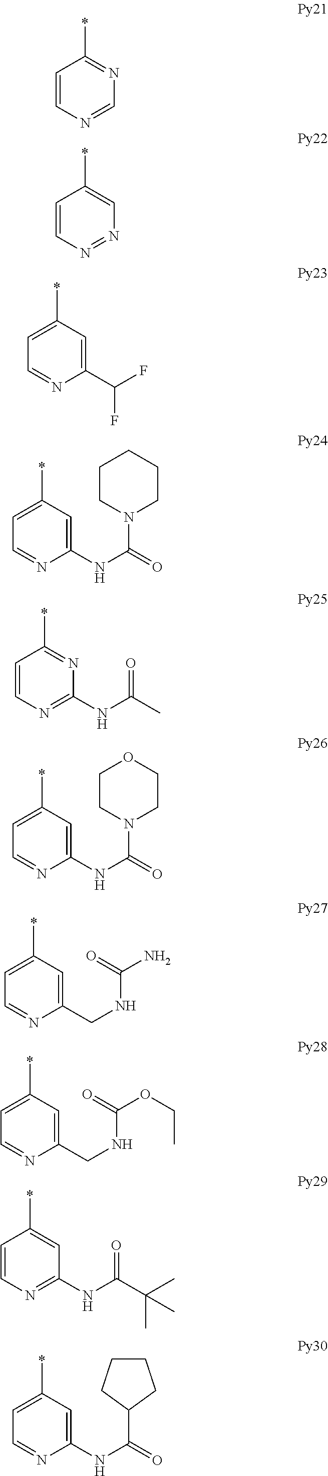

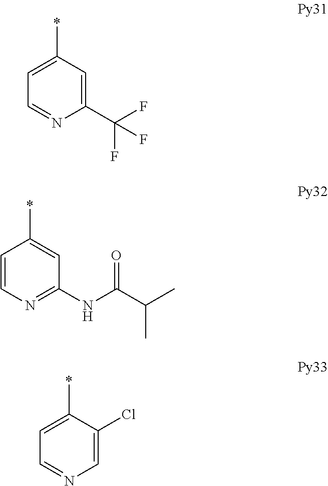 [SMILES: *c1ccnc(C(F)(F)F)c1 ; *c1ccnc(NC(=O)C(C)C)c1 ; *c1ccncc1Cl]

Compounds of general formula (I) are preferably of general formula (III) or (III-A)

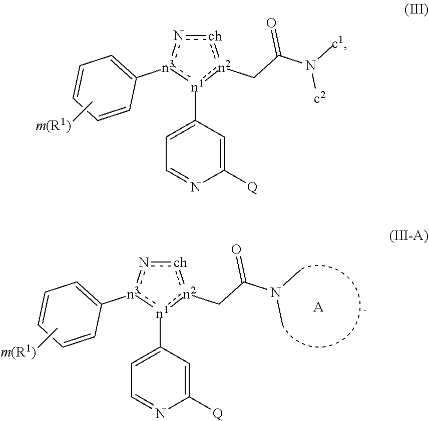

Amido Moiety of the Compound

Compounds according to the invention have an amido moiety that is attached to n² of the central ring of the compound according to the invention. It is substituted with R³ and the amide is N,N′-disusbstituted with c¹ and c².

R³ is in each instance independently selected from H, halogen, or C₁₋₄alkyl. In preferred embodiments, R³ is in each instance independently selected from halogen or C₁₋₄alkyl. In preferred embodiments, R³ is in each instance independently selected from halogen or C₁₋₃alkyl. In preferred embodiments, R³ is halogen. In preferred embodiments, R³ is C₁₋₄alkyl. In more preferred embodiments, R³ is in each instance independently selected from H or C₁₋₄alkyl. In the most preferred embodiments, R³ is H. It is particularly preferred that at least one instance of R³ is H.

For R³, halogen is preferably chlorine or fluorine, more preferably fluorine. For R³, —C₁₋₄alkyl is preferably —C₁₋₃alkyl, more preferably methyl or isopropyl, most preferably methyl.

c¹ is H, C₁₋₆alkyl, (C₁₋₂alkyl)₀₋₁C₃₋₆cycloalkyl, or (C₁₋₂alkyl)₀₋₁C₄₋₆heterocycloalkyl, preferably c¹ is H and c² is C₄₋₈cycloalkyl, C₄₋₈heterocycloalkyl, C₄₋₈cycloalkyl-C₁₋₃alkyl, C₄₋₈heterocycloalkyl-C₁₋₃alkyl, C₁₋₃alkyl-C₄₋₈cycloalkyl, or C₁₋₃alkyl-C₄₋₈heterocycloalkyl, or c¹ and c² together form cyclic structure A; when c¹ is H, it is preferred that c² is pyridyl, —CH₂-pyridyl, piperidinyl, N-methylpiperidinyl, —CH₂-piperidinyl, —CH₂-(N-methylpiperidinyl), cyclopentyl, hydroxycyclopentyl, —CH₂-cyclopentyl, —CH₂-hydroxycyclopentyl, pyrrolidinyl, N-methylpyrrolidinyl, substituted piperidinyl such as hydroxylpiperidinyl (such as piperidin-3-ol-5-yl) or alkylated piperidinyl (such as 1-methylpiperidin-3-yl), alkylated pyrrolidinyl such as 1-(2,2-difluoroethyl)pyrrolidin-3-yl or 1-methylpyrrolidin-3-yl or 4,4-difluoro-1-methylpyrrolidin-3-yl, oxolanyl such as oxolan-3-yl, —CH₂-pyrrolidinyl, or —CH₂-(N-methylpyrrolidinyl). Most preferably c¹ and c² together form cyclic structure A.

In c², C₁₋₃alkyl is preferably —CH₂CH₂— or —CH₂—, most preferably —CH₂—. In c², alkyl is preferably not unsaturated or substituted. In preferred embodiments C₄₋₈cycloalkyl and C₄₋₈ heterocycloalkyl are unsaturated when comprised in c². In preferred embodiments C₄₋₈cycloalkyl and C₄₋₈heterocycloalkyl are not unsaturated when comprised in c². In preferred embodiments C₄₋₈ cycloalkyl and C₄₋₈heterocycloalkyl are not substituted when comprised in c². In preferred embodiments C₄₋₈cycloalkyl and C₄₋₈heterocycloalkyl are substituted as described elsewhere herein when comprised in c².

When c¹ is H or —CH₃, more preferably H, preferred embodiments for c² are shown below, with a reference name shown below each structure. In preferred embodiments c² is C2_1-C2_4. In preferred embodiments c² is C2_5-C2_8. In preferred embodiments c² is C2_3-C2_7. In preferred embodiments c² is C2_1-C2_3 or C2_8. In preferred embodiments c² is C2_1-C2_3.

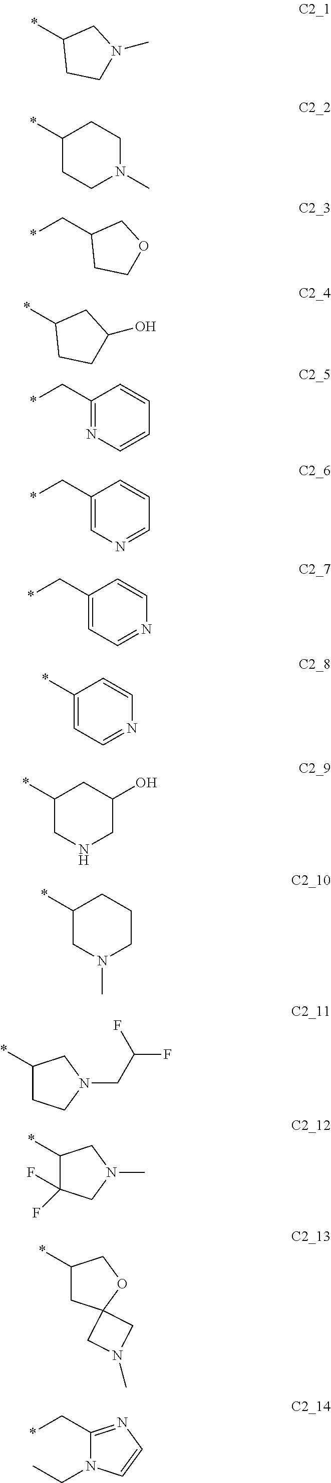

In a preferred embodiment, C2_1 is 2-[4-(4-chlorophenyl)-5-(pyridin-4-yl)-1H-imidazol-1-yl]-N-[(3R)-1-methylpyrrolidin-3-yl]acetamide (C2_1_R) or 2-[4-(4-chlorophenyl)-5-(pyridin-4-yl)-1H-imidazol-1-yl]-N-[(3S)-1-methylpyrrolidin-3-yl]acetamide (C2_1_S).

In a preferred embodiment C2_3 is 2-[4-(4-chlorophenyl)-5-(pyridin-4-yl)-1H-imidazol-1-yl]-N-[(3R)-oxolan-3-yl]acetamide (C2_3_R) or 2-[4-(4-chlorophenyl)-5-(pyridin-4-yl)-1H-imidazol-1-yl]-N-[(3S)-oxolan-3-yl]acetamide (C2_3_S).

In a preferred embodiment C2_10 is 2-[4-(4-chlorophenyl)-5-(pyridin-4-yl)-1H-imidazol-1-yl]-N-[(3R)-1-methylpiperidin-3-yl]acetamide (C2_10_R) or 2-[4-(4-chlorophenyl)-5-(pyridin-4-yl)-1H-imidazol-1-yl]-N-[(3S)-1-methylpiperidin-3-yl]acetamide (C2_10_S).

In a preferred embodiment C2_11 is 2-[4-(4-chlorophenyl)-5-(pyridin-4-yl)-1H-imidazol-1-yl]-N-[(3R)-1-(2,2-difluoroethyl)pyrrolidin-3-yl]acetamide (C2_11_R) or 2-[4-(4-chlorophenyl)-5-(pyridin-4-yl)-1H-imidazol-1-yl]-N-[(3S)-1-(2,2-difluoroethyl)pyrrolidin-3-yl]acetamide (C2_11_S).

A is a C₄₋₁₂heterocycloalkyl that can be cyclic, bicyclic, and tricyclic, and which is optionally unsaturated, and which is optionally substituted with halogen, C₁₋₆alkyl, —O—C₁₋₄alkyl, hydroxyl, —NH₂, —NH(C₁₋₄alkyl), or —N(C₁₋₄alkyl)₂. In preferred embodiments there are no such optional substitutions. In these optional substitutions, alkyl is preferably C₃₋₄(halo)cycloalkyl, C₃₋₄(halo)heterocycloalkyl or C₁₋₃(halo)alkyl, more preferably C₁₋₂alkyl or C₁₋₂haloalkyl or oxetane, still more preferably oxetane or —CH₃, most preferably —CH₃. Multicyclic structures can be fused, bridged, or spiro. In preferred embodiments, A is not multicyclic. In preferred embodiments, A is cyclic or multicyclic wherein it is fused or bridged. In preferred embodiments, A is cyclic or multicyclic wherein it is fused or spiro. In preferred embodiments, A is cyclic or multicyclic wherein it is spiro or bridged. In preferred embodiments, A is cyclic or multicyclic wherein it is fused. A moiety attached as a spiro-cycle is preferably 3- or 4-membered. A cycle that is fused to A is preferably 4-6-membered, more preferably 5-6-membered. A bridging moiety is preferably 1 or 2 atoms long, most preferably 1. It should be understood that when A is unsaturated it can be a C₅₋₁₂heteroaryl. In preferred embodiments, A is a C₄₋₁₂heterocycloalkyl or a C₅₋₁₂heteroaryl that can be cyclic, bicyclic, and tricyclic, and which is optionally substituted with halogen, C₁₋₆alkyl, —O—C₁₋₄alkyl, hydroxyl, —NH₂, —NH(C₁₋₄alkyl), or —N(C₁₋₄alkyl)₂. Here, C₄₋₁₂ is preferably C₅₋₁₀, more preferably C₅₋₈, most preferably C₁₋₆. In preferred embodiments, for determining the amount of C in an A moiety, only the carbon atoms in the single ring comprising the N of the amide of general structure (I) are counted. In other preferred embodiments all carbon atoms in all cycles of moiety A are counted. In other preferred embodiments all carbon atoms in the entire moiety A are counted.

Preferably, A is selected from optionally substituted and optionally unsaturated azetidinyl, pyrrolidinyl, imidazolidinyl, oxazolidinyl, piperidinyl, piperazinyl, morpholinyl, azacycloheptyl, diazacycloheptyl, or oxoazacycloheptyl; wherein each optional substitution can be a substitution with halogen, C₁₋₆alkyl, C₃₋₆cycloalkyl, C₃₋₆heterocycloalkyl, —O—C₁₋₄alkyl, hydroxyl, —NH₂, —NH(C₁₋₄alkyl), or —N(C₁₋₄alkyl)₂; preferably each optional substitution is independently selected from methyl, dimethylamine, methoxyl, propyl, hydroxyl, a bridging C₁₋₃alkyl moiety, spiro azetidinyl, spiro N-methylazetidinyl, spiro oxetanyl, oxetanyl, spiro piperidinyl, difluoropiperidinyl, spiro N-methylpiperidinyl, spiro cyclopropyl, fused pyrrolidinyl, or fused N-methylpyrrolidinyl. In more preferred embodiments, A is not substituted and not unsaturated. In other more preferred embodiments, A is substituted and not unsaturated. In other more preferred embodiments, A is not substituted and is unsaturated. In other more preferred embodiments, A is substituted and unsaturated. Preferably A is not aromatic.

In preferred embodiments the cyclic structure A is as shown below, with a reference name shown below each structure. A1-A22 and A44-A51 are particularly preferred, A1-A22 are preferred, A1-A20 are even more preferred, A1-A19 are still more preferred, and A1 and A11 are most preferred. In some preferred embodiments A is A1. In other preferred embodiments A is A11. In other preferred embodiments, cyclic structure A comprises an amine or basic nitrogen, more preferably cyclic structure A is selected from A1, A2, A4, A5, A7, A8, A10-A13, A16-A38, A41, and A43-A51, more preferably from A1, A2, A4, A5, A7, A8, A10-A13, A16-A38, A41, and A43. More preferred such cyclic structures A are A1, A10, A11, and A23-A31. In other preferred such embodiments A is A1 or A11; in other preferred such embodiments A is A10 or A23-A31. In other preferred embodiments, cyclic structure A comprises a second heteroatom, more preferably cyclic structure A is selected from A1, A2, A4-A43, even more preferably from A1, A2, and A4-A24. In other preferred embodiments, cyclic structure A is bicyclic, spiro-cyclic, or bridged, preferably selected from A4, A7, A8, A10, A12, A13, A15-A19, A21-A35, and A37-A42, more preferably from A4, A7, A8, A10, A12, A13, A15-A19, A21, A22, A32-A35, and A37-A42; even more preferably it is bicyclic or bridged, preferably selected from A8, A10, A21, A23-A31, A33, and A41, more preferably from A8, A10, A21, A23-A31, and A33, most preferably from A8, A10, A21, and A33. A1-A51 as defined below can be optionally methylated, preferably N-methylated, wherein N-methylation is preferably at a nitrogen that is not attached to the bicyclic core.

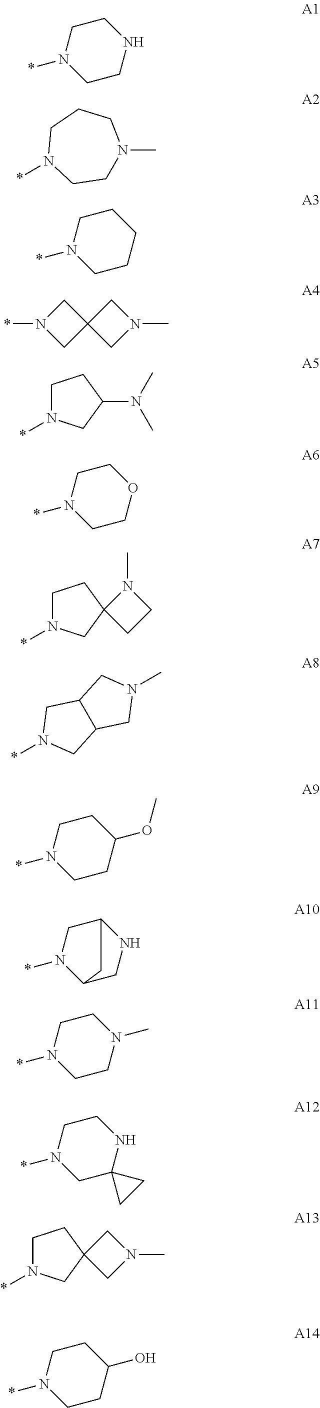

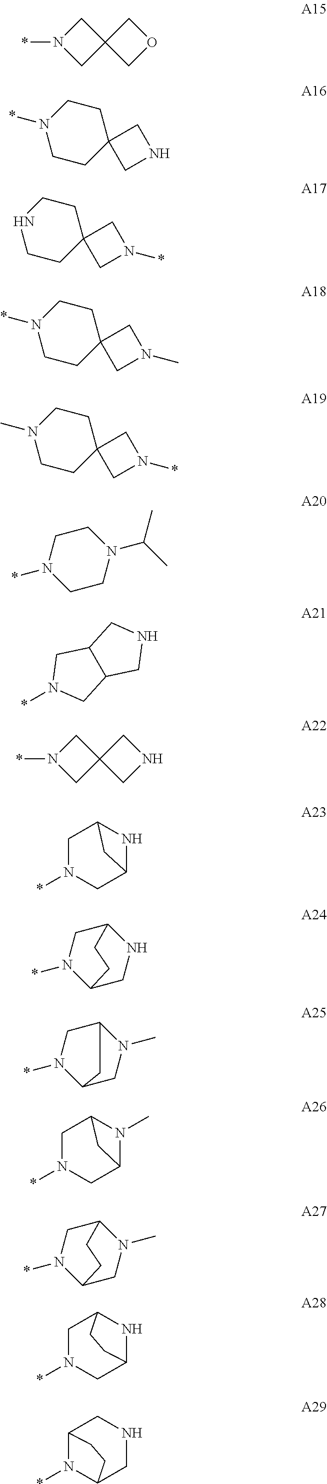

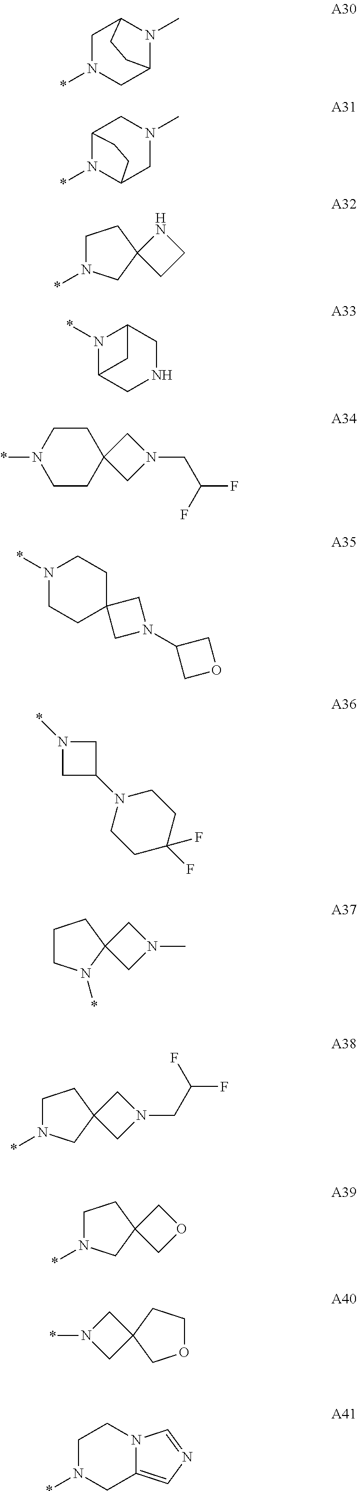

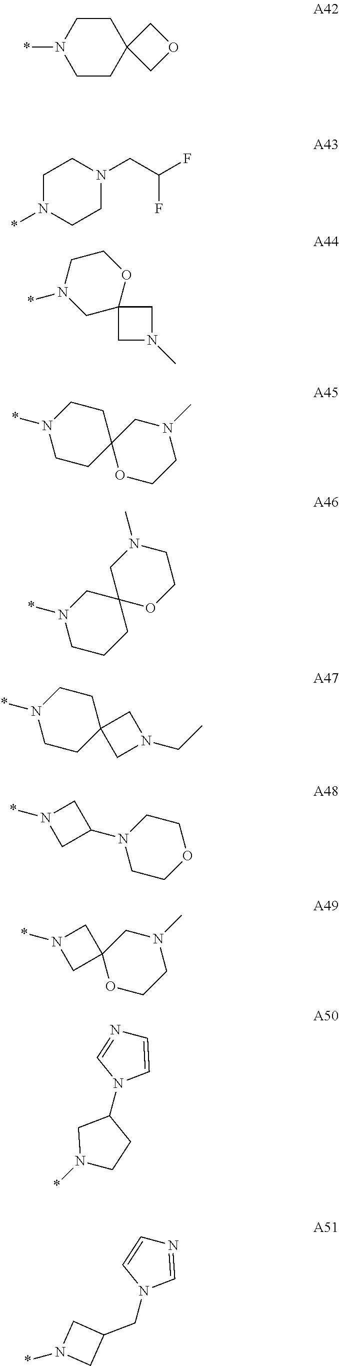

In a preferred embodiment, A27 is 2-[4-(4-chlorophenyl)-5-(pyridin-4-yl)-1H-imidazol-1-yl]-1-[(1R,4R)-5-methyl-2,5-diazabicyclo[2.2.2]octan-2-yl]ethan-1-one (A27_RR) or 2-[4-(4-chlorophenyl)-5-(pyridin-4-yl)-1H-imidazol-1-yl]-1-[(1S,4S)-5-methyl-2,5-diazabicyclo[2.2.2]octan-2-yl]ethan-1-one (A27_SS).

In a preferred embodiment, A25 is 2-[4-(4-chlorophenyl)-5-(pyridin-4-yl)-1H-imidazol-1-yl]-1-[(1R,4R)-5-methyl-2,5-diazabicyclo[2.2.1]heptan-2-yl]ethan-1-one (A25_RR) or 2-[4-(4-chlorophenyl)-5-(pyridin-4-yl)-1H-imidazol-1-yl]-1-[(1S,4S)-5-methyl-2,5-diazabicyclo[2.2.1]heptan-2-yl]ethan-1-one (A25_SS).

### Further Definitions of the Compound

In preferred embodiments is provided the compound according to the invention, wherein
n² is N and n¹ is C and n³ is C;
ch is CH, C(Cl), C(CH₃), C(isopropyl), C(CF₃), O, NH, or N(CH₃);
R¹ is H, fluorine, chlorine, —CH₃, —CF₃, —O—CH₃, or nitrile;
m is 0 or 1;
R² is H, fluorine, chlorine, or forms a bridging moiety;
n is 0;
R³ is H or —CH₃;
X¹ is C(Q);
X² is CH;
Q is H, F, —CH₃, —CH₂F, —CHF₂, —CF₃, —OCH₃, —OCH₂F, —OCHF₂, —OCF₃, —NH—C(O)—CH₃, —NH—C(O)-cyclopropyl, —NH—C(O)-phenyl, —NH—C(O)-halophenyl, —NH—C(O)-piperidinyl, —NH—C(O)-pyridinyl, —NH—C(O)-morpholinyl, —NH—C(O)-oxanyl, —NH₂, —NH(CH₃), —NH(cyclopentyl), —CH₂—NH—C(O)—CH₃, —CH₂—N(CH₃)₂, —CH₂—NH₂, —CH₂—NH—(CH₃), —CH₂—NH-(cyclopentyl), or together with R² forms —NH—CH═CH—; and/or wherein
c¹ is H and c² is pyridyl, —CH₂-pyridyl, piperidinyl, N-methylpiperidinyl, —CH₂-piperidinyl, —CH₂-(N-methylpiperidinyl), cyclopentyl, hydroxycyclopentyl, —CH₂-cyclopentyl, —CH₂-hydroxycyclopentyl, pyrrolidinyl, N-methylpyrrolidinyl, —CH₂-pyrrolidinyl, —CH₂-(N-methylpyrrolidinyl), or c¹ and c² together form cyclic structure A.

In preferred embodiments is provided the compound according to the invention, wherein Q is H, F, —NH—C(O)—CH₃, —NH—C(O)-cyclopropyl, —NH—C(O)-phenyl, —NH—C(O)-halophenyl, —NH₂, —NH(CH₃), —NH(cyclopentyl), —CH₂—NH—C(O)—CH₃, —CH₂—N(CH₃)₂, —CH₂—NH₂, —CH₂—NH—(CH₃), —CH₂—NH-(cyclopentyl), or together with R² forms —NH—CH═CH—; and/or wherein R³ is H; and/or wherein R¹ is H, fluorine, chlorine, —CH₃, —CF₃, or —O—CH₃.

In preferred embodiments, the compound according to the invention comprises:
i) a cyclic ring A selected from A1-A51 or c¹ is as defined elsewhere herein, preferably H, and c² is selected from C2_1-C2_14; preferably the compound comprises a cyclic ring A selected from A1-A51;
ii) a pyridinic moiety selected from Py1-Py33;
iii) a phenylic moiety selected from Ph1-Ph21; and/or
iv) a central ring selected from CR1-CR17.
   In more preferred embodiments, both i) and ii) apply. In other more preferred embodiments, both i) and iii) apply. In other more preferred embodiments, both i) and iv) apply. In other more preferred embodiments, both ii) and iii) apply. In other more preferred embodiments, both ii) and iv) apply. In other more preferred embodiments, both iii) and iv) apply. In even more preferred embodiments, each of i), ii) and iii) apply. In other even more preferred embodiments, each of i), ii), and iv) apply. In other even more preferred embodiments, each of i), iii), and iv) apply. In other even more preferred embodiments, each of ii), iii), and iv) apply. In the most preferred embodiments each of i), ii), iii), and iv) apply.

In other preferred embodiments, the compound according to the invention is of general formula (IV), (IV-A), (V), or (V-A), more preferably (V) or (V-A), most preferably (V-A):

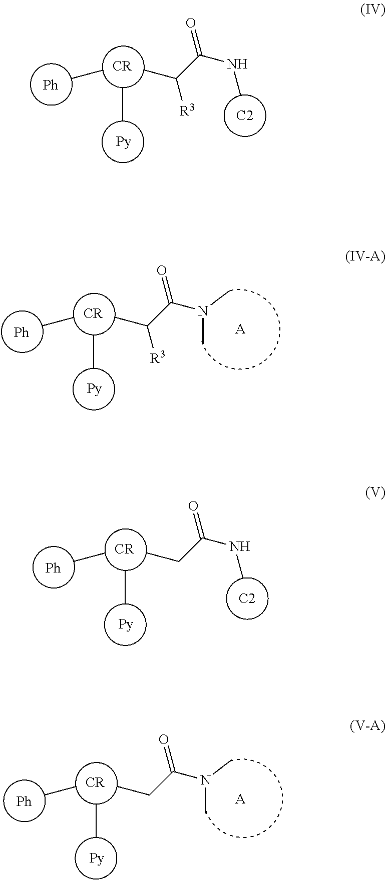

wherein R³ is as defined above, preferably R³ is H or —CH₃, more preferably it is H;

wherein the cyclic structure A is as defined above, preferably it is selected from A1-A51, preferably from A1-A24, more preferably from A1-A22, even more preferably from A1-A20, still more preferably from A1-A19, even more preferably A1 or A11, most preferably it is A1;

wherein c² is as defined above, preferably it is selected from C2_1-C2_14, more preferably it is C2_1-C₂₋₄ or C2_5-C₂₋₈ or C2_3-C2_7, most preferably it is C2_1-C2_3;

wherein the pyridinic moiety Py is as defined above, preferably it is selected from Py1-Py33, more preferably from Py1-Py26, even more preferably from Py1-Py15, still more preferably from Py1-Py12, most preferably it is Py1;

wherein phenylic moiety Ph is as defined above, preferably it is selected from Ph1-Ph21, more preferably from Ph1-Ph9 and Ph11-Ph19, even more preferably from Ph1-Ph9, even more preferably from Ph1-Ph8, more preferably it is Ph5 or Ph8, most preferably it is Ph8;

wherein the central ring CR is as defined above, preferably it is selected from CR1-CR17, more preferably from CR1-CR10, most preferably it is CR10.

In preferred embodiments the compounds according to the invention are compounds 1-105 and 109-168 listed in table 1 shown below, or salts thereof. Preferred are compounds 1-105. More preferred compounds are compounds 1-80, even more preferred are compounds 1-66, still more preferred are compounds 1-62, even more preferred are compounds 1-55, still more preferred are compounds 1-23, most preferred are compounds 1-8. In other preferred embodiments, the compound is selected from compounds 2, 5, 10, 13, 14, 16, 18, 22, 28, 34, 40, 43, 45, 48, 49, 50, 51, 53, 55, 56, 57, 61, 63, 64, 90, 99, 3, 4, 6, 7, 8, 9, 11, 12, 15, 17, 19, 20, 21, 23, 24, 25, 26, 27, 29, 30, 31, 32, 33, 35, 36, 37, 38, 39, 41, 42, 44, 46, 47, 52, 58, 59, 62, 65, 81, 82, 83, 84, 85, 86, 87, 88, 89, 91, 92, 93, 94, 95, 96, 97, 98, 100, 101, 102, 103, and 105 as listed in table 1, more preferably from compounds 3, 4, 6, 7, 8, 9, 11, 12, 15, 17, 19, 20, 21, 23, 24, 25, 26, 27, 29, 30, 31, 32, 33, 35, 36, 37, 38, 39, 41, 42, 44, 46, 47, 52, 58, 59, 62, 65, 81, 82, 83, 84, 85, 86, 87, 88, 89, 91, 92, 93, 94, 95, 96, 97, 98, 100, 101, 102, 103, and 105 as listed in table 1. Indications of stereochemistry in table 1 are preferred embodiments and serve as examples only. Compounds 106, 107, and 108 are reference compounds.

TABLE 1
preferred compounds according to the invention

| | |
|---|---|
| 1 | 2-[4-(4-chlorophenyl)-5-(pyridin-4-yl)-1H-imidazol-1-yl]-1-(piperidin-1-yl)ethan-1-one |
| 2 | N-{4-[4-(4-fluorophenyl)-1-[2-oxo-2-(piperazin-1-yl)ethyl]-1H-imidazol-5-yl]pyridin-2-yl}benzamide |
| 3 | 2-{5-[2-(cyclopentylamino)pyridin-4-yl]-4-(4-methoxyphenyl)-1H-imidazol-1-yl}-1-(piperazin-1-yl)ethan- |
| | 1-one |
| 4 | 2-fluoro-N-{4-[4-(4-fluorophenyl)-1-[2-(4-methylpiperazin-1-yl)-2-oxoethyl]-1H-imidazol-5-yl]pyridin-2- |
| | yljbenzamide |
| 5 | 2-[4-(4-chlorophenyl)-5-(pyridin-4-yl)-1H-imidazol-1-yl]-1-{5-methyl-octahydropyrrolo[3,4-c]pyrrol-2- |
| | yl}ethan-1-one |
| 6 | N-{4-[4-(4-chlorophenyl)-1-[2-oxo-2-(piperazin-1-yl)ethyl]-1H-imidazol-5-yl]pyridin-2-yl}benzamide |
| 7 | 4-fluoro-N-{4-[4-(4-fluorophenyl)-1-[2-(4-methylpiperazin-1-yl)-2-oxoethyl]-1H-imidazol-5-yl]pyridin-2- |
| | yljbenzamide |
| 8 | 2-[4-(4-chlorophenyl)-5-(pyridin-4-yl)-1H-imidazol-1-yl]-1-(4-methylpiperazin-1-yl)ethan-1-one |
| 9 | 2-[4-(4-chlorophenyl)-5-(pyridin-4-yl)-1H-imidazol-1-yl]-1-{1-methyl-1,6-diazaspiro[3.4]octan-6-yl}ethan- |
| | 1-one |
| 10 | 2-[4-(4-fluorophenyl)-5-(2-fluoropyridin-4-yl)-1H-imidazol-1-yl]-1-(piperazin-1-yl)ethan-1-one |
| 11 | 1-[(3RS,6RS)-5-methyl-octahydropyrrolo[3,4-c]pyrrol-2-yl]-2-[4-(4-chlorophenyl)-5-[2- |
| | (difluoromethyl)pyridin-4-yl]-1H-imidazol-1-yl]ethan-1-one |
| 12 | 2-[4-(4-chlorophenyl)-5-(pyridin-4-yl)-1H-imidazol-1-yl]-1-{2-methyl-2,7-diazaspiro[3.5]nonan-7- |
| | yl}ethan-1-one |
| 13 | 2-[4-(4-chlorophenyl)-5-(pyridin-4-yl)-1H-imidazol-1-yl]-1-[3-(dimethylamino)pyrrolidin-1-yl]ethan-1-one |
| 14 | 2-[4-(4-chlorophenyl)-5-(pyridin-4-yl)-1H-imidazol-1-yl]-1-{4,7-diazaspiro[2.5]octan-7-yl}ethan-1-one |
| 15 | 2-[4-(4-chlorophenyl)-5-(pyridin-4-yl)-1H-imidazol-1-yl]-1-(4-methyl-1,4-diazepan-1-yl)ethan-1-one |
| 16 | 2-[4-(4-chlorophenyl)-5-(pyridin-4-yl)-1H-imidazol-1-yl]-1-{6-methyl-2,6-diazaspiro[3.3]heptan-2- |
| | yl}ethan-1-one |
| 17 | 2-[4-(4-chlorophenyl)-5-(pyridin-4-yl)-1H-imidazol-1-yl]-1-{2-oxa-6-azaspiro[3.3]heptan-6-yl}ethan-1- |
| | one |
| 18 | 2-[4-(4-chlorophenyl)-5-(pyridin-4-yl)-1H-imidazol-1-yl]-1-(piperazin-1-yl)ethan-1-one |
| 19 | 2-[4-(4-chlorophenyl)-5-(pyridin-4-yl)-1H-imidazol-1-yl]-1-{2-methyl-2,6-diazaspiro[3.4]octan-6-yl}ethan- |
| | 1-one |
| 20 | 2-[4-(4-chlorophenyl)-5-(pyridin-4-yl)-1H-imidazol-1-yl]-N-(1-methylpiperidin-4-yl)acetamide |
| 21 | N-{4-[4-(4-fluorophenyl)-1-[2-oxo-2-(piperazin-1-yl)ethyl]-1H-imidazol-5-yl]pyridin-2- |
| | yljcyclopropanecarboxamide |
| 22 | 2-[4-(4-fluorophenyl)-5-(pyridin-4-yl)-1H-imidazol-1-yl]-1-(4-methylpiperazin-1-yl)ethan-1-one |
| 23 | 2-[3-(4-fluorophenyl)-4-(pyridin-4-yl)-1H-pyrazol-5-yl]-1-(piperazin-1-yl)ethan-1-one |
| 24 | 2-[4-(4-chlorophenyl)-5-(pyridin-4-yl)-1H-imidazol-1-yl]-N-(3-hydroxycyclopentyl)acetamide |
| 25 | N-{4-[4-(4-fluorophenyl)-1-[2-(morpholin-4-yl)-2-oxoethyl]-1H-imidazol-5-yl]pyridin-2-yl}benzamide |
| 26 | N-{4-[4-(4-fluorophenyl)-1-[2-oxo-2-(piperazin-1-yl)ethyl]-1H-imidazol-5-yl]pyridin-2-yl}acetamide |
| 27 | N-{4-[4-(4-chlorophenyl)-1-[2-(4-methylpiperazin-1-yl)-2-oxoethyl]-1H-imidazol-5-yl]pyridin-2- |
| | yljbenzamide |
| 28 | 2-[4-(4-chlorophenyl)-5-{1H-pyrrolo[2,3-b]pyridin-4-yl}-1H-imidazol-1-yl]-1-(4-methylpiperazin-1- |
| | yl)ethan-1-one |
| 29 | 2-[4-(4-chlorophenyl)-5-(pyridin-4-yl)-1H-imidazol-1-yl]-1-{2,7-diazaspiro[3.5]nonan-2-yl}ethan-1-one |
| 30 | 2-[4-(4-fluorophenyl)-5-(pyridin-4-yl)-1H-imidazol-1-yl]-1-(piperazin-1-yl)ethan-1-one |
| 31 | 2-[4-(4-fluorophenyl)-2-methyl-5-(pyridin-4-yl)-1H-imidazol-1-yl]-1-(piperazin-1-yl)ethan-1-one |
| 32 | 2-[4-(4-chlorophenyl)-5-(pyridin-4-yl)-1H-imidazol-1-yl]-1-{2,7-diazaspiro[3.5]nonan-7-yl}ethan-1-one |
| 33 | 2-[4-(4-fluorophenyl)-5-[2-(methylamino)pyridin-4-yl]-1H-imidazol-1-yl]-1-(piperazin-1-yl)ethan-1-one |
| 34 | 2-[4-(4-chlorophenyl)-5-(pyridin-4-yl)-1H-imidazol-1-yl]-1-(4-methoxypiperidin-1-yl)ethan-1-one |
| 35 | 2-[4-(4-chlorophenyl)-5-(pyridin-4-yl)-1H-imidazol-1-yl]-N-[(oxolan-3-yl)methyl]acetamide |
| 36 | 2-[4-(4-chlorophenyl)-5-(pyridin-4-yl)-1H-imidazol-1-yl]-1-{7-methyl-2,7-diazaspiro[3.5]nonan-2- |
| | yl}ethan-1-one |
| 37 | 1-[(3RS,6RS)-octahydropyrrolo[3,4-c]pyrrol-2-yl]-2-[4-(4-chlorophenyl)-5-[2-(difluoromethyl)pyridin-4- |
| | yl]-1H-imidazol-1-yl]ethan-1-one |
| 38 | 2-[2-chloro-4-(4-fluorophenyl)-5-(pyridin-4-yl)-1H-imidazol-1-yl]-1-(piperazin-1-yl)ethan-1-one |
| 39 | 2-[4-(4-chlorophenyl)-5-(pyridin-4-yl)-1H-imidazol-1-yl]-1-(4-hydroxypiperidin-1-yl)ethan-1-one |
| 40 | 2-[3-(4-fluorophenyl)-4-(pyridin-4-yl)-1,2-oxazol-5-yl]-1-(4-methylpiperazin-1-yl)ethan-1-one |
| 41 | 2-[4-(4-chlorophenyl)-5-(pyridin-4-yl)-1H-imidazol-1-yl]-N-[(pyridin-3-yl)methyl]acetamide |
| 42 | 2-[4-(4-chlorophenyl)-5-(pyridin-4-yl)-1H-imidazol-1-yl]-N-[(pyridin-4-yl)methyl]acetamide |
| 43 | 2-[1-(4-fluorophenyl)-5-(pyridin-4-yl)-1H-pyrazol-4-yl]-1-(piperazin-1-yl)ethan-1-one |
| 44 | 2-[3-(4-fluorophenyl)-4-(pyridin-4-yl)-1,2-oxazol-5-yl]-1-(piperazin-1-yl)ethan-1-one |
| 45 | 2-[4-(4-methylphenyl)-5-(pyridin-4-yl)-1H-imidazol-1-yl]-1-(piperazin-1-yl)ethan-1-one |
| 46 | 2-[4-(4-chlorophenyl)-5-(pyridin-4-yl)-1H-imidazol-1-yl]-N-[(pyridin-2-yl)methyl]acetamide |
| 47 | 2-[4-(4-fluorophenyl)-5-(pyridin-4-yl)-1H-imidazol-1-yl]-1-[4-(propan-2-yl)piperazin-1-yl]ethan-1-one |
| 48 | 2-[4-(4-fluorophenyl)-5-(pyridin-4-yl)-1H-imidazol-1-yl]-1-(morpholin-4-yl)ethan-1-one |
| 49 | 1-[(1S,4S)-2,5-diazabicyclo[2.2.1]heptan-2-yl]-2-[4-(4-fluorophenyl)-5-(pyridin-4-yl)-1H-imidazol-1- |
| | yl]ethan-1-one |
| 50 | 2-[4-(4-fluorophenyl)-5-{1H-pyrrolo[2,3-b]pyridin-4-yl}-1H-imidazol-1-yl]-1-(piperazin-1-yl)ethan-1-one |
| 51 | 2-[4-(4-fluorophenyl)-5-(pyridin-4-yl)-2-(trifluoromethyl)-1H-imidazol-1-yl]-1-(piperazin-1-yl)ethan-1-one |
| 52 | 2-[4-(4-fluorophenyl)-2-(propan-2-yl)-5-(pyridin-4-yl)-1H-imidazol-1-yl]-1-(piperazin-1-yl)ethan-1-one |
| 53 | 2-[4-(4-fluorophenyl)-5-{1H-pyrrolo[2,3-b]pyridin-4-yl}-1H-imidazol-1-yl]-1-(4-methylpiperazin-1- |
| | yl)ethan-1-one |
| 54 | 2-[2-(4-chlorophenyl)-1-(pyridin-4-yl)-1H-imidazol-5-yl]-1-(piperazin-1-yl)ethan-1-one |
| 55 | 2-[4-(4-fluorophenyl)-5-(pyridin-4-yl)-1H-imidazol-1-yl]-1-(piperazin-1-yl)propan-1-one |
| 56 | 2-[4-(4-methoxyphenyl)-5-(pyridin-4-yl)-1H-imidazol-1-yl]-1-(piperazin-1-yl)ethan-1-one |
| 57 | 1-(piperazin-1-yl)-2-[5-(pyridin-4-yl)-4-[4-(trifluoromethyl)phenyI]-1H-imidazol-1-yl]ethan-1-one |
| 58 | 2-[4-(4-fluorophenyl)-5-{1H-pyrrolo[2,3-b]pyridin-4-yl}-1H-imidazol-1-yl]-1-(morpholin-4-yl)ethan-1-one |
| 59 | 2-{5-[2-(cyclopentylamino)pyridin-4-yl]-4-[4-(trifluoromethyl)phenyl]-1H-imidazol-1-yl}-1-(piperazin-1- |
| | yl)ethan-1-one |
| 60 | 2-[2-(4-chlorophenyl)-1-(pyridin-4-yl)-1H-imidazol-5-yl]-1-(4-methylpiperazin-1-yl)ethan-1-one |
| 61 | 2-[5-(2-aminopyridin-4-yl)-4-(4-fluorophenyl)-1H-imidazol-1-yl]-1-(piperazin-1-yl)ethan-1-one |
| 62 | 2-{5-[2-(cyclopentylamino)pyridin-4-yl]-4-(4-fluorophenyl)-1H-imidazol-1-yl}-1-(piperazin-1-yl)ethan-1- |
| | one |
| 63 | 2-[1-(4-fluorophenyl)-5-(pyridin-4-yl)-1H-pyrazol-4-yl]-1-(4-methylpiperazin-1-yl)ethan-1-one |
| 64 | 4-[4-(4-fluorophenyl)-1-[1-(1-methylpiperidine-4-carbonyl)azetidin-3-yl]-1H-imidazol-5-yl]pyridine |
| 65 | 2-[4-(4-chlorophenyl)-5-{2-[(cyclopentylamino)methyl]pyridin-4-yl}-1H-imidazol-1-yl]-1-(piperazin-1- |
| | yl)ethan-1-one |
| 66 | 2-[4-(4-chlorophenyl)-5-{2-[(methylamino)methyl]pyridin-4-yl}-1H-imidazol-1-yl]-1-(piperazin-1-yl)ethan- |
| | 1-one |
| 67 | 2-{5-[2-(aminomethyl)pyridin-4-yl]-4-(4-chlorophenyl)-1H-imidazol-1-yl}-1-(piperazin-1-yl)ethan-1-one |
| 68 | 2-[4-(4-chlorophenyl)-5-{2-[(dimethylamino)methyl]pyridin-4-yl}-1H-imidazol-1-yl]-1-(piperazin-1- |
| | yl)ethan-1-one |
| 69 | 4-{1-[2-oxo-2-(piperazin-1-yl)ethyl]-5-(pyridin-4-yl)-1H-imidazol-4-yl}benzonitrile |
| 70 | N-({4-[4-(4-chlorophenyl)-1-[2-oxo-2-(piperazin-1-yl)ethyl]-1H-imidazol-5-yl]pyridin-2- |
| | yl}methyl)acetamide |
| 71 | 2-[5-(4-fluorophenyl)-1-methyl-4-(pyridin-4-yl)-1H-pyrazol-3-yl]-1-(4-methylpiperazin-1-yl)ethan-1-one |
| 72 | N-{4-[4-(4-fluorophenyl)-1-(2-{2-oxa-6-azaspiro[3.3]heptan-6-yl}-2-oxoethyl)-1H-imidazol-5-yl]pyridin-2- |
| | yljbenzamide |
| 73 | 2-[4-phenyl-5-(pyridin-4-yl)-1H-imidazol-1-yl]-1-(piperazin-1-yl)ethan-1-one |
| 74 | 2-[4-(4-chlorophenyl)-5-(pyridin-4-yl)-1H-imidazol-1-yl]-N-(1-methylpyrrolidin-3-yl)acetamide |
| 75 | N-{4-[4-(4-fluorophenyl)-1-[2-(morpholin-4-yl)-2-oxoethyl]-1H-imidazol-5-yl]pyridin-2-yl}-2,2- |
| | dimethylpropanamide |
| 76 | 2-[4-(4-fluorophenyl)-5-(pyrimidin-4-yl)-1H-imidazol-1-yl]-1-(4-methylpiperazin-1-yl)ethan-1-one |
| 77 | 2-[2-chloro-4-(4-fluorophenyl)-5-(pyridin-4-yl)-1H-imidazol-1-yl]-1-(4-methylpiperazin-1-yl)ethan-1-one |
| 78 | 2-[4-(4-chlorophenyl)-5-(pyridin-4-yl)-1H-imidazol-1-yl]-N-(pyridin-4-yl)acetamide |
| 79 | N-{4-[4-(4-fluorophenyl)-1-[2-(morpholin-4-yl)-2-oxoethyl]-1H-imidazol-5-yl]pyridin-2- |
| | yljcyclopentanecarboxamide |
| 80 | 2-[2-chloro-4-(4-fluorophenyl)-5-(pyridin-4-yl)-1H-imidazol-1-yl]-1-{2-methyl-2,7-diazaspiro[3.5]nonan- |
| | 7-yl}ethan-1-one |
| 81 | 2-[3-(4-fluorophenyl)-1-methyl-4-(pyridin-4-yl)-1H-pyrazol-5-yl]-1-(4-methylpiperazin-1-yl)ethan-1-one |
| 82 | 2-[3-(4-fluorophenyl)-4-(pyridin-4-yl)-1H-pyrazol-5-yl]-1-(4-methylpiperazin-1-yl)ethan-1-one |
| 83 | 2-[4-(4-chlorophenyl)-5-(pyridin-4-yl)-1H-imidazol-1-yl]-1-[2-(2,2-difluoroethyl)-2,6-diazaspiro[3.4]octan- |
| | 6-yl]ethan-1-one |
| 84 | 2-[4-(4-chlorophenyl)-5-[2-(trifluoromethyl)pyridin-4-yl]-1H-imidazol-1-yl]-1-{2-methyl-2,7- |
| | diazaspiro[3.5]nonan-7-yl}ethan-1-one |
| 85 | 2-[4-(4-chlorophenyl)-5-[2-(difluoromethyl)pyridin-4-yl]-1H-imidazol-1-yl]-1-{2-methyl-2,7- |
| | diazaspiro[3.5]nonan-7-yl}ethan-1-one |
| 86 | 2-[4-(4-chlorophenyl)-5-[2-(difluoromethyl)pyridin-4-yl]-1H-imidazol-1-yl]-1-{2-methyl-2,5- |
| | diazaspiro[3.4]octan-5-yl}ethan-1-one |
| 87 | N-{4-[4-(4-fluorophenyl)-1-[2-(morpholin-4-yl)-2-oxoethyl]-1H-imidazol-5-yl]pyridin-2-yl}-2- |
| | methylpropanamide |
| 88 | 2-[4-(4-chlorophenyl)-5-[2-(difluoromethyl)pyridin-4-yl]-1H-imidazol-1-yl]-1-{2-oxa-6- |
| | azaspiro[3.3]heptan-6-yl}ethan-1-one |
| 89 | 2-[4-(4-chlorophenyl)-5-[2-(trifluoromethyl)pyridin-4-yl]-1H-imidazol-1-yl]-1-{2-methyl-2,5- |
| | diazaspiro[3.4]octan-5-yl}ethan-1-one |
| 90 | 2-[4-(4-chlorophenyl)-5-[2-(trifluoromethyl)pyridin-4-yl]-1H-imidazol-1-yl]-1-{2-oxa-6- |
| | azaspiro[3.3]heptan-6-yl}ethan-1-one |
| 91 | 2-[4-(2-fluorophenyl)-5-(pyridin-4-yl)-1H-imidazol-1-yl]-1-(4-methylpiperazin-1-yl)ethan-1-one |
| 92 | 2-[4-(3-fluorophenyl)-5-(pyridin-4-yl)-1H-imidazol-1-yl]-1-(4-methylpiperazin-1-yl)ethan-1-one |
| 93 | 2-[4-(4-chlorophenyl)-5-[2-(difluoromethyl)pyridin-4-yl]-1H-imidazol-1-yl]-N-[(3R)-1-methylpyrrolidin-3- |
| | yl]acetamide |
| 94 | 2-[4-(2-chlorophenyl)-5-(pyridin-4-yl)-1H-imidazol-1-yl]-1-(4-methylpiperazin-1-yl)ethan-1-one |
| 95 | N-{4-[4-(4-fluorophenyl)-1-(2-{2-oxa-6-azaspiro[3.4]octan-6-yl}-2-oxoethyl)-1H-imidazol-5-yl]pyridin-2- |
| | yljbenzamide |
| 96 | N-{4-[4-(4-fluorophenyl)-1-(2-{6-oxa-2-azaspiro[3.4]octan-2-yl}-2-oxoethyl)-1H-imidazol-5-yl]pyridin-2- |
| | yljbenzamide |
| 97 | 2-[4-(4-chlorophenyl)-5-(pyridin-4-yl)-1H-imidazol-1-yl]-1-{5H,6H,7H,8H-imidazo[1,5-a]pyrazin-7- |
| | yl}ethan-1-one |
| 98 | 2-[4-(4-chlorophenyl)-5-(pyridin-4-yl)-1H-imidazol-1-yl]-1-[2-(oxetan-3-yl)-2,7-diazaspiro[3.5]nonan-7- |
| | yl]ethan-1-one |
| 99 | 2-[4-(4-chlorophenyl)-5-(pyridin-4-yl)-1H-imidazol-1-yl]-1-[3-(4,4-difluoropiperidin-1-yl)azetidin-1- |
| | yl]ethan-1-one |
| 100 | 2-[4-(4-chlorophenyl)-5-(pyridin-4-yl)-1H-imidazol-1-yl]-N-[(3R)-1-methylpyrrolidin-3-yl]acetamide |
| 101 | 2-[4-(4-chlorophenyl)-5-(pyridin-4-yl)-1H-imidazol-1-yl]-N-[(3S)-1-methylpyrrolidin-3-yl]acetamide |
| 102 | 2-[4-(4-chlorophenyl)-5-(pyridin-4-yl)-1H-imidazol-1-yl]-N-[(3S)-oxolan-3-yl]acetamide |
| 103 | 2-[4-(4-chlorophenyl)-5-(pyridin-4-yl)-1H-imidazol-1-yl]-N-[(3R)-oxolan-3-yl]acetamide |
| 104 | 2-[4-(4-fluorophenyl)-5-{2-[(oxetan-3-yl)amino]pyridin-4-yl}-1H-imidazol-1-yl]-1-(4-methylpiperazin-1- |
| | yl)ethan-1-one |
| 105 | 2-[4-(4-chlorophenyl)-5-[2-(difluoromethyl)pyridin-4-yl]-1H-imidazol-1-yl]-N-[(3S)-1-methylpyrrolidin-3- |
| | yl]acetamide |
| 106 | 2-[1-tert-butyl-5-(4-fluorophenyl)-4-(pyridin-4-yl)-1H-pyrazol-3-yl]-1-(4-methylpiperazin-1-yl)ethan-1-one |
| 107 | 2-[4-(4-fluorophenyl)-5-(pyridin-3-yl)-1H-imidazol-1-yl]-1-(4-methylpiperazin-1-yl)ethan-1-one |
| 108 | 2-[4-(4-fluorophenyl)-5-(pyridin-3-yl)-1H-imidazol-1-yl]-1-(piperazin-1-yl)ethan-1-one |
| 109 | 2-[4-(4-chlorophenyl)-5-(pyridin-4-yl)-1H-imidazol-1-yl]-N-(1-methylpyrrolidin-3-yl)acetamide |
| 110 | N-{4-[4-(4-fluorophenyl)-1-[2-(morpholin-4-yl)-2-oxoethyl]-1H-imidazol-5-yl]pyridin-2-yl}-2,2- |
| | dimethylpropanamide |
| 111 | 2-[4-(4-fluorophenyl)-5-(pyrimidin-4-yl)-1H-imidazol-1-yl]-1-(4-methylpiperazin-1-yl)ethan-1-one |
| 112 | 2-[2-chloro-4-(4-fluorophenyl)-5-(pyridin-4-yl)-1H-imidazol-1-yl]-1-(4-methylpiperazin-1-yl)ethan-1-one |
| 113 | 2-[4-(4-chlorophenyl)-5-(pyridin-4-yl)-1H-imidazol-1-yl]-N-(pyridin-4-yl)acetamide |
| 114 | N-{4-[4-(4-fluorophenyl)-1-[2-(morpholin-4-yl)-2-oxoethyl]-1H-imidazol-5-yl]pyridin-2- |
| | yljcyclopentanecarboxamide |
| 115 | 2-[2-chloro-4-(4-fluorophenyl)-5-(pyridin-4-yl)-1H-imidazol-1-yl]-1-{2-methyl-2,7-diazaspiro[3.5]nonan- |
| | 7-yl}ethan-1-one |
| 116 | N-{4-[4-(4-fluorophenyl)-1-(2-{2-oxa-6-azaspiro[3.3]heptan-6-yl}-2-oxoethyl)-1H-imidazol-5-yl]pyridin-2- |
| | yljbenzamide |
| 117 | 2-[3-(4-fluorophenyl)-1-methyl-4-(pyridin-4-yl)-1H-pyrazol-5-yl]-1-(4-methylpiperazin-1-yl)ethan-1-one |
| 118 | 2-[3-(4-fluorophenyl)-4-(pyridin-4-yl)-1H-pyrazol-5-yl]-1-(4-methylpiperazin-1-yl)ethan-1-one |
| 119 | 2-[4-(4-chlorophenyl)-5-(pyridin-4-yl)-1H-imidazol-1-yl]-1-[2-(2,2-difluoroethyl)-2,6-diazaspiro[3.4]octan- |
| | 6-yl]ethan-1-one |
| 120 | 2-[4-(4-chlorophenyl)-5-[2-(trifluoromethyl)pyridin-4-yl]-1H-imidazol-1-yl]-1-{2-methyl-2,7- |
| | diazaspiro[3.5]nonan-7-yl}ethan-1-one |
| 121 | 2-[4-(4-chlorophenyl)-5-[2-(difluoromethyljpyridin-4-yl]-1H-imidazol-1-yl]-1-{2-methyl-2,7- |
| | diazaspiro[3.5]nonan-7-yl}ethan-1-one |
| 122 | 2-[4-(4-chlorophenyl)-5-[2-(difluoromethyl)pyridin-4-yl]-1H-imidazol-1-yl]-1-{2-methyl-2,5- |
| | diazaspiro[3.4]octan-5-yl}ethan-1-one |
| 123 | N-{4-[4-(4-fluorophenyl)-1-[2-(morpholin-4-yl)-2-oxoethyl]-1H-imidazol-5-yl]pyridin-2-yl}-2- |
| | methylpropanamide |
| 124 | 2-[4-(4-chlorophenyl)-5-[2-(difluoromethyl)pyridin-4-yl]-1H-imidazol-1-yl]-1-{2-oxa-6- |
| | azaspiro[3.3]heptan-6-yl}ethan-1-one |
| 125 | 2-[4-(4-chlorophenyl)-5-[2-(trifluoromethyl)pyridin-4-yl]-1H-imidazol-1-yl]-1-{2-methyl-2,5- |
| | diazaspiro[3.4]octan-5-yl}ethan-1-one |
| 126 | 2-[4-(4-chlorophenyl)-5-[2-(trifluoromethyl)pyridin-4-yl]-1H-imidazol-1-yl]-1-{2-oxa-6- |
| | azaspiro[3.3]heptan-6-yl}ethan-1-one |
| 127 | 2-[4-(2-fluorophenyl)-5-(pyridin-4-yl)-1H-imidazol-1-yl]-1-(4-methylpiperazin-1-yl)ethan-1-one |
| 128 | 2-[4-(3-fluorophenyl)-5-(pyridin-4-yl)-1H-imidazol-1-yl]-1-(4-methylpiperazin-1-yl)ethan-1-one |
| 129 | 2-[4-(2-chlorophenyl)-5-(pyridin-4-yl)-1H-imidazol-1-yl]-1-(4-methylpiperazin-1-yl)ethan-1-one |
| 130 | N-{4-[4-(4-fluorophenyl)-1-(2-{2-oxa-6-azaspiro[3.4]octan-6-yl}-2-oxoethyl)-1H-imidazol-5-yl]pyridin-2- |
| | yljbenzamide |
| 131 | N-{4-[4-(4-fluorophenyl)-1-(2-{6-oxa-2-azaspiro[3.4]octan-2-yl}-2-oxoethyl)-1H-imidazol-5-yl]pyridin-2- |
| | yljbenzamide |
| 132 | 2-[4-(4-chlorophenyl)-5-(pyridin-4-yl)-1H-imidazol-1-yl]-1-{5H,6H,7H,8H-imidazo[1,5-a]pyrazin-7- |
| | yl}ethan-1-one |
| 133 | 2-[4-(4-chlorophenyl)-5-(pyridin-4-yl)-1H-imidazol-1-yl]-1-[2-(oxetan-3-yl)-2,7-diazaspiro[3.5]nonan-7- |
| | yl]ethan-1-one |
| 134 | 2-[4-(4-chlorophenyl)-5-(pyridin-4-yl)-1H-imidazol-1-yl]-N-[1-methylpyrrolidin-3-yl]acetamide |
| 134-R | 2-[4-(4-chlorophenyl)-5-(pyridin-4-yl)-1H-imidazol-1-yl]-N-[(3R)-1-methylpyrrolidin-3-yl]acetamide |
| 134-S | 2-[4-(4-chlorophenyl)-5-(pyridin-4-yl)-1H-imidazol-1-yl]-N-[(3S)-1-methylpyrrolidin-3-yl]acetamide |
| 135 | 2-[4-(4-chlorophenyl)-5-(pyridin-4-yl)-1H-imidazol-1-yl]-N-[oxolan-3-yl]acetamide |
| 135-S | 2-[4-(4-chlorophenyl)-5-(pyridin-4-yl)-1H-imidazol-1-yl]-N-[(3S)-oxolan-3-yl]acetamide |
| 135-R | 2-[4-(4-chlorophenyl)-5-(pyridin-4-yl)-1H-imidazol-1-yl]-N-[(3R)-oxolan-3-yl]acetamide |
| 136 | 2-[4-(4-chlorophenyl)-5-[2-(difluoromethyl)pyridin-4-yl]-1H-imidazol-1-yl]-N-[1-methylpyrrolidin-3- |
| | yl]acetamide |
| 136-S | 2-[4-(4-chlorophenyl)-5-[2-(difluoromethyl)pyridin-4-yl]-1H-imidazol-1-yl]-N-[(3S)-1-methylpyrrolidin-3- |
| | yl]acetamide |
| 136-R | 2-[4-(4-chlorophenyl)-5-[2-(difluoromethyl)pyridin-4-yl]-1H-imidazol-1-yl]-N-[(3R)-1-methylpyrrolidin-3- |
| | yl]acetamide |
| 137 | 2-[4-(4-chlorophenyl)-5-(pyridin-4-yl)-1H-imidazol-1-yl]-N-(4,4-difluoro-1-methylpyrrolidin-3- |
| | yl)acetamide |
| 138 | 2-[4-(4-chlorophenyl)-5-(pyridin-4-yl)-1H-imidazol-1-yl]-N-[1-methylpiperidin-3-yl]acetamide |
| 138-R | 2-[4-(4-chlorophenyl)-5-(pyridin-4-yl)-1H-imidazol-1-yl]-N-[(3R)-1-methylpiperidin-3-yl]acetamide |
| 138-S | 2-[4-(4-chlorophenyl)-5-(pyridin-4-yl)-1H-imidazol-1-yl]-N-[(3S)-1-methylpiperidin-3-yl]acetamide |
| 139 | 2-[4-(4-chlorophenyl)-5-(pyridin-4-yl)-1H-imidazol-1-yl]-1[2-(2,2-difluoroethyl)-2,7- |
| | diazaspiro[3.5]nonan-7-yl]ethan-1-one |
| 140 | 2-[4-(4-chlorophenyl)-5-(3-chloropyridin-4-yl)-1H-imidazol-1-yl]-N-[1-methylpyrrolidin-3-yl]acetamide |
| 140-R | 2-[4-(4-chlorophenyl)-5-(3-chloropyridin-4-yl)-1H-imidazol-1-yl]-N-[(3R)-1-methylpyrrolidin-3- |
| | yl]acetamide |
| 140-S | 2-[4-(4-chlorophenyl)-5-(3-chloropyridin-4-yl)-1H-imidazol-1-yl]-N-[(3S)-1-methylpyrrolidin-3- |
| | yl]acetamide |
| 141 | 2-[4-(4-chlorophenyl)-2-(1-methyl-1H-pyrazol-4-yl)-5-(pyridin-4-yl)-1H-imidazol-1-yl]-1-(4- |
| | methylpiperazin-1-yl)ethan-1-one |
| 142 | 2-[4-(4-chlorophenyl)-5-(pyridin-4-yl)-1H-imidazol-1-yl]-N-[1-(2,2-difluoroethyl)pyrrolidin-3-yl]acetamide |
| 142-S | 2-[4-(4-chlorophenyl)-5-(pyridin-4-yl)-1H-imidazol-1-yl]-N-[(3S)-1-(2,2-difluoroethyl)pyrrolidin-3- |
| | yl]acetamide |
| 142-R | 2-[4-(4-chlorophenyl)-5-(pyridin-4-yl)-1H-imidazol-1-yl]-N-[(3R)-1-(2,2-difluoroethyl)pyrrolidin-3- |
| | yl]acetamide |
| 143 | 2-[4-(4-chlorophenyl)-5-(pyridin-4-yl)-1H-imidazol-1-yl]-N-{2-methyl-5-oxa-2-azaspiro[3.4]octan-7- |
| | yl}acetamide |
| 144 | 2-[4-(4-chlorophenyl)-2-(4-methoxyphenyl)-5-(pyridin-4-yl)-1H-imidazol-1-yl]-1-(4-methylpiperazin-1- |
| | yl)ethan-1-one |
| 145 | 2-[4-(4-chlorophenyl)-5-(pyridin-4-yl)-1H-imidazol-1-yl]-1-{2-methyl-5-oxa-2,8-diazaspiro[3.5]nonan-8- |
| | yl}ethan-1-one |
| 146 | 2-[4-(4-chlorophenyl)-5-(pyridin-4-yl)-1H-imidazol-1-yl]-N-methyl-N-{2-methyl-5-oxa-2- |
| | azaspiro[3.4]octan-7-yl}acetamide |
| 147 | 2-[4-(4-chlorophenyl)-2-(6-methoxypyridin-3-yl)-5-(pyridin-4-yl)-1H-imidazol-1-yl]-1-(4-methylpiperazin- |
| | 1-yl)ethan-1-one |
| 148 | 2-[4-(4-chlorophenyl)-5-(pyridin-4-yl)-1H-imidazol-1-yl]-N-methyl-N-[1-methylpyrrolidin-3-yl]acetamide |
| 148-R | 2-[4-(4-chlorophenyl)-5-(pyridin-4-yl)-1H-imidazol-1-yl]-N-methyl-N-[(3R)-1-methylpyrrolidin-3- |
| | yl]acetamide |
| 148-S | 2-[4-(4-chlorophenyl)-5-(pyridin-4-yl)-1H-imidazol-1-yl]-N-methyl-N-[(3S)-1-methylpyrrolidin-3- |
| | yl]acetamide |
| 149 | 2-[4-(4-chlorophenyl)-5-(pyridin-4-yl)-1H-imidazol-1-yl]-1-{4-methyl-1-oxa-4,9-diazaspiro[5.5]undecan- |
| | 9-yl}ethan-1-one |
| 150 | 2-[4-(4-chlorophenyl)-5-(pyridin-4-yl)-1H-imidazol-1-yl]-1-{4-methyl-1-oxa-4,8-diazaspiro[5.5]undecan- |
| | 8-yl}ethan-1-one |
| 151 | 2-[2-chloro-4-(4-fluorophenyl)-5-(pyridin-4-yl)-1H-imidazol-1-yl]-1-{2-ethyl-2,7-diazaspiro[3.5]nonan-7- |
| | yl}ethan-1-one |
| 152 | 2-[2-chloro-4-(4-chlorophenyl)-5-[2-(difluoromethyl)pyridin-4-yl]-1H-imidazol-1-yl]-1-{2-methyl-2,7- |
| | diazaspiro[3.5]nonan-7-yl}ethan-1-one |
| 153 | 2-[4-(4-chlorophenyl)-5-(pyridin-4-yl)-1H-imidazol-1-yl]-1-[(1R,5S)-3-methyl-3,8- |
| | diazabicyclo[3.2.1]octan-8-yl]ethan-1-one |
| 154 | 2-[4-(4-chlorophenyl)-5-(pyridin-4-yl)-1H-imidazol-1-yl]-1-[3-(morpholin-4-yl)azetidin-1-yl]ethan-1-one |
| 155 | 2-[4-(4-chloro-3-fluorophenyl)-5-(pyridin-4-yl)-1H-imidazol-1-yl]-1-(4-methylpiperazin-1-yl)ethan-1-one |
| 156 | 2-[4-(4-chloro-2-fluorophenyl)-5-(pyridin-4-yl)-1H-imidazol-1-yl]-1-(4-methylpiperazin-1-yl)ethan-1-one |
| 157 | 2-[4-(4-chlorophenyl)-5-(pyridin-4-yl)-1H-imidazol-1-yl]-1-[5-methyl-2,5-diazabicyclo[2.2.2]octan-2- |
| | yl]ethan-1-one |
| 157-RR | 2-[4-(4-chlorophenyl)-5-(pyridin-4-yl)-1H-imidazol-1-yl]-1-[(1R,4R)-5-methyl-2,5- |
| | diazabicyclo[2.2.2]octan-2-yl]ethan-1-one |
| 157-SS | 2-[4-(4-chlorophenyl)-5-(pyridin-4-yl)-1H-imidazol-1-yl]-1-[(1S,4S)-5-methyl-2,5- |
| | diazabicyclo[2.2.2]octan-2-yl]ethan-1-one |
| 158 | 2-[4-(4-chlorophenyl)-2-cyclopropyl-5-(pyridin-4-yl)-1H-imidazol-1-yl]-1-(4-methylpiperazin-1-yl)ethan- |
| | 1-one |
| 159 | 2-[4-(2,4-difluorophenyl)-5-(pyridin-4-yl)-1H-imidazol-1-yl]-1-(4-methylpiperazin-1-yl)ethan-1-one |
| 160 | 2-[4-(4-chlorophenyl)-5-(pyridin-4-yl)-1H-imidazol-1-yl]-1-{8-methyl-5-oxa-2,8-diazaspiro[3.5]nonan-2- |
| | yl}ethan-1-one |
| 161 | 2-[4-(4-chlorophenyl)-5-[2-(difluoromethyl)pyridin-4-yl]-2-hydroxy-1H-imidazol-1-yl]-1-{2-methyl-2,7- |
| | diazaspiro[3.5]nonan-7-yl}ethan-1-one |
| 162 | 2-[4-(4-chlorophenyl)-5-(pyridin-4-yl)-1H-imidazol-1-yl]-N-[(1-ethyl-1H-imidazol-2-yl)methyl]-N- |
| | methylacetamide |
| 163 | 2-[4-(4-chlorophenyl)-5-(pyridin-4-yl)-1H-imidazol-1-yl]-1-[3-(1H-imidazol-1-yl)pyrrolidin-1-yl]ethan-1- |
| | one |
| 164 | 2-[4-(4-chlorophenyl)-5-(pyridin-4-yl)-1H-imidazol-1-yl]-1-{3-[(1H-imidazol-1-yl)methyl]azetidin-1- |
| | yl}ethan-1-one |
| 165 | 2-[2-bromo-4-(4-chlorophenyl)-5-(pyridin-4-yl)-1H-imidazol-1-yl]-1-{2-methyl-2,7-diazaspiro[3.5]nonan- |
| | 7-yl}ethan-1-one |
| 166 | 2-[4-(4-chlorophenyl)-5-(pyridin-4-yl)-1H-imidazol-1-yl]-1-[5-methyl-2,5-diazabicyclo[2.2.1]heptan-2- |
| | yl]ethan-1-one |
| 166-RR | 2-[4-(4-chlorophenyl)-5-(pyridin-4-yl)-1H-imidazol-1-yl]-1-[(1R,4R)-5-methyl-2,5- |
| | diazabicyclo[2.2.1]heptan-2-yl]ethan-1-one |
| 167 | 2-[4-(4-chlorophenyl)-2-cyclopropyl-5-(pyridin-4-yl)-1H-imidazol-1-yl]-1-{2-methyl-2,7- |
| | diazaspiro[3.5]nonan-7-yl}ethan-1-one |
| 168 | 2-[3-(4-fluorophenyl)-1-methyl-4-(pyridin-4-yl)-1H-pyrazol-5-yl]-N-methyl-N-[1-methylpyrrolidin-3- |
| | yl]acetamide |
| 168-R | 2-[3-(4-fluorophenyl)-1-methyl-4-(pyridin-4-yl)-1H-pyrazol-5-yl]-N-methyl-N-[(3R)-1-methylpyrrolidin-3- |
| | yl]acetamide |

In a preferred embodiment 134 is 134-R or 134-S, more preferably 134-R. In a preferred embodiment, 135 is 135-R or 135-S, more preferably 135-R. In a preferred embodiment 136 is 136-R or 136-S, preferably 136-S. In a preferred embodiment, 138 is 138-R or 138-S, preferably 138-R. In a preferred embodiment 140 is 140-R or 140-S, preferably 140-R. In a preferred embodiment, 142 is 142-R or 142-S, preferably 142-R. In a preferred embodiment, 148 is 148-R or 148-S, preferably 148-R. In a preferred embodiment, 157 is 157-RR or 157-SS, preferably 157-RR. In a preferred embodiment, 166 is 166-RR. In a preferred embodiment, 168 is 168-R.

In the context of the invention, a salt of a compound according to the invention is preferably a pharmaceutically acceptable salt. Such salts include salts derived from inorganic bases such as Li, Na, K, Ca, Mg, Fe, Cu, Zn and Mn; salts of organic bases such as N,N′-diacetylethylenediamine, glucamine, triethylamine, choline, dicyclohexylamine, benzylamine, trialkylamine, thiamine, guanidine, diethanolamine, alpha-phenylethylamine, piperidine, morpholine, pyridine, hydroxyethylpyrrolidine, hydroxyethylpiperidine, and the like. Such salts also include amino acid salts such as glycine, alanine, cystine, cysteine, lysine, arginine, phenylalanine, guanidine, etc. Such salts may include acid addition salts where appropriate, which are for example sulphates, nitrates, phosphates, perchlorates, borates, hydrohalides such as HCl or HBr salts, acetates, trifluoroacetates, tartrates, maleates, citrates, succinates, palmoates, methanesulphonates, tosylates, benzoates, salicylates, hydroxynaphthoates, benzenesulfonates, ascorbates, glycerophosphates, ketoglutarates and the like. Preferred salts are HCl salts, formic acid salts, acetic acid salts, and trifluoroacetic acid salts. More preferred salts are HCl salts, acetic acid salts and formic acid salts, most preferably HCl salts.

The compound according to the invention is preferably a hydrate or a solvate. In the context of the invention a hydrate refers to a solvate wherein the solvent is water. The term solvate, as used herein, refers to a crystal form of a substance which contains solvent. Solvates are preferably pharmaceutically acceptable solvates and may be hydrates or may comprise other solvents of crystallization such as alcohols, ether, and the like.

Each instance of acyl, alkyl, cycloalkyl, or heterocycloalkyl individually is optionally unsaturated, and optionally substituted with halogen, oxy, hydroxyl, methyl, ethyl, propyl, methoxy, ethoxy, trifluoromethyl, or optionally interrupted by one or more heteroatoms. A skilled person will understand that the valency of atoms is always to be fulfilled. In this context, heterocycloalkyl is to be interpreted as cycloalkyl that has been interrupted by one or more heteroatoms. In the context of this invention, acyl moieties are alkyl moieties wherein the proximal carbon atom is substituted by an oxo moiety (═O). In this context, haloalkyl is to be interpreted as alkyl that has been substituted with halogen. A preferred haloalkyl is a fluorinated alkyl, more preferably a perfluorinated alkyl, most preferably trifluoromethyl. In the context of the invention, halogen is fluorine (F), chlorine (Cl), bromine (Br), or iodine (I). Preferred halogens for compounds according to the invention are fluorine, chlorine, and bromine, more preferred halogens are fluorine or chlorine, a most preferred halogen is fluorine.

In the context of this invention, the number of carbon atoms in a moiety such as alkyl, acyl, cycloalkyl, heterocycloalkyl, is indicated as for example C₁₋₆, in this non-limiting case indicating that from 1 to 6 carbon atoms are envisaged, such as 1, 2, 3, 4, 5, or 6 carbon atoms. Similarly C₂₋₄alkyl has 2, 3, or 4 carbon atoms. The number of carbon atoms can be expressed as the total number of carbon atoms not counting further substitutions, the total number of carbon atoms, or as the number of carbon atoms that can be found in the longest continuous internal sequence of carbon atoms. Preferably, the number of carbon atoms is expressed as the total number of carbon atoms not counting further substitutions.

In the context of this invention, a bridging moiety connects two sites. A bridging moiety is connected to a compound according to the invention on two locations. When a bridging moiety is asymmetric, it can be present in a compound according to the invention in both orientations; preferably, it is present in a compound according to the invention in the orientation in which it is presented, wherein the left side corresponds to the constituent substituent that is first named as forming the bridging moiety, and the right side corresponds to the constituent substituent that is last named as forming the bridging moiety.

In the context of this invention, unsubstituted alkyl groups have the general formula CₙH₂ₙ₊₁ and may be linear or branched. Unsubstituted alkyl groups may also contain a cyclic moiety, and thus have the concomitant general formula CₙH₂ₙ₋₁. Optionally, the alkyl groups are substituted by one or more substituents further specified in this document. Examples of suitable alkyl groups include, but are not limited to, —CH₃, —CH₂CH₃, —CH₂CH₂CH₃, —CH(CH₃)₂, —CH(CH₃)CH₂CH₃, —CH₂CH(CH₃)₂, —CH₂CH₂CH₂CH₃, —C(CH₃)₃, 1-hexyl and the like. Preferred alkyl groups are linear or branched, most preferably, linear. Cycloalkyl groups are cyclic alkyl groups; preferred cycloalkyl groups are cyclopropyl, cyclobutyl, cyclopentyl, and cyclohexyl, most preferably cyclopentyl. Heterocycloalkyl groups are cycloalkyl groups wherein at least one CH₂ moiety is replaced by a heteroatom. Preferred heteroatoms are S, O, and N. Preferred heterocycloalkyl groups are pyrrolidinyl, piperidinyl, oxiranyl, and oxolanyl. Preferred C₁₋₄alkyl groups are —CH₃, —CH₂CH₃, —CH₂CH₂CH₃, —CH(CH₃)₂, —CH(CH₃)CH₂CH₃, —CH₂CH(CH₃)₂, —CH₂CH₂CH₂CH₃, —C(CH₃)₃, cyclopropyl, and cyclobutyl, more preferably, —CH₃, —CH₂CH₃, —CH₂CH₂CH₃, —CH(CH₃)₂, —CH(CH₃)CH₂CH₃, —CH₂CH(CH₃)₂, —CH₂CH₂CH₂CH₃, and —C(CH₃)₃.

Alkyl groups of the invention are optionally unsaturated. In preferred embodiments, alkyl is not unsaturated. Unsaturated alkyl groups are preferably alkenyl or alkynyl groups. In the context of this invention, unsubstituted alkenyl groups have the general formula CₙH₂ₙ₋₁, and may be linear or branched. Examples of suitable alkenyl groups include, but are not limited to, ethenyl, propenyl, isopropenyl, butenyl, pentenyl and the like. Unsubstituted alkenyl groups may also contain a cyclic moiety, and thus have the concomitant general formula CₙH₂ₙ₋₃. Preferred alkenyl groups are linear or branched, most preferably, linear. Highly preferred unsaturated cycloalkyl groups are aryl groups, such as phenyl.

In the context of this invention, unsubstituted alkynyl groups have the general formula CₙH₂ₙ₋₃ and may be linear or branched. Unsubstituted alkynyl groups may also contain a cyclic moiety, and thus have the concomitant general formula CₙH₂ₙ₋₅. Optionally, the alkynyl groups are substituted by one or more substituents further specified in this document. Examples of suitable alkynyl groups include, but are not limited to, ethynyl, propargyl, n-but-2-ynyl, n-but-3-ynyl, and octyne such as cyclooctyne. Preferred alkyl groups are linear or branched, most preferably linear.

In the context of this invention, aryl groups are aromatic and generally comprise at least six carbon atoms and may include monocyclic, bicyclic and polycyclic structures. Optionally, the aryl groups may be substituted by one or more substituents further specified in this document. Examples of aryl groups include groups such as phenyl, naphthyl, anthracyl and the like. A heteroaryl group is aromatic and comprises one to four heteroatoms selected from the group consisting of S, O, and N. Due to the heteroatoms it can have a smaller ring size than six.

In this invention, each instance of alkyl, acyl, cycloalkyl, and heterocycloalkyl is optionally substituted, preferably with one or more moieties selected from halogen, oxy, hydroxyl, methyl, ethyl, propyl, methoxy, ethoxy, trifluoromethyl, wherein each instance can also be interrupted by a heteroatom such as N, O, or S, and wherein each instance of alkyl, acyl, alkoxyl, cyclyl, and heterocyclyl is optionally unsaturated. Interruption by a heteroatom means interruption by one or more heteroatoms. In this context, preferably no more than 20, more preferably 1, 2, 3, 4, or 5 heteroatoms interrupt, even more preferably 1, 2, or 3, preferably 1 or 2, most preferably 1 heteroatom interrupts. Preferably all interrupting heteroatoms are of the same element. As a non-limiting example, the C₅alkyl —CH₂—CH₂—CH₂—CH₂—CH₃ when interrupted by heteroatoms can be —CH₂—CH₂—O—CH₂—CH₂—O—CH₃. In preferred embodiments, there is no optional substitution. In preferred embodiments, there is both substitution and unsaturation.

In preferred embodiments, C₁₋₆alkyl when optionally unsaturated and optionally substituted can be C₁₋₆alkyl, C₁₋₆acyl, C₂₋₆alkenyl, C₂₋₆alkynyl, C₃₋₆cycloalkyl, C₃₋₆heterocycloalkyl, or C₅₋₆aryl, optionally substituted with one or more moieties selected from halogen, oxy, hydroxyl, methyl, ethyl, propyl, methoxy, ethoxy, and trifluoromethyl. In preferred embodiments, C₁₋₄alkyl when optionally unsaturated and optionally substituted can be C₁₋₄alkyl, C₁₋₄acyl, C₂₋₄alkenyl, C₂₋₄alkynyl, C₃₋₄cycloalkyl, or C₃₋₄heterocycloalkyl, optionally substituted with one or more moieties selected from halogen, oxy, hydroxyl, methyl, ethyl, propyl, methoxy, ethoxy, and trifluoromethyl.

Molecules provided in this invention can be optionally substituted. Suitable optional substitutions are replacement of —H by a halogen. Preferred halogens are F, Cl, Br, and I, most preferably F. Further suitable optional substitutions are substitutions of one or more —H by oxy, hydroxyl, methyl, ethyl, propyl, methoxy, ethoxy, and trifluoromethyl.

### Compositions and Combinations

In a further aspect, the invention provides a composition comprising at least one compound of general formula I, and a pharmaceutically acceptable excipient, preferably for use according to the invention (use is described elsewhere herein). Such a composition is referred to herein as a composition according to the invention. Preferred compositions according to the invention are pharmaceutical compositions. In preferred embodiments, the composition according to the invention is formulated for oral, sublingual, parenteral, intravascular, intravenous, subcutaneous, or transdermal administration, optionally for administration by inhalation; preferably for oral administration. More features and definitions of administration methods are provided in the section on formulation and administration.

The invention also provides combinations of compounds according to the invention with further measures known for treating or ameliorating diseases or conditions associated with DUX4, for example known for treatments of FSHD or cancer. In preferred embodiments of such combinations is provided a combination of a compound according to the invention and a chemotherapeutic agent. Chemotherapeutic agents are widely known. In another preferred combination, the compound according to the invention is combined with a p38 inhibitor, a β2 adrenergic receptor agonist, a CK1 inhibitor, and/or a BET inhibitor. In some preferred combinations the compound may be combined with clinical management, for example involving physical therapy, aerobic exercise, respiratory function therapy, or orthopedic interventions.

### Compound for Use

Following the central role of DUX4 in the consensus disease hypothesis for FSHD, a therapeutic approach with a disease-modifying potential is expected to rely on the inhibition of DUX4. The inventors have identified the compounds according to the invention as being able to achieve DUX4 repression in muscle cells. This invention has been made using primary FSHD patient-derived muscle cells. Because of the primate-specificity of the FSHD locus and questionable relevance of recombinant, immortalized, or tumorigenic cell or animal models to study endogenous DUX4 regulatory mechanisms, primary patient-derived muscle cells are the most relevant disease model. Assays based on immortalized cells bear the risk of altered epigenomes, thereby limiting their relevance in studying the endogenous regulation of DUX4 expression. Particularly the subtelomeric location of D4Z4 and the importance of the D4Z4 epigenome in the stability of DUX4 repression (Stadler et al., 2013, DOI: 10.1038/nsmb.2571) underscore the necessity of using primary muscle cells to discover physiologically relevant drug targets that regulate the expression of DUX4.

DUX4 has historically been regarded as being challenging to detect in FSHD muscle. Its expression in primary myoblasts from patients with FSHD has been shown to be stochastic. Studies have reported that only 1 in 1000 or 1 in 200 nuclei is DUX4 positive in proliferating FSHD myoblasts and during myoblast differentiation, respectively. Due to this particularly low abundance of DUX4, detection of DUX4 protein has been reported to be a technical challenge. While primary FSHD muscle cells have been used extensively in the FSHD literature, none of the reports appear to be applicable beyond a bench scale level. The limitations posed by using primary cells and the recognised complexity of detecting the low levels of endogenous DUX4 illustrate the challenges associated with applying primary FSHD muscle cells to higher throughput formats. Although DUX4 expression increases upon in vitro differentiation of proliferating FSHD myoblasts into multinucleated myotubes, the levels remain low and the dynamic variability is widely accepted to be extremely challenging for robust large-scale screening approaches (Campbell et al., 2017).

The invention thus provides compound according to the invention for use in the treatment of a disease or condition associated with (undue) DUX4 expression, wherein the compound reduces DUX4 expression. The invention provides a compound of general formula (I), or a composition according to the invention, for use as a medicament, wherein the medicament is preferably for use in the treatment of a disease or condition associated with DUX4 expression, and wherein the compound of general formula (I) reduces DUX4 expression, wherein more preferably said disease or condition associated with DUX4 expression is a muscular dystrophy or cancer, even more preferably wherein said disease or condition associated with DUX4 expression is a muscular dystrophy, most preferably facioscapulohumeral muscular dystrophy (FSHD). Such a compound is referred to herein as a compound for use according to the invention.

The medical use herein described is formulated as a compound for use as a medicament for treatment of the stated condition(s) (e.g. by administration of an effective amount of the compound), but could equally be formulated as i) a method of treatment of the stated condition(s) using a compound as defined herein comprising a step of administering to a subject an effective amount of the compound, ii) a compound as defined herein for use in the manufacture of a medicament to treat the stated condition(s), wherein preferably the compound is to be administered in an effective amount, and iii) use of a compound as defined herein for the treatment of the stated condition(s), preferably by administering an effective amount. Such medical uses are all envisaged by the present invention. Preferred subjects are subjects in need of treatment. Treatment preferably leads to delay, amelioration, alleviation, stabilization, cure, or prevention of a disease or condition. In other words, a compound for use according to the invention can be a compound for the treatment, delay, amelioration, alleviation, stabilization, cure, or prevention of the stated disease or condition.

The compound according to the invention reduces DUX4 expression. This DUX4 expression is preferably the overall DUX4 expression of the subject that is treated. DUX4 expression can be determined using methods known in the art or exemplified in the examples. As is known in the art, DUX4 expression can also be determined by determining the expression of its target genes. For example, DUX4 expression can be determined using PCR techniques such as RT-PCR, or using immunostaining, mass spectrometry, or ELISA, for example on a sample containing cells or cell extracts, preferably obtained from the subject. In this context, a reduction is preferably a reduction as compared to either a predetermined value, or to a reference value. A preferred reference value is a reference value obtained by determining DUX4 expression in an untreated sample containing cells or cell extracts. This untreated sample can be from the same subject or from a different and healthy subject, more preferably it is a sample that was obtained in the same way, thus containing the same type of cells. Conveniently, both the test sample and the reference sample can be part of a single larger sample that was obtained. Alternately, the test sample was obtained from the subject before treatment commenced. A highly preferred reference value is the expression level of DUX4 in a sample obtained from a subject prior to the first administration of the compound according to the invention. Another preferred reference value is a fixed value that represents an absence of DUX4 expression.

A reduction of DUX4 expression preferably means that expression is reduced by at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%. If expression of DUX4 is reduced by for example 100%, it may be that expression of DUX4 can no longer be detected. Reduction can be assessed at the protein level, for example through immunostaining, ELISA, or mass spectrometry, or it can be assessed at the mRNA level, for example through PCR techniques such as RT-PCR. In preferred embodiments, the invention provides a compound for use according to the invention, wherein the reduction of DUX4 expression is determined using PCR or immunostaining, wherein a preferred PCR technique is RT-PCR. In preferred embodiments the invention provides a compound for use according to the invention, wherein DUX4 expression is reduced by at least 20%, 40%, 60%, 80%, or more, more preferably by at least 30%, 40%, 60%, 80%, or more. In further preferred embodiments, DUX4 expression is reduced by at least 10%. In further preferred embodiments, DUX4 expression is reduced by at least 20%. In further preferred embodiments, DUX4 expression is reduced by at least 30%. In further preferred embodiments, DUX4 expression is reduced by at least 40%. In further preferred embodiments, DUX4 expression is reduced by at least 50%. In further preferred embodiments, DUX4 expression is reduced by at least 60%. In further preferred embodiments, DUX4 expression is reduced by at least 70%. In further preferred embodiments, DUX4 expression is reduced by at least 80%. In further preferred embodiments, DUX4 expression is reduced by at least 90%. In further preferred embodiments, DUX4 expression is reduced by at least 95%. In the most preferred embodiments, DUX4 expression is reduced by about 100%, preferably by 100%.

In preferred embodiments, the invention provides a compound for use according to the invention, wherein the compound reduces DUX4 expression in muscle cells, immune cells, or cancer cells, preferably in muscle cells or immune cells, most preferably in muscle cells. Preferred muscle cells are myoblasts, satellite cells, myotubes, and myofibers. Preferred immune cells are B cells, T cells, dendritic cells, neutrophils, natural killer cells, granulocytes, innate lymphoid cells, megakaryocytes, myeloid-derived suppressor cells, monocytes/macrophages, and thymocytes, and optionally mast cells. Other preferred cells are platelets and red blood cells. In other embodiments, DUX4 expression is reduced in cancer cells.

In preferred embodiments, a compound according to the invention is for the treatment of patients suffering from both a DUX4-related condition and from muscle inflammation. Muscle inflammation contributes to the pathophysiology of muscular dystrophies such as FSHD. It precedes muscle destruction and fatty replacement, thereby representing an early marker for disease activity. Muscle inflammation can be identified using means known in the art. Preferably, muscle inflammation is identified by at least one of using biopsies and using MRI sequences with short TI inversion recovery (STIR), preferably using MRI with STIR. STIR hyperintensities (STIR+) visualize edema, which correlates with inflammation. A preferred inflamed muscle is a STIR+ muscle. A preferred muscle biopsy is a biopsy from a STIR+ muscle. A preferred muscle inflammation is MAPK-associated muscle inflammation, more preferably a muscle inflammation associated with the transcription and translation of inflammatory response-associated genes that encode proteins such as TNF-a, IL-1 b, IL-6, and IL-8. Muscle inflammation predicts a faster fat replacement of muscle.

A preferred subject suffering from muscle inflammation has at least one inflamed muscle, more preferably at least 2, even more preferably at least 3, even more preferably at least 4, even more preferably at least 5, most preferably at least 6, 7, 8, 9, 10, or 11. Preferably the inflamed muscle is a skeletal muscle, more preferably it is a skeletal muscle of the face, scapula, or upper arms. A preferred subject suffering from muscle inflammation is a subject also suffering from muscular dystrophy, more preferably also suffering from FSHD. Preferably, such a subject suffering from FSHD has at least one inflamed muscle, more preferably at least one STIR+ muscle.

The invention provides a compound according to the invention for use in the treatment of a disease or condition associated with DUX4 expression in a subject, wherein the subject suffers from muscle inflammation. In preferred embodiments, the invention provides compound according to the invention for use in the treatment of FSHD, wherein the subject suffers from muscle inflammation. In preferred embodiments, the invention provides a compound according to the invention for use in the treatment of FSHD, wherein the subject has at least one inflamed muscle, preferably at least one inflamed skeletal muscle of the face, scapula, or upper arms. This muscle is preferably STIR+. Muscle inflammation is known to precede fatty infiltration. Accordingly, the invention provides a compound according to the invention for preventing or delaying fatty infiltration in a muscle of a subject suffering from FSHD.

In preferred embodiments, a compound according to the invention or a combination as defined herein is for the promotion of myogenic fusion and/or for the promotion of myogenic differentiation. The inventors have identified that compounds according to the invention promote both of these important characteristics of healthy or recovering muscles. The use in promoting myogenic fusion and/or myogenic differentiation aids with muscle regeneration.

Skeletal muscle is an example of a tissue that deploys a self-renewing stem cell, the satellite cell, to effect regeneration. These satellite cells remain adjacent to a skeletal muscle fiber, situated between the sarcolemma and the basement membrane of the endomysium (the connective tissue investment that divides the muscle fascicles into individual fibers). To activate myogenesis, the satellite cells must be stimulated to differentiate into new fibers. The satellite cells show asymmetric divisions to renew rare “immortal” stem cells and generate a clonal population of differentiation-competent myoblasts. The myoblast is thus a type of muscle progenitor cell that arises from myogenic satellite cells. Myoblasts differentiate to give rise to muscle cells. Differentiation is regulated by myogenic regulatory factors, including but not limited to MyoD, Myf5, myogenin, and MRF4. GATA4 and GATA6 also play a role in myocyte differentiation. Skeletal muscle fibers are made when myoblasts fuse together or to existing myofibers; muscle fibers therefore are cells with multiple nuclei, known as myonuclei. The myogenic fusion process is specific to skeletal muscle (e.g., biceps brachii) and not cardiac muscle or smooth muscle. The inventors have identified that compounds according to the invention promote this differentiation of satellite cells, thus ultimately promoting myotube formation and myogenesis.

The invention provides a compound according to the invention for use in the treatment of a disease or condition associated with DUX4 expression in a subject, wherein the compound is for promoting myogenic fusion and/or differentiation. Such promoted fusion and differentiation help reinstate healthy skeletal muscle biology. In preferred embodiments, the compound according to the invention is for promoting myogenic fusion. Myogenic fusion is quintessential to muscle formation and muscle regeneration, and it can be assessed using any known method. Preferably, it is assessed using image analysis, more preferably using high content image analysis. In preferred embodiments, the compound according to the invention for promoting myogenic fusion increases myogenic fusion with at least 1, 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 90, 95, 100% or more, preferably with at least 10% or more, more preferably with at least 30% or more, even more preferably with at least 50% or more. It can be that no myogenic fusion was present in a subject or in a muscle or in a sample. In such a case the compound according to the invention for promoting myogenic fusion preferably reinstates myogenic fusion, more preferably to at least 1%, 5%, 10%, 20%, 25%, 30%, 35%, 40%, 45%, 50% or more of a healthy control, even more preferably to at least 5% of a healthy control, more preferably still to at least 15%, most preferably to at least 25% of a healthy control.

In preferred embodiments the compound according to the invention is for promoting myogenic differentiation, which can be in vitro, in vivo, or ex vivo, preferably in vitro or ex vivo, more preferably in vitro. In these embodiments, a cell is preferably a primary cell. In these embodiments, a cell is preferably not an immortalized cell. Myogenic differentiation can be assessed using methods known in the art, such as quantification of myogenic differentiation markers such as MYH2, MyoD, Myf5, myogenin, and 15 MRF4, preferably such as myogenin or MYH2. In preferred embodiments, the compound according to the invention for promoting myogenic differentiation increases myogenic differentiation with at least 1, 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 90, 95, 100% or more, preferably with at least 10% or more, more preferably with at least 30% or more, even more preferably with at least 50% or more. It can be that no myogenic differentiation was present in a subject or in a muscle or in a sample. In such a case the compound according to the invention for promoting myogenic differentiation preferably reinstates myogenic differentiation, more preferably to at least 1%, 5%, 10%, 20%, 25%, 30%, 35%, 40%, 45%, 50% or more of a healthy control, even more preferably to at least 5% of a healthy control, more preferably still to at least 15%, most preferably to at least 25% of a healthy control.

In preferred embodiments, the compound according to the invention is for promoting myogenic fusion, wherein features and definitions are as defined elsewhere herein. In preferred embodiments the compound according to the invention is for promoting myogenic differentiation, wherein features and definitions are as defined elsewhere herein. In preferred embodiments, the compound according to the invention is for promoting myogenic fusion and/or differentiation, wherein features and definitions are as defined elsewhere herein.

In preferred embodiments the invention provides the compounds for use according to the invention, wherein said disease or condition associated with DUX4 expression is a muscular dystrophy or cancer or systemic cachexia, preferably wherein said disease or condition associated with DUX4 expression is a muscular dystrophy, most preferably facioscapulohumeral muscular dystrophy (FSHD). In other preferred embodiments, the compound according to the invention is for treating, ameliorating, or preventing systemic cachexia.

In this context, a preferred muscular dystrophy is FSHD; a preferred cancer is prostate cancer (WO2014081923), multiple myeloma (US20140221313), lung cancer (Lang et al., 2014, DOI: 10.14205/2310-8703.2014.02.01.1), colon cancer (Paz et al., 2003, DOI: 10.1093/hmg/ddg226) sarcoma, or leukemia; a preferred sarcoma is small round cell sarcoma (Oyama et al., 2017 DOI: 10.1038/s41598-017-04967-0; Bergerat et al., 2017, DOI: 10.1016/j.prp.2016.11.015; Chebib and Jo, 2016, DOI: 10.1002/cncy.21685); a preferred leukemia is acute lymphoblastic leukemia (ALL), more particularly B-cell precursor ALL (Yasuda et al., 2016, doi: 10.1038/ng.3535; Lilljebjorn & Fioretos, 2017, DOI: 10.1182/blood-2017-05-742643; Zhang et al., 2017, DOI:10.1038/ng.3691).

Accordingly, in preferred embodiments, the invention provides the compounds for use according to the invention, wherein said disease or condition associated with DUX4 expression is a muscular dystrophy or cancer, preferably wherein said disease or condition associated with DUX4 expression is FSHD, prostate cancer, multiple myeloma, lung cancer, colon cancer (preferably colorectal carcinoma), sarcoma (preferably small round cell sarcoma), leukemia (preferably acute lymphoblastic leukemia, more preferably B-cell precursor acute lymphoblastic leukemia), preferably said disease or condition associated with DUX4 expression is FSHD. In more preferred embodiments, the invention provides the compounds for use according to the invention, wherein said disease or condition associated with DUX4 expression is a muscular dystrophy or cancer, preferably wherein said disease or condition associated with DUX4 expression is FSHD or cancer, wherein cancer is preferably prostate cancer, multiple myeloma, lung cancer, colon cancer (preferably colorectal carcinoma), sarcoma (preferably small round cell sarcoma), leukemia (preferably acute lymphoblastic leukemia, more preferably B-cell precursor acute lymphoblastic leukemia), wherein cancer is more preferably sarcoma, most preferably small round cell sarcoma.

In a preferred embodiment, the invention provides the compounds for use according to the invention, wherein said disease or condition associated with DUX4 expression is cancer, wherein cancer is preferably prostate cancer, multiple myeloma, lung cancer, colon cancer (preferably colorectal carcinoma), sarcoma (preferably small round cell sarcoma), leukemia (preferably acute lymphoblastic leukemia, more preferably B-cell precursor acute lymphoblastic leukemia), wherein cancer is more preferably sarcoma, most preferably small round cell sarcoma.

Other DUX4 targets are known as “cancer testis antigens” (CTAs), which are genes that are normally expressed only in testis, but which are de-repressed in some cancers, eliciting an immune response. These observations imply that DUX4 de-repression in cancers mediates the activation of HSATII, CTAs and/or THE1B promoters (Young et al., 2013, doi:10.1371/journal.pgen.1003947). In line with this, Dmitriev et al. (2014, DOI: 10.1111/jcmm.12182) demonstrate a similarity between FSHD and cancer cell expression profiles, suggesting a common step in the pathogenesis of these diseases.

Expression of DUX4 is known to be associated with immune suppression in tumors (Guo-Liang Chew et al., 2019, Developmental Cell 50, 658-671, DOI: 10.1016/j.devcel.2019.06.011). DUX4 is re-expressed in many cancers, where it suppresses anti-cancer immune activity by blocking interferon-γ-mediated induction of MHC class I and is associated with reduced efficacy of immune checkpoint blockade therapy. DUX4-expressing cancers are characterized by low antitumor immune activity. DUX4 blocks interferon-γ-mediated induction of MHC class I and antigen presentation. As a result, DUX4 is significantly associated with failure to respond to anti-CTLA-4 therapy.

In preferred embodiments, a compound or composition according to the invention is for use in the treatment of cancer, wherein the compound or composition increases the immune response to cancer cells. This may mean that it initiates an immune response in cases where no immune response was present. In this application, a preferred cancer is a cancer with DUX4 expression, more preferably a cancer with reduced MHC class I expression.

In more preferred embodiments for increasing immune response, the compound or composition according to the invention is for increasing the production of immune system activating cytokines, such as interferon-γ. Preferably, cytokine production is increased by 1%, 5%, 10%, 15%, 20%, 25%, 30%, 40%, 50%, 55%, 60%, 65%, 70% or 75%, or more, and is preferably detected through FACS. The increase in cytokines leads to increased immune suppression of cancers and can lead to immune-mediated suppression or partial immune-mediated suppression of cancers that would otherwise not be susceptible to immune-mediated suppression. In preferred embodiments, the compound or composition according to the invention is for increasing T-cell function, such as increasing production of interferon-γ.

In preferred embodiments for increasing immune response, the compound or composition according to the invention is for increasing T-cell frequency. Preferably, such an increase is by 1%, 5%, 10%, 15%, 20%, 25%, 30%, 40%, 50%, 55%, 60%, 65%, 70% or 75%, or more. Such an increase can be determined by measuring CD8 or CD4. For example as described in Guo-Liang Chew et al. In other preferred embodiments for increasing immune response, the compound or composition according to the invention is for increasing specific T-cell subsets. Such subsets can be determined by TCR sequencing. In preferred embodiments for increasing immune response, the compound or composition according to the invention is for inducing T-cell function, preferably for inducing T-cell function by inducing IFNγ production. Most preferably, the compound or composition according to the invention is for increasing T-cell frequency and simultaneously inducing T-cell function, preferably while simultaneously decreasing regulatory T cell population. Tumors with decreased Tregs and with increased CD8+T effector cells are referred to as ‘hot’ tumors, which are tumors that do not have an immunosuppressed microenvironment. Conversely, tumors in an immunosuppressed microenvironment are referred to as ‘cold’ tumors.

Additionally, compounds and compositions according to the invention can reduce expression of immune suppressive target genes such as, but not limited to, CTLA-4 or PD-1 or PD-1L. Such a reduction is preferably by 1%, 5%, 10%, 15%, 20%, 25%, 30%, 40%, 50%, 55%, 60%, 65%, 70% or 75%, or more. Expression can be determined via qPCR. CTLA-4 and PD-1 are T cell inhibitory receptors on which immune checkpoint blockade therapies can act. Such therapy induces durable responses across diverse cancers in susceptible patients. In preferred embodiments, the compound or composition according to the invention is for reducing expression of CTLA-4 or of PD-1 or for reducing expression of CTLA-4 and PD-1.

Additionally, compounds and compositions according to the invention can be combined with compounds that inhibit immune checkpoints such as, but not limited to, CTLA-4, PD-1, or PD-L1. In preferred embodiments, a combination is provided comprising the compound or composition according to the invention and a further compound is for inhibiting CTLA-4, PD-1, or PD-L1. Examples of such further agents are pembrolizumab, spartalizumab, nivolumab (PD-1 inhibitors), and ipilimumab (CTLA-4 inhibitor). Such inhibition is preferably by 1%, 5%, 10%, 15%, 20%, 25%, 30%, 40%, 50%, 55%, 60%, 65%, 70% or 75%, or more. Inhibition can be determined via methods known in the art, such as described or referred to in Guo-Liang Chew et al., 2019.

The compounds of the present invention are also adapted to therapeutic use as antiproliferative agents (e.g., cancer), antitumor (e.g., effect against solid tumors) in mammals, particularly in humans. In particular, the compounds of the present invention are useful in the prevention and treatment of a variety of human hyperproliferative disorders including both malignant and benign abnormal cell growth. The compounds, compositions and methods provided herein are useful for the treatment of cancer and preparation of a medicament to treat cancer including but are not limited to cancer of:

the circulatory system, for example, heart (sarcoma [angiosarcoma, fibrosarcoma, rhabdomyosarcoma, liposarcoma], myxoma, rhabdomyoma, fibroma, lipoma and teratoma), mediastinum and pleura, and other intrathoracic organs, vascular tumors and tumor-associated vascular tissue;

respiratory tract, for example, nasal cavity and middle ear, accessory sinuses, larynx, trachea, bronchus and lung such as small cell lung cancer (SCLC), non-small cell lung cancer (NSCLC), bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; gastrointestinal, for example, esophagus (squamous cell carcinoma, adenocarcinoma, leiomyosarcoma, lymphoma), stomach (carcinoma, lymphoma, leiomyosarcoma), gastric, pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma);

genitourinary tract, for example, kidney (adenocarcinoma, Wilm's tumor [nephroblastoma], lymphoma, leukemia), bladder and/or urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma);

liver, for example, hepatoma (hepatocellular carcinoma), cholangiocarcinoma, hepatoblastoma, angiosarcoma, hepatocellular adenoma, hemangioma, pancreatic endocrine tumors (such as pheochromocytoma, insulinoma, vasoactive intestinal peptide tumor, islet cell tumor and glucagonoma);

bone, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors;

nervous system, for example, neoplasms of the central nervous system (CNS), primary CNS lymphoma, skull cancer (osteoma, hemangioma, granuloma, xanthoma, osteitis deformans), meninges (meningioma, meningiosarcoma, gliomatosis), brain cancer (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma);

reproductive system, for example, gynecological, uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminonna, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma) and other sites associated with female genital organs; placenta, penis, prostate, testis, and other sites associated with male genital organs;

hematologic, for example, blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma];

oral cavity, for example, lip, tongue, gum, floor of mouth, palate, and other parts of mouth, parotid gland, and other parts of the salivary glands, tonsil, oropharynx, nasopharynx, pyriform sinus, hypopharynx, and other sites in the lip, oral cavity and pharynx;

skin, for example, malignant melanoma, cutaneous melanoma, basal cell carcinoma, squamous cell carcinoma, Karposi's sarcoma, moles dysplastic nevi, lipoma, angioma, dermatofibroma, and keloids;

adrenal glands: neuroblastoma; and

cancers involving other tissues including connective and soft tissue, retroperitoneum and peritoneum, eye, intraocular melanoma, and adnexa, breast, head or/and neck, anal region, thyroid, parathyroid, adrenal gland and other endocrine glands and related structures, secondary and unspecified malignant neoplasm of lymph nodes, secondary malignant neoplasm of respiratory and digestive systems and secondary malignant neoplasm of other sites.

More specifically, examples of “cancer” when used herein in connection with the present invention include cancer selected from lung cancer (NSCLC and SCLC), cancer of the head or neck, ovarian cancer, colon cancer, rectal cancer, cancer of the anal region, stomach cancer, breast cancer, cancer of the kidney or ureter, renal cell carcinoma, carcinoma of the renal pelvis, neoplasms of the central nervous system (CNS), primary CNS lymphoma, non-Hodgkins's lymphoma, spinal axis tumors, or a combination of one or more of the foregoing cancers. Still more specifically, examples of “cancer” when used herein in connection with the present invention include cancer selected from lung cancer (NSCLC and SCLC), breast cancer, ovarian cancer, colon cancer, rectal cancer, cancer of the anal region, or a combination of one or more of the foregoing cancers. In one embodiment of the present invention the non-cancerous conditions include such hyperplastic conditions such as benign hyperplasia of the skin (e.g., psoriasis) and benign hyperplasia of the prostate (e.g., BPH).

In another embodiment the present invention provides a compound of general formula (I) for use in methods of treating neurological and psychiatric disorders comprising: administering to a mammal an amount of a compound of general formula (I) effective in treating such disorders, or a pharmaceutically acceptable salt thereof. Neurological and psychiatric disorders include but are not limited to: acute neurological and psychiatric disorders such as cerebral deficits subsequent to cardiac bypass surgery and grafting, stroke, cerebral ischemia, spinal cord trauma, head trauma, perinatal hypoxia, cardiac arrest, hypoglycemic neuronal damage, dementia, AIDS-induced dementia, vascular dementia, mixed dementias, age-associated memory impairment, Alzheimer's disease, Huntington's Chorea, amyotrophic lateral sclerosis, ocular damage, retinopathy, cognitive disorders, including cognitive disorders associated with schizophrenia and bipolar disorders, idiopathic and drug-induced Parkinson's disease, muscular spasms and disorders associated with muscular spasticity including tremors, epilepsy, convulsions, migraine, migraine headache, urinary incontinence, substance tolerance, substance withdrawal, withdrawal from opiates, nicotine, tobacco products, alcohol, benzodiazepines, cocaine, sedatives, and hypnotics, psychosis, mild cognitive impairment, amnestic cognitive impairment, multi-domain cognitive impairment, obesity, schizophrenia, anxiety, generalized anxiety disorder, social anxiety disorder, panic disorder, post-traumatic stress disorder, obsessive compulsive disorder, mood disorders, depression, mania, bipolar disorders, trigeminal neuralgia, hearing loss, tinnitus, macular degeneration of the eye, emesis, brain edema, pain, acute and chronic pain states, severe pain, intractable pain, neuropathic pain, post-traumatic pain, tardive dyskinesia, sleep disorders, narcolepsy, attention deficit/hyperactivity disorder, autism, Asperger's disease, and conduct disorder in a mammal. Accordingly, in one embodiment, the invention provides a method for treating a condition in a mammal, such as a human, selected from the conditions above, comprising administering a compound of general formula (I) to the mammal. The mammal is preferably a mammal in need of such treatment. As examples, the invention provides a compound of general formula (I) for use in method for treating or preparation of a medicament to treat attention deficit/hyperactivity disorder, schizophrenia and Alzheimer's Disease.

The invention relates to a compound of general formula (I) for use in a method of treating a mood disorder selected from the group consisting of a depressive disorder and a bipolar disorder. In another embodiment of the invention, the depressive disorder is major depressive disorder. In a further embodiment of the invention, the mood disorder is a bipolar disorder. In another embodiment, the bipolar disorder is selected from the group consisting of bipolar I disorder and bipolar II disorder.

The compound of general formula (I) can also be for use in treating a condition selected from the group consisting of neurological and psychiatric disorders, including but not limited to: acute neurological and psychiatric disorders such as cerebral deficits subsequent to cardiac bypass surgery and grafting, stroke, cerebral ischemia, spinal cord trauma, head trauma, perinatal hypoxia, cardiac arrest, hypoglycemic neuronal damage, dementia, AIDS-induced dementia, vascular dementia, mixed dementias, age-associated memory impairment, Alzheimer's disease, Huntington's Chorea, amyotrophic lateral sclerosis, ocular damage, retinopathy, cognitive disorders, including cognitive disorders associated with schizophrenia and bipolar disorders, idiopathic and drug-induced Parkinson's disease, muscular spasms and disorders associated with muscular spasticity including tremors, epilepsy, convulsions, migraine, migraine headache, urinary incontinence, substance tolerance, substance withdrawal, withdrawal from opiates, nicotine, tobacco products, alcohol, benzodiazepines, cocaine, sedatives, and hypnotics, psychosis, mild cognitive impairment, amnestic cognitive impairment, multi-domain cognitive impairment, obesity, schizophrenia, anxiety, generalized anxiety disorder, social anxiety disorder, panic disorder, post-traumatic stress disorder, obsessive compulsive disorder, mood disorders, depression, mania, bipolar disorders, trigeminal neuralgia, hearing loss, tinnitus, macular degeneration of the eye, emesis, brain edema, pain, acute and chronic pain states, severe pain, intractable pain, neuropathic pain, post-traumatic pain, tardive dyskinesia, sleep disorders, narcolepsy, attention deficit/hyperactivity disorder, autism, Asperger's disease, and conduct disorder in a mammal, comprising administering an effective amount of a compound of general formula (I) or pharmaceutically acceptable salt thereof, and a pharmaceutically acceptable carrier. The composition optionally further comprises an atypical antipsychotic, a cholinesterase inhibitor, Dimebon, or NMDA receptor antagonist. Such atypical antipsychotics include, but are not limited to, ziprasidone, clozapine, olanzapine, risperidone, quetiapine, aripiprazole, paliperidone; such NMDA receptor antagonists include but are not limited to memantine; and such cholinesterase inhibitors include but are not limited to donepezil and galantamine.

Compounds according to the invention can also be used for treating auto-immune disorders. Particularly suitable disorders in this context are such as rheumatoid arthritis, asthma, psoriasis, chronic pulmonary inflammation, chronic obstructive pulmonary disease, asthma, glomerulonephritis, Crohn's disease, ICF (immunodeficiency, centromeric region instability and facial anomalies), and myositis such as myositis ossificans, (idiopathic) inflammatory myopathies, dermatomyositis, juvenile dermatomyositis, polymyositis, inclusion body myositis, benign acute childhood myositis, statin-associated autoimmune myopathy, and pyomyositis. Preferred in this context are ICF and myositis, wherein myositis is most preferred.

Many targets are known to be associated with DUX4 repression. Examples are BET proteins (such as BRD2, BRD3, BRD4, BRDT) and β2-adrenergic receptor (Campbell et al., Skeletal Muscle. 2017 Sep. 4; 7(1)); SMCHD1 (Balog et al., Epigenetics. 2015; 10(12): 1133-42); PARP1 (Sharma V et al., J. Genetic syndromes and Gene Therapy. 2016 August; 7(4)); WNT signalling proteins (such as WNT1-16, Axin, beta-catenin, Frizzled, and GSK3) and tankyrase (Block et al., Hum Mol Genet. 2013 Dec. 1; 22(23):4661-72) PRC2/EZH2 and SUV39H1 (Haynes et al., Epigenetics & Chromatin. 2018, 11 (47)); MBD2/NuRD complex, MBD1/CAF-1, TRIM28, SETDB1, KDM1A, SIN3 complex (Campbell et al., eLife. 2018, 7:e31023); ASH1L, BAP1, BAZ1A, BAZ1B, BAZ2A, BPTF, BRD2, BRD3, BRD4, BRDT, BRPF1, BRPF3, CARM1, KDM4A, KDM4B, KDM4C, KDM4D, KDM6A, KDM6B, KMT2A, KMT2C, KMT2E, MYSM1, NEK6, PHF2, PRMT1, SETD1A, SETD1B, SF3B1, SMARCA5, SMARCB1, SMYD3, UFL1, USP3, USP7, USP16 (Himeda et al., Molecular Therapy. 2018 Apr. 20, 26 (7)); Src family (such as Src, Yes, Fyn, and Fgr, Lck, Hck, Blk, Lyn, Frk, WO2019084499); Syk family (such as Syk, WO2019084499); AbN family (such as Abl1, WO2019084499); Tie family (such as Tie1, Tie2, TEK, WO2019084499); Flt family (such as VEGFR1, WO2019084499); CK1 (such as CK1d, CK1e, WO2019115711); ErbB family (such as Her1 (EGFR, ErbB1), Her2 (Neu, ErbB2), Her3 (ErbB3), and Her4 (ErbB4), WO2019084499); p38 (WO2019071147); Trk family (such as TrkA, TrkB, TrkC, WO2019084499); and PI3K family (such as ATM, ATR, PRKDC, mTOR, SMG1, TRRAP, WO2019084499).

In light of the above, in preferred embodiments the compound is for use in modulating BET protein activity; in other preferred embodiments the compound is for use in modulating β2-adrenergic receptor activity; in other preferred embodiments the compound is for use in modulating SMCHD1 activity; in other preferred embodiments the compound is for use in modulating PARP1 activity; in other preferred embodiments the compound is for use in modulating WNT signaling activity; in other preferred embodiments the compound is for use in modulating tankyrase activity; in other preferred embodiments the compound is for use in modulating PRC2/EZH2 activity; in other preferred embodiments the compound is for use in modulating SUV39H1 activity; in other preferred embodiments the compound is for use in modulating MBD2/NuRD complex activity; in other preferred embodiments the compound is for use in modulating MBD1/CAF-1 activity; in other preferred embodiments the compound is for use in modulating TRIM28 activity; in other preferred embodiments the compound is for use in modulating SETDB1 activity; in other preferred embodiments the compound is for use in modulating KDM1A activity; in other preferred embodiments the compound is for use in modulating SIN3 complex activity; in other preferred embodiments the compound is for use in modulating ASH1L activity; in other preferred embodiments the compound is for use in modulating BAP1 activity; in other preferred embodiments the compound is for use in modulating BAZ1A activity; in other preferred embodiments the compound is for use in modulating BAZ1 B activity; in other preferred embodiments the compound is for use in modulating BAZ2A activity; in other preferred embodiments the compound is for use in modulating BPTF activity; in other preferred embodiments the compound is for use in modulating BRD2 activity; in other preferred embodiments the compound is for use in modulating BRD3 activity; in other preferred embodiments the compound is for use in modulating BRD4 activity; in other preferred embodiments the compound is for use in modulating BRDT activity; in other preferred embodiments the compound is for use in modulating BRPF1 activity; in other preferred embodiments the compound is for use in modulating BRPF3 activity; in other preferred embodiments the compound is for use in modulating CARM1 activity; in other preferred embodiments the compound is for use in modulating KDM4A activity; in other preferred embodiments the compound is for use in modulating KDM4B activity; in other preferred embodiments the compound is for use in modulating KDM4C activity; in other preferred embodiments the compound is for use in modulating KDM4D activity; in other preferred embodiments the compound is for use in modulating KDM6A activity; in other preferred embodiments the compound is for use in modulating KDM6B activity; in other preferred embodiments the compound is for use in modulating KMT2A activity; in other preferred embodiments the compound is for use in modulating KMT2C activity; in other preferred embodiments the compound is for use in modulating KMT2E activity; in other preferred embodiments the compound is for use in modulating MYSM1 activity; in other preferred embodiments the compound is for use in modulating NEK6 activity; in other preferred embodiments the compound is for use in modulating PHF2 activity; in other preferred embodiments the compound is for use in modulating PRMT1 activity; in other preferred embodiments the compound is for use in modulating SETD1A activity; in other preferred embodiments the compound is for use in modulating SETD1B activity; in other preferred embodiments the compound is for use in modulating SF3B1 activity; in other preferred embodiments the compound is for use in modulating SMARCA5 activity; in other preferred embodiments the compound is for use in modulating SMARCB1 activity; in other preferred embodiments the compound is for use in modulating SMYD3 activity; in other preferred embodiments the compound is for use in modulating UFL1 activity; in other preferred embodiments the compound is for use in modulating USP3 activity; in other preferred embodiments the compound is for use in modulating USP7 activity; in other preferred embodiments the compound is for use in modulating USP16 activity; in other preferred embodiments the compound is for use in modulating Src family activity; in other preferred embodiments the compound is for use in modulating Syk family activity; in other preferred embodiments the compound is for use in modulating AbN family activity; in other preferred embodiments the compound is for use in modulating Tie family activity; in other preferred embodiments the compound is for use in modulating Flt family activity; in other preferred embodiments the compound is for use in modulating CK1 activity; in other preferred embodiments the compound is for use in modulating ErbB family activity; in other preferred embodiments the compound is for use in modulating p38 activity; in other preferred embodiments the compound is for use in modulating Trk family activity; in other preferred embodiments the compound is for use in modulating PI3K family activity. In this context, modulation of activity is preferably inhibition of activity. Modulation and inhibition can be assayed as described in the respective sources cited above.

### Formulation and Administration

The compositions comprising the compounds as described above, can be prepared as a medicinal or cosmetic preparation or in various other media, such as foods for humans or animals, including medical foods and dietary supplements. A “medical food” is a product that is intended for the specific dietary management of a disease or condition for which distinctive nutritional requirements exist. By way of example, not limitation, medical foods may include vitamin and mineral formulations fed through a feeding tube (referred to as enteral administration). A “dietary supplement” shall mean a product that is intended to supplement the human diet and is typically provided in the form of a pill, capsule, tablet or like formulation. By way of example, not limitation, a dietary supplement may include one or more of the following ingredients: vitamins, minerals, herbs, botanicals; amino acids, dietary substances intended to supplement the diet by increasing total dietary intake, and concentrates, metabolites, constituents, extracts or combinations of any of the foregoing. Dietary supplements may also be incorporated into food, including, but not limited to, food bars, beverages, powders, cereals, cooked foods, food additives and candies; or other functional foods designed to promote health or to prevent or halt the progression of a degenerative disease associated with DUX4 expression or activity.

The subject compounds and compositions may be compounded with other physiologically acceptable materials that can be ingested including, but not limited to, foods. In addition, or alternatively, the compositions as described herein may be administered orally in combination with (the separate) administration of food.

The compositions or compound according to the invention may be administered alone or in combination with other pharmaceutical or cosmetic agents and can be combined with a physiologically acceptable carrier thereof. In particular, the compounds described herein can be formulated as pharmaceutical or cosmetic compositions by formulation with additives such as pharmaceutically or physiologically acceptable excipients carriers, and vehicles. Suitable pharmaceutically or physiologically acceptable excipients, carriers and vehicles include processing agents and drug delivery modifiers and enhancers, such as, for example, calcium phosphate, magnesium stearate, talc, monosaccharides, disaccharides, starch, gelatin, cellulose, methyl cellulose, sodium carboxymethyl cellulose, dextrose, hydroxypropyl-P-cyclodextrin, polyvinylpyrrolidinone, low melting waxes, ion exchange resins, and the like, as well as combinations of any two or more thereof. Other suitable pharmaceutically acceptable excipients are described in “Remington's Pharmaceutical Sciences,” Mack Pub. Co., New Jersey (1991), and “Remington: The Science and Practice of Pharmacy,” Lippincott Williams & Wilkins, Philadelphia, 20th edition (2003), 21^{st} edition (2005) and 22^{nd} edition (2012), incorporated herein by reference.

Compositions for use according to the invention may be manufactured by processes well known in the art; e.g., by means of conventional mixing, dissolving, granulating, dragee-making, levigating, emulsifying, encapsulating, entrapping or lyophilizing processes, which may result in liposomal formulations, coacervates, oil-in-water emulsions, nanoparticulate/microparticulate powders, or any other shape or form. Compositions for use in accordance with the invention thus may be formulated in a conventional manner using one or more physiologically acceptable carriers comprising excipients and auxiliaries that facilitate processing of the active compounds into preparations which can be used pharmaceutically. Proper formulation is dependent on the route of administration chosen.

For injection, the compounds and compositions for use according to the invention may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

Oral and parenteral administration may be used where the compounds and compositions for use are formulated by combining them with pharmaceutically acceptable carriers well known in the art, or by using them as a food additive. Such strategies enable the compounds and compositions for use according to the invention to be formulated as tablets, pills, dragées, capsules, liquids, gels, syrups, slurries, suspensions and the like, for oral ingestion by a subject to be treated.

Preparations or pharmacological preparations for oral use may be made with the use of a solid excipient, optionally grinding the resulting mixture, and processing the mixture of granules, after adding suitable auxiliaries, if desired, to obtain tablets or dragée cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as cross-linked polyvinyl pyrrolidone, agar, or alginic acid or a salt thereof such as sodium alginate. Additionally, coformulations may be made with uptake enhancers known in the art.

Dragée cores are provided with suitable coatings. For this purpose, concentrated sugar solutions may be used, which may optionally contain gum arabic, talc, PVP, carbopol gel, polyethylene glycol, and/or titanium dioxide, lacquer solution, and suitable organic solvents or solvent mixtures. Polymethacrylates can be used to provide pH-responsive release profiles so as to pass the stomach. Dyestuffs or pigments may be added to the tablets or dragée coatings for identification or to characterize different combinations of active compound doses.

Compounds and compositions which can be administered orally include push-fit capsules made of gelatin, as well as soft, sealed capsules made of gelatin and a plasticizer, such as glycerol or sorbitol. The push-fit capsules may contain the active ingredients in admixture with a filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compounds and compositions for use according to the invention may be administered in the form of tablets or lozenges formulated in a conventional manner.

The compounds and compositions for use according to the invention may be formulated for parenteral administration by injection, e.g., by bolus injection or continuous infusion. In this way it is also possible to target a particular organ, tissue, tumor site, site of inflammation, etc. Formulations for infection may be presented in unit dosage form, e.g., in ampoules or in multi-dose container, with an added preservative. The compositions may take such forms as suspensions, solutions or emulsions in oily or aqueous vehicles, and may contain formulatory agents such as suspending, stabilizing and/or dispersing agents. This formulation is preferred because it enables specific targeting of muscle tissue.

Compositions for parenteral administration include aqueous solutions of the compositions in water soluble form. Additionally, suspensions may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compositions to allow for the preparation of highly concentrated solutions.

Alternatively, one or more components of the composition may be in powder form for constitution with a suitable vehicle, e.g., sterile pyrogen-free water, before use.

The compositions for use according to the invention may also be formulated in rectal compositions such as suppositories or retention enemas, e.g., containing conventional suppository bases such as cocoa butter or other glycerides.

In addition to the formulations described previously, the compounds and compositions for use according to the invention may also be formulated as a depot preparation. Such long acting formulations may be administered by implantation (for example subcutaneously or intramuscularly) or by intramuscular injection. Thus, for example, they may be formulated with suitable polymeric or hydrophobic materials (for example as an emulsion in an acceptable oil), or as part of a solid or semi-solid implant that may or may not be auto-degrading in the body, or ion exchange resins, or one or more components of the composition can be formulated as sparingly soluble derivatives, for example, as a sparingly soluble salt. Examples of suitable polymeric materials are known to the person skilled in the art and include PLGA and polylactones such as polycaproic acid.

The compositions for use according to the invention also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

The compositions for use according to the invention may also be comprised in a transdermal patch. Preferred transdermal patches for use according to the invention are selected from single-layer drug-in-adhesive patch, or multi-layer drug-in-adhesive patch, or reservoir patch, or matrix patch, or vapour patch.

Compositions for use according to the invention include compounds and compositions wherein the active ingredients are contained in an amount effective to achieve their intended purposes. More specifically, a therapeutically effective amount means an amount of compound effective to prevent, stabilize, alleviate, revert, or ameliorate causes or symptoms of disease, or prolong the survival, mobility, or independence of the subject being treated. Determination of a therapeutically effective amount is within the capability of those skilled in the art, especially in light of the detailed disclosure provided herein. For any compounds and compositions used in the invention, the therapeutically effective amount or dose can be estimated initially from cell culture assays, for example as exemplified herein. Dosage may vary within this range depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics” Ch. 1 p. 1). The amount of compound and compositions administered will, of course, be dependent on the subject being treated, on the subject's weight, the severity of the affliction, the manner of administration and the judgment of the prescribing physician.

A composition for use according to the invention may be supplied such that a compound for use according to the invention and one or more of the other components as defined herein are in the same container, either in solution, in suspension, or in powder form. A composition for use according to the invention may also be provided with all components provided separately from one another, for example to be mixed with one another prior to administration, or for separate or sequential administration. Various packaging options are possible and known to the ones skilled in the art, depending, among others, on the route and mechanism of administration. In light of the methods of administration described above, the invention provides a compound for use according to the invention, or a composition for use according to the invention, characterized in that it is administered orally, sublingually, intravascularly, intravenously, subcutaneously, transdermally, or optionally by inhalation; preferably orally.

An “effective amount” of a compound or composition is an amount which, when administered to a subject, is sufficient to reduce or eliminate either one or more symptoms of a disease, or to retard the progression of one or more symptoms of a disease, or to reduce the severity of one or more symptoms of a disease, or to suppress the manifestation of a disease, or to suppress the manifestation of adverse symptoms of a disease. An effective amount can be given in one or more administrations.

The “effective amount” of that may be combined with the carrier materials to produce a single dosage form will vary depending upon the host to which the active ingredient is administered and the particular mode of administration. The unit dosage chosen is usually fabricated and administered to provide a desired final concentration of the compound in the blood.

The effective amount (i.e. the effective total daily dose), preferably for adults, is herein defined as a total daily dose of about 0.01 to 2000 mg, or about 0.01 to 1000 mg, or about 0.01 to 500 mg, or about 5 to 1000 mg, or about 20 to 800 mg, or about 30 to 800 mg or about 30 to 700 mg, or about 20 to 700 mg or about 20 to 600 mg, or about 30 to 600 mg, or about 30 to 500 mg, about 30 to 450 mg or about 30 to 400 mg, or about 30 to 350 mg or about 30 to 300 mg or about 50 to 600 mg, or about 50 to 500 mg, or about 50 to 450 mg, or about 50 to 400 mg or about 50 to 300 mg, or about 50 to 250 mg, or about 100 to 250 mg or about 150 to 250 mg. In the most preferred embodiment, the effective amount is about 200 mg. In preferred embodiments, the invention provides a compound for use according to the invention, or a composition for use according to the invention, characterized in that it is administered to a subject in an amount ranging from 0.1 to 1500 mg/day, preferably from 0.1 to 1000 mg/day, more preferably from 0.1 to 400 mg/day, still more preferably from 0.25 to 150 mg/day, such as about 100 mg/day.

Alternatively, the effective amount of the compound, preferably for adults, preferably is administered per kg body weight. The total daily dose, preferably for adults, is therefore about 0.05 to about 40 mg/kg, about 0.1 to about 20 mg/kg, about 0.2 mg/kg to about 15 mg/kg, or about 0.3 mg/kg to about 15 mg/kg or about 0.4 mg/kg to about 15 mg/kg or about 0.5 mg/kg to about 14 mg/kg or about 0.3 mg/kg to about 14 mg/kg or about 0.3 mg/kg to about 13 mg/kg or about 0.5 mg/kg to about 13 mg/kg or about 0.5 mg/kg to about 11 mg/kg.

The total daily dose for children is preferably at most 200 mg. More preferably the total daily dose is about 0.1 to 200 mg, about 1 to 200 mg, about 5 to 200 mg about 20 to 200 mg about 40 to 200 mg, or about 50 to 200 mg. Preferably, the total daily dose for children is about 0.1 to 150 mg, about 1 to 150 mg, about 5 to 150 mg about 10 to 150 mg about 40 to 150 mg, or about 50 to 150 mg. More preferably, the total daily dose is about 5 to 100 mg, about 10 to 100 mg, about 20 to 100 mg about 30 to 100 mg about 40 to 100 mg, or about 50 to 100 mg. Even more preferably, the total daily dose is about 5 to 75 mg, about 10 to 75 mg, about 20 to 75 mg about 30 to 75 mg about 40 to 75 mg, or about 50 to 75 mg.

Alternative examples of dosages which can be used are an effective amount of the compounds for use according to the invention within the dosage range of about 0.1 μg/kg to about 300 mg/kg, or within about 1.0 μg/kg to about 40 mg/kg body weight, or within about 1.0 μg/kg to about 20 mg/kg body weight, or within about 1.0 μg/kg to about 10 mg/kg body weight, or within about 10.0 μg/kg to about 10 mg/kg body weight, or within about 100 μg/kg to about 10 mg/kg body weight, or within about 1.0 mg/kg to about 10 mg/kg body weight, or within about 10 mg/kg to about 100 mg/kg body weight, or within about 50 mg/kg to about 150 mg/kg body weight, or within about 100 mg/kg to about 200 mg/kg body weight, or within about 150 mg/kg to about 250 mg/kg body weight, or within about 200 mg/kg to about 300 mg/kg body weight, or within about 250 mg/kg to about 300 mg/kg body weight. Other dosages which can be used are about 0.01 mg/kg body weight, about 0.1 mg/kg body weight, about 1 mg/kg body weight, about 10 mg/kg body weight, about 20 mg/kg body weight, about 30 mg/kg body weight, about 40 mg/kg body weight, about 50 mg/kg body weight, about 75 mg/kg body weight, about 100 mg/kg body weight, about 125 mg/kg body weight, about 150 mg/kg body weight, about 175 mg/kg body weight, about 200 mg/kg body weight, about 225 mg/kg body weight, about 250 mg/kg body weight, about 275 mg/kg body weight, or about 300 mg/kg body weight.

Compounds or compositions for use according to the present invention may be administered in a single daily dose, or the total daily dosage may be administered in divided dosage of two, three or four times daily.

In a preferred embodiment of the invention, “subject”, “individual”, or “patient” is understood to be an individual organism, preferably a vertebrate, more preferably a mammal, even more preferably a primate and most preferably a human.

In a further preferred embodiment of the invention, the human is an adult, e.g. a person that is 18 years or older. In addition, it is herein understood that the average weight of an adult person is 62 kg, although the average weight is known to vary between countries. In another embodiment of the invention the average weight of an adult person is therefore between about 50-90 kg. It is herein understood that the effective dose as defined herein is not confined to subjects having an average weight. Preferably, the subject has a BMI (Body Mass Index) between 18.0 to 40.0 kg/m², and more preferably a BMI between 18.0 to 30.0 kg/m².

Alternatively, the subject to be treated is a child, e.g. a person that is 17 years or younger. In addition, the subject to be treated may be a person between birth and puberty or between puberty and adulthood. It is herein understood that puberty starts for females at the age of 10-11 years and for males at the age of 11-12 year. Furthermore, the subject to be treated may be a neonate (first 28 days after birth), an infant (0-1 year), a toddler (1-3 years), a preschooler (3-5 years); a school-aged child (5-12 years) or an adolescent (13-18 years).

To maintain an effective range during treatment, the compound or composition may be administered once a day, or once every two, three, four, or five days. However preferably, the compound may be administered at least once a day. Hence in a preferred embodiment, the invention pertains to a compound for use according to the invention, or a composition for use according to the invention, characterized in that it is administered to a subject 4, 3, 2, or 1 times per day or less, preferably 1 time per day. The total daily dose may be administered as a single daily dose. Alternatively, the compound is administered at least twice daily. Hence, the compound as defined herein may be administered once, twice, three, four or five times a day. As such, the total daily dose may be divided over the several doses (units) resulting in the administration of the total daily dose as defined herein. In a preferred embodiment, the compound is administered twice daily. It is further understood that the terms “twice daily”, “bid” and “bis in die” can be used interchangeable herein.

In a preferred embodiment, the total daily dose is divided over several doses per day. These separate doses may differ in amount. For example, for each total daily dose, the first dose may have a larger amount of the compound than the second dose or vice versa. However preferably, the compound is administered in similar or equal doses. Therefore, in a most preferred embodiment, the compound is administered twice daily in two similar or equal doses.

In a further preferred embodiment of the invention, the total daily dose of the compound as defined herein above is administered in at least two separate doses. The interval between the administration of the at least two separate doses is at least about 0.5, 1, 2, 3, 4, 5, 6, 7, 8, 9, 10, 11 or 12 hours, preferably the interval between the at least two separate doses is at least about 4, 5, 6, 7, 8, 9, 10, 11 or 12 hours and more preferably the interval between the at least two separate doses is at least about 8, 9, 10, 11 or 12 hours.

### Use

In one aspect of the invention, the use is provided of either a compound of general formula I, or of a composition according to the invention. Said use is for the treatment of a disease or condition associated with DUX4 expression of a subject in need thereof, and comprises administration to the subject of an effective dose of a compound of general formula I or composition according to the invention, wherein the compound of general formula I or composition are as defined earlier herein.

In one embodiment of this aspect, the use is provided of either a compound of general formula I, or of a composition according to the invention. Said use is for the treatment of muscular dystrophy or cancer in a subject in need thereof, and comprises administration to the subject of an effective dose of a compound of general formula I or composition according to the invention, wherein the compound of general formula I or composition are as defined earlier herein. Further features and definitions are preferably as defined elsewhere herein, particularly for diseases or conditions to be treated, or for uses such as use of the compounds for the promotion of myogenic fusion and/or for the promotion of myogenic differentiation, which can be in vitro, in vivo, or ex vivo.

### Method

One aspect of the invention provides an in vivo, in vitro, or ex vivo method for reducing DUX4 expression, the method comprising the step of contacting a cell with a compound of general formula I as defined earlier herein, or with a composition as defined earlier herein. Preferably, said method is for treating a disease or condition associated with DUX4 expression, such as a muscular dystrophy or cancer, most preferably said disease or condition is facioscapulohumeral muscular dystrophy (FSHD). The method preferably comprises use as defined earlier herein. Preferred methods comprise contacting a cell with a compound of general formula I or composition as defined earlier herein. In the context of the invention, contacting a cell with a compound of general formula I or a composition can comprise adding such a compound of general formula I or composition to a medium in which a cell is cultured. Contacting a cell with a compound of general formula I or a composition can also comprise adding such a compound of general formula I or composition to a medium, buffer, or solution in which a cell is suspended, or which covers a cell. Other preferred methods of contacting a cell comprise injecting a cell with a compound of general formula I or composition, or exposing a cell to a material comprising a compound of general formula I or composition according to the invention. Further methods for administration are defined elsewhere herein. Preferred cells are cells known to express DUX4, cells suspected of expressing DUX4, or cells known to be affected by a disease or condition as defined earlier herein.

In one embodiment of this aspect, the method is an in vitro method. In a further embodiment of this aspect, the method is an ex vivo method. In a further embodiment of this aspect, the method is an in vivo method. In a preferred embodiment of this aspect, the method is an in vitro or an ex vivo method.

Within the embodiments of this aspect, the cell may be a cell from a sample obtained from a subject. Such a sample may be a sample that has been previously obtained from a subject. Within the embodiments of this aspect, samples may have been previously obtained from a human subject. Within the embodiments of this aspect, samples may have been obtained from a non-human subject. In a preferred embodiment of this aspect, obtaining the sample is not part of the method according to the invention.

In preferred embodiments, the method according to the invention is a method for reducing DUX4 expression in a subject in need thereof, the method comprising the step of administering an effective amount of a compound of general formula I as defined earlier herein, or a composition as defined earlier herein. In more preferred embodiments, the method is for the treatment of a disease or condition associated with DUX4 expression, preferably a muscular dystrophy or cancer, most preferably said disease or condition is facioscapulohumeral muscular dystrophy (FSHD). Further features and definitions are preferably as defined elsewhere herein. The method can be for any use, preferably for any non-medical use as described herein, such as for the promotion of myogenic fusion and/or for the promotion of myogenic differentiation, which can be in vitro, in vivo, or ex vivo.

### General Definitions

In this document and in its claims, the verb “to comprise” and its conjugations is used in its non-limiting sense to mean that items following the word are included, but items not specifically mentioned are not excluded. In addition, the verb “to consist” may be replaced by “to consist essentially of” meaning that a combination or a composition as defined herein may comprise additional component(s) than the ones specifically identified, said additional component(s) not altering the unique characteristic of the invention. In addition, reference to an element by the indefinite article “a” or “an” does not exclude the possibility that more than one of the element is present, unless the context clearly requires that there be one and only one of the elements. The indefinite article “a” or “an” thus usually means “at least one”.

When a structural formula or chemical name is understood by the skilled person to have chiral centers, yet no chirality is indicated, for each chiral center individual reference is made to all three of either the racemic mixture, the pure R enantiomer, or the pure S enantiomer.

Whenever a parameter of a substance is discussed in the context of this invention, it is assumed that unless otherwise specified, the parameter is determined, measured, or manifested under physiological conditions. Physiological conditions are known to a person skilled in the art, and comprise aqueous solvent systems, atmospheric pressure, pH-values between 6 and 8, a temperature ranging from room temperature to about 37° C. (from about 20° C. to about 40° C.), and a suitable concentration of buffer salts or other components.

The use of a substance as a medicament as described in this document can also be interpreted as the use of said substance in the manufacture of a medicament. Similarly, whenever a substance is used for treatment or as a medicament, it can also be used for the manufacture of a medicament for treatment. Products for use as a medicament described herein can be used in methods of treatments, wherein such methods of treatment comprise the administration of the product for use. Compound of general formula I or compositions according to this invention are preferably for use in methods or uses according to this invention.

Throughout this application, expression is considered to be the transcription of a gene into functional mRNA, leading to a polypeptide such as an enzyme or transcription factor or for example DUX4 polypeptide. A polypeptide can assert an effect or have an activity. In this context, increased or decreased expression or activity of a polypeptide can be considered an increased or decreased level of mRNA encoding said polypeptide, an increased or decreased level or amount of polypeptide molecules, or an increased or decreased total activity of said polypeptide molecules. Preferably, an increased or decreased expression of a polypeptide results in an increased or decreased activity of said polypeptide, respectively, which can be caused by increased or decreased levels or amounts of polypeptide molecules. More preferably, a reduction of DUX4 expression is a reduction of transcription of a DUX4 gene, destabilisation or degradation of DUX4 mRNA, reduction of the amount of DUX4 polypeptide molecules, reduction of DUX4 polypeptides molecule activity, destabilisation or degradation of DUX4 polypeptide, or combinations thereof. A destabilized mRNA leads to lower expression of its encoded polypeptide, possibly it cannot lead to such expression. A degraded mRNA is destroyed and cannot lead to expression of its encoded polypeptide. A destabilized polypeptide asserts less of an effect or has lower activity than the same polypeptide that has not been destabilized, possibly it asserts no effect or has no activity. A destabilized polypeptide can be denatured or misfolded. A degraded polypeptide is destroyed and does not assert an effect or have an activity.

In the context of this invention, a decrease or increase of a parameter to be assessed means a change of at least 5% of the value corresponding to that parameter. More preferably, a decrease or increase of the value means a change of at least 10%, even more preferably at least 20%, at least 30%, at least 40%, at least 50%, at least 70%, at least 90%, or 100%. In this latter case, it can be the case that there is no longer a detectable value associated with the parameter.

The word “about” or “approximately” when used in association with a numerical value (e.g. about 10) preferably means that the value may be the given value (of 10) more or less 5% of the value.

Each embodiment as identified herein may be combined together unless otherwise indicated. The invention has been described above with reference to a number of embodiments. A skilled person could envision trivial variations for some elements of the embodiments. These are included in the scope of protection as defined in the appended claims. All patent and literature references cited are hereby incorporated by reference in their entirety.

### EXAMPLES

### Example 1—Synthesis of Compounds of General Formula (I)

### 1.1—General Methods

All reagents, for which the synthesis is not described in the experimental part, are either commercially available, or are known compounds or may be formed from known compounds by known methods by a person skilled in the art. The compounds and intermediates produced according to the methods of the invention may require purification. Purification of organic compounds is well known to a person skilled in the art and there may be several ways of purifying the same compound. In some cases, no purification may be necessary. In some cases, the compounds may be purified by crystallization. In some cases, impurities may be stirred out using a suitable solvent. In some cases, the compounds may be purified by chromatography, particularly flash column chromatography, using for Compound prepacked silica gel cartridges, e.g. Biotage SNAP cartridges KP-Sil® or KP-NH® in combination with a Biotage autopurifier system (SP4® or Isolera Four®) and eluents such as gradients of hexane/EtOAc or DCM/MeOH. In some cases, the compounds may be purified by preparative HPLC using methods as described. Purification methods as described herein may provide compounds of the present invention which possess a sufficiently basic or acidic functionality in the form of a salt, such as, in the case of a compound of the present invention which is sufficiently basic, a trifluoroacetate or formate salt, or, in the case of a compound of the present invention which is sufficiently acidic, an ammonium salt. A salt of this type can either be transformed into its free base or free acid form, respectively, by various methods known to a person skilled in the art, or be used as salts in subsequent biological assays. It is to be understood that the specific form of a compound of the present invention as isolated and as described herein is not necessarily the only form in which said compound can be applied to a biological assay in order to quantify the specific biological activity.

All the starting materials and reagents are commercially available and were used as is. 1H Nuclear magnetic resonance (NMR) spectroscopy was carried out using a Bruker instrument operating at 400 MHz or 500 MHz as specified, using the stated solvent at around room temperature unless otherwise stated. In all cases, NMR data were consistent with the proposed structures. Characteristic chemical shifts (6) are given in parts-per-million using conventional abbreviations for designation of major peaks: e.g. s, singlet; d, doublet; t, triplet; q, quartet; dd, doublet of doublets; dt, doublet of triplets; m, multiplet; br, broad. Preparative HPLC purification was performed by reverse phase HPLC using a Waters Fractionlynx preparative HPLC system (2525 pump, 2996/2998 UV/VIS detector, 2767 liquid handler) or an equivalent HPLC system such as a Gilson Trilution UV directed system. The Waters 2767 liquid handler acted as both auto-sampler and fraction collector. The columns used for the preparative purification of the compounds were a Waters Sunfire OBD Phenomenex Luna Phenyl Hexyl (10 μm 21.2×150 mm, 10 μm) or Waters Xbridge Phenyl (10 μm 19×150 mm, 5 μm). Appropriate focused gradients were selected based on acetonitrile and methanol solvent systems under either acidic or basic conditions. The modifiers used under acidic/basic conditions were formic acid (0.1% V/V) and ammonium bicarbonate (10 mM) respectively. The purification was controlled by Waters Fractionlynx software through monitoring at 210-400 nm, and triggered a threshold collection value at 260 nm and, when using the Fractionlynx, the presence of target molecular ion as observed under APi conditions. Collected fractions were analysed by LCMS (Waters Acquity systems with Waters SQD). Normal phase flash column chromatography was performed utilizing a Biotage Isolera system. The silica gel columns were purchased from either Interchim or Biotage. The mobile phase was either ethyl acetate in hexanes or methanol in dichloromethane with various ratios, and the fraction collection was triggered by UV absorbance at 254 nm. Analytical high-performance liquid chromatography-mass spectrometry (HPLC-MS) was performed utilizing HP or Waters DAD+Micromass ZQ, single quadrupole LC-MS or Quattro Micro LC-MS-MS. Method 1: The RP-HPLC column was Phenomenex Luna 5 μm C18 (2), (100×4.6 mm). Mobile phase 5-95% acetonitrile in water (0.1% formic acid) gradient, flow rate 2.0 mL/min, and 6.5 min run time. Method 2: The RP-HPLC column was Waters Xterra MS 5 μm C18, 100×4.6 mm. Mobile phase 5-95% acetonitrile in water (10 mM ammonium bicarbonate (ammonium hydrogen carbonate)). Chemical names were generated using the JChem for Excel naming software (Version 16.7.1800.1000) by Chem Axon Ltd. In some cases, generally accepted names of commercially available reagents were used in place of names generated by the naming software.

Analytical LC-MS methods: Method A Column: Phenomenex Kinetix-XB C18 1.2×100 mm, 1.7 μm; eluent A: water+0.1 vol % formic acid, eluent B: acetonitrile+0.1 vol % formic acid; gradient: 0-5.3 min 5-100% B, 5.3-5.8 min 100% B, 5.8-5.82 min 100-5% B, 5.82-7.00 min 5% B; flow 0.6 mL/min; injection volume 1 μL; temperature: 40° C.; UV scan: 215 nm; PDA Spectrum range: 200-400 nm step: 1 nm; MSD signal settings-scan pos: 150-850. —Method B Column: Waters UPLC® BEHTM C18 2.1×100 mm, 1.7 μm; eluent A: 2 mM ammonium bicarbonate, buffered to pH10, eluent B: acetonitrile; gradient: 0-5.3 min 5-100% B, 5.3-5.8 min 100% B, 5.8-5.82 min 100-5% B, 5.8-7.0 min 5% B; flow 0.6 mL/min; injection volume 2 μL; temperature: 40° C.; UV scan: 215 nm; PDA Spectrum range: 200-400 nm step: 1 nm; MSD signal settings-scan pos: 150-850. —Method C Column: Phenomenex Gemini-NX C18 2.01×100 mm, 3 μm; eluent A: 2 mM ammonium bicarbonate, buffered to pH10, eluent B: acetonitrile; gradient: 0-5.5 min 5-100% B, 5.5-5.9 min 100% B, 5.9-5.92 min 100-5% B, 5.92-7.00 min 5% B; flow 0.6 mL/min; injection volume 3 μL; temperature: 40° C.; UV scan: 215 nm; PDA Spectrum range: 210-400 nm step: 1 nm; MSD signal settings-scan pos: 150-850. —Method D Column: Waters Atlantis dC18 2.1×100 mm, 3 μm eluent A: water+0.1 vol % formic acid, eluent B: acetonitrile+0.1 vol % formic acid; gradient: 0-5.0 min 5-100% B, 5.0-5.4 min 100% B, 5.4-5.42 min 100-5% B, 5.42-7.00 min 5% B; flow 0.6 mL/min; injection volume 3 μL; temperature: 40° C.; UV scan: 215 nm; PDA Spectrum range: 200-400 nm step: 1 nm; MSD signal settings-scan pos: 150-1000. —Method E Column: Kinetex Core-Shell C18 2.1×50 mm, 5 μm eluent A: water+0.1 vol % formic acid, eluent B: acetonitrile+0.1 vol % formic acid; gradient: 0-1.2 min 5-100% B, 1.3-1.3 min 100% B, 1.3-1.31 min 100-5% B, 1.31-1.65 min 5% B; flow 1.2 mL/min; injection volume 3 μL; temperature: 40° C.; UV scan: 215 nm; PDA Spectrum range: 210-420 nm step: 1 nm; MSD signal settings-scan pos: 100-1000. —Method F Column: Waters UPLCO CSHTM C18 2.1×100 mm, 1.7 μm; eluent A: water+0.1 vol % formic acid, eluent B: acetonitrile+0.1 vol % formic acid; gradient: 0-1.1 min 5-100% B, 1.1-1.35 min 100% B, 1.35-1.4 min 100-5% B, 1.4-1.5 min 5% B; flow 0.9 mL/min; injection volume 2 μL; temperature: 40° C.; UV scan: 215 nm; PDA Spectrum range: 200-400 nm step: 1 nm; MSD signal settings-scan pos: 150-850. —Method G Column: Phenomenex Gemini-NX C18 2.0×50 mm, 3 μm; eluent A: 2 mM ammonium hydroxide, buffered to pH10, eluent B: acetonitrile; gradient: 0-1.8 min 1-100% B, 1.8-2.1 min 100% B, 2.1-2.3 min 100-1% B; flow 1 mL/min; injection volume 3 μL; temperature: 40° C.; UV scan: 215 nm; PDA Spectrum range: 210-420 nm step: 1 nm; MSD signal settings-scan pos: 150-850. —Method H Column: Waters UPLCO BEHTM C18 2.1×30 mm, 1.7 μm; eluent A: 2 mM ammonium bicarbonate, buffered to pH10, eluent B: acetonitrile; gradient: 0-0.75 min 5-100% B, 0.75-0.85 min 100% B, 0.85-0.9 min 100-5% B, 0.9-1.0 min 5% B; flow 1 mL/min; injection volume 2 μL; temperature: 40° C.; UV scan: 215 nm; PDA Spectrum range: 200-400 nm step: 1 nm; MSD signal settings-scan pos: 100-1000. Method/Column: Waters UPLCO BEHTM C18 2.1×30 mm, 1.7 μm; eluent A: 2 mM ammonium bicarbonate, buffered to pH10, eluent B: acetonitrile; gradient: 0-1.1 min 1-100% B, 1.1-1.35 min 100% B, 1.35-1.40 min 100-1% B, 1.40-1.8 min 1% B; flow 1 mL/min; injection volume 1 μL; temperature: 40° C.; UV scan: 215 nm; PDA Spectrum range: 200-400 nm step: 1 nm; MSD signal settings-scan pos: 100-1000. Method J Column: Waters UPLCO BEHTM C18 2.1×50 mm, 1.7 μm; eluent A: water+0.1 vol % formic acid, eluent B: acetonitrile+0.1 vol % formic acid; gradient: 0-1.1 min 5-100% B, 1.1-1.35 min 100% B, 1.35-1.4 min 100-5% B, 1.4-1.5 min 5% B; flow 0.9 mL/min; injection volume 1 μL; temperature: 40° C.; UV scan: 215 nm; PDA Spectrum range: 200-400 nm step: 1 nm; MSD signal settings-scan pos: 100-1000.

Purification Methods:

Biotage Isolera™ chromatography system (see www.biotage.com/product-area/flash-purification) using pre-packed silica and pre-packed modified silica cartridges.

Preparative HPLC, Method A1: Instrument: pump: Gilson 331 & 332; auto injector: Gilson GX281; UV detector: Gilson 159; collector: Gilson GX281 or pump: Gilson 333 & 334; auto injector: Gilson GX281; UV detector: Gilson 155; collector: Gilson GX281; Column: Waters Xbridge C18 30×100 mm, 10 μm; eluent A: water+0.2 vol % ammonium hydroxide, eluent B: acetonitrile+0.2 vol % ammonium hydroxide; gradient: 0-0.8 min 10% B, 0.8-14.5 min 10-95% B, 14.5-16.7 min 95% B; flow 40 mL/min; injection volume 1500 μL; temperature: 25° C.; UV scan: 215 nm.

Preparative HPLC, Method A2: Instrument: pump: Gilson 331 & 332; auto injector: Gilson GX281; UV detector: Gilson 159; collector: Gilson GX281 or pump: Gilson 333 & 334; auto injector: Gilson GX281; UV detector: Gilson 155; collector: Gilson GX281; Column: Waters Xbridge C18 30×100 mm, 10 μm; eluent A: water+0.2 vol % ammonium hydroxide, eluent B: acetonitrile+0.2 vol % ammonium hydroxide; gradient: 0-1.1 min 30% B, 1.1-10.05 min 30-95% B, 10.05-11.5 min 95% B; flow 40 mL/min; injection volume 1500 μL; temperature: 25° C.; UV scan: 215 nm.

Preparative HPLC, Method B1: Instrument pump: Gilson 331 & 332; auto injector: Gilson GX281; UV detector: Gilson 159; collector: Gilson GX281; Column: Waters Sunfire C18 30×100 mm, 10 μm; eluent A: water+0.1 vol % formic acid, eluent B: acetonitrile+0.1 vol % formic acid; gradient: 0-0.8 min 10% B, 0.8-14.5 min 5-95% B, 14.5-16.7 min 95% B; flow 40 mL/min; injection volume 1500 μL; temperature: 25° C.; UV scan: 215 nm.

Preparative HPLC, Method B2: Instrument pump: Gilson 331 & 332; auto injector: Gilson GX281; UV detector: Gilson 159; collector: Gilson GX281; Column: Waters Sunfire C18 30×100 mm, 10 μm; eluent A: water+0.1 vol % formic acid, eluent B: acetonitrile+0.1 vol % formic acid; gradient: 0-1.1 min 30% B, 1.1-10.05 min 30-95% B, 10.05-11.5 min 95% B; flow 40 mL/min; injection volume 1500 μL; temperature: 25° C.; UV scan: 215 nm.

### Example 1.2—Synthesis of Intermediates

### Synthesis of tert-butyl 2-[4-(4-fluorophenyl)-1H-imidazol-1-yl]acetate/Intermediate 1-1

NaH (60%, 407 mg, 10.2 mmol) was added portionwise to an ice-cold solution of 4-(4-fluorophenyl)-1H-imidazole (1.50 g, 9.25 mmol) in THF (40 mL). The reaction was stirred for 15 min then tert-butyl bromoacetate (1.5 mL, 10.2 mmol) was added slowly. The reaction was stirred for 90 min then quenched into water. The aqueous layer was extracted into EtOAc, washed with brine, dried over MgSO₄ and concentrated in vacuo. The residue was purified by flash chromatography (50 g, silica), eluting with 20-80% EtOAc/heptane to yield the title compound (2.0 g, 78% yield). 1H NMR (400 MHz, Chloroform-d) δ 7.78-7.73 (m, 2H), 7.53 (d, J=1.2 Hz, 1H), 7.20 (d, J=1.3 Hz, 1H), 7.08 (t, J=8.8 Hz, 2H), 4.62 (s, 2H), 1.51 (s, 9H). LCMS (Analytical Method E) Rt=0.92 min, MS (ESIpos): m/z 277.0 [M+H]+, Purity=99%.

### Synthesis of tert-butyl 2-[5-bromo-4-(4-fluorophenyl)-1H-imidazol-1-yl]acetate/Intermediate 1-2

NBS (1.42 g, 7.96 mmol) was added to an ice-cold solution of tert-butyl 2-[4-(4-fluorophenyl)imidazol-1-yl]acetate (Intermediate 1-1) (2.00 g, 7.24 mmol) in DCM (50 mL). The reaction was stirred for 90 min then quenched into water. The aqueous layer was extracted into DCM (3×), the combined organics washed with brine, dried over MgSO₄ and concentrated in vacuo. The residue was purified by flash chromatography (50 g, silica), eluting with 0-70% EtOAc/heptane to yield the title compound as a white solid (1.96 g, 76% yield). 1H NMR (500 MHz, Chloroform-d) δ 7.93 (dd, J=8.9, 5.4 Hz, 2H), 7.67 (s, 1H), 7.10 (t, J=8.8 Hz, 2H), 4.62 (s, 2H), 1.49 (s, 9H). LCMS (Analytical Method E) Rt=1.22 min, MS (ESIpos): m/z 354.9, 356.9.0 [M+H]+, Purity=96%.

### Synthesis of tert-butyl 2-[4-(4-fluorophenyl)-5-(pyridin-4-yl)-1H-imidazol-1-yl]acetate/Intermediate 1-3

A mixture of tert-butyl 2-[5-bromo-4-(4-fluorophenyl)imidazol-1-yl]acetate (Intermediate 1-2) (500 mg, 1.41 mmol), pyridin-4-ylboronic acid (173 mg, 1.41 mmol), Pd(PPh₃)₄ (81 mg, 0.0704 mmol) and 2 M Na₂CO₃ (3.5 mL, 7.04 mmol) in DME (13.8 mL) was degassed by sparging with nitrogen. The mixture was heated to 100° C. under microwave irradiation for 2.5 h, then heated to 110° C. under microwave irradiation for 1.5 h. The reaction was cooled and quenched into water. The aqueous layer was extracted into EtOAc, washed with brine, dried over MgSO₄ and concentrated in vacuo. The residue was purified by flash chromatography (25 g, silica), eluting with 0-100% EtOAc/heptane to afford the title compound (410 mg, 66% yield). 1H NMR (400 MHz, Chloroform-d) δ 8.71-8.67 (m, 2H), 7.64 (s, 1H), 7.41 (dd, J=8.9, 5.4 Hz, 2H), 7.25-7.22 (m, 2H), 6.93 (t, J=8.8 Hz, 2H), 4.47 (s, 2H), 1.39 (s, 9H). LCMS (Analytical Method E) Rt=0.97 min, MS (ESIpos): m/z 354.1 [M+H]+, Purity=80%.

### Synthesis of 2-[4-(4-fluorophenyl)-5-(pyridin-4-yl)-1H-imidazol-1-yl]acetic acid/Intermediate 1

TFA (2.2 mL, 29.0 mmol) was added to a solution of tert-butyl 2-[4-(4-fluorophenyl)-5-(4-pyridyl)imidazol-1-yl]acetate (Intermediate 1-3) (410 mg, 1.16 mmol) in DCM (8 mL). The reaction was stirred for 3 h. Additional TFA (2.2 mL, 29.0 mmol) was added and stirring continued for 2 h. The mixture was concentrated in vacuo and the residue repeatedly taken up in toluene and concentrated in vacuo to yield the title compound as a TFA salt (600 mg, 70% yield), which was used in the next step without further purification. 1H NMR (500 MHz, DMSO-d6) δ 8.80-8.76 (m, 2H), 8.51 (s, 1H), 7.56 (dd, J=4.8, 1.5 Hz, 2H), 7.43-7.38 (m, 2H), 7.20 (t, J=8.9 Hz, 2H), 4.97 (s, 2H). LCMS (Analytical Method E) Rt=0.70 min, MS (ESIpos): m/z 298.0 [M+H]+, Purity=72%.

### Synthesis of tert-butyl 2-[4-(4-chlorophenyl)-1H-imidazol-1-yl]acetate/Intermediate 2-1

NaH (60%, 1168 mg, 29.2 mmol) was added portionwise to an ice-cold solution of 4-(4-chlorophenyl)-1H-imidazole (4.99 g, 26.5 mmol) in anhydrous THF (118 mL). The reaction was stirred for 15 min then tert-butyl bromoacetate (4.3 mL, 29.2 mmol) was added slowly. The reaction was stirred for 90 min. The reaction was slowly quenched with water. The aqueous layer was extracted into EtOAc (3×), the combined organics washed with brine, dried over MgSO₄ and concentrated in vacuo. The residue was purified by flash chromatography (100 g, silica), eluting with 0-80% EtOAc/heptane to afford the title compound (6.5 g, 85% yield). 1H NMR (500 MHz, Chloroform-d) 7.75-7.70 (m, 2H), 7.54 (d, J=1.1 Hz, 1H), 7.37-7.33 (m, 2H), 7.25 (d, J=1.3 Hz, 1H), 4.63 (s 2H), 1.51 (s, 9H). LCMS (Analytical Method E) Rt=0.77 min, MS (ESIpos): m/z 293.0 [M+H]+, Purity=100%.

### Synthesis of tert-butyl 2-[5-bromo-4-(4-chlorophenyl)-1H-imidazol-1-yl]acetate/Intermediate 2-2

NBS (4.35 g, 24.4 mmol) was added to an ice-cold solution of tert-butyl 2-[4-(4-chlorophenyl)imidazol-1-yl]acetate (Intermediate 2-1) (6.50 g, 22.2 mmol) in DCM (150 mL), and the reaction was stirred for 90 min. The mixture was then quenched into water, extracted into DCM (2×), dried over MgSO₄, filtered and concentrated in vacuo. The residue was purified by flash chromatography (100 g, silica) eluting with 0-40% EtOAc/heptane to yield the title compound (6.3 g, 76% yield). 1H NMR (400 MHz, Chloroform-d) δ 7.97-7.91 (m, 2H), 7.70 (s, 1H), 7.43-7.37 (m, 2H), 4.65 (s, 2H), 1.51 (s, 9H). LCMS (Analytical Method E) Rt=1.09 min, MS (ESIpos): m/z 371.0 [M+H]+, Purity=100%.

### Synthesis of tert-butyl 2-[4-(4-chlorophenyl)-5-(pyridin-4-yl)-1H-imidazol-1-yl]acetate/Intermediate 2-3

A mixture of tert-butyl 2-[5-bromo-4-(4-chlorophenyl)imidazol-1-yl]acetate (Intermediate 2-2) (1.00 g, 2.69 mmol), pyridin-4-yl boronic acid (331 mg, 2.69 mmol), Pd(PPh₃)₄ (155 mg, 0.135 mmol) and 2 M Na₂CO₃ (4.0 mL, 8.07 mmol) in DME (15 mL) was degassed by sparging with nitrogen. The mixture was heated to 125° C. under microwave irradiation for 3 h. Additional pyridin-4-yl boronic acid (165 mg, 1.85 mmol) was added and the mixture heated at 125° C. under microwave irradiation for 1.5 h. The reaction was cooled and quenched into water. The aqueous layer was extracted into EtOAc, washed with brine, dried over MgSO₄ and concentrated in vacuo. The residue was purified by flash chromatography (25 g, silica) eluting with 0-10% MeOH/DCM followed by another flash chromatography (25 g, silica) eluting with 0-70% EtOAc/heptane to yield the title compound as an off-white solid (669 mg, 56% yield). 1H NMR (400 MHz, Chloroform-d) δ 8.76-8.67 (m, 2H), 7.42-7.37 (m, 2H), 7.29-7.21 (m, 4H), 4.48 (s, 2H), 1.41 (s, 9H). LCMS (Analytical Method F) Rt=0.81 min, MS (ESIpos): m/z 370.2 [M+H]+, Purity=83%.

### Synthesis of 2-[4-(4-chlorophenyl)-5-(pyridin-4-yl)-1H-imidazol-1-yl]acetic acid/Intermediate 2a

TFA (5.5 mL, 74.6 mmol) was added to a solution of tert-butyl 2-[4-(4-chlorophenyl)-5-(4-pyridyl)imidazol-1-yl]acetate (Intermediate 2-3) (665 mg, 1.49 mmol) in DCM (11 mL) and the resulting mixture stirred at RT overnight. The solvent was evaporated under reduced pressure and Et₂O was added and evaporated multiple times to yield the title compound as a TFA salt (985 mg, 87% yield). 1H NMR (400 MHz, DMSO-d6) δ 8.81-8.75 (m, 2H), 8.34 (s, 1H), 7.57-7.54 (m, 2H), 7.39 (s, 4H), 4.94 (s, 2H). LCMS (Analytical Method F) Rt=0.51 min, MS (ESIpos): m/z 314.1 [M+H]+, Purity=71%.

### Synthesis of sodium 2-[4-(4-chlorophenyl)-5-(pyridin-4-yl)-1H-imidazol-1-yl]acetate/Intermediate 2b

A mixture of tert-butyl 2-[5-bromo-4-(4-chlorophenyl)imidazol-1-yl]acetate (Intermediate 2-2) (1.50 g, 4.04 mmol), pyridin-4-yl boronic acid (595 mg, 4.84 mmol), Pd(PPh₃)₄ (233 mg, 0.202 mmol) and 2 M Na₂CO₃ (10 mL, 20.2 mmol) in DME (30 mL) was degassed by sparging with nitrogen. The mixture was heated at 95° C. overnight. The reaction was cooled and quenched into water and both layers were separated. The aqueous layer was evaporated and the solid triturated with IPA. The IPA solution was collected by decanting the salts, and evaporated under reduced pressure to yield the title compound (925 mg, 41% yield). 1H NMR (500 MHz, DMSO-d6) δ 8.66-8.57 (m, 2H), 7.74 (s, 1H), 7.39-7.27 (m, 6H), 4.11 (s, 2H). LCMS (Analytical Method F) Rt=0.50 min, MS (ESIpos): m/z 314.1 [M+H]+, Purity=60%.

### Synthesis of tert-butyl 2-[4-(4-fluorophenyl)-5-{1H-pyrrolo[2,3-b]pyridin-4-yl}-1H-imidazol-1-yl]acetate/Intermediate 3-1

4-(4,4,5,5-tetramethyl-1,3,2-dioxaborolan-2-yl)-1H-pyrrolo[2,3-b]pyridine (200 mg, 0.819 mmol), tert-butyl 2-[5-bromo-4-(4-fluorophenyl)imidazol-1-yl]acetate (Intermediate 1-2) (250 mg, 0.704 mmol) and Na₂CO₃ (220 mg, 2.08 mmol) were suspended in DME (4 mL) and water (1 mL). The mixture was degassed for 5 min with nitrogen then Pd(PPh₃)₄ (80 mg, 0.069 mmol) was added. The mixture was sealed and heated at 100° C. with under microwave irradiation for 3 h. The mixture was filtered and the filtrate was concentrated in vacuo. The residue was purified by flash chromatography (10 g, silica) eluting with 0-10% MeOH/DCM. The resulting product was purified by ion exchange chromatography using a Biotage SCX-2 column, washing the column with DCM/MeOH, and then eluting the product with 7N NH₃ in MeOH to yield the title compound (112 mg, 33% yield). 1H NMR (500 MHz, Chloroform-d) δ 10.25 (s, 1H), 8.39 (d, J=4.9 Hz, 1H), 7.73 (s, 1H), 7.43-7.39 (m, 2H), 7.34-7.32 (m, 1H), 7.05 (d, J=4.9 Hz, 1H), 6.87-6.81 (m, 2H), 6.16 (dd, J=3.4, 1.5 Hz, 1H), 4.49 (d, J=17.6 Hz, 1H), 4.36 (d, J=17.6 Hz, 1H), 1.32 (s, 9H). LCMS (Analytical Method F) Rt=0.71 min, MS (ESIpos): m/z 393.2 [M+H]+, Purity=81%.

### Synthesis of 2-[4-(4-fluorophenyl)-5-{1H-pyrrolo[2,3-b]pyridin-4-yl}-1H-imidazol-1-yl]acetic acid/Intermediate 3

tert-Butyl 2-[4-(4-fluorophenyl)-5-(1H-pyrrolo[2,3-b]pyridin-4-yl)imidazol-1-yl]acetate (Intermediate 3-1) (106 mg, 0.219 mmol) was dissolved in DCM (0.9 mL) then TFA (0.3 mL) was added. The reaction was stirred at RT for 2 days, then concentrated in vacuo to yield the title compound as a TFA salt (119 mg, 77% yield). 1H NMR (400 MHz, DMSO-d6) δ 12.03 (s, 1H), 9.10 (s, 1H), 8.37 (d, J=4.8 Hz, 1H), 7.55-7.52 (m, 1H), 7.38-7.31 (m, 2H), 7.19-7.09 (m, 3H), 6.06-6.03 (m, 1H), 5.00-4.71 (m, 2H). LCMS (Analytical Method F) Rt=0.50 min, MS (ESIpos): m/z 337.1 [M+H]+, Purity=80%.

### Synthesis of tert-butyl 4-{2-[4-(4-chlorophenyl)-5-(pyridin-4-yl)-1H-imidazol-1-yl]acetyl}piperazine-1-carboxylate/Intermediate 4

A mixture of tert-butyl 4-[2-[5-bromo-4-(4-chlorophenyl)imidazol-1-yl]acetyl]piperazine-1-carboxylate (Intermediate 2-2) (100 mg, 0.207 mmol), pyridin-4-ylboronic acid (25 mg, 0.207 mmol), Pd(PPh₃)₄ (12 mg, 0.0103 mmol) and 2 M Na₂CO₃ (0.5 mL, 1.03 mmol) in DME (1.7 mL) was degassed by sparging with nitrogen. The reaction was heated to 100° C. for 1 h under microwave irradiation. The reaction was cooled and quenched into water. The aqueous layer was extracted into EtOAc (2×) and the combined organics washed with brine, dried over MgSO₄ and concentrated in vacuo. The residue was purified by flash chromatography (10 g, silica) eluting with 0-5% MeOH/DCM to yield the title compound (42 mg, 40% yield). 1H NMR (400 MHz, Chloroform-d) δ 8.69 (d, J=6.0 Hz, 2H), 7.65 (s, 1H), 7.37 (d, J=8.6 Hz, 2H), 7.25-7.18 (m, 4H), 4.61 (s, 2H), 3.62-3.55 (m, 2H), 3.45-3.34 (m, 4H), 3.32-3.24 (m, 2H), 1.47 (s, 9H). LCMS (Analytical Method E) Rt=1.02 min, MS (ESIpos): m/z 482.1, 484.2 [M+H]+, Purity=94%.

### Synthesis of tert-butyl (1S,4S)-5-{2-[4-(4-fluorophenyl)-5-(pyridin-4-yl)-1H-imidazol-1-yl]acetyl}-2,5-diazabicyclo[2.2.1]heptane-2-carboxylate/Intermediate 5

To a stirred solution of 2-[4-(4-fluorophenyl)-5-(pyridin-4-yl)-1H-imidazol-1-yl]acetic acid TFA salt (Intermediate 1) (50 mg, 0.0952 mmol) and tert-butyl (1R,4R)-2,5-diazabicyclo[2.2.1]heptane-2-carboxylate (25 mg, 0.126 mmol) in EtOAc (2.5 mL), DIPEA (50 μL, 0.286 mmol) and T3P (50%, 70 μL, 0.118 mmol) were added, and the reaction was stirred at RT for 1 h. The reaction was quenched with 1 M NaOH and extracted with EtOAc, dried over MgSO₄ and concentrated in vacuo. The residue was purified by preparative HPLC (Method B1) to yield the title compound (30 mg, 65% yield). 1H NMR (400 MHz, Chloroform-d) δ 8.71-8.65 (m, 2H), 7.66 (s, 1H), 7.44-7.37 (m, 2H), 7.25-7.21 (m, 2H), 6.97-6.89 (m, 2H), 4.93-4.85 (m, 1H), 4.62-4.41 (m, 3H), 3.39-3.30 (m, 2H), 3.30-3.21 (m, 2H), 1.94-1.82 (m, 1H), 1.71-1.67 (m, 1H), 1.47 (s, 9H). LCMS (Analytical Method E) Rt=0.97 min, MS (ESIpos): m/z 478.8 [M+H]+, Purity=98%.

### Synthesis of tert-butyl 7-{2-[4-(4-chlorophenyl)-5-(pyridin-4-yl)-1H-imidazol-1-yl]acetyl}-4,7-diazaspiro[2.5]octane-4-carboxylate/Intermediate 6

A solution of sodium 2-[4-(4-chlorophenyl)-5-(4-pyridyl)imidazol-1-yl]acetate (Intermediate 2b) (90 mg, 0.161 mmol), T3P (50%, 0.19 mL, 0.322 mmol), and DIPEA (0.11 mL, 0.606 mmol) in DMF (1.6 mL) was stirred for 10 min, then tert-butyl 4,7-diazaspiro[2.5]octane-4-carboxylate (53 mg, 0.241 mmol) was added. The mixture was stirred for 10 min, then DMF (1.6 mL) was added, followed by EtOAc (0.5 mL). The mixture was stirred at RT for 16 h, then at 60° C. for 3 h. T3P (50%, 0.19 mL, 0.322 mmol) and DIPEA (0.11 mL, 0.606 mmol) were added and the reaction was stirred at 60° C. for 3 h. Water was added and the mixture was extracted with EtOAc. The organics were dried over MgSO₄, filtered and concentrated in vacuo. The residue was purified by flash chromatography (10 g, silica) eluting with 0-100% EtOAc/DCM to yield the title compound (62 mg, 64% yield). 1H NMR (500 MHz, Chloroform-d) δ 8.69 (d, J=5.6 Hz, 2H), 7.67-7.62 (m, 1H), 7.36 (d, J=8.5 Hz, 2H), 7.25-7.16 (m, 4H), 4.66-4.44 (m, 2H), 3.67-3.02 (m, 6H), 1.05-0.92 (m, 2H), 0.82-0.51 (m, 2H). LCMS (Analytical Method F) Rt=0.82 min, MS (ESIpos): m/z 508.2 [M+H]+, Purity=85%.

### Synthesis of tert-butyl 7-{2-[4-(4-chlorophenyl)-5-(pyridin-4-yl)-1H-imidazol-1-yl]acetyl}-2,7-diazaspiro[3.5]nonane-2-carboxylate/Intermediate 7

To a stirred solution of sodium 2-[4-(4-chlorophenyl)-5-(4-pyridyl)imidazol-1-yl]acetate (Intermediate 2b) (120 mg, 0.214 mmol) in EtOAc (2.8 mL), DIPEA (0.15 mL, 0.858 mmol) and T3P (50%, 0.26 mL, 0.429 mmol) were added, and the resulting mixture was allowed to stir at RT for 16 h. The mixture was stirred at 60° C. for 8 h. DMF (2 mL) and T3P (50%, 0.26 mL, 0.429 mmol) were added and the mixture stirred at 60° C. for 8 h. The mixture was cooled to RT and stirred for 16 h. Water was added and the mixture was extracted with DCM, dried over MgSO₄, filtered and concentrated in vacuo. The residue was purified by preparative HPLC (Method B1) to yield the title compound (40 mg, 33% yield). 1H NMR (400 MHz, Chloroform-d) δ 8.74-8.66 (m, 2H), 7.66 (s, 1H), 7.41-7.37 (m, 2H), 7.27-7.25 (m, 2H), 7.25-7.21 (m, 2H), 4.61 (s, 2H), 3.75-3.61 (m, 4H), 3.54 (br s, 2H), 3.25 (t, J=5.3 Hz, 2H), 1.73 (t, J=4.5 Hz, 2H), 1.66 (br s, 2H), 1.47 (s, 9H). LCMS (Analytical Method F) Rt=0.83 min, MS (ESIpos): m/z 522.3, 524.3 [M+H]+, Purity=93%.

### Synthesis of tert-butyl 2-{2-[4-(4-chlorophenyl)-5-(pyridin-4-yl)-1H-imidazol-1-yl]acetyl}-2,7-diazaspiro[3.5]nonane-7-carboxylate/Intermediate 8

To a stirred solution of sodium 2-[4-(4-chlorophenyl)-5-(4-pyridyl)imidazol-1-yl]acetate (Intermediate 2b) (120 mg, 0.214 mmol) in EtOAc (2.8 mL), DIPEA (150 μL, 0.858 mmol) and T3P (50%, 255 μL, 0.429 mmol) were added, and the mixture was stirred at RT for 18 h. The mixture was stirred at 60° C. for 8 h. DMF (2 mL) and T3P (50%, 0.26 mL, 0.429 mmol) were added and the mixture stirred at 60° C. for 8 h, then at RT for 16 h. Water was added and the mixture was extracted with DCM, dried over MgSO₄, filtered and concentrated in vacuo. The residue was purified by preparative HPLC (Method B1) to yield the title compound (60 mg, 54% yield). 1H NMR (400 MHz, Chloroform-d) δ 8.77-8.70 (m, 2H), 7.70 (s, 1H), 7.42-7.36 (m, 2H), 7.32-7.29 (m, 2H), 7.26-7.20 (m, 2H), 4.41 (s, 2H), 3.73 (s, 2H), 3.62 (br s, 2H), 3.45-3.34 (m, 2H), 3.31 (ddd, J=13.7, 7.3, 4.2 Hz, 2H), 1.75-1.62 (m, 4H), 1.47 (s, 9H). LCMS (Analytical Method F) Rt=0.84 min, MS (ESIpos): m/z 522.3, 524.3 [M+H]+, Purity=93%.

### Synthesis of tert-butyl 4-{2-[4-(4-fluorophenyl)-5-(pyridin-4-yl)-1H-imidazol-1-yl]acetyl}piperazine-1-carboxylate/Intermediate 9

2-[4-(4-fluorophenyl)-5-(pyridin-4-yl)-1H-imidazol-1-yl]acetic acid TFA salt (Intermediate 1) (40 mg, 0.112 mmol), HATU (75 mg, 0.197 mmol) and DIPEA (60 μL, 0.344 mmol) were dissolved in DMF (1 mL) and stirred at RT for 10 min, then tert-butyl piperazine-1-carboxylate (25 mg, 0.134 mmol) was added and the reaction was stirred for 1 h. The reaction was diluted with water and extracted with EtOAc, dried over MgSO₄, filtered and concentrated in vacuo. The residue was purified by preparative HPLC (Method A1) to afford the title compound (14 mg, 27% yield). 1H NMR (400 MHz, DMSO-d6) δ 8.66-8.60 (m, 2H), 7.80 (s, 1H), 7.39-7.35 (m, 2H), 7.29-7.24 (m, 2H), 7.09 (t, J=9.0 Hz, 2H), 4.93 (s, 2H), 3.43-3.34 (m, 4H), 3.23 (s, 4H), 1.40 (s, 9H). LCMS (Analytical Method E) Rt=0.98 min, MS (ESIpos): m/z 466.23 [M+H]+, Purity=90%.

### Synthesis of 2-(difluoromethyl)-4-(4,4,5,5-tetramethyl-1,3,2-dioxaborolan-2-yl)pyridine/Intermediate 10-1

4-bromo-2-(difluoromethyl)pyridine (150 mg, 0.721 mmol), KOAc (150 mg, 1.51 mmol) and 4,4,5,5-tetramethyl-2-(4,4,5,5-tetramethyl-1,3,2-dioxaborolan-2-yl)-1,3,2-dioxaborolane (200 mg, 0.788 mmol) were suspended in 1,4-dioxane (5 mL) and the mixture was degassed with nitrogen for 5 min. Then Pd(dppf)Cl₂ (60 mg, 0.0733 mmol) was added and the reaction was sealed and stirred at 80° C. for 16 h. The mixture was diluted with water and extracted with DCM, dried over MgSO₄, filtered and concentrated in vacuo. The residue was purified by flash chromatography (25 g, silica) eluting with 0-100% EtOAc/heptane to yield the title compound (169 mg, 83% yield). 1H NMR (500 MHz, DMSO-d6) δ 8.74 (d, J=4.7 Hz, 1H), 7.81 (s, 1H), 7.75-7.72 (m, 1H), 6.99 (t, J=54.9 Hz, 1H), 1.32 (s, 12H). LCMS (Analytical Method F) Rt=0.44 min, MS (ESIpos): m/z 256.2 [M+H]+, Purity=90%.

### Synthesis of tert-butyl 2-[4-(4-chlorophenyl)-5-[2-(difluoromethyl)pyridin-4-yl]-1H-imidazol-1-yl]acetate/Intermediate 10-2

tert-Butyl 2-[5-bromo-4-(4-chlorophenyl)imidazol-1-yl]acetate (Intermediate 2-2) (200 mg, 0.538 mmol), 2-(difluoromethyl)-4-(4,4,5,5-tetramethyl-1,3,2-dioxaborolan-2-yl)pyridine (Intermediate 10-1) (155 mg, 0.608 mmol) and Na₂CO₃ (168 mg, 1.59 mmol) were suspended in DME (2 mL) and water (0.5 mL). The mixture was degassed with nitrogen for 5 min then Pd(PPh₃)₄ (31 mg, 0.0269 mmol) was added. The mixture was degassed for 5 min then sealed and heated at 100° C. for 2 h under microwave irradiation. The reaction was diluted with water and extracted with EtOAc, dried over MgSO₄, filtered and concentrated in vacuo. The residue was purified by flash chromatography (25 g, silica) eluting with 0-10% MeOH/DCM, then by preparative HPLC (Method A2) to yield the title compound (133 mg, 57% yield). 1H NMR (400 MHz, DMSO-d6) δ 8.76 (d, J=5.0 Hz, 1H), 7.94 (s, 1H), 7.63-7.60 (m, 1H), 7.54-7.49 (m, 1H), 7.39-7.31 (m, 4H), 6.98 (t, J=54.7 Hz, 1H), 4.88 (s, 2H), 1.22 (s, 9H). LCMS (Analytical Method F) Rt=0.99 min, MS (ESIpos): m/z 420.2, 422.2 [M+H]+, Purity=96%.

### Synthesis of 2-[4-(4-chlorophenyl)-5-[2-(difluoromethyl)pyridin-4-yl]-1H-imidazol-1-yl]acetic acid/Intermediate 10

To a stirred solution of tert-butyl 2-[4-(4-chlorophenyl)-5-[2-(difluoromethyl)-4-pyridyl]imidazol-1-yl]acetate (Intermediate 10-2) (130 mg, 0.297 mmol) in DCM (2 mL), TFA (0.5 ml) was added, and the mixture was stirred at RT for 16 h. Additional TFA (0.5 ml) was added and the reaction was stirred for 4 h. The reaction was concentrated in vacuo to yield the title compound (140 mg, 91%), which was used in the next step without further purification. 1H NMR (500 MHz, MeOH-d4) δ 9.04 (s, 1H), 8.82-8.78 (m, 1H), 7.74 (s, 1H), 7.60-7.56 (m, 1H), 7.45-7.41 (m, 2H), 7.39-7.34 (m, 2H), 6.77 (t, J=55.0 Hz, 1H), 5.04 (s, 2H). LCMS (Analytical Method F) Rt=0.67 min, MS (ESIpos): m/z 364.1, 366.1 [M+H]+, Purity=92%.

### Synthesis of tert-butyl 2-[5-(2-{[(tert-butoxy)carbonyl]amino}pyridin-4-yl)-4-(4-fluorophenyl)-1H-imidazol-1-yl]acetate/Intermediate 11-1

tert-Butyl 2-[5-bromo-4-(4-fluorophenyl)imidazol-1-yl]acetate (Intermediate 1-2) (300 mg, 0.845 mmol), tert-butyl [4-(4,4,5,5-tetramethyl-1,3,2-dioxaborolan-2-yl)pyridin-2-yl]carbamate (320 mg, 0.979 mmol) and Na₂CO₃ (264 mg, 2.49 mmol) were suspended in DME (3.6 mL) and water (1 mL), and the mixture was degassed with nitrogen for 5 min. Pd(PPh₃)₄ (50 mg, 0.0433 mmol) was added, and the mixture was degassed for a further 5 min, then sealed and stirred at 100° C. for 2 h. The mixture was diluted with water and 1 M NaOH, extracted with EtOAc, dried over MgSO₄, filtered and concentrated in vacuo. The residue was purified by flash chromatography (25 g, silica) eluting with 0-8% MeOH/DCM to afford the title compound (595 mg, 54% yield). LCMS (Analytical Method H) Rt=0.66 min, MS (ESIpos): m/z 469.4 [M+H]+, Purity=36%.

### Synthesis of methyl 2-[5-(2-aminopyridin-4-yl)-4-(4-fluorophenyl)-1H-imidazol-1-yl]acetate/Intermediate 11-2

tert-Butyl 2-[5-[2-(tert-butoxycarbonylamino)-4-pyridyl]-4-(4-fluorophenyl)imidazol-1-yl]acetate (Intermediate 11-1) (590 mg, 0.881 mmol) was suspended in 4 M HCl in dioxane (5 mL) and MeOH (3 mL) and stirred at RT for 19 h. The reaction was concentrated in vacuo, then partitioned between 1 M aq. NaOH and DCM. The organic phase was separated, dried over MgSO₄, filtered, and concentrated in vacuo. The residue was purified by flash chromatography (25 g, silica) eluting with 0-10% (7N NH₃ in MeOH)/DCM to afford the title compound (35 mg, 10% yield). LCMS (Analytical Method H) Rt=0.43 min, MS (ESIpos): m/z 327.3 [M+H]+, Purity=79%.

### Synthesis of 2-{5-[2-(2-fluorobenzamido)pyridin-4-yl]-4-(4-fluorophenyl)-1H-imidazol-1-yl}acetic acid/Intermediate 11

To a stirred solution of methyl 2-[5-(2-amino-4-pyridyl)-4-(4-fluorophenyl)imidazol-1-yl]acetate (Intermediate 11-2) (32 mg, 0.098 mmol) in THF (1 mL), DIPEA (55 μL, 0.315 mmol) and 2-fluorobenzoyl chloride (25 μL, 0.209 mmol) were added, and the reaction was stirred at RT for 1 h. 2 M NaOH (1.0 mL, 2.00 mmol) was added and the reaction was stirred for 1 h. The reaction was diluted with water and extracted with DCM. The organic layer was dried over MgSO₄, filtered, and concentrated in vacuo to yield the title compound (45 mg, 91% yield), which was used in the next step without further purification. 1H NMR (400 MHz, DMSO-d6) δ 10.86 (s, 1H), 8.44-8.40 (m, 1H), 8.10 (s, 1H), 7.76 (s, 1H), 7.67 (td, J=7.6, 1.7 Hz, 1H), 7.60-7.54 (m, 1H), 7.46-7.39 (m, 2H), 7.35-7.27 (m, 2H), 7.14 (dd, J=5.1, 1.5 Hz, 1H), 7.13-7.06 (m, 2H), 4.31 (s, 2H). LCMS (Analytical Method H) Rt=0.35 min, MS (ESIpos): m/z 435.2 [M+H]+, Purity=87%.

### Synthesis of tert-butyl 2-[5-(2-aminopyridin-4-yl)-4-(4-fluorophenyl)-1H-imidazol-1-yl]acetate/Intermediate 12-1

tert-Butyl [4-(4,4,5,5-tetramethyl-1,3,2-dioxaborolan-2-yl)pyridin-2-yl]carbamate (285 mg, 0.872 mmol), tert-butyl 2-[5-bromo-4-(4-fluorophenyl)imidazol-1-yl]acetate (Intermediate 1-2) (270 mg, 0.760 mmol) and Na₂CO₃ (240 mg, 2.26 mmol) were suspended in DME (5 mL) and water (1 mL), and the mixture was degassed with nitrogen for 10 min. Pd(PPh₃)₄ (70 mg, 0.0606 mmol) was then added, and the reaction was sealed under nitrogen and stirred at 100° C. for 16 h. The mixture was diluted with water and extracted with DCM, dried over MgSO₄, filtered, and concentrated in vacuo. The residue was purified by flash chromatography (25 g, silica) eluting with 0-5% MeOH/DCM to yield the title compound (144 mg, 50% yield). LCMS (Analytical Method F) Rt=0.66 min, MS (ESIpos): m/z 369.3 [M+H]+, Purity=98%.

### Synthesis of 2-{5-[2-(4-fluorobenzamido)pyridin-4-yl]-4-(4-fluorophenyl)-1H-imidazol-1-yl}acetic acid/Intermediate 12

To a stirred solution of tert-butyl 2-[5-(2-amino-4-pyridyl)-4-(4-fluorophenyl)imidazol-1-yl]acetate (Intermediate 12-1) (130 mg, 0.353 mmol) in THF (2 mL), DIPEA (190 μL, 1.09 mmol) and 4-fluorobenzoyl chloride (90 μL, 0.762 mmol) were added, and the reaction was stirred at RT for 1 h. 2 M aq. NaOH (2.0 mL, 4.00 mmol) was added and the reaction was stirred for 5 h. The reaction was neutralised with 2 M HCl (aq.) and extracted with EtOAc, dried over MgSO₄, filtered, and concentrated in vacuo. The residue was purified by preparative HPLC (Method B1) to yield the title compound (134 mg, 57% yield). 1H NMR (400 MHz, DMSO-d6) δ 11.00 (s, 1H), 8.48 (dd, J=5.1, 0.6 Hz, 1H), 8.15-8.13 (m, 1H), 8.11-8.05 (m, 2H), 8.04-7.97 (m, 2H), 7.91 (s, 1H), 7.49-7.42 (m, 2H), 7.15-7.06 (m, 3H), 4.77 (s, 2H). LCMS (Analytical Method B) Rt=2.23 min, MS (ESIpos): m/z 400.3 [M+H]+, Purity=97%.

### Synthesis of tert-butyl 4-{2-[2-chloro-4-(4-fluorophenyl)-5-(pyridin-4-yl)-1H-imidazol-1-yl]acetyl}piperazine-1-carboxylate/Intermediate 13

N-Chlorosuccinimide (26 mg, 0.197 mmol) was added to a solution of tert-butyl 4-[2-[4-(4-fluorophenyl)-5-(4-pyridyl)imidazol-1-yl]acetyl]piperazine-1-carboxylate (Intermediate 9) (90 mg, 0.164 mmol) in anhydrous THF (2.5 mL) and the mixture was stirred at RT overnight. The mixture was diluted with EtOAc, washed with sat. NaHCO₃ and brine, dried over MgSO₄ and concentrated in vacuo. The residue was purified by flash chromatography (10 g, KP-NH) eluting with 0-100% EtOAc/heptane to afford the title compounds as a brown oil (19 mg, 23% yield). LCMS (Analytical Method E) Rt=1.12 min, MS (ESIpos): m/z 500.2 [M+H]+, Purity=100%.

### Synthesis of tert-butyl 4-(2-chloroacetyl)piperazine-1-carboxylate/Intermediate 14-1

To an ice-cold solution of tert-butyl piperazine-1-carboxylate (2.50 g, 13.0 mmol) in DCM (58 mL), DIPEA (2.7 mL, 15.6 mmol) was added followed by chloroacetyl chloride (1.0 mL, 13.0 mmol), and the mixture was allowed to stir at 0° C. for 90 min. The reaction was quenched with water and the aqueous layer was extracted with DCM (3×), dried over MgSO₄ and concentrated in vacuo. The residue was purified by flash chromatography (100 g, silica), eluting with 20-80% EtOAc/heptane to yield the title compound as an off-white solid (3.11 g, 91% yield). ¹H NMR (400 MHz, Chloroform-d) δ 4.07 (s, 2H), 3.64-3.57 (m, 2H), 3.50 (s, 4H), 3.47-3.41 (m, 2H), 1.47 (s, 9H). LCMS (Analytical Method F) Rt=0.76 min, MS (ESIpos): m/z 207.0 [M-tBu+H]⁺, Purity=100%.

### Synthesis of tert-butyl 4-{2-[4-(4-chlorophenyl)-1H-imidazol-1-yl]acetyl}piperazine-1-carboxylate/Intermediate 14-2

NaH (60%, 123 mg, 3.08 mmol) was added to an ice-cold solution of 4-(4-chlorophenyl)-1H-imidazole (0.50 g, 2.80 mmol) in THF (12 mL). The reaction was stirred for 15 min and then tert-butyl 4-(2-chloroacetyl)piperazine-1-carboxylate (Intermediate 14-1) (735 mg, 2.80 mmol) was added and stirring continued for 2 h. The reaction was quenched into water. The aqueous layer was extracted into EtOAc (3×), the combined organics washed with brine, dried over MgSO₄ and concentrated in vacuo. The residue was purified by flash chromatography (25 g, silica) eluting with 0-8% MeOH/DCM to yield the title compound as a tan solid (800 mg, 71% yield). 1H NMR (500 MHz, Chloroform-d) δ 7.70 (d, J=8.6 Hz, 2H), 7.52 (d, J=1.2 Hz, 1H), 7.33 (d, J=8.6 Hz, 2H), 7.23 (d, J=1.2 Hz, 1H), 4.81 (s, 2H), 3.63 (d, J=5.0 Hz, 2H), 3.51-3.42 (m, 6H), 1.48 (s, 9H). LCMS (Analytical Method E) Rt=0.98 min, MS (ESIpos): m/z 405.1, 407.1 [M+H]+, Purity=99%.

### Synthesis of N-(4-bromopyridin-2-yl)benzamide/Intermediate 14-3

Benzoyl chloride (1.2 mL, 10.0 mmol) was added to a solution of 4-bromopyridin-2-amine (1.57 g, 9.09 mmol) and pyridine (1.1 mL, 13.6 mmol) in THF (28 mL), and the reaction mixture was stirred at RT for 18 h. MeOH (16 mL) and 2 M NaOH (23 mL, 45.5 mmol) were added and the reaction stirred at RT for 2 h. The mixture was diluted with water (3 mL) and extracted with EtOAc (3×20 mL). The organic extracts were combined, washed with brine (20 mL), dried over Na₂SO₄, filtered and concentrated in vacuo to afford the title compound as an off-white solid (2.3 g, 69% yield), which was used in the next step without further purification. 1H NMR (500 MHz, DMSO-d6) δ 11.05 (s, 1H), 8.47 (d, J=1.7 Hz, 1H), 8.31 (d, J=5.3 Hz, 1H), 8.03 (dd, J=8.3, 1.2 Hz, 2H), 7.65-7.59 (m, 1H), 7.56-7.49 (m, 2H), 6.67-6.64 (m, 1H). LCMS (Analytical Method E) Rt=1.20 min, MS (ESIpos): m/z 277, 279 [M+H]+, Purity=92%.

### Synthesis of tert-butyl 4-{2-[5-bromo-4-(4-chlorophenyl)-1H-imidazol-1-yl]acetyl}piperazine-1-carboxylate/Intermediate 14-4

NBS (0.39 g, 2.17 mmol) was added to an ice-cold solution of tert-butyl 4-{2-[4-(4-chlorophenyl)-1H-imidazol-1-yl]acetyl}piperazine-1-carboxylate (Intermediate 14-2) (800 mg, 1.98 mmol) in DCM (13 mL). The reaction was stirred for 1 h and then quenched into water. The aqueous layer was extracted into DCM (3×), the combined organics washed with brine, dried over MgSO₄ and concentrated in vacuo. The residue was purified by flash chromatography (25 g, silica) eluting with 0-5% MeOH/DCM to yield the title compound as a tan solid (502 mg, 47% yield). 1H NMR (500 MHz, Chloroform-d) δ 7.93 (d, J=8.6 Hz, 2H), 7.72 (s, 1H), 7.39 (d, J=8.6 Hz, 2H), 4.83 (s, 2H), 3.70-3.64 (m, 2H), 3.60-3.53 (m, 4H), 3.51 (d, J=5.0 Hz, 2H), 1.51 (s, 9H). LCMS (Analytical Method E) Rt=1.20 min, MS (ESIpos): m/z 483.0, 484.8, 487.0 [M+H]+, Purity=89%.

### Synthesis of (2-benzamidopyridin-4-yl)boronic acid/Intermediate 14-5

A mixture of N-(4-bromopyridin-2-yl)benzamide (Intermediate 14-3) (750 mg, 2.03 mmol), 4,4,5,5-tetramethyl-2-(4,4,5,5-tetramethyl-1,3,2-dioxaborolan-2-yl)-1,3,2-dioxaborolane (773 mg, 3.04 mmol) and KOAc (403 mg, 4.06 mmol) in anhydrous 1,4-dioxane (6.75 mL) was sparged with nitrogen for 2 min. Then Pd(dppf)Cl₂ (83 mg, 0.101 mmol) was added and the mixture sparged with nitrogen for a further 2 min, before it was heated at 100° C. for 3 h in a sealed tube. The reaction mixture was then diluted with water (15 mL), extracted with EtOAc (3×20 mL). The combined organics were washed with brine (10 mL), dried over Na₂SO₄, filtered and concentrated in vacuo to afford the title compound (1.421 g, quant.), which was used in the next step without further purification. 1H NMR (500 MHz, DMSO-d6) δ 10.79 (s, 1H), 8.47 (s, 1H), 8.43 (dd, J=4.7, 0.8 Hz, 1H), 8.06-8.00 (m, 2H), 7.63-7.58 (m, 1H), 7.54-7.49 (m, 2H), 7.34 (dd, J=4.7, 0.8 Hz, 1H). LCMS (Analytical Method E) Rt=0.77 min, MS (ESIpos): m/z 142.9 [M+H]+, Purity=63%.

### Synthesis of tert-butyl 4-{2-[5-(2-benzamidopyridin-4-yl)-4-(4-chlorophenyl)-1H-imidazol-1-yl]acetyl}piperazine-1-carboxylate/Intermediate 14

A mixture of tert-butyl 4-{2-[5-bromo-4-(4-chlorophenyl)-1H-imidazol-1-yl]acetyl}piperazine-1-carboxylate (Intermediate 14-4) (100 mg, 0.207 mmol), (2-benzamidopyridin-4-yl)boronic acid (Intermediate 14-5) (186 mg, 0.269 mmol), Na₂CO₃ (66 mg, 0.620 mmol), and Pd(PPh₃)₄ (12 mg, 0.0103 mmol) in 1,4-dioxane (2 mL) was degassed by sparging with nitrogen. The reaction was heated at 100° C. for 2 h in a sealed tube. Pd(PPh₃)₄ (12 mg, 0.0103 mmol) and (2-benzamidopyridin-4-yl)boronic acid (Intermediate 14-5) (186 mg, 0.269 mmol) were added again, the mixture sparged with nitrogen for 1 min and then heated at 100° C. for 2 h in a sealed tube. Pd(PPh₃)₄ (12 mg, 0.0103 mmol) and (2-benzamidopyridin-4-yl)boronic acid (Intermediate 14-5) (186 mg, 0.269 mmol) were added again, the mixture sparged with nitrogen for 1 min and then heated at 100° C. for 2 h in a sealed tube. The reaction was cooled to RT, quenched into water (10 mL), and the aqueous layer was extracted with EtOAc (3×20 mL). The combined organics were washed with brine (10 mL), dried over Na₂SO₄ and concentrated in vacuo. The residue was purified by flash chromatography (25 g, silica) using a 0-100% IPA/TBME gradient and the resulting product was further purified by preparative HPLC (Method A2) to afford the title compound (100 mg, 47% yield). LCMS (Analytical Method E) Rt=1.14 min, MS (ESIpos): m/z 601.1 [M+H]+, Purity=59%.

### Synthesis of tert-butyl 4-{2-[4-(4-fluorophenyl)-1H-imidazol-1-yl]acetyl}piperazine-1-carboxylate/Intermediate 15-1

NaH (60%, 250 mg, 6.25 mmol) was added to an ice-cold solution of 4-(4-fluorophenyl)-1H-imidazole (1.0 g, 6.17 mmol) in THF (20 mL). The mixture was stirred for 5 min and then tert-butyl 4-(2-chloroacetyl)piperazine-1-carboxylate (intermediate 14-1) (1.63 g, 6.22 mmol) was added, and the reaction was stirred for 1 h. The reaction was quenched with water and extracted with DCM, dried over MgSO₄ and concentrated in vacuo. The residue was purified by flash chromatography (25 g, silica) using 0-10% MeOH/DCM to afford the title compound as a pale yellow solid (2.02 g, 83% yield). 1H NMR (400 MHz, Chloroform-d) δ 7.75-7.69 (m, 2H), 7.51 (d, J=1.2 Hz, 1H), 7.18 (d, J=1.3 Hz, 1H), 7.08-7.01 (m, 2H), 4.80 (s, 2H), 3.66-3.61 (m, 2H), 3.48-3.43 (m, 6H), 1.47 (s, 9H). LCMS (Analytical Method H) Rt=0.54 min, MS (ESIpos): m/z 389.3 [M+H]+, Purity=98%.

### Synthesis of tert-butyl 4-{2-[5-bromo-4-(4-fluorophenyl)-1H-imidazol-1-yl]acetyl}piperazine-1-carboxylate/Intermediate 15-2

NBS (925 mg, 5.20 mmol) was added to an ice-cold solution of tert-butyl 4-{2-[4-(4-fluorophenyl)-1H-imidazol-1-yl]acetyl}piperazine-1-carboxylate (Intermediate 15-1) (2.03 g, 5.18 mmol) in DCM (20 mL), and the mixture was stirred at 0° C. for 1 h. The reaction was quenched with water and extracted with DCM (2×). The combined organics were dried over MgSO₄ and concentrated in vacuo. The residue was purified by flash chromatography (25 g, silica) using 0-10% MeOH/DCM as a yellow solid (1.61 g, 58% yield). 1H NMR (400 MHz, DMSO-d6) δ 7.96-7.91 (m, 2H), 7.87 (s, 1H), 7.29-7.23 (m, 2H), 5.08 (s, 2H), 3.58-3.53 (m, 2H), 3.48-3.42 (m, 4H), 3.36-3.33 (m, 2H), 1.42 (s, 9H). LCMS (Analytical Method F) Rt=0.90 min, MS (ESIpos): m/z 467.1, 469.1 [M+H]+, Purity=88%.

### Synthesis of tert-butyl 4-{2-[4-(4-fluorophenyl)-5-(2-fluoropyridin-4-yl)-1H-imidazol-1-yl]acetyl}piperazine-1-carboxylate/Intermediate 15

A mixture of tert-butyl 4-{2-[5-bromo-4-(4-fluorophenyl)-1H-imidazol-1-yl]acetyl}piperazine-1-carboxylate (Intermediate 15-2) (600 mg, 1.13 mmol), (2-fluoropyridin-4-yl)boronic acid (250 mg, 1.77 mmol), and Na₂CO₃ (360 mg, 3.40 mmol) in DME (10 mL) and water (2 mL) was degassed with nitrogen for 5 min. Then Pd(PPh₃)₄ (130 mg, 0.112 mmol) was added and after degassing for a further 5 min the mixture was stirred at 100° C. for 2 h in a sealed tube. The reaction was retreated with (2-fluoropyridin-4-yl)boronic acid (25 mg, 0.18 mmol) and Pd(PPh₃)₄ (13 mg, 0.011 mmol) and stirred for 3 h. The reaction was filtered through celite and the filtrate was concentrated in vacuo. The residue was taken up in EtOAc, washed with sat. NaHCO₃, dried over MgSO₄ and concentrated in vacuo. The residue was purified by flash chromatography (25 g, silica) using 0-10% MeOH/DCM to afford the title compound as a yellow solid (518 mg, 86% yield). 1H NMR (400 MHz, Chloroform-d) δ 8.26 (d, J=5.1 Hz, 1H), 7.64 (s, 1H), 7.42-7.37 (m, 2H), 7.13-7.09 (m, 1H), 6.98-6.93 (m, 2H), 6.89 (s, 1H), 4.64 (s, 2H), 3.62-3.56 (m, 2H), 3.43-3.39 (m, 4H), 3.36-3.30 (m, 2H), 1.47 (s, 9H). LCMS (Analytical Method F) Rt=0.83 min, MS (ESIpos): m/z 484.3 [M+H]+, Purity=91%.

### Synthesis of 4-bromo-2-(bromomethyl)pyridine/Intermediate 16-1

To a stirred solution of (4-bromopyridin-2-yl)methanol (1.00 g, 5.32 mmol) and carbon tetrabromide (2.82 g, 8.51 mmol) in DCM (20 mL) at 0° C., triphenylphosphine (1.67 g, 6.38 mmol) was added portion-wise, and the mixture was allowed to stir at 0° C. for 1 h, then at RT overnight. The solvent was evaporated under reduced pressure and the residue was purified by flash chromatography (100 g, silica) eluting with 0-100% EtOAc/heptane to yield the title compound as a dark purple liquid (829 mg, 50% yield). 1H NMR (400 MHz, Chloroform-d) δ 8.40 (d, J=5.3 Hz, 1H), 7.63 (d, J=1.7 Hz, 1H), 7.40 (dd, J=5.3, 1.8 Hz, 1H), 4.50 (s, 2H). LCMS (Analytical Method F) Rt=0.81 min, MS (ESIpos): m/z 249.9 [M+H]+, Purity=58%.

### Synthesis of tert-butyl N-[(4-bromopyridin-2-yl)methyl]-N-methylcarbamate/Intermediate 16-2

NaH (69 mg, 2.88 mmol) was added portionwise to an ice-cold solution of tert-butyl methylcarbamate (377 mg, 2.88 mmol) in THF (13 mL), and the mixture was allowed to stir at RT for 1 h. Then, the mixture was cooled down to 0° C. and a solution of 4-bromo-2-(bromomethyl)pyridine (Intermediate 16-1) (820 mg, 2.61 mmol) in THF (13 mL) was added dropwise and the reaction stirred at RT overnight. The mixture was carefully quenched with water, extracted with EtOAc (2×), dried over MgSO₄, filtered and evaporated under reduced pressure. The residue was purified by flash chromatography (25 g, silica) eluting with 0-40% EtOAc/heptane to yield the title compound as a yellow oil (439 mg, 52% yield). 1H NMR (500 MHz, Chloroform-d) δ 8.35 (d, J=5.3 Hz, 1H), 7.43-7.32 (m, 2H), 4.59-4.45 (m, 2H), 3.03-2.84 (m, 3H), 1.55-1.36 (m, 9H). LCMS (Analytical Method F) Rt=0.94 min, MS (ESIpos): m/z 301 [M+H]+, Purity=94%.

### Synthesis of [2-({[(tert-butoxy)carbonyl](methyl)amino}methyl)pyridin-4-yl]boronic acid/Intermediate 16-3

tert-Butyl N-[(4-bromopyridin-2-yl)methyl]-N-methylcarbamate (Intermediate 16-2) (40 mg, 0.102 mmol), KOAc (20 mg, 0.205 mmol) and 4,4,5,5-tetramethyl-2-(4,4,5,5-tetramethyl-1,3,2-dioxaborolan-2-yl)-1,3,2-dioxaborolane (39 mg, 0.153 mmol) were dissolved in anhydrous 1,4-dioxane (0.385 mL) and sparged with nitrogen for 2 min. Then, Pd(dppf)Cl₂ (4.2 mg, 5.11 μmol) was added and the mixture sparged with nitrogen for a further 2 min before it was heated at 75° C. for 2 h and at 100° C. for 4 h in a sealed tube. The reaction mixture was then diluted with water (10 mL) and extracted with EtOAc (3×20 mL). The organic extracts were combined, washed with brine (10 mL), dried over Na₂SO₄, filtered and concentrated in vacuo to afford the title compound as brown oil (76 mg, quant.), which was used in the next reaction without further purification. LCMS (Analytical Method F) Rt=0.46 min, MS (ESIpos): m/z 267.2 [M+H]+, Purity=43%.

### Synthesis of tert-butyl 4-(2-{5-[2-({[(tert-butoxy)carbonyl](methyl)amino}methyl)pyridin-4-yl]-4-(4-chlorophenyl)-1H-imidazol-1-yl}acetyl)piperazine-1-carboxylate/Intermediate 16

A mixture of tert-butyl 4-{2-[5-bromo-4-(4-chlorophenyl)-1H-imidazol-1-yl]acetyl}piperazine-1-carboxylate (Intermediate 14-4) (75 mg, 0.155 mmol), [2-({[(tert-butoxy)carbonyl](methyl)amino}methyl)pyridin-4-yl]boronic acid (Intermediate 16-3) (76 mg, 0.122 mmol), 2 M Na₂CO₃ (0.39 mL, 0.775 mmol), and Pd(PPh₃)₄ (9.0 mg, 7.75 μmol) in 1,4-dioxane (0.9974 mL) was degassed by sparging with nitrogen. The mixture was heated to 100° C. for 2 h in a sealed tube. The reaction was quenched into water (10 mL), and the aqueous layer was extracted with EtOAc (3×20 mL). The combined organics were washed with brine (10 mL), dried over Na₂SO₄ and concentrated in vacuo. The residue was purified by flash chromatography (25 g, silica) eluting with 0-10% MeOH/DCM, followed by preparative HPLC (Method B1) to afford the title compound as an orange gum (11 mg, 10% yield). LCMS (Analytical Method E) Rt=1.18 min, MS (ESIpos): m/z 625.1 [M+H]+, Purity=97%.

### Synthesis of tert-butyl 4-{2-[4-(4-fluorophenyl)-5-{1H-pyrrolo[2,3-b]pyridin-4-yl}-1H-imidazol-1-yl]acetyl}piperazine-1-carboxylate/Intermediate 17

A mixture of tert-butyl 4-{2-[5-bromo-4-(4-fluorophenyl)-1H-imidazol-1-yl]acetyl}piperazine-1-carboxylate (Intermediate 15-2) (120 mg, 0.218 mmol), 4-(4,4,5,5-tetramethyl-1,3,2-dioxaborolan-2-yl)-1H-pyrrolo[2,3-b]pyridine (55 mg, 0.225 mmol) and K₃PO₄ (140 mg, 0.650 mmol) in dioxane/water (4:1, 1 mL) was degassed with nitrogen for 10 min. Then Pd(dppf)Cl₂ (20 mg, 0.024 mmol) was added and the reaction was stirred at 100° C. for 5 h in a sealed tube. Additional Pd(dppf)Cl₂ (20 mg, 0.024 mmol) was added and the reaction was stirred for 2 h. The reaction mixture was filtered and the filtrate was diluted with water and extracted with DCM (2×). The combined organics were dried over MgSO₄, filtered and concentrated in vacuo. The residue was purified by preparative HPLC (Method B1) to yield the title compound as a white solid (46 mg, 41% yield). 1H NMR (400 MHz, Chloroform-d) δ 10.68 (s, 1H), 8.38 (d, J=4.9 Hz, 1H), 7.77 (s, 1H), 7.43-7.37 (m, 2H), 7.35 (d, J=3.5 Hz, 1H), 7.04 (d, J=4.9 Hz, 1H), 6.87-6.80 (m, 2H), 6.19 (d, J=3.5 Hz, 1H), 4.65-4.51 (m, 2H), 3.53-3.28 (m, 4H), 3.17-2.98 (m, 4H), 1.43 (s, 9H). LCMS (Analytical Method E) Rt=0.94 min, MS (ESIpos): m/z 505.25 [M+H]+, Purity=100%.

### Synthesis of tert-butyl 4-[2-(4-phenyl-1H-imidazol-1-yl)acetyl]piperazine-1-carboxylate/Intermediate 18-1

NaH (60%, 56 mg, 1.41 mmol) was added to an ice-cold solution of 4-phenyl-1H-imidazole (200 mg, 1.39 mmol) in THF (4.5 mL). The reaction was stirred for 10 min then tert-butyl 4-(2-chloroacetyl)piperazine-1-carboxylate (intermediate 14-1) (367 mg, 1.40 mmol) was added and the reaction stirred for 30 min. The reaction was cautiously quenched with water and extracted with DCM. The organic layer was washed with brine, dried over MgSO₄ and concentrated in vacuo. The residue was purified by flash chromatography (10 g, silica), eluting with 0-10% MeOH/DCM to yield the title compound as an off-white solid (480 mg, 93% yield). 1H NMR (400 MHz, DMSO-d6) δ 7.73 (dd, J=8.3, 1.2 Hz, 2H), 7.58 (d, J=1.1 Hz, 1H), 7.50 (d, J=1.2 Hz, 1H), 7.34 (t, J=7.7 Hz, 2H), 7.21-7.14 (m, 1H), 5.05 (s, 2H), 3.52-3.39 (m, 6H), 3.34 (s, 2H), 1.42 (s, 9H). LCMS (Analytical Method H) Rt=0.53 min, MS (ESIpos): m/z 371.4 [M+H]+, Purity=100%.

### Synthesis of tert-butyl 4-[2-(5-bromo-4-phenyl-1H-imidazol-1-yl)acetyl]piperazine-1-carboxylate/Intermediate 18-2

NBS (231 mg, 1.30 mmol) was added to an ice-cold solution of tert-butyl 4-[2-(4-phenylimidazol-1-yl)acetyl]piperazine-1-carboxylate (Intermediate 18-1) (480 mg, 1.30 mmol) in DCM (5 mL). The reaction was allowed to warm up to RT and stirred for 1 h. The reaction was quenched with water, extracted with DCM, dried over MgSO₄ and concentrated in vacuo. The residue was purified by flash chromatography (11 g, KP-NH), eluting with 0-100% EtOAc/heptane to yield the title compound as a yellow solid (274 mg, 46% yield). 1H NMR (500 MHz, DMSO-d6) δ 7.91 (dd, J=8.4, 1.2 Hz, 2H), 7.87 (s, 1H), 7.47-7.38 (m, 2H), 7.33-7.26 (m, 1H), 5.09 (s, 2H), 3.55 (d, J=5.2 Hz, 2H), 3.46 (dt, J=10.1, 4.9 Hz, 4H), 3.34 (s, 2H), 1.43 (s, 9H). LCMS (Analytical Method H) Rt=0.58 min, MS (ESIpos): m/z 449.2, 451.3 [M+H]+, Purity=97%.

### Synthesis of tert-butyl 4-{2-[4-phenyl-5-(pyridin-4-yl)-1H-imidazol-1-yl]acetyl}piperazine-1-carboxylate/Intermediate 18

A mixture of tert-butyl 4-[2-(5-bromo-4-phenyl-imidazol-1-yl)acetyl]piperazine-1-carboxylate (Intermediate 18-2) (270 mg, 0.601 mmol), pyridin-4-yl boronic acid (89 mg, 0.72 mmol), Pd(PPh₃)₄ (35 mg, 0.030 mmol) and Na₂CO₃ (191 mg, 1.80 mmol) in DME (3 mL) and water (1.3 mL) was degassed by sparging with nitrogen and then heated to 100° C. under microwave irradiation for 2 h. The reaction was cooled and diluted with water. The aqueous layer was extracted with DCM, and the organic layer filtered through a Telos phase separator and concentrated in vacuo. The residue was purified by flash chromatography (28 g, KP-NH), eluting with 0-100% EtOAc/heptane to yield the title compound as a pale-yellow solid (230 mg, 79% yield). 1H NMR (500 MHz, DMSO-d6) δ 8.68-8.61 (m, 2H), 7.80 (s, 1H), 7.39-7.33 (m, 2H), 7.28-7.22 (m, 4H), 7.20-7.15 (m, 1H), 4.93 (s, 2H), 3.45-3.34 (m, 4H), 3.27-3.12 (m, 4H), 1.41 (s, 9H). LCMS (Analytical Method H) Rt=0.50 min, MS (ESIpos): m/z 448.4 [M+H]+, Purity=100%.

### Synthesis of tert-butyl 4-{2-[4-(4-fluorophenyl)-5-[2-(methylamino)pyridin-4-yl]-1H-imidazol-1-yl]acetyl}piperazine-1-carboxylate/Intermediate 19

DIPEA (80 μL, 0.458 mmol) was added to a stirred solution of tert-butyl 4-{2-[4-(4-fluorophenyl)-5-(2-fluoropyridin-4-yl)-1H-imidazol-1-yl]acetyl}piperazine-1-carboxylate (Intermediate 15) (75 mg, 0.141 mmol) and methylamine hydrochloride (20 mg, 0.296 mmol) in DMSO (1 mL), and the mixture was heated under microwave irradiation at 80° C. for 1 h and at 100° C. for 4 h. Additional methylamine hydrochloride (40 mg, 0.592 mmol) and DIPEA (120 μL, 0.687 mmol) were added and the reaction was stirred for 8 h at 110° C. The reaction was concentrated in vacuo and the residue purified by preparative HPLC (Method B1) to afford the title compound as a brown oil (68 mg, 76% yield). 1H NMR (500 MHz, DMSO-d6) δ 8.05 (d, J=5.2 Hz, 1H), 7.73 (s, 1H), 7.48-7.43 (m, 2H), 7.13-7.07 (m, 2H), 6.64-6.59 (m, 1H), 6.32 (dd, J=5.2, 1.3 Hz, 1H), 6.27 (s, 1H), 4.82 (s, 2H), 3.41-3.39 (m, 2H), 3.36-3.34 (m, 2H), 3.26-3.23 (m, 2H), 3.22-3.20 (m, 2H), 2.73 (d, J=4.7 Hz, 3H), 1.41 (s, 9H). LCMS (Analytical Method F) Rt=0.61 min, MS (ESIpos): m/z 495.3 [M+H]+, Purity=100%.

### Synthesis of tert-butyl 4-(2-{5-[2-(cyclopentylamino)pyridin-4-yl]-4-(4-fluorophenyl)-1H-imidazol-1-yl}acetyl)piperazine-1-carboxylate/Intermediate 20

DIPEA (60 μL, 0.344 mmol) was added to a stirred solution of tert-butyl 4-{2-[4-(4-fluorophenyl)-5-(2-fluoropyridin-4-yl)-1H-imidazol-1-yl]acetyl}piperazine-1-carboxylate (Intermediate 15) (75 mg, 0.141 mmol) and cyclopentanamine (30 μL, 0.304 mmol) in DMSO (1 mL), and the mixture was heated under microwave irradiation at 80° C. for 1 h and at 100° C. for 4 h. Additional cyclopentanamine (120 μL, 1.22 mmol) and DIPEA (250 μL, 1.43 mmol) were added and the reaction was stirred for 8 h at 110° C. The reaction was concentrated in vacuo and the residue purified by preparative HPLC (Method B1) to afford the title compound as a yellow solid (66 mg, 69% yield). 1H NMR (500 MHz, DMSO-d6) δ 8.03 (d, J=5.1 Hz, 1H), 7.73 (s, 1H), 7.49-7.44 (m, 2H), 7.13-7.08 (m, 2H), 6.64 (d, J=6.6 Hz, 1H), 6.30 (dd, J=5.2, 1.2 Hz, 1H), 6.27 (s, 1H), 4.81 (s, 2H), 4.08-4.00 (m, 1H), 3.43-3.34 (m, 4H), 3.26-3.21 (m, 2H), 3.19-3.14 (m, 2H), 1.89-1.82 (m, 2H), 1.67-1.59 (m, 2H), 1.55-1.47 (m, 2H), 1.40 (s, 9H), 1.39-1.35 (m, 2H). LCMS (Analytical Method F) Rt=1.01 min, MS (ESIpos): m/z 549.3 [M+H]+, Purity=81%.

### Synthesis of benzyl 4-(2-chloroacetyl)piperazine-1-carboxylate/Intermediate 21-1

Chloroacetyl chloride (181 μL, 2.27 mmol) was added dropwise to an ice-cold solution of benzyl piperazine-1-carboxylate (500 mg, 2.27 mmol) and DIPEA (476 μL, 2.72 mmol) in DCM (10 mL). The reaction was stirred for 1 h then quenched into water. The aqueous layer was extracted into EtOAc (3×), the combined organics washed with brine, dried over MgSO₄ and concentrated in vacuo. The residue was purified by flash chromatography (25 g, silica), eluting with 0-100% EtOAc/heptane to yield the title compound as a colourless oil (517 mg, 77% yield). 1H NMR (400 MHz, Chloroform-d) δ 7.41-7.30 (m, 5H), 5.16 (s, 2H), 4.07 (s, 2H), 3.64-3.57 (m, 4H), 3.57-3.48 (m, 4H). LCMS (Analytical Method E) Rt=1.04 min, MS (ESIpos): m/z 297.0, 299.0 [M+H]+, Purity=100%.

### Synthesis of benzyl 4-{2-[4-(4-fluorophenyl)-1H-imidazol-1-yl]acetyl}piperazine-1-carboxylate/Intermediate 21-2

NaH (43 mg, 1.77 mmol) was added to an ice-cold solution of 4-(4-fluorophenyl)-1H-imidazole (284 mg, 1.75 mmol) in THF (6 mL). The reaction was stirred for 10 min then a solution of benzyl 4-(2-chloroacetyl)piperazine-1-carboxylate (Intermediate 21-1) (519 mg, 1.75 mmol) in THF (6 mL) was added and the reaction stirred for 18 h. The reaction was cautiously quenched into water. The aqueous layer was extracted into EtOAc (3×), the combined organics washed with brine, dried over MgSO₄ and concentrated in vacuo. The residue was purified by flash chromatography (25 g, silica), eluting with 0-10% MeOH/DCM to yield the title compound as a colourless oil (514 mg, 70% yield). 1H NMR (500 MHz, Chloroform-d) δ 7.74-7.69 (m, 2H), 7.49 (d, J=1.1 Hz, 1H), 7.39-7.32 (m, 5H), 7.16 (d, J=1.2 Hz, 1H), 7.07-7.01 (m, 2H), 5.15 (s, 2H), 4.77 (s, 2H), 3.66-3.61 (m, 2H), 3.56-3.52 (m, 4H), 3.49-3.41 (m, 2H). LCMS (Analytical Method E) Rt=0.98 min, MS (ESIpos): m/z 423.2 [M+H]+, Purity=99%.

### Synthesis of benzyl 4-{2-[5-bromo-4-(4-fluorophenyl)-1H-imidazol-1-yl]acetyl}piperazine-1-carboxylate/Intermediate 21-3

NBS (670 mg, 3.76 mmol) was added to an ice-cold solution of benzyl 4-{2-[4-(4-fluorophenyl)-1H-imidazol-1-yl]acetyl}piperazine-1-carboxylate (Intermediate 21-2) (1.68 g, 3.74 mmol) in DCM (8 mL). The reaction was stirred for 1 h then additional NBS (67 mg, 0.376 mmol) was added and the mixture stirred at RT for one more h. The reaction was quenched with 1 M NaOH then extracted with DCM, dried over MgSO₄ and concentrated in vacuo. The residue was purified by flash chromatography (50 g, silica), eluting with 20-100% EtOAc/heptane then 0-10% MeOH/EtOAc to yield the title compound as an orange solid (1.5 g, 73% yield). 1H NMR (400 MHz, DMSO-d6) δ 7.96-7.90 (m, 2H), 7.87 (s, 1H), 7.41-7.33 (m, 5H), 7.29-7.23 (m, 2H), 5.12 (s, 2H), 5.09 (s, 2H), 3.62-3.57 (m, 2H), 3.57-3.49 (m, 4H), 3.47-3.40 (m, 2H). LCMS (Analytical Method F) Rt=0.92 min, MS (ESIpos): m/z 501.1, 503.0 [M+H]+, Purity=94%.

### Synthesis of benzyl 4-{2-[5-(2-{[(tert-butoxy)carbonyl]amino}pyridin-4-yl)-4-(4-fluorophenyl)-1H-imidazol-1-yl]acetyl}piperazine-1-carboxylate/Intermediate 21-4

A mixture of benzyl 4-{2-[5-bromo-4-(4-fluorophenyl)-1H-imidazol-1-yl]acetyl}piperazine-1-carboxylate (Intermediate 21-3) (102 mg, 0.203 mmol), tert-butyl [4-(4,4,5,5-tetramethyl-1,3,2-dioxaborolan-2-yl)pyridin-2-yl]carbamate (72 mg, 0.224 mmol), Pd(PPh₃)₄ (12 mg, 0.0102 mmol) and 2 M Na₂CO₃ (aq., 509 μL, 1.02 mmol) in DME (2 mL) was degassed by sparging with nitrogen and then heated to 100° C. under microwave irradiation for 2 h. Additional tert-butyl [4-(4,4,5,5-tetramethyl-1,3,2-dioxaborolan-2-yl)pyridin-2-yl]carbamate (39 mg, 0.122 mmol) and Pd(PPh₃)₄ (7.1 mg, 6.10 μmol) were added, the mixture degassed by sparging with nitrogen and heated to 100° C. under microwave irradiation for 90 min. The reaction was cooled and quenched into water. The aqueous layer was extracted into EtOAc (3×), the combined organics washed with brine, dried over MgSO₄ and concentrated in vacuo. The residue was purified by flash chromatography (10 g, silica), eluting with 0-10% MeOH/DCM to yield the title compound as a cream solid (126 mg, 96% yield). 1H NMR (400 MHz, DMSO-d6) δ 9.89 (s, 1H), 8.28-8.24 (m, 1H), 7.77 (s, 1H), 7.64 (s, 1H), 7.44-7.32 (m, 8H), 7.10 (t, J=9.0 Hz, 2H), 6.82 (dd, J=5.1, 1.5 Hz, 1H), 5.09 (s, 2H), 4.89 (s, 2H), 3.50-3.35 (m, 8H), 1.39 (s, 9H). LCMS (Analytical Method E) Rt=1.10 min, MS (ESIpos): m/z 615.2 [M+H]+, Purity=95%.

### Synthesis of benzyl 4-{2-[5-(2-aminopyridin-4-yl)-4-(4-fluorophenyl)-1H-imidazol-1-yl]acetyl}piperazine-1-carboxylate/Intermediate 21

Benzyl 4-{2-[5-(2-{[(tert-butoxy)carbonyl]amino}pyridin-4-yl)-4-(4-fluorophenyl)-1H-imidazol-1-yl]acetyl}piperazine-1-carboxylate (Intermediate 21-4) (486 mg, 0.514 mmol) was dissolved in 4 M HCl in dioxane (5 mL) and the mixture was stirred at RT for 3 days. The reaction mixture was concentrated in vacuo and purified by preparative HPLC (Method A2) to afford the title compound as a white solid (110 mg, 42% yield). 1H NMR (500 MHz, Chloroform-d) δ 8.14 (d, J=5.2 Hz, 1H), 7.61 (s, 1H), 7.47 (dd, J=8.9, 5.5 Hz, 2H), 7.40-7.33 (m, 5H), 6.94 (t, J=8.8 Hz, 2H), 6.56 (dd, J=5.2, 1.3 Hz, 1H), 6.40 (s, 1H), 5.15 (s, 2H), 4.60 (s, 2H), 4.57 (s, 2H), 3.59 (s, 2H), 3.52-3.42 (m, 4H), 3.31 (s, 2H). LCMS (Analytical Method E) Rt=0.91 min, MS (ESIpos): m/z 515.1 [M+H]+, Purity=100%.

### Synthesis of benzyl 4-{2-[4-(4-chlorophenyl)-1H-imidazol-1-yl]acetyl}piperazine-1-carboxylate/Intermediate 22-1

NaH (60%, 270 mg, 6.74 mmol) was added to an ice-cold solution of 4-(4-chlorophenyl)-1H-imidazole (1.20 g, 6.74 mmol) in THF (12 mL). The reaction was stirred for 10 min then a solution of benzyl 4-(2-chloroacetyl)piperazine-1-carboxylate (Intermediate 21-1) (2.00 g, 6.74 mmol) in THF (6 mL) was added and the reaction stirred for 18 h. The reaction was cautiously quenched into water. The aqueous layer was extracted into EtOAc (3×), the combined organics washed with brine, dried over MgSO₄ and concentrated in vacuo. The residue was purified by flash chromatography (25 g, silica), eluting with 0-20% MeOH/DCM to yield the title compound as an off-white solid (2.6 g, 79% yield). 1H NMR (500 MHz, DMSO-d6) δ 7.79-7.72 (m, 2H), 7.60 (d, J=1.1 Hz, 1H), 7.56 (d, J=1.1 Hz, 1H), 7.42-7.37 (m, 6H), 7.36-7.31 (m, 1H), 5.12 (s, 2H), 5.08 (s, 2H), 3.56-3.39 (m, 8H). LCMS (Analytical Method E) Rt=0.99 min, MS (ESIpos): m/z 438.9 [M+H]+, Purity=100%.

### Synthesis of benzyl 4-{2-[5-bromo-4-(4-chlorophenyl)-1H-imidazol-1-yl]acetyl}piperazine-1-carboxylate/Intermediate 22-2

NBS (1.05 g, 5.88 mmol) was added to an ice-cold solution of benzyl 4-{2-[4-(4-chlorophenyl)-1H-imidazol-1-yl]acetyl}piperazine-1-carboxylate (Intermediate 22-1) (2.58 g, 5.34 mmol) in DCM (35 mL). The reaction was stirred for 1 h at 0° C. then quenched into water (15 mL). The aqueous layer was extracted into DCM (3×15 mL), the combined organics washed with brine (10 mL), dried over Na₂SO₄ and concentrated in vacuo. The residue was purified by flash chromatography (100 g, silica), eluting with 0-10% MeOH/DCM followed by a second purification by flash chromatography (100 g, silica) eluting with 0-50% MeOH/TBME to afford the title compound as an off white solid (1.2 g. 37% yield). 1H NMR (500 MHz, DMSO-d6) δ 7.97-7.92 (m, 2H), 7.89 (s, 1H), 7.52-7.47 (m, 2H), 7.40-7.30 (m, 5H), 5.12 (s, 2H), 5.10 (s, 2H), 3.61-3.41 (m, 8H). LCMS (Analytical Method F) Rt=1.22 min, MS (ESIpos): m/z 517.0, 518.7 [M+H]+, Purity=95%.

### Synthesis of benzyl 4-{2-[4-(4-chlorophenyl)-5-{1H-pyrrolo[2,3-b]pyridin-4-yl}-1H-imidazol-1-yl]acetyl}piperazine-1-carboxylate/Intermediate 22-3

A mixture of benzyl 4-{2-[5-bromo-4-(4-chlorophenyl)-1H-imidazol-1-yl]acetyl}piperazine-1-carboxylate (Intermediate 22-2) (300 mg, 0.487 mmol), 4-(4,4,5,5-tetramethyl-1,3,2-dioxaborolan-2-yl)-1H-pyrrolo[2,3-b]pyridine (168 mg, 0.688 mmol), and Na₂CO₃ (151 mg, 1.43 mmol) were suspended in DME (4 mL) and water (1 mL). The mixture was degassed with nitrogen for 5 min then Pd(PPh₃)₄ (50 mg, 0.0433 mmol) was added. The mixture was sealed and heated at 100° C. for 2 h under microwave irradiation. The reaction was cooled to RT and filtered off, then the filtrate was concentrated in vacuo. The residue was purified by flash chromatography (10 g, silica) eluting with 0-10% MeOH/DCM to yield the title compound as an off-white solid (260 mg, 77% yield). 1H NMR (400 MHz, Chloroform-d) δ 10.49 (s, 1H), 8.36 (d, J=4.9 Hz, 1H), 7.76 (s, 1H), 7.37-7.34 (m, 3H), 7.34-7.30 (m, 5H), 7.10 (d, J=8.6 Hz, 2H), 7.02 (d, J=4.9 Hz, 1H), 6.19-6.16 (m, 1H), 5.10 (s, 2H), 4.63-4.50 (m, 2H), 3.55-3.49 (m, 2H), 3.39-3.34 (m, 2H), 3.21-3.13 (m, 2H), 3.07-3.00 (m, 2H). LCMS (Analytical Method F) Rt=0.78 min, MS (ESIpos): m/z 555.2, 557.2 [M+H]+, Purity=94%.

### Synthesis of 2-[4-(4-chlorophenyl)-5-{1H-pyrrolo[2,3-b]pyridin-4-yl}-1H-imidazol-1-yl]-1-(piperazin-1-yl)ethan-1-one/Intermediate 22

Benzyl 4-{2-[4-(4-chlorophenyl)-5-{1H-pyrrolo[2,3-b]pyridin-4-yl}-1H-imidazol-1-yl]acetyl}piperazine-1-carboxylate (Intermediate 22-3) (260 mg, 0.440 mmol) was dissolved in 12 M aqueous HCl (1.5 mL, 18.0 mmol) and the mixture was stirred at RT for 20 min and at 50° C. for 30 min. MeOH (1.5 mL) was added and the reaction was stirred at 50° C. for 2 hh and at RT for 24 h. The reaction was diluted with MeOH (1 mL) and additional 12 M aqueous HCl (1.5 mL, 18.0 mmol) was added. The mixture was transferred to a sealed tube and stirred at 65° C. for 1 h. The reaction was cooled to 0° C. and basified with NaOH. The solution was extracted with DCM, dried over Na₂SO₄ and concentrated in vacuo. The residue was purified by preparative HPLC (Method A2) to afford the title compound as an orange solid (93 mg, 45% yield). 1H NMR (400 MHz, MeOH-d4) δ 8.29 (d, J=5.0 Hz, 1H), 7.92 (s, 1H), 7.40 (d, J=3.5 Hz, 1H), 7.32-7.27 (m, 2H), 7.16-7.11 (m, 2H), 7.04 (d, J=5.0 Hz, 1H), 6.11 (d, J=3.5 Hz, 1H), 4.96-4.89 (m, 1H), 4.78-4.71 (m, 1H), 3.42-3.37 (m, 2H), 3.19-3.14 (m, 2H), 2.63-2.57 (m, 2H), 2.49-2.42 (m, 2H). LCMS (Analytical Method G) Rt=1.28 min, MS (ESIpos): m/z 421.3, 423.3 [M+H]+, Purity=96%.

### Synthesis of tert-butyl N-[(4-bromopyridin-2-yl)methyl]carbamate/Intermediate 23-1

1 M Borane (THF complex, 6.8 mL, 6.83 mmol) was added to a stirred solution of 4-bromopyridine-2-carbonitrile (250 mg, 1.37 mmol) in anhydrous THF (2.5 mL), and the reaction was stirred at RT for 18 h. Then 2 M HCl (7 mL) was added dropwise and the mixture was heated at 100° C. for 30 min. The reaction mixture was then cooled to RT, basified with 2 M NaOH (10 mL), treated with tert-butoxycarbonyl tert-butyl carbonate (447 mg, 2.05 mmol) and stirred at RT for 2 h. The reaction mixture was diluted with water, extracted with EtOAc (3×20 mL), washed with brine (10 mL), dried over Na₂SO₄, filtered and concentrated in vacuo. The residue was purified by flash chromatography (10 g, silica) eluting with 0-100% TBME/heptane to afford the title compound as a pale yellow oil (318 mg, 74% yield). 1H NMR (500 MHz, DMSO-d6) δ 8.39 (d, J=5.3 Hz, 1H), 7.55 (dd, J=5.3, 1.8 Hz, 1H), 7.52-7.44 (m, 2H), 4.23 (d, J=6.1 Hz, 2H), 1.41 (s, 9H). LCMS (Analytical Method E) Rt=1.08 min, MS (ESIpos): m/z 287.1, 289.1 [M+H]+, Purity=100%.

### Synthesis of [2-({[(tert-butoxy)carbonyl]amino}methyl)pyridin-4-yl]boronic acid/Intermediate 23-2

A mixture of tert-butyl N-[(4-bromopyridin-2-yl)methyl]carbamate (Intermediate 23-1) (750 mg, 2.01 mmol), KOAc (399 mg, 4.02 mmol) and 4,4,5,5-tetramethyl-2-(4,4,5,5-tetramethyl-1,3,2-dioxaborolan-2-yl)-1,3,2-dioxaborolane (766 mg, 3.02 mmol) in anhydrous 1,4-dioxane (7.5 mL) was sparged with nitrogen for 2 min. Then Pd(dppf)Cl₂ (82 mg, 0.101 mmol) was added and the mixture sparged with nitrogen for a further 2 min before it was heated at 100° C. for 4 h using a sealed tube. The reaction mixture was diluted with water (15 mL), and extracted with EtOAc (3×30 mL). The organic extracts were combined, washed with brine (20 mL), dried over Na₂SO₄, filtered, and concentrated in vacuo to afford the title compound (1.49 g, quant.), which was used in the next step without further purification. LCMS (Analytical Method E) Rt=0.65 min, MS (ESIpos): m/z 253.0 [M+H]+, Purity=52%.

### Synthesis of benzyl 4-(2-{5-[2-({[(tert-butoxy)carbonyl]amino}methyl)pyridin-4-yl]-4-(4-chlorophenyl)-1H-imidazol-1-yl}acetyl)piperazine-1-carboxylate/Intermediate 23-3

A mixture of benzyl 4-{2-[5-bromo-4-(4-chlorophenyl)-1H-imidazol-1-yl]acetyl}piperazine-1-carboxylate (Intermediate 22-2) (500 mg, 0.966 mmol), [2-({[(tert-butoxy)carbonyl]amino}methyl)pyridin-4-yl]boronic acid (Intermediate 23-2) (695 mg, 0.966 mmol), Pd(PPh₃)₄ (56 mg, 0.0483 mmol), and Na₂CO₃ (307 mg, 2.90 mmol) in 1,4-dioxane (10 mL) was degassed by sparging with nitrogen. The reaction was heated at 100° C. in a sealed tube. Then, additional Pd(PPh₃)₄ (56 mg, 0.0483 mmol) and [2-({[(tert-butoxy)carbonyl]amino}methyl)pyridin-4-yl]boronic acid (Intermediate 23-2) (904 mg, 1.26 mmol) were added, and the mixture was allowed to stir at 100° C. in a sealed tube for 18 h. The reaction was cooled and quenched into water (10 mL). The aqueous layer was extracted with EtOAc (3×20 mL), the combined organics washed with brine (10 mL), dried over Na₂SO₄, and concentrated in vacuo. The residue was purified by flash chromatography (25 g, silica) eluting with 0-10% MeOH/DCM, followed by preparative HPLC (Method B1) to afford the title compound as an orange gum (11 mg, 10% yield). LCMS (Analytical Method E) Rt=1.14 min, MS (ESIpos): m/z 645.1 [M+H]+, Purity=55%.

### Synthesis of benzyl 4-(2-{5-[2-(aminomethyl)pyridin-4-yl]-4-(4-chlorophenyl)-1H-imidazol-1-yl}acetyl)piperazine-1-carboxylate/Intermediate 23-4

TFA (2.3 mL, 30.3 mmol) was added to a stirred solution of benzyl 4-(2-{5-[2-({[(tert-butoxy)carbonyl]amino}methyl)pyridin-4-yl]-4-(4-chlorophenyl)-1H-imidazol-1-yl}acetyl)piperazine-1-carboxylate (Intermediate 23-3) (1.20 g, 0.836 mmol) in dry DCM (4.5 mL), and the mixture was stirred at RT for 1 h. The solvent was evaporated under reduced pressure, and the residue dissolved in MeOH (2 mL) and loaded onto an SCX column (10 g). The column was washed with MeOH (7 CV), and the product eluted with 3M NH₃ in MeOH (7 CV). The solvent was evaporated under reduced pressure, and the residue was purified by flash chromatography (28 g, KP-NH) eluting with 0-100% IPA/DCM to afford the title compound as a brown oil (302 mg, 53% yield). 1H NMR (400 MHz, DMSO-d6) δ 8.53 (d, J=5.0 Hz, 1H), 7.81 (s, 1H), 7.40-7.30 (m, 10H), 7.06 (dd, J=5.0, 1.6 Hz, 1H), 5.09 (s, 2H), 4.91 (s, 2H), 3.81 (s, 2H), 3.45-3.34 (m, 8H). LCMS (Analytical Method E) Rt=0.89 min, MS (ESIpos): m/z 545.2 [M+H]+, Purity=83%.

### Synthesis of benzyl 4-{2-[4-(4-chlorophenyl)-5-[2-(formamidomethyl)pyridin-4-yl]-1H-imidazol-1-yl]acetyl}piperazine-1-carboxylate and benzyl 4-{2-[4-(4-chlorophenyl)-5-{2-[(dimethylamino)methyl]pyridin-4-yl}-1H-imidazol-1-yl]acetyl}piperazine-1-carboxylate/Intermediates 23 and 24

13 M formaldehyde (37% aq., 6.8 μL, 0.088 mmol) was added to a solution of benzyl 4-(2-{5-[2-(aminomethyl)pyridin-4-yl]-4-(4-chlorophenyl)-1H-imidazol-1-yl}acetyl)piperazine-1-carboxylate (Intermediate 23-4) (50 mg, 0.0734 mmol) in DMF (1 mL), followed by NaBH(OAc)₃ (47 mg, 0.220 mmol), and the mixture was stirred at RT for 72 h. The reaction was diluted with water (10 mL) and extracted with EtOAc (3×20 mL). The organic extracts were combined, dried over Na₂SO₄, filtered, and concentrated in vacuo. The residue was purified by preparative HPLC (Method B1) to afford Intermediate 23 (7.4 mg, 18% yield) and Intermediate 24 (18 mg, 21% yield). Intermediate 23: LCMS (Analytical Method A) Rt=2.28 min, MS (ESIpos): m/z 573.2 [M+H]+, Purity=93%. Intermediate 24: LCMS (Analytical Method A) Rt=1.82 min, MS (ESIpos): m/z 573.2 [M+H]+, Purity=90%.

### Synthesis of benzyl 4-{2-[4-(4-chlorophenyl)-5-{2-[(cyclopentylamino)methyl]pyridin-4-yl}-1H-imidazol-1-yl]acetyl}piperazine-1-carboxylate/Intermediate 25

NaBH(OAc)₃ (65 mg, 0.308 mmol) was added to a solution of benzyl 4-(2-{5-[2-(aminomethyl)pyridin-4-yl]-4-(4-chlorophenyl)-1H-imidazol-1-yl}acetyl)piperazine-1-carboxylate (Intermediate 23-4) (70 mg, 0.103 mmol) and cyclopentanone (11 mg, 0.134 mmol) in anhydrous THF (1 mL), and the mixture was stirred at RT for 72 h. The reaction was diluted with water (10 mL) and extracted with EtOAc (3×20 mL). The organic extracts were combined, dried over Na₂SO₄, filtered, and concentrated in vacuo. The residue was purified by preparative HPLC (Method B1) to afford the title compound as an off-white gum (64.6 mg, 82% yield). LCMS (Analytical Method E) Rt=0.97 min, MS (ESIpos): m/z 613.1 [M+H]+, Purity=85%.

### Synthesis of benzyl 4-{2-[5-(2-benzamidopyridin-4-yl)-4-(4-fluorophenyl)-1H-imidazol-1-yl]acetyl}piperazine-1-carboxylate/Intermediate 26

Benzoyl chloride (3.5 μL, 0.0299 mmol) was added to a solution of benzyl 4-{2-[5-(2-aminopyridin-4-yl)-4-(4-fluorophenyl)-1H-imidazol-1-yl]acetyl}piperazine-1-carboxylate (Intermediate 21) (14 mg, 0.0272 mmol) and Et₃N (5.7 μL, 0.0408 mmol) in THF (1 mL), and the mixture was stirred at RT for 60 h. Additional Et₃N (5.7 μL, 0.0408 mmol) and benzoyl chloride (3.5 μL, 0.0299 mmol) were added, and stirring continued for 2 h. MeOH (0.5 mL) was added followed by 2 M NaOH (68 μL, 0.136 mmol) and the reaction stirred for 1 h. The mixture was diluted with water, the aqueous layer was extracted into EtOAc (3×), the combined organics washed with brine, dried over MgSO₄ and concentrated in vacuo. The residue was purified by flash chromatography (10 g, silica), eluting with 0-10% MeOH/DCM to yield the title compound as a white solid (12 mg, 71% yield). 1H NMR (500 MHz, Chloroform-d) δ 8.50 (s, 1H), 8.20 (d, J=5.2 Hz, 1H), 8.17 (s, 1H), 7.83-7.79 (m, 1H), 7.61 (s, 1H), 7.56-7.51 (m, 1H), 7.45 (d, J=7.9 Hz, 1H), 7.44-7.39 (m, 2H), 7.32-7.22 (m, 4H), 6.94-6.86 (m, 3H), 5.05 (s, 2H), 4.81 (s, 2H), 3.63-3.55 (m, 2H), 3.50-3.40 (m, 3H), 3.40-3.32 (m, 2H). LCMS (Analytical Method E) Rt=1.10 min, MS (ESIpos): m/z 619.2 [M+H]+, Purity=100%.

### Synthesis of benzyl 4-{2-[5-(2-cyclopropaneamidopyridin-4-yl)-4-(4-fluorophenyl)-1H-imidazol-1-yl]acetyl}piperazine-1-carboxylate/Intermediate 27

Cyclopropanecarbonyl chloride (11 μL, 0.121 mmol) was added to a solution of benzyl 4-{2-[5-(2-aminopyridin-4-yl)-4-(4-fluorophenyl)-1H-imidazol-1-yl]acetyl}piperazine-1-carboxylate (Intermediate 21) (30 mg, 0.0583 mmol) and DIPEA (22 μL, 0.126 mmol) in DCM (1 mL), and the mixture was stirred at RT for 1 h. Additional DIPEA (22 μL, 0.126 mmol) and cyclopropanecarbonyl chloride (11 μL, 0.121 mmol) were added, and the mixture was stirred for 16 h. The reaction mixture was concentrated in vacuo then taken up in MeOH (1 mL). 2 M NaOH (1.0 mL, 2.00 mmol) was added and the mixture was stirred at 50° C. for 1 h. The mixture was diluted with water, the aqueous layer was extracted into DCM (3×), the combined organics dried over MgSO₄ and concentrated in vacuo to yield the title compound as a white solid (33 mg, 91% yield), which was used in the next step without further purification. 1H NMR (400 MHz, Chloroform-d) δ 8.19 (d, J=5.1 Hz, 1H), 8.04 (s, 1H), 7.63 (s, 1H), 7.46-7.41 (m, 2H), 7.35 (s, 6H), 6.97-6.90 (m, 2H), 6.88 (dd, J=5.2, 1.3 Hz, 1H), 5.15 (s, 2H), 4.80 (s, 2H), 3.58 (s, 2H), 3.48 (m, 2H), 3.43 (s, 2H), 3.34 (s, 2H), 2.81 (s, 1H), 1.60-1.53 (m, 1H), 1.02-0.98 (m, 2H), 0.90-0.84 (m, 2H). LCMS (Analytical Method E) Rt=1.06 min, MS (ESIpos): m/z 583.3 [M+H]+, Purity=94%.

### Synthesis of benzyl 4-{2-[5-(2-acetamidopyridin-4-yl)-4-(4-fluorophenyl)-1H-imidazol-1-yl]acetyl}piperazine-1-carboxylate/Intermediate 28

Acetyl chloride (15 μL, 0.210 mmol) was added to an ice-cold solution of benzyl 4-{2-[5-(2-aminopyridin-4-yl)-4-(4-fluorophenyl)-1H-imidazol-1-yl]acetyl}piperazine-1-carboxylate (Intermediate 21) (50 mg, 0.0972 mmol) and DIPEA (40 μL, 0.229 mmol) in DCM (1.5 mL), and the mixture was stirred at RT for 1 h. Additional DIPEA (40 μL, 0.229 mmol) and acetyl chloride (15 μL, 0.210 mmol) were added, and the mixture was stirred for 1 h. The reaction mixture was concentrated in vacuo then taken up in MeOH (1 mL). 2 M NaOH (1.5 mL, 3.00 mmol) was added and the mixture was stirred at RT for 1 h. The mixture was diluted with water, the aqueous layer was extracted into DCM (3×), the combined organics dried over MgSO₄ and concentrated in vacuo to yield the title compound as a pale-yellow solid (65 mg, 96% yield), which was used in the next step without further purification. 1H NMR (400 MHz, DMSO-d6) δ 10.63 (s, 1H), 8.34 (d, J=5.1 Hz, 1H), 7.94 (s, 1H), 7.78 (s, 1H), 7.44-7.39 (m, 2H), 7.37-7.36 (m, 5H), 7.13-7.07 (m, 2H), 6.91 (dd, J=5.1, 1.4 Hz, 1H), 5.10 (s, 2H), 4.89 (s, 2H), 3.46-3.42 (m, 8H), 2.04 (s, 3H). LCMS (Analytical Method E) Rt=1.00 min, MS (ESIpos): m/z 557.7 [M+H]+, Purity=80%.

### Synthesis of benzyl 4-{2-[4-(4-chlorophenyl)-5-[2-(acetamidomethyl)pyridin-4-yl]-1H-imidazol-1-yl]acetyl}piperazine-1-carboxylate/Intermediate 29

Acetic anhydride (16 mg, 0.154 mmol) was added to a solution of benzyl 4-(2-{5-[2-(aminomethyl)pyridin-4-yl]-4-(4-chlorophenyl)-1H-imidazol-1-yl}acetyl)piperazine-1-carboxylate (Intermediate 23-4) (70 mg, 0.103 mmol) and Et₃N (43 μL, 0.308 mmol) in anhydrous THF (1 mL), and the mixture was stirred at RT for 72 h. The mixture was quenched with water (10 mL) and extracted with EtOAc (3×15 mL). The combined organics were washed with brine, dried over MgSO₄ and concentrated in vacuo. The residue was purified by preparative HPLC (Method B1) to yield the title compound as an off-white gum (87.3 mg, 87% yield). LCMS (Analytical Method E) Rt=1.01 min, MS (ESIpos): m/z 587.2 [M+H]+, Purity=84%.

### Synthesis of tert-butyl 4-[2-(4-bromo-1H-imidazol-1-yl)acetyl]piperazine-1-carboxylate/Intermediate 30-1

NaH (60%, 299 mg, 7.48 mmol) was added to an ice-cold solution of 4-bromo-1H-imidazole (1.00 g, 6.80 mmol) in anhydrous THF (40 mL). The reaction was stirred for 15 min before tert-butyl 4-(2-chloroacetyl)piperazine-1-carboxylate (intermediate 14-1) (1.79 g, 6.80 mmol) was added, and the reaction was stirred at 0° C. for 2 h. The reaction was carefully quenched into water. The aqueous layer was extracted into EtOAc (2×), the combined organics washed with brine, dried over MgSO₄ and concentrated in vacuo. The residue was purified by flash chromatography (50 g, silica), eluting with 0-10% 1.5M NH₃ in MeOH/DCM to yield the title compound as a white solid (1.83 g, 72% yield). 1H NMR (500 MHz, Chloroform-d) δ 7.37 (d, J=1.5 Hz, 1H), 6.93 (d, J=1.5 Hz, 1H), 4.74 (s, 2H), 3.64-3.60 (m, 2H), 3.52-3.41 (m, 6H), 1.48 (s, 9H). LCMS (Analytical Method F) Rt=0.71 min, MS (ESIpos): m/z 438.9 [M+H]+, Purity=100%.

### Synthesis of tert-butyl 4-[2-(4-bromo-5-iodo-1H-imidazol-1-yl)acetyl]piperazine-1-carboxylate/Intermediate 30-2

N-Iodosuccinimide (1.45 g, 6.43 mmol) was added to a stirred solution of tert-butyl 4-[2-(4-bromo-1H-imidazol-1-yl)acetyl]piperazine-1-carboxylate (Intermediate 30-1) (0.80 g, 2.14 mmol) in anhydrous MeCN (20 mL) and the resulting mixture was refluxed overnight. The mixture was diluted with EtOAc and washed with 1 M aq. Na₂S2O₃ (3×). The combined aqueous layers were extracted with EtOAc (2×), and the combined organics were washed with brine, dried over Na₂SO₄, and concentrated in vacuo. The residue was purified by flash chromatography (25 g, silica), eluting with 0-5% MeOH/DCM to yield the title compound as a pale-yellow solid (750 mg, 70% yield). 1H NMR (500 MHz, Chloroform-d) δ 7.65 (s, 1H), 4.75 (s, 2H), 3.67-3.60 (m, 2H), 3.58-3.43 (m, 6H), 1.48 (s, 9H). LCMS (Analytical Method F) Rt=0.82 min, MS (ESIpos): m/z 499.0 [M+H]+, Purity=100%.

### Synthesis of tert-butyl 4-{2-[4-bromo-5-(pyridin-4-yl)-1H-imidazol-1-yl]acetyl}piperazine-1-carboxylate/Intermediate 30-3

A mixture of tert-butyl 4-[2-(4-bromo-5-iodo-1H-imidazol-1-yl)acetyl]piperazine-1-carboxylate (Intermediate 30-2) (750 mg, 1.50 mmol), pyridin-4-yl boronic acid (185 mg, 1.50 mmol), 2 M Na₂CO₃ (3.8 mL, 7.51 mmol) and Pd(PPh₃)₄ (87 mg, 0.0751 mmol) in DME (12 mL) was degassed by sparging with nitrogen. The reaction was heated to 110° C. under microwave irradiation for 4 h. The reaction mixture was diluted with EtOAc, washed with water, dried over Na₂SO₄, filtered and evaporated under reduced pressure. The residue was purified by flash chromatography (25 g, silica) eluting with 0-6% MeOH/DCM to yield the title compound as a white solid (413 mg, 61% yield). 1H NMR (400 MHz, Chloroform-d) δ 8.75-8.67 (m, 2H), 7.54 (s, 1H), 7.34-7.28 (m, 2H), 4.68 (s, 2H), 3.65-3.53 (m, 2H), 3.47-3.23 (m, 7H), 1.47 (s, 9H). LCMS (Analytical Method F) Rt=0.63 min, MS (ESIpos): m/z 450.1 [M+H]+, Purity=100%.

### Synthesis of tert-butyl 4-{2-[4-(4-methylphenyl)-5-(pyridin-4-yl)-1H-imidazol-1-yl]acetyl}piperazine-1-carboxylate/Intermediate 30

A mixture of tert-butyl 4-{2-[4-bromo-5-(pyridin-4-yl)-1H-imidazol-1-yl]acetyl}piperazine-1-carboxylate (Intermediate 30-3) (54 mg, 0.120 mmol), p-tolylboronic acid (16 mg, 0.120 mmol), Pd(PPh₃)₄ (6.9 mg, 6.00 μmol) and 2 M Na₂CO₃ (0.30 mL, 0.60 mmol) in DME (1.2 mL) was degassed by sparging with nitrogen. The reaction was heated to 125° C. under microwave irradiation for 1 h. The reaction mixture was diluted with EtOAc, washed with water, dried over Na₂SO₄, filtered and evaporated under reduced pressure. The residue was purified by flash chromatography (10 g, silica) eluting with 0-6% MeOH/DCM to yield the title compound as a pale-yellow solid (37.2 mg, 66% yield). 1H NMR (400 MHz, Chloroform-d) δ 8.70-8.64 (m, 2H), 7.65 (s, 1H), 7.33 (d, J=8.1 Hz, 2H), 7.26-7.22 (m, 2H), 7.05 (d, J=8.0 Hz, 2H), 4.61 (s, 2H), 3.58 (s, 2H), 3.44-3.22 (m, 6H), 2.31 (s, 3H), 1.47 (s, 9H). LCMS (Analytical Method G) Rt=0.71 min, MS (ESIpos): m/z 462.3 [M+H]+, Purity=99%.

### Synthesis of tert-butyl 4-{2-[4-(4-methoxyphenyl)-5-(pyridin-4-yl)-1H-imidazol-1-yl]acetyl}piperazine-1-carboxylate/Intermediate 31

A mixture of tert-butyl 4-{2-[4-bromo-5-(pyridin-4-yl)-1H-imidazol-1-yl]acetyl}piperazine-1-carboxylate (Intermediate 30-3) (75 mg, 0.167 mmol), (4-methoxyphenyl)boronic acid (25 mg, 0.167 mmol), Pd(PPh₃)₄ (10 mg, 8.33 μmol) and 2 M Na₂CO₃ (0.42 mL, 0.833 mmol) in DME (1.7 mL) was degassed by sparging with nitrogen. The reaction was heated to 125° C. under microwave irradiation for 1 h. The reaction mixture was diluted with EtOAc, washed with water, dried over Na₂SO₄, filtered and evaporated under reduced pressure. The residue was purified by flash chromatography (10 g, silica) eluting with 0-6% MeOH/DCM to yield the title compound as a yellow solid (58 mg, 73% yield). 1H NMR (400 MHz, Chloroform-d) δ 8.67 (d, J=6.0 Hz, 2H), 7.64 (s, 1H), 7.37 (d, J=8.9 Hz, 2H), 7.28-7.21 (m, 2H), 6.79 (d, J=8.9 Hz, 2H), 4.61 (s, 2H), 3.78 (s, 3H), 3.58 (s, 2H), 3.46-3.22 (m, 6H), 1.47 (s, 9H). LCMS (Analytical Method G) Rt=0.68 min, MS (ESIpos): m/z 478.3 [M+H]+, Purity=100%.

### Synthesis of tert-butyl 4-{2-[5-(pyridin-4-yl)-4-[4-(trifluoromethyl)phenyl]-1H-imidazol-1-yl]acetyl}piperazine-1-carboxylate/Intermediate 32

A mixture of tert-butyl 4-{2-[4-bromo-5-(pyridin-4-yl)-1H-imidazol-1-yl]acetyl}piperazine-1-carboxylate (Intermediate 30-3) (75 mg, 0.167 mmol), [4-(trifluoromethyl)phenyl]boronic acid (32 mg, 0.167 mmol), Pd(PPh₃)₄ (10 mg, 8.33 μmol) and 2 M Na₂CO₃ (0.42 mL, 0.833 mmol) in DME (1.7 mL) was degassed by sparging with nitrogen. The reaction was heated to 125° C. under microwave irradiation for 2 h. The reaction mixture was diluted with EtOAc, washed with water, dried over Na₂SO₄, filtered and evaporated under reduced pressure. The residue was purified by flash chromatography (10 g, silica) eluting with 0-6% MeOH/DCM to yield the title compound as a yellow solid (69 mg, 63% yield). 1H NMR (500 MHz, Chloroform-d) δ 8.74-8.69 (m, 2H), 7.68 (s, 1H), 7.58-7.52 (m, 2H), 7.48 (d, J=8.4 Hz, 2H), 7.27-7.25 (m, 2H), 4.62 (s, 2H), 3.61-3.54 (m, 2H), 3.45-3.22 (m, 6H), 1.47 (s, 9H). LCMS (Analytical Method F) Rt=0.83 min, MS (ESIpos): m/z 516.2 [M+H]+, Purity=88%.

### Synthesis of tert-butyl 4-{2-[4-bromo-5-(2-fluoropyridin-4-yl)-1H-imidazol-1-yl]acetyl}piperazine-1-carboxylate/Intermediate 33-1

A mixture of tert-butyl 4-[2-(4-bromo-5-iodo-1H-imidazol-1-yl)acetyl]piperazine-1-carboxylate (Intermediate 30-2) (330 mg, 0.615 mmol), (2-fluoropyridin-4-yl)boronic acid (88 mg, 0.627 mmol), 2 M Na₂CO₃ (0.92 mL, 1.84 mmol) and Pd(PPh₃)₄ (36 mg, 0.0307 mmol) in DME (3.5 mL) was degassed by sparging with nitrogen. The reaction was heated to 125° C. under microwave irradiation for 3 h. The reaction mixture was diluted with EtOAc, washed with water, dried over Na₂SO₄, filtered and evaporated under reduced pressure. The residue was purified by flash chromatography (25 g, silica) eluting with 0-6% MeOH/DCM to yield the title compound as a white solid (210 mg, 66% yield). 1H NMR (400 MHz, Chloroform-d) δ 8.34 (d, J=5.2 Hz, 1H), 7.57 (s, 1H), 7.25 (dt, J=5.1, 1.6 Hz, 1H), 6.98 (t, J=1.3 Hz, 1H), 4.73 (s, 2H), 3.68-3.58 (m, 2H), 3.50-3.43 (m, 4H), 3.41-3.32 (m, 2H), 1.50 (s, 9H).

### Synthesis of tert-butyl 4-(2-{4-bromo-5-[2-(cyclopentylamino)pyridin-4-yl]-1H-imidazol-1-yl}acetyl)piperazine-1-carboxylate/Intermediate 33-2

DIPEA (0.78 mL, 4.48 mmol) was added to a stirred solution of tert-butyl 4-{2-[4-bromo-5-(2-fluoropyridin-4-yl)-1H-imidazol-1-yl]acetyl}piperazine-1-carboxylate (Intermediate 33-1) (210 mg, 0.448 mmol) and cyclopentanamine (0.44 mL, 4.48 mmol) in DMSO (3.2 mL), and the mixture was heated at 110° C. overnight in a sealed tube. The reaction was concentrated in vacuo and the residue triturated with Et₂O and recovered by filtration to yield the title compound (240 mg, 65% yield), which was used in the next step without further purification. 1H NMR (400 MHz, Chloroform-d) δ 8.10 (d, J=5.2 Hz, 1H), 7.52 (s, 1H), 6.44 (dd, J=5.2, 1.4 Hz, 1H), 6.40 (s, 1H), 4.79 (d, J=6.6 Hz, 1H), 4.69 (s, 2H), 3.93 (h, J=6.4 Hz, 1H), 3.62-3.53 (m, 2H), 3.48-3.23 (m, 6H), 2.12-1.97 (m, 2H), 1.78-1.57 (m, 4H), 1.54-1.40 (m, 11H). LCMS (Analytical Method F) Rt=0.59 min, MS (ESIpos): m/z 533.3 [M+H]+, Purity=83%.

### Synthesis of tert-butyl 4-(2-{5-[2-(cyclopentylamino)pyridin-4-yl]-4-(4-methoxyphenyl)-1H-imidazol-1-yl}acetyl)piperazine-1-carboxylate/Intermediate 33

A mixture of tert-butyl 4-(2-{4-bromo-5-[2-(cyclopentylamino)pyridin-4-yl]-1H-imidazol-1-yl}acetyl)piperazine-1-carboxylate (Intermediate 33-2) (120 mg, 0.146 mmol), (4-methoxyphenyl)boronic acid (25 mg, 0.165 mmol), Pd(PPh₃)₄ (8.0 mg, 6.92 μmol) and 2 M Na₂CO₃ (0.34 mL, 0.680 mmol) in DME (1.4 mL) was degassed by sparging with nitrogen. The reaction was heated to 125° C. under microwave irradiation for 2 h. The reaction mixture was diluted with EtOAc, washed with water, dried over Na₂SO₄, filtered and evaporated under reduced pressure. The residue was purified by flash chromatography (25 g, silica) eluting with 0-5% MeOH/DCM to yield the title compound (75 mg, 82% yield). 1H NMR (500 MHz, Chloroform-d) δ 8.11 (d, J=5.1 Hz, 1H), 7.61 (s, 1H), 7.50-7.44 (m, 2H), 6.83-6.77 (m, 2H), 6.47 (dd, J=5.1, 1.3 Hz, 1H), 6.30 (s, 1H), 4.67 (d, J=6.7 Hz, 1H), 4.61 (s, 2H), 3.85 (h, J=6.5 Hz, 1H), 3.78 (s, 3H), 3.62-3.52 (m, 2H), 3.44-3.24 (m, 6H), 2.00-1.87 (m, 2H), 1.71 (dq, J=14.7, 7.7 Hz, 2H), 1.47 (s, 9H), 1.45-1.35 (m, 2H). LCMS (Analytical Method F) Rt=0.75 min, MS (ESIpos): m/z 561.4 [M+H]+, Purity=90%.

### Synthesis of tert-butyl 4-(2-{5-[2-(cyclopentylamino)pyridin-4-yl]-4-[4-(trifluoromethyl)phenyl]-1H-imidazol-1-yl}acetyl)piperazine-1-carboxylate/Intermediate 34

A mixture of tert-butyl 4-(2-{4-bromo-5-[2-(cyclopentylamino)pyridin-4-yl]-1H-imidazol-1-yl}acetyl)piperazine-1-carboxylate (Intermediate 33-2) (120 mg, 0.146 mmol), [4-(trifluoromethyl)phenyl]boronic acid (31 mg, 0.162 mmol), Pd(PPh₃)₄ (7.8 mg, 6.75 μmol) and 2 M Na₂CO₃ (0.34 mL, 0.680 mmol) in DME (1.3 mL) was degassed by sparging with nitrogen. The reaction was heated to 125° C. under microwave irradiation for 2 h. The reaction mixture was diluted with EtOAc, washed with water, dried over Na₂SO₄, filtered and evaporated under reduced pressure. The residue was purified by flash chromatography (25 g, silica) eluting with 0-5% MeOH/DCM to yield the title compound (67 mg, 69% yield). 1H NMR (500 MHz, Chloroform-d) δ 8.08 (d, J=5.1 Hz, 1H), 7.63-7.56 (m, 3H), 7.43 (d, J=8.3 Hz, 2H), 6.41 (dd, J=5.1, 1.3 Hz, 1H), 6.22 (s, 1H), 4.67 (d, J=6.2 Hz, 1H), 4.56 (s, 2H), 3.77 (h, J=6.6 Hz, 1H), 3.55-3.46 (m, 2H), 3.38-3.16 (m, 6H), 1.92-1.82 (m, 2H), 1.69-1.59 (m, 2H), 1.40 (s, 9H), 1.38-1.27 (m, 2H). LCMS (Analytical Method F) Rt=0.86 min, MS (ESIpos): m/z 599.3 [M+H]+, Purity=90%.

### Synthesis of N-[2-(4-fluorophenyl)-2-oxoethyl]acetamide/Intermediate 35-1

2-Amino-1-(4-fluorophenyl)ethanone hydrochloride (1:1) (500 mg, 2.64 mmol), acetic anhydride (260 μL, 2.76 mmol) and DIPEA (470 μL, 2.69 mmol) were suspended in THF (6 mL) and the mixture was stirred at RT for 1 h. The reaction was cooled to RT and quenched with water, then extracted with DCM, dried over MgSO₄, filtered and concentrated in vacuo. The residue was purified by flash chromatography (silica), eluting with 0-100% EtOAc/heptane then 0-20% MeOH/EtOAc to yield the title compound (338 mg, 63% yield). 1H NMR (400 MHz, Chloroform-d) δ 8.04-7.98 (m, 2H), 7.20-7.13 (m, 2H), 6.57 (s, 1H), 4.73 (d, J=4.3 Hz, 2H), 2.10 (s, 3H). LCMS (Analytical Method D) Rt=0.83 min, MS (ESIpos): m/z 196.0 [M+H]+, Purity=96%.

### Synthesis of 4-(4-fluorophenyl)-2-methyl-1H-imidazole/Intermediate 35-2

N-[2-(4-Fluorophenyl)-2-oxo-ethyl]acetamide (Intermediate 35-1) (320 mg, 1.57 mmol) and ammonium acetate (1.22 g, 15.8 mmol) were dissolved in acetic acid (6 mL) and the mixture was stirred at 120° C. for 18 h. The reaction was cooled to RT and partitioned between 2 M NaOH and DCM. The organic layer was separated and the aqueous extracted with DCM. The organics were combined dried over MgSO₄, filtered and concentrated in vacuo. The residue was purified by flash chromatography (silica) eluting with 0-100% EtOAc/heptane then 0-40% MeOH/EtOAc. The resulting product was purified by preparative HPLC (Method B1) to yield the title compound (133 mg, 47% yield). 1H NMR (400 MHz, DMSO-d6) δ 11.82 (s, 1H), 7.76-7.67 (m, 2H), 7.38 (s, 1H), 7.19-7.09 (m, 2H), 2.30 (s, 3H). LCMS (Analytical Method D) Rt=0.73 min, MS (ESIpos): m/z 177.0 [M+H]+, Purity=100%.

### Synthesis of tert-butyl 4-{2-[4-(4-fluorophenyl)-2-methyl-1H-imidazol-1-yl]acetyl}piperazine-1-carboxylate/Intermediate 35-3

To a solution of 4-(4-fluorophenyl)-2-methyl-1H-imidazole (Intermediate 35-2) (95 mg, 0.539 mmol) in THF (3 mL) at 0° C. was added NaH (60%, 25 mg, 0.625 mmol). The slurry was stirred for 10 min, then tert-butyl 4-(2-bromoacetyl)piperazine-1-carboxylate (165 mg, 0.537 mmol) was added and the reaction was stirred for 2 h. The reaction was quenched with water then extracted with EtOAc, dried over MgSO₄, filtered and concentrated in vacuo. The residue was purified by flash chromatography (25 g, silica) eluting with 0-100% EtOAc/heptane then 0-40% MeOH/EtOAc to yield the title compound (207 mg, 86% yield). 1H NMR (400 MHz, Chloroform-d) δ 7.70-7.64 (m, 2H), 7.05-6.97 (m, 3H), 4.66 (s, 2H), 3.62 (s, 2H), 3.51-3.38 (m, 6H), 2.36 (s, 3H), 1.47 (s, 9H). LCMS (Analytical Method D) Rt=0.86 min, MS (ESIpos): m/z 403.1 [M+H]+, Purity=92%.

### Synthesis of tert-butyl 4-{2-[5-bromo-4-(4-fluorophenyl)-2-methyl-1H-imidazol-1-yl]acetyl}piperazine-1-carboxylate/Intermediate 35-4

To an ice-cold solution of tert-butyl 4-[2-[4-(4-fluorophenyl)-2-methyl-imidazol-1-yl]acetyl]piperazine-1-carboxylate (Intermediate 35-3) (190 mg, 0.434 mmol) in DCM (4 mL), NBS (85 mg, 0.478 mmol) was added, and the reaction was stirred at 0° C. for 1 h. The reaction mixture was partitioned between 1 M NaOH and DCM. The organic phase was separated, dried over MgSO₄, filtered and concentrated in vacuo. The residue was purified by flash chromatography (silica) eluting with 0-5% MeOH/DCM to yield the title compound (98 mg, 46% yield). 1H NMR (400 MHz, Chloroform-d) δ 7.92-7.85 (m, 2H), 7.10-7.03 (m, 2H), 4.73 (s, 2H), 3.67-3.61 (m, 2H), 3.59-3.52 (m, 4H), 3.50-3.45 (m, 2H), 2.42 (s, 3H), 1.49 (s, 9H). LCMS (Analytical Method D) Rt=1.08 min, MS (ESIpos): m/z 481.1, 483.1 [M+H]+, Purity=98%.

### Synthesis of tert-butyl 4-{2-[4-(4-fluorophenyl)-2-methyl-5-(pyridin-4-yl)-1H-imidazol-1-yl]acetyl}piperazine-1-carboxylate/Intermediate 35

A suspension of tert-butyl 4-[2-[5-bromo-4-(4-fluorophenyl)-2-methyl-imidazol-1-yl]acetyl]piperazine-1-carboxylate (Intermediate 35-4) (85 mg, 0.173 mmol), pyridin-4-yl boronic acid (32 mg, 0.260 mmol) and CS₂CO₃ (113 mg, 0.346 mmol) in 1,4-dioxane (0.8 mL) and water (0.2 mL) was degassed with nitrogen for 5 min before Pd(dppf)Cl₂ (14 mg, 0.0173 mmol) was added. The mixture was sealed and stirred at 80° C. for 6 h. The reaction was retreated with Pd(dppf)Cl₂ (14 mg, 0.0173 mmol) and pyridin-4-yl boronic acid (32 mg, 0.260 mmol) and stirred at 80° C. for 16 h. The reaction was cooled to RT and filtered through celite, washing with EtOAc. The filtrate was concentrated in vacuo and the residue was purified by preparative HPLC (Method A2) to yield the title compound (46 mg, 52% yield). 1H NMR (400 MHz, Chloroform-d) δ 8.67-8.61 (m, 2H), 7.39-7.33 (m, 2H), 7.21-7.15 (m, 2H), 6.94-6.86 (m, 2H), 4.48 (s, 2H), 3.64-3.55 (m, 2H), 3.45-3.35 (m, 4H), 3.34-3.26 (m, 2H), 2.42 (s, 3H), 1.47 (s, 9H). LCMS (Analytical Method D) Rt=0.93 min, MS (ESIpos): m/z 480.2 [M+H]+, Purity=94%.

### Synthesis of 2,2,2-trifluoro-N-[2-(4-fluorophenyl)-2-oxoethyl]acetamide/Intermediate 36-1

To an ice-cold solution of 2-amino-1-(4-fluorophenyl)ethanone hydrochloride (1:1) (500 mg, 2.64 mmol) and DIPEA (0.51 mL, 2.90 mmol) in DCM (10 mL), (2,2,2-trifluoroacetyl) 2,2,2-trifluoroacetate (0.37 mL, 2.64 mmol) was added dropwise, and the solution was stirred at this temperature for 1 h, then stirred at RT overnight. The mixture was partitioned between water and DCM. The organic phase was separated, dried over MgSO₄, filtered and concentrated in vacuo. The residue was purified by flash chromatography (silica) eluting with 0-80% EtOAc/heptane to yield the title compound (450 mg, 61% yield). 1H NMR (400 MHz, Chloroform-d) δ 8.06-7.99 (m, 2H), 7.47 (s, 1H), 7.25-7.18 (m, 2H), 4.80 (d, J=4.1 Hz, 2H). LCMS (Analytical Method G) Rt=1.46 min, MS (ESIpos): m/z 267.1 [M+H]+, Purity=89%.

### Synthesis of 4-(4-fluorophenyl)-2-(trifluoromethyl)-1H-imidazole/Intermediate 36-2

2,2,2-trifluoro-N-[2-(4-Fluorophenyl)-2-oxo-ethyl]acetamide (Intermediate 36-1) (440 mg, 1.57 mmol) and ammonium acetate (1218 mg, 15.8 mmol) were dissolved in acetic acid (6 mL) and the mixture was stirred at 120° C. for 16 h. The reaction was cooled to RT and quenched with water, then extracted with DCM. The organics were dried over MgSO₄, filtered and concentrated in vacuo. The residue was purified by flash chromatography (10 g, silica) eluting with 0-8% MeOH/DCM to yield the title compound (158 mg, 44% yield). 1H NMR (400 MHz, DMSO-d6) δ 13.66 (s, 1H), 7.89-7.80 (m, 3H), 7.27-7.18 (m, 2H). LCMS (Analytical Method D) Rt=1.11 min, MS (ESIpos): m/z 231.0 [M+H]+, Purity=100%.

### Synthesis of tert-butyl 4-{2-[4-(4-fluorophenyl)-2-(trifluoromethyl)-1H-imidazol-1-yl]acetyl}piperazine-1-carboxylate/Intermediate 36-3

To an ice-cold solution of 4-(4-fluorophenyl)-2-(trifluoromethyl)-1H-imidazole (Intermediate 36-2) (145 mg, 0.630 mmol) in THF (3 mL), NaH (60%, 26 mg, 0.650 mmol) was added. The mixture was stirred for 5 min, then tert-butyl 4-(2-bromoacetyl)piperazine-1-carboxylate (200 mg, 0.651 mmol) was added and the reaction was stirred for 2 h, then quenched with water and extracted with EtOAc, dried over MgSO₄, filtered and concentrated in vacuo. The residue was purified by flash chromatography (10 g, silica) eluting with 0-10% MeOH/DCM to yield the title compound (250 mg, 76% yield). 1H NMR (400 MHz, Chloroform-d) δ 7.72-7.65 (m, 2H), 7.21 (s, 1H), 7.06-6.99 (m, 2H), 4.87 (s, 2H), 3.63-3.55 (m, 2H), 3.53-3.47 (m, 2H), 3.47-3.38 (m, 4H), 1.46 (s, 9H). LCMS (Analytical Method D) Rt=1.22 min, MS (ESIpos): m/z 457.1 [M+H]+, Purity=88%.

### Synthesis of tert-butyl 4-{2-[5-bromo-4-(4-fluorophenyl)-2-(trifluoromethyl)-1H-imidazol-1-yl]acetyl}piperazine-1-carboxylate/Intermediate 36-4

To an ice-cold solution of tert-butyl 4-[2-[4-(4-fluorophenyl)-2-(trifluoromethyl)imidazol-1-yl]acetyl]piperazine-1-carboxylate (Intermediate 36-3) (250 mg, 0.482 mmol) in DCM (3 mL), NBS (90 mg, 0.506 mmol) was added, and the mixture was stirred for 1 h. The reaction was quenched with water and extracted with DCM, dried over MgSO₄, filtered and concentrated in vacuo. The residue was purified by flash chromatography (silica) eluting with 0-5% MeOH/DCM to yield the title compound (255 mg, 95% yield). 1H NMR (400 MHz, Chloroform-d) δ 7.95-7.88 (m, 2H), 7.14-7.07 (m, 2H), 4.95 (s, 2H), 3.68-3.42 (m, 8H), 1.49 (s, 9H). LCMS (Analytical Method D) Rt=1.36 min, MS (ESIpos): m/z 535.1, 537.1 [M+H]+, Purity=98%.

### Synthesis of tert-butyl 4-{2-[4-(4-fluorophenyl)-5-(pyridin-4-yl)-2-(trifluoromethyl)-1H-imidazol-1-yl]acetyl}piperazine-1-carboxylate/Intermediate 36

Pyridin-4-yl boronic acid (85 mg, 0.692 mmol), tert-butyl 4-[2-[5-bromo-4-(4-fluorophenyl)-2-(trifluoromethyl)imidazol-1-yl]acetyl]piperazine-1-carboxylate (Intermediate 36-4) (245 mg, 0.458 mmol) and Na₂CO₃ (146 mg, 1.37 mmol) were suspended in DME/water (4:1, 2 mL) and the mixture was degassed with nitrogen for 10 min. Pd(PPh₃)₄ (50 mg, 0.0433 mmol) was then added, and the reaction was sealed and stirred at 100° C. for 9 h. Additional Pd(PPh₃)₄ (20 mg, 0.0173 mmol) and pyridin-4-ylboronic acid (30 mg, 0.244 mmol) were added and the reaction was stirred at 100° C. for 1 h. The reaction was partitioned between water and EtOAc. The organic phase was separated, dried over MgSO₄, filtered and concentrated in vacuo. The residue was purified by preparative HPLC (Method A2) to yield the title compound (118 mg, 46% yield). 1H NMR (400 MHz, DMSO-d6) δ 8.78-8.71 (m, 2H), 7.40-7.31 (m, 4H), 7.18-7.09 (m, 2H), 4.96 (s, 2H), 3.44-3.33 (m, 4H), 3.25-3.20 (m, 2H), 3.17-3.14 (m, 2H), 1.41 (s, 9H). LCMS (Analytical Method D) Rt=1.18 min, MS (ESIpos): m/z 534.1 [M+H]+, Purity=100%.

### Synthesis of 4-(4-fluorophenyl)-2-(propan-2-yl)-1H-imidazole/Intermediate 37-1

A mixture of 4-bromo-2-isopropyl-1H-imidazole (250 mg, 1.32 mmol), (4-fluorophenyl)boronic acid (222 mg, 1.59 mmol) and K₂CO₃ (365 mg, 2.64 mmol) in 1,4-dioxane (8 mL) and water (1 mL) was degassed with nitrogen for 10 min. Pd(dppf)Cl₂ (100 mg, 0.136 mmol) was then added and the mixture was degassed for further 5 min before being sealed and stirred at 90° C. for 16 h. The mixture was cooled to RT and filtered through celite. The filtrate was diluted with water and extracted with EtOAc, dried over MgSO₄, filtered and concentrated in vacuo. The residue was purified by preparative HPLC (Method A1) to yield the title compound (61 mg, 20% yield). 1H NMR (400 MHz, Chloroform-d) δ 9.15 (s, 1H), 7.78-7.65 (m, 2H), 7.13 (s, 1H), 7.06-6.99 (m, 2H), 3.18-3.08 (m, 1H), 1.36 (d, J=7.0 Hz, 6H). LCMS (Analytical Method D) Rt=0.76 min, MS (ESIpos): m/z 205.1 [M+H]+, Purity=100%.

### Synthesis of tert-butyl 4-{2-[4-(4-fluorophenyl)-2-(propan-2-yl)-1H-imidazol-1-yl]acetyl}piperazine-1-carboxylate/Intermediate 37-2

To an ice-cold solution of 4-(4-fluorophenyl)-2-isopropyl-1H-imidazole (Intermediate 37-1) (55 mg, 0.269 mmol) in THF (1.5 mL), NaH (60%, 12 mg, 0.300 mmol) was added. The reaction was stirred for 5 min then tert-butyl 4-(2-chloroacetyl)piperazine-1-carboxylate (intermediate 14-1) (75 mg, 0.285 mmol) was added and the mixture was stirred for 2 h. The reaction was quenched with water, then extracted with DCM, dried over MgSO₄, filtered and concentrated in vacuo. The residue was purified by flash chromatography (10 g, silica) eluting with 0-100% EtOAc/heptane to yield the title compound (109 mg, 94% yield). 1H NMR (500 MHz, Chloroform-d) δ 7.71-7.66 (m, 2H), 7.02-6.97 (m, 2H), 6.95 (s, 1H), 4.66 (s, 2H), 3.64-3.56 (m, 2H), 3.48-3.38 (m, 6H), 2.83 (hept, J=6.8 Hz, 1H), 1.46 (s, 9H), 1.33 (d, J=6.8 Hz, 6H). LCMS (Analytical Method D) Rt=0.96 min, MS (ESIpos): m/z 431.5 [M+H]+, Purity=100%.

### Synthesis of tert-butyl 4-{2-[5-bromo-4-(4-fluorophenyl)-2-(propan-2-yl)-1H-imidazol-1-yl]acetyl}piperazine-1-carboxylate/Intermediate 37-3

To an ice-cold solution of tert-butyl 4-[2-[4-(4-fluorophenyl)-2-isopropyl-imidazol-1-yl]acetyl]piperazine-1-carboxylate (Intermediate 37-2) (109 mg, 0.253 mmol) in DCM (2 mL), NBS (50 mg, 0.281 mmol) was added and the reaction was stirred for 30 min. The reaction was quenched with water and extracted with DCM, dried over MgSO₄, filtered and concentrated in vacuo. The residue was purified by flash chromatography (10 g, silica) eluting with 0-10% MeOH/DCM. The resulting product was further purified by preparative HPLC (Method B1) to yield the title compound (66 mg, 49% yield). 1H NMR (400 MHz, Chloroform-d) δ 7.93-7.86 (m, 2H), 7.09-7.02 (m, 2H), 4.76 (s, 2H), 3.67-3.60 (m, 2H), 3.60-3.52 (m, 4H), 3.51-3.43 (m, 2H), 2.88 (p, J=6.8 Hz, 1H), 1.48 (s, 9H), 1.34 (d, J=6.8 Hz, 6H). LCMS (Analytical Method D) Rt=1.17 min, MS (ESIpos): m/z 509.2, 511.2 [M+H]+, Purity=95%.

### Synthesis of tert-butyl 4-{2-[4-(4-fluorophenyl)-2-(propan-2-yl)-5-(pyridin-4-yl)-1H-imidazol-1-yl]acetyl}piperazine-1-carboxylate/Intermediate 37

A suspension of tert-butyl 4-[2-[5-bromo-4-(4-fluorophenyl)-2-isopropyl-imidazol-1-yl]acetyl]piperazine-1-carboxylate (Intermediate 37-3) (66 mg, 0.123 mmol), pyridin-4-ylboronic acid (22 mg, 0.179 mmol) and Na₂CO₃ (40 mg, 0.377 mmol) in DME (0.8 mL) and water (0.2 mL) was degassed with nitrogen for 5 min. Pd(PPh₃)₄ (15 mg, 0.0130 mmol) was added and the mixture was degassed for 5 min, then sealed and stirred at 100° C. for 1.5 h. After cooling to RT, the reaction was filtered through celite and the filtrate was concentrated in vacuo. The residue was purified by flash chromatography (10 g, silica) eluting with 0-10% MeOH/DCM to yield the title compound (46 mg, 65% yield). 1H NMR (400 MHz, Chloroform-d) δ 8.64-8.61 (m, 2H), 7.40-7.34 (m, 2H), 7.21-7.19 (m, 2H), 6.93-6.86 (m, 2H), 4.52 (s, 2H), 3.63-3.55 (m, 2H), 3.43-3.34 (m, 4H), 3.34-3.27 (m, 2H), 2.84 (p, J=6.8 Hz, 1H), 1.46 (s, 9H), 1.40 (d, J=6.8 Hz, 6H). LCMS (Analytical Method D) Rt=1.01 min, MS (ESIpos): m/z 508.4 [M+H]+, Purity=88%.

### Synthesis of tert-butyl 2-[4-(4-fluorophenyl)imidazol-1-yl]propanoate/Intermediate 38-1

To an ice-cold solution of 4-(4-fluorophenyl)-1H-imidazole (500 mg, 3.08 mmol) in THF (10 mL), NaH (60%, 130 mg, 3.25 mmol) was added. The mixture was stirred for 10 min then tert-butyl 2-bromopropanoate (670 mg, 3.20 mmol) was added and the reaction was stirred for 1.5 h. The mixture was quenched with water, extracted with EtOAc, dried over MgSO₄, filtered and concentrated in vacuo. The residue was purified by flash chromatography (25 g, silica) eluting with 0-100% EtOAc/heptane to yield the title compound (750 mg, 84% yield). 1H NMR (400 MHz, Chloroform-d) δ 7.76-7.69 (m, 2H), 7.57 (d, J=1.3 Hz, 1H), 7.24 (d, J=1.3 Hz, 1H), 7.08-7.01 (m, 2H), 4.73 (q, J=7.3 Hz, 1H), 1.73 (d, J=7.3 Hz, 3H), 1.45 (s, 9H). LCMS (Analytical Method D) Rt=0.97 min, MS (ESIpos): m/z 291.3 [M+H]+, Purity=100%.

### Synthesis of 2-[4-(4-fluorophenyl)-1H-imidazol-1-yl]propanoic acid/Intermediate 38-2

tert-Butyl 2-[4-(4-fluorophenyl)imidazol-1-yl]propanoate (Intermediate 38-1) (750 mg, 2.58 mmol) was dissolved in DCM (12 mL) and TFA (4 mL). The solution was stirred at RT for 16 h and then concentrated in vacuo. The residue was azeotroped with toluene and concentrated in vacuo to yield the title compound as a TFA salt (822 mg, 82% yield), which was used in the next step without further purification. 1H NMR (400 MHz, Chloroform-d) δ 8.95 (d, J=1.6 Hz, 1H), 7.64-7.57 (m, 2H), 7.37 (d, J=1.5 Hz, 1H), 7.19-7.11 (m, 2H), 5.11 (q, J=7.4 Hz, 1H), 1.88 (d, J=7.4 Hz, 3H). LCMS (Analytical Method D) Rt=0.74 min, MS (ESIpos): m/z 235.1 [M+H]+, Purity=98%.

### Synthesis of tert-butyl 4-{2-[4-(4-fluorophenyl)-1H-imidazol-1-yl]propanoyl}piperazine-1-carboxylate/Intermediate 38-3

A solution of 2-[4-(4-fluorophenyl)imidazol-1-yl]propanoic acid TFA salt (Intermediate 38-2) (822 mg, 2.31 mmol), DIPEA (1.25 mL, 7.16 mmol) and HATU (1.0 g, 2.63 mmol) in DMF (8 mL) was stirred at RT for 15 min. tert-Butyl piperazine-1-carboxylate (475 mg, 2.55 mmol) was then added and the reaction was stirred for 16 h. The reaction was diluted with water and extracted with EtOAc, dried over MgSO₄, filtered and concentrated in vacuo. The residue was purified by flash chromatography (25 g, silica) eluting with 0-10% MeOH/DCM to yield the title compound (939 mg, 91% yield). 1H NMR (400 MHz, Chloroform-d) δ 7.74 (s, 1H), 7.70-7.63 (m, 2H), 7.34 (d, J=1.1 Hz, 1H), 7.06-6.99 (m, 2H), 5.24 (d, J=6.9 Hz, 1H), 3.76-3.66 (m, 1H), 3.57-3.44 (m, 5H), 3.39-3.29 (m, 1H), 3.26-3.17 (m, 1H), 1.70 (d, J=6.9 Hz, 3H), 1.44 (s, 9H). LCMS (Analytical Method D) Rt=0.94 min, MS (ESIpos): m/z 403.5 [M+H]+, Purity=90%.

### Synthesis of tert-butyl 4-{2-[5-bromo-4-(4-fluorophenyl)-1H-imidazol-1-yl]propanoyl}piperazine-1-carboxylate/Intermediate 38-4

To an ice-cold solution of tert-butyl 4-[2-[4-(4-fluorophenyl)imidazol-1-yl]propanoyl]piperazine-1-carboxylate (Intermediate 38-3) (920 mg, 2.06 mmol) in DCM (10 mL), NBS (370 mg, 2.08 mmol) was added and the reaction was stirred at 0° C. for 1 h. The reaction was quenched with water and extracted with DCM, dried over MgSO₄, filtered and concentrated in vacuo. The residue was purified by flash chromatography (25 g, silica) eluting with 0-10% MeOH/DCM to yield the title compound (1.07 g, 93% yield). 1H NMR (400 MHz, Chloroform-d) δ 7.93-7.87 (m, 2H), 7.83 (s, 1H), 7.12-7.06 (m, 2H), 5.26 (q, J=7.0 Hz, 1H), 3.83-3.74 (m, 1H), 3.55-3.44 (m, 5H), 3.37-3.29 (m, 1H), 3.25-3.17 (m, 1H), 1.71 (d, J=7.0 Hz, 3H), 1.45 (s, 9H). LCMS (Analytical Method D) Rt=1.18 min, MS (ESIpos): m/z 481.0, 483.0 [M+H]+, Purity=86%.

### Synthesis of tert-butyl 4-{2-[4-(4-fluorophenyl)-5-(pyridin-4-yl)-1H-imidazol-1-yl]propanoyl}piperazine-1-carboxylate/Intermediate 38

tert-Butyl 4-[2-[5-bromo-4-(4-fluorophenyl)imidazol-1-yl]propanoyl]piperazine-1-carboxylate (Intermediate 38-4) (400 mg, 0.715 mmol), pyridin-4-yl boronic acid (130 mg, 1.06 mmol) and Na₂CO₃ (225 mg, 2.12 mmol) were dissolved in DME (4 mL) and water (1 mL) and the mixture was degassed with nitrogen for 10 min. Pd(PPh₃)₄ (86 mg, 0.0744 mmol) was added and the mixture was degassed for 5 min then sealed and stirred at 100° C. for 3 h under microwave irradiation. The mixture was filtered through celite, washing with EtOAc, and the filtrate was concentrated in vacuo. The residue was purified by flash chromatography (10 g, silica) eluting with 0-10% MeOH/DCM to yield the title compound (270 mg, 78% yield). 1H NMR (400 MHz, Chloroform-d) δ 8.75-8.69 (m, 2H), 7.88 (s, 1H), 7.38-7.32 (m, 2H), 7.23-7.17 (m, 2H), 6.94-6.86 (m, 2H), 4.85 (q, J=7.0 Hz, 1H), 3.69-3.60 (m, 1H), 3.43-3.30 (m, 3H), 3.25-3.15 (m, 1H), 3.13-3.04 (m, 1H), 3.04-2.95 (m, 1H), 2.94-2.84 (m, 1H), 1.73 (d, J=7.1 Hz, 3H), 1.43 (s, 9H). LCMS (Analytical Method D) Rt=0.97 min, MS (ESIpos): m/z 480.1 [M+H]+, Purity=92%.

### Synthesis of 1-(4-fluorophenyl)-2-(pyridin-4-yl)ethan-1-one/Intermediate 39-1

To an ice-cold solution of methyl 4-fluorobenzoate (1.00 g, 6.49 mmol) and 4-methylpyridine (0.64 mL, 6.49 mmol) in anhydrous THF (10 mL) 1 M LiHMDS in THF (13 mL, 13.0 mmol) was added, and the resulting mixture was stirred for 1 h. The reaction was quenched with water, extracted with EtOAc, dried over MgSO₄, filtered and concentrated in vacuo. The residue was purified by flash chromatography (25 g, silica) eluting with 0-50% EtOAc/DCM to yield the title compound (1.29 g, 92% yield). 1H NMR (500 MHz, Chloroform-d) δ 8.60-8.54 (m, 2H), 8.07-7.99 (m, 2H), 7.21-7.13 (m, 4H), 4.26 (s, 2H). LCMS (Analytical Method F) Rt=0.50 min, MS (ESIpos): m/z 216.1 [M+H]+, Purity=100%.

### Synthesis of N-[1-(4-fluorophenyl)-2-(pyridin-4-yl)ethylidene]hydroxylamine/Intermediate 39-2

A mixture of 1-(4-fluorophenyl)-2-(4-pyridyl)ethanone (Intermediate 39-1) (1.27 g, 5.90 mmol), hydroxylamine hydrochloride (1.31 g, 18.9 mmol), and sodium acetate (2.16 g, 26.0 mmol) in MeOH (6 mL) and water (6 mL) was refluxed for 1.5 h. The mixture was cooled to 0° C. and the precipitate was collected by filtration, washed with water and dried under vacuo to yield the title compound (520 mg, 38% yield). 1H NMR (500 MHz, Chloroform-d) δ 8.53-8.47 (m, 2H), 8.33 (s, 1H), 7.62-7.55 (m, 2H), 7.21-7.16 (m, 2H), 7.08-6.99 (m, 2H), 4.18 (s, 2H). LCMS (Analytical Method F) Rt=0.47 min, MS (ESIpos): m/z 231.1 [M+H]+, Purity=100%.

### Synthesis of 2-[3-(4-fluorophenyl)-4-(pyridin-4-yl)-1,2-oxazol-5-yl]acetic acid/Intermediate 39

To a stirred solution of 1-(4-fluorophenyl)-2-(4-pyridyl)ethanone oxime (Intermediate 39-2) (50 mg, 0.217 mmol) in anhydrous THF (1 mL) at −25° C., 2.5 M BuLi (0.26 mL, 0.651 mmol) was added dropwise. The mixture was stirred for 1.5 h then ethyl 3,3-diethoxyacrylate (0.044 mL, 0.228 mmol) was added. The mixture was stirred for 1 h at −25° C. then 2 h at RT. Water (0.2 mL) and MeOH (0.4 mL) were added and the mixture was heated at reflux for 1 h. After cooling to RT, the reaction mixture was poured into water, acidified with dilute HCl and washed with EtOAc. The aqueous layer was neutralized to pH˜5 with 5% Na₂CO₃ and then extracted into EtOAc, dried over MgSO₄, filtered, and concentrated in vacuo to yield the title compound (22 mg, 26% yield). 1H NMR (400 MHz, DMSO-d6) δ 13.04 (s, 1H), 8.63 (s, 2H), 7.46-7.41 (m, 2H), 7.34-7.26 (m, 3H), 7.26-7.20 (m, 2H), 3.97 (s, 2H). LCMS (Analytical Method F) Rt=0.58 min, MS (ESIpos): m/z 299.1 [M+H]+, Purity=99%.

### Synthesis of tert-butyl 4-{2-[3-(4-fluorophenyl)-4-(pyridin-4-yl)-1,2-oxazol-5-yl]acetyl}piperazine-1-carboxylate/Intermediate 40

A mixture of 2-[3-(4-fluorophenyl)-4-(4-pyridyl)isoxazol-5-yl]acetic acid (Intermediate 39) (21 mg, 0.0704 mmol), tert-butyl piperazine-1-carboxylate (20 mg, 0.106 mmol), HATU (32 mg, 0.0845 mmol) and DIPEA (12 μL, 0.0704 mmol) in DCM (0.7 mL) was stirred at RT for 4.5 h. The reaction mixture was diluted with DCM, washed with NaHCO₃ (aq), dried over MgSO₄, filtered, and concentrated in vacuo. The residue was purified by flash chromatography (10 g, silica) eluting with 0-4% MeOH/DCM to yield the title compound (43 mg, 76% yield). LCMS (Analytical Method H) Rt=0.59 min, MS (ESIpos): m/z 467.3 [M+H]+, Purity=59%.

### Synthesis of 4-chloro-N-(pyridin-4-yl)benzene-1-carboximidamide/Intermediate 41-1

To an ice-cold solution of pyridin-4-amine (1.10 g, 11.7 mmol) in DMSO (5 mL), NaH (60%, 0.64 g, 15.9 mmol) was added, and the mixture was stirred at this temperature for 5 min. 4-Chlorobenzonitrile (1.46 g, 10.6 mmol) was then added, and the reaction mixture was stirred at 0° C. for 2 h. The reaction was diluted with water and the resulting precipitate collected under vacuum filtration to afford the title compound pure (1.95 g, 79% yield). 1H NMR (500 MHz, DMSO-d6) δ 8.37 (d, J=5.7 Hz, 2H), 8.00-7.87 (m, 2H), 7.51 (d, J=8.6 Hz, 2H), 6.80 (d, J=4.7 Hz, 2H), 6.73 (s, 2H). LCMS (Analytical Method E) Rt=0.66 min, MS (ESIpos): m/z 231.9 [M+H]+, Purity=100%.

### Synthesis of methyl 4-oxobutanoate/Intermediate 41-2

Methyl 4,4-dimethoxybutyrate (4.70 g, 29.0 mmol) was dissolved in Et₂O (25 mL) and treated with 1.2 M HCl (12 mL, 14.4 mmol), then stirred at RT for 18 h. The aqueous phase was extracted with DCM and the organic extracts combined, washed with brine, dried over Na₂SO₄, filtered and concentrated in vacuo to yield the title compound (3.3 g, 57% yield), which was used in the next step without further purification. 1H NMR (500 MHz, DMSO-d6) δ 9.81 (s, 1H), 3.69 (s, 3H), 2.80 (t, J=6.6 Hz, 2H), 2.63 (t, J=6.6 Hz, 2H).

### Synthesis of 2-[2-(4-chlorophenyl)-1-(pyridin-4-yl)-1H-imidazol-5-yl]acetic acid/Intermediate 41

Methyl 4-oxobutanoate (Intermediate 41-2) (0.90 g, 6.47 mmol) was dissolved in DCM (5.5 mL) and 1,4-dioxane (2.5 mL) and treated with molecular bromine (0.33 mL, 6.47 mmol) at 0° C. The reaction was warmed to RT and then stirred for 3 h. After which it was treated with K₂CO₃ (1.49 g, 10.8 mmol), 4-chloro-N-(4-pyridyl)benzamidine (Intermediate 41-1) (0.50 g, 2.16 mmol) and water (1 mL) and stirred at RT for 2 h. The reaction was diluted with DCE (5 mL) and heated at 80° C. for 18 h. The reaction was quenched with 2 M NaOH and the aqueous layer extracted with DCM. The organic extracts were discarded and the aqueous layer was acidified to pH 3 with 2 M HCl (˜6 mL), then extracted with (1:1) DCM/IPA. The organic extracts were combined, dried over Na₂SO₄, filtered and concentrated in vacuo to afford the title compound (285 mg, 14% yield), which was used in the next step without further purification. 1H NMR (500 MHz, DMSO-d6) δ 12.40 (s, 1H), 8.74-8.68 (m, 2H), 7.40-7.33 (m, 4H), 7.29-7.22 (m, 2H), 7.14 (s, 1H), 3.64 (s, 2H). LCMS (Analytical Method E) Rt=0.78 min, MS (ESIpos): m/z 313.8 [M+H]+, Purity=69%.

### Synthesis of tert-butyl 4-{2-[2-(4-chlorophenyl)-1-(pyridin-4-yl)-1H-imidazol-5-yl]acetyl}piperazine-1-carboxylate/Intermediate 42

HATU (85 mg, 0.224 mmol) and DIPEA (78 μL, 0.448 mmol) were added to a solution of tert-butyl piperazine-1-carboxylate (111 mg, 0.597 mmol) and 2-[2-(4-chlorophenyl)-3-(4-pyridyl)imidazol-4-yl]acetic acid (Intermediate 41) (142 mg, 0.149 mmol) in DCM (1.5 mL), and the reaction mixture was stirred at RT for 18 h. The solvent was evaporated under reduced pressure and the residue was purified by preparative HPLC (Method A2) to afford the title compound (60 mg, 83% yield). LCMS (Analytical Method A) Rt=2.17 min, MS (ESIpos): m/z 482.3 [M+H]+, Purity=99%.

### Synthesis of 2-[4-(4-fluorophenyl)-1H-imidazol-1-yl]-1-(morpholin-4-yl)ethan-1-one/Intermediate 43-1

NaH (60%, 95 mg, 2.37 mmol) was added to an ice-cold solution of 4-(4-fluorophenyl)-1H-imidazole (350 mg, 2.16 mmol) in anhydrous THF (7 mL). The reaction was stirred for 15 min before 2-chloro-1-morpholino-ethanone (0.28 mL, 2.16 mmol) was added, and the reaction was stirred at 0° C. for 2 h and at RT for 2 additional h. The reaction was carefully quenched into water, extracted with EtOAc (2×), washed with brine, dried over MgSO₄ and concentrated in vacuo. The residue was purified by flash chromatography (25 g, silica), eluting with 0-6% MeOH/DCM and the resulting product was triturated with DCM to yield the title compound as a white solid (253 mg, 40% yield). 1H NMR (500 MHz, DMSO-d6) δ 7.79-7.73 (m, 2H), 7.59 (d, J=1.1 Hz, 1H), 7.50 (d, J=1.1 Hz, 1H), 7.21-7.14 (m, 2H), 5.05 (s, 2H), 3.65 (t, J=4.7 Hz, 2H), 3.60 (t, J=4.7 Hz, 2H), 3.51 (t, J=4.6 Hz, 2H), 3.47 (t, J=4.6 Hz, 2H). LCMS (Analytical Method F) Rt=0.47 min, MS (ESIpos): m/z 290.1 [M+H]+, Purity=100%.

### Synthesis of 2-[5-bromo-4-(4-fluorophenyl)-1H-imidazol-1-yl]-1-(morpholin-4-yl)ethan-1-one/Intermediate 43

NBS (169 mg, 0.951 mmol) was added to an ice-cold solution of 2-[4-(4-fluorophenyl)-1H-imidazol-1-yl]-1-(morpholin-4-yl)ethan-1-one (Intermediate 43-1) (250 mg, 0.864 mmol) in MeCN (6 mL), and the reaction was stirred for 90 min The reaction was quenched with into water, extracted into EtOAc (3×), dried over MgSO₄, filtered and concentrated in vacuo. The residue was purified by flash chromatography (25 g, silica), eluting with 0-5% MeOH/DCM to yield the title compound as an off-white solid (132 mg, 35% yield). 1H NMR (400 MHz, Chloroform-d) δ 8.00-7.90 (m, 2H), 7.73 (s, 1H), 7.16-7.07 (m, 2H), 4.82 (s, 2H), 3.85-3.65 (m, 6H), 3.65-3.54 (m, 2H). LCMS (Analytical Method H) Rt=0.48 min, MS (ESIpos): m/z 368.1 [M+H]+, Purity=85%.

### Synthesis of tert-butyl 4-(3-oxobutanoyl)piperazine-1-carboxylate/Intermediate 44-1

A solution of tert-butyl 3-oxobutanoate (500 mg, 3.16 mmol) and tert-butyl piperazine-1-carboxylate (589 mg, 3.16 mmol) in toluene (5 mL) was heated at 100° C. for 18 h. The mixture was quenched with sat. aq. NH₄Cl and extracted with EtOAc, dried over MgSO₄, filtered and concentrated in vacuo to afford the title compound (766 mg, 72% yield), which was used in the next step without further purification. 1H NMR (500 MHz, DMSO-d6) δ 3.62-3.59 (m, 2H), 3.58 (s, 2H), 3.43 (dd, J=8.3, 4.1 Hz, 4H), 3.38 (dd, J=6.4, 3.5 Hz, 2H), 2.28 (s, 3H), 1.46 (s, 9H).

### Synthesis of tert-butyl 4-[5-(4-fluorophenyl)-3,5-dioxopentanoyl]piperazine-1-carboxylate/Intermediate 44-2

To an ice-cold solution of tert-butyl 4-(3-oxobutanoyl)piperazine-1-carboxylate (Intermediate 44-1) (750 mg, 2.77 mmol) in THF (6 mL), 2 M LDA (4.2 mL, 8.32 mmol) was added dropwise, and the mixture was stirred at 0° C. for 2 h. A solution of methyl 4-fluorobenzoate (655 mg, 4.16 mmol) in THF (6 mL) was then added dropwise over 5 min, and the resulting reaction mixture was allowed to stir at RT for 18 h. The mixture was treated with 2 M HCl and the pH adjusted to ˜6. The aqueous layer was extracted with Et₂O and the organic washed with sat. aq. NaHCO₃and water, dried over Na₂SO₄, filtered and concentrated in vacuo to afford the title compound (1.2 g, 100% yield), which was used in the next step without further purification. LCMS (Analytical Method E) Rt=1.10 min, MS (ESIpos): m/z 336.9 [M-tButyl+H]+, Purity=32%.

### Synthesis of tert-butyl 4-{2-[3-(4-fluorophenyl)-1H-pyrazol-5-yl]acetyl}piperazine-1-carboxylate/Intermediate 44-3

Hydrazine hydrate (156 mg, 3.12 mmol) was added to a solution of tert-butyl 4-[5-(4-fluorophenyl)-3,5-dioxo-pentanoyl]piperazine-1-carboxylate (Intermediate 44-2) (1.22 g, 3.12 mmol) in MeOH (5 mL) and acetic acid (0.5 mL). The resulting reaction mixture was heated at 70° C. for 1.5 h. The reaction was quenched with sat. aq. NaHCO₃ and extracted with DCM. The organic layer was washed with brine, dried over Na₂SO₄, filtered and concentrated in vacuo. The residue was purified by flash chromatography (50 g, silica) eluting with 0-100% TBME/heptane, then 0-20% MeOH/TBME to yield the title compound (241 mg, 18% yield). 1H NMR (400 MHz, DMSO-d6) δ 12.85 (m, 1H), 7.78 (s, 2H), 7.34-7.12 (m, 2H), 6.50 (s, 1H), 3.84-3.63 (m, 2H), 3.56-3.49 (m, 2H), 3.49-3.43 (m, 2H), 3.32-3.24 (m, 4H), 1.40 (s, 9H). LCMS (Analytical Method E) Rt=1.10 min, MS (ESIpos): m/z 389.0 [M+H]+, Purity=91%.

### Synthesis of tert-butyl 4-{2-[4-bromo-3-(4-fluorophenyl)-1H-pyrazol-5-yl]acetyl}piperazine-1-carboxylate/Intermediate 44-4

NBS (119 mg, 0.667 mmol) was added to an ice-cold solution of tert-butyl 4-[2-[3-(4-fluorophenyl)-1H-pyrazol-5-yl]acetyl]piperazine-1-carboxylate (Intermediate 44-3) (240 mg, 0.556 mmol) in DCM (4 mL), and the reaction was stirred for 1 h. Water was added and the organic layer was separated. The aqueous layer was extracted into DCM and the organic extracts were combined, dried over MgSO₄, filtered and concentrated in vacuo. The residue was purified by flash chromatography (25 g, silica) eluting with 0-5% MeOH/DCM to yield the title compound (165 mg, 56% yield). 1H NMR (400 MHz, Chloroform-d) δ 7.84-7.77 (m, 2H), 7.16-7.10 (m, 2H), 3.81 (s, 2H), 3.69-3.62 (m, 2H), 3.62-3.55 (m, 2H), 3.52-3.43 (m, 4H), 1.48 (s, 9H). LCMS (Analytical Method F) Rt=0.96 min, MS (ESIpos): m/z 467.2, 469.2 [M+H]+, Purity=90%.

### Synthesis of tert-butyl 4-{2-[4-bromo-5-(4-fluorophenyl)-1-{[2-(trimethylsilyl)ethoxy]methyl}-1H-pyrazol-3-yl]acetyl}piperazine-1-carboxylate/Intermediate 44-5

To an ice-cold solution of tert-butyl 4-[2-[4-bromo-3-(4-fluorophenyl)-1H-pyrazol-5-yl]acetyl]piperazine-1-carboxylate (Intermediate 44-4) (97 mg, 0.187 mmol) in THF (3 mL) NaH (60%, 8.2 mg, 0.205 mmol) was added, and the mixture was stirred for 10 min. 2-(chloromethoxy)ethyl-trimethyl-silane (36 μL, 0.205 mmol) was added and the reaction was stirred at RT for 4 h. The mixture was quenched with water, extracted with EtOAc, dried over MgSO₄, filtered, and concentrated in vacuo. The crude product was purified by flash chromatography (10 g, silica) eluting with 0-4% MeOH/DCM to yield the title compound as a 4:1 mixture of regioisomers (80 mg, 62% yield). 1H NMR (400 MHz, Chloroform-d) δ 7.63-7.55 (m, 2H), 7.22-7.15 (m, 2H), 5.27 (s, 2H), 3.79 (s, 2H), 3.72-3.40 (m, 8H), 1.48 (s, 9H), 0.96-0.86 (m, 2H), -0.00 (s, 9H). LCMS (Analytical Method F) Rt=1.26, 1.28 min, MS (ESIpos): m/z 599.2 [M+H]+, Purity=87%.

### Synthesis of tert-butyl 4-{2-[5-(4-fluorophenyl)-4-(pyridin-4-yl)-1-{[2-(trimethylsilyl)ethoxy]methyl}-1H-pyrazol-3-yl]acetyl}piperazine-1-carboxylate/Intermediate 44

A mixture of tert-butyl 4-[2-[4-bromo-5-(4-fluorophenyl)-1-(2-trimethylsilylethoxymethyl)pyrazol-3-yl]acetyl]piperazine-1-carboxylate (Intermediate 44-5) (70 mg, 0.116 mmol), pyridin-4-ylboronic acid (27 mg, 0.219 mmol), Pd(PPh₃)₄ (6.7 mg, 5.82 μmol) and 2 M Na₂CO₃ (0.29 mL, 0.582 mmol) in DME (1 mL) was degassed by sparging with nitrogen. The reaction was heated to 125° C. for 1 h under microwave irradiation. The mixture was diluted with EtOAc, washed with water, dried, filtered and concentrated in vacuo. The residue was purified by flash chromatography (10 g, silica) eluting with 0-4% MeOH/DCM to yield the title compound (40 mg, 48% yield). 1H NMR (500 MHz, Chloroform-d) δ 8.47 (d, J=5.4 Hz, 2H), 7.36-7.28 (m, 2H), 7.13-7.09 (m, 2H), 7.09-7.02 (m, 2H), 5.26 (s, 2H), 3.76-3.70 (m, 4H), 3.59 (dd, J=10.5, 5.5 Hz, 4H), 3.50-3.38 (m, 4H), 1.46 (s, 9H), 0.99-0.91 (m, 2H), 0.00 (s, 9H). LCMS (Analytical Method F) Rt=1.11 min, MS (ESIpos): m/z 596.4 [M+H]+, Purity=92%.

### Synthesis of ethyl 4-oxo-4-(pyridin-4-yl)butanoate/Intermediate 45-1

To a stirred solution of ethyl prop-2-enoate (4.6 mL, 42.5 mmol), and 2-(3-benzyl-4-methyl-thiazol-3-ium-5-yl)ethanol; chloride (1.17 g, 4.25 mmol) in DMF (20 mL), was added a solution of Et₃N (3.0 mL, 21.2 mmol) and pyridine-4-carbaldehyde (2.0 mL, 21.2 mmol) in DMF (20 mL) over 1.5 h. The mixture was then stirred at RT for 1 h. Water was added and the mixture was extracted with EtOAc, washed with brine, dried over MgSO₄, filtered and concentrated in vacuo. The crude product was triturated with MeCN and the solid was removed by filtration. The filtrate was evaporated and the residue was purified by flash chromatography (100 g, silica) eluting with 0-100% EtOAc/heptane to yield the title compound (890 mg, 17% yield). 1H NMR (500 MHz, Chloroform-d) δ 8.86-8.82 (m, 2H), 7.80-7.76 (m, 2H), 4.19 (q, J=7.1 Hz, 2H), 3.32 (t, J=6.5 Hz, 2H), 2.81 (t, J=6.5 Hz, 2H), 1.29 (t, J=7.1 Hz, 3H). LCMS (Analytical Method F) Rt=0.58 min, MS (ESIpos): m/z 208.1 [M+H]+, Purity=85%.

### Synthesis of ethyl (3E)-4-(dimethylamino)-3-[(E)-pyridine-4-carbonyl]but-3-enoate/Intermediate 45-2

To a solution of ethyl 4-oxo-4-(4-pyridyl)butanoate (Intermediate 45-1) (790 mg, 3.24 mmol) in dry toluene (7.9 mL), 1,1-dimethoxy-N,N-dimethyl-methanamine (2.6 mL, 19.4 mmol) was added, and the resulting mixture was stirred at reflux for 7 h. The solvent was evaporated in vacuo to yield the title compound (556 mg, 55% yield), which was used in the next step without further purification. 1H NMR (400 MHz, Chloroform-d) δ 8.66-8.61 (m, 2H), 7.32-7.28 (m, 2H), 6.90 (s, 1H), 4.18 (q, J=7.1 Hz, 2H), 3.68 (s, 2H), 3.05 (s, 6H), 1.32-1.24 (m, 3H). LCMS (Analytical Method F) Rt=0.45 min, MS (ESIpos): m/z 263.1 [M+H]+, Purity=84%.

### Synthesis of ethyl 2-[1-(4-fluorophenyl)-5-(pyridin-4-yl)-1H-pyrazol-4-yl]acetate/Intermediate 45-3

To a stirred solution of ethyl 4-(dimethylamino)-3-(pyridine-4-carbonyl)but-3-enoate (Intermediate 45-2) (550 mg, 1.76 mmol) in EtOH (5 mL) and water (5 mL), (4-fluorophenyl)hydrazine hydrochloride (301 mg, 1.80 mmol) was added, and the mixture was stirred at 50° C. for 1.5 h. Solvent was evaporated under reduced pressure, and the residue was dissolved in DCM, filtered through a Telos phase separator and purified by flash chromatography (100 g, silica) eluting with 0-50% of EtOAc/heptane to yield the title compound (442 mg, 67% yield). 1H NMR (400 MHz, Chloroform-d) δ 8.58-8.52 (m, 2H), 7.71 (s, 1H), 7.15-7.10 (m, 2H), 7.09-7.05 (m, 2H), 6.98-6.92 (m, 2H), 4.10 (q, J=7.1 Hz, 2H), 3.43 (s, 2H), 1.19 (t, J=7.1 Hz, 3H). LCMS (Analytical Method H) Rt=0.52 min, MS (ESIpos): m/z 326.2 [M+H]+, Purity=87%.

### Synthesis of lithium 2-[1-(4-fluorophenyl)-5-(pyridin-4-yl)-1H-pyrazol-4-yl]acetate/Intermediate 45-4

To a stirred solution of ethyl 2-[1-(4-fluorophenyl)-5-(4-pyridyl)pyrazol-4-yl]acetate (Intermediate 45-3) (440 mg, 1.18 mmol) in MeOH (15 mL) and water (8 mL), LiOH (247 mg, 5.88 mmol) was added, and the resulting mixture was allowed to stir at RT for 1 h. Solvent was evaporated under reduced pressure. Acetone was added, and the white precipitate was filtered off. The filtrate was evaporated under reduced pressure to provide the title compound (380 mg, 86% yield) as a pale-brown solid, which was used in the next step without further purification. 1H NMR (500 MHz, DMSO-d6) δ 8.56-8.50 (m, 2H), 7.67 (s, 1H), 7.38-7.34 (m, 2H), 7.27-7.18 (m, 4H), 3.00 (s, 2H). LCMS (Analytical Method F) Rt=0.54 min, MS (ESIpos): m/z 298.1 [M+H]+, Purity=81%.

### Synthesis of tert-butyl 4-{2-[1-(4-fluorophenyl)-5-(pyridin-4-yl)-1H-pyrazol-4-yl]acetyl}piperazine-1-carboxylate/Intermediate 45

To a stirred solution of lithium 2-[1-(4-fluorophenyl)-5-(4-pyridyl)pyrazol-4-yl]acetate (Intermediate 45-4) (250 mg, 0.099 mmol) in DMF (1.5 mL) was added HATU (56 mg, 0.148 mmol). The mixture was stirred at RT for 10 min, then tert-butyl piperazine-1-carboxylate (28 mg, 0.148 mmol) and DIPEA (0.05 mL, 0.297 mmol) were added and the reaction was stirred at RT for 2 h. The mixture was concentrated in vacuo and the residue was taken up in DCM and washed with brine. The organics were collected using a Telos phase separator, then concentrated in vacuo. The residue was purified by flash chromatography (10 g, silica) eluting with 0-4% MeOH/DCM to yield the title compound (26 mg, 43% yield). LCMS (Analytical Method F) Rt=0.77 min, MS (ESIpos): m/z 466.3 [M+H]+, Purity=76%.

### 1.3—Synthesis of 2-[4-(4-fluorophenyl)-5-(pyridin-4-yl)-1H-imidazol-1-yl]-1-[4-(propan-2-yl)piperazin-1-yl]ethan-1-one/Compound 47 of Table 1

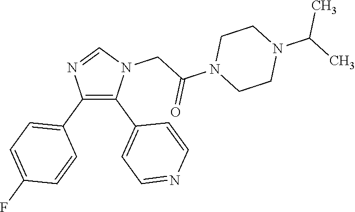 [SMILES: CC(C)N1CCN(C(=O)Cn2cnc(-c3ccc(F)cc3)c2-c2ccncc2)CC1]

A solution of 2-[4-(4-fluorophenyl)-5-(pyridin-4-yl)-1H-imidazol-1-yl]acetic acid TFA salt (Intermediate 1) (40 mg, 0.0761 mmol), HATU (43 mg, 0.114 mmol) and DIPEA (60 μL, 0.343 mmol) in DMF (1 mL) was stirred at RT for 10 min. N-Isopropylpiperazine (16 μL, 0.114 mmol) was added and stirring continued for 1 h. The reaction was quenched into water. The aqueous layer was extracted into EtOAc (3×), the combined organics washed with brine, dried over MgSO₄ and concentrated in vacuo. The residue was purified by preparative HPLC (Method A1) to afford the title compound as a white solid (10 mg, 32% yield). 1H NMR (500 MHz, DMSO-d6) δ 8.64 (d, J=6.0 Hz, 2H), 7.80 (s, 1H), 7.37 (dd, J=8.9, 5.6 Hz, 2H), 7.27 (d, J=6.0 Hz, 2H), 7.09 (t, J=8.9 Hz, 2H), 4.90 (s, 2H), 2.66-2.62 (m, 1H), 2.27 (d, J=4.4 Hz, 4H), 0.94 (d, J=6.6 Hz, 5H). LCMS (Analytical Method B) Rt=2.38 min, MS (ESIpos): m/z 408.4 [M+H]+, Purity=98%.

### 1.4—Synthesis of 2-[4-(4-fluorophenyl)-5-(pyridin-4-yl)-1H-imidazol-1-yl]-1-(4-methylpiperazin-1-yl)ethan-1-one/Compound 22 of Table 1

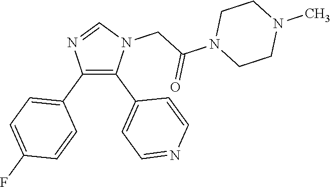 [SMILES: CN1CCN(C(=O)Cn2cnc(-c3ccc(F)cc3)c2-c2ccncc2)CC1]

DIPEA (130 μL, 0.744 mmol) was added to a solution of 2-[4-(4-fluorophenyl)-5-(pyridin-4-yl)-1H-imidazol-1-yl]acetic acid TFA salt (Intermediate 1) (130 mg, 0.247 mmol) and N-methylpiperazine (37 μL, 0.329 mmol) in EtOAc (2.6 mL). T3P (50%, 182 μL, 0.306 mmol) was added and the reaction stirred for 45 min. The reaction was quenched into water and the aqueous layer was extracted into EtOAc, dried over MgSO₄ and concentrated in vacuo. The residue was purified by preparative HPLC (Method A1) to afford the title compound as a white solid (30 mg, 32% yield). 1H NMR (500 MHz, DMSO-d6) δ 8.64 (d, J=6.0 Hz, 2H), 7.80 (s, 1H), 7.37 (dd, J=8.9, 5.6 Hz, 2H), 7.27 (d, J=6.0 Hz, 2H), 7.09 (t, J=8.9 Hz, 2H), 4.90 (s, 2H), 2.66-2.62 (m, 1H), 2.27 (d, J=4.4 Hz, 4H), 0.94 (d, J=6.6 Hz, 6H). LCMS (Analytical Method B) Rt=2.38 min, MS (ESIpos): m/z 408.4 [M+H]+, Purity=98%.

### Example 1.5—Synthesis of Other Compounds of General Formula (I)

Compounds listed in Table 1 were prepared according to the method of Example 1.3 or of Example 1.4, using the intermediates listed in table 2 in the “Synthesis” column for such compounds. The final compounds were purified by preparative HPLC.

TABLE 2
synthesis of several compounds from table 1

| # in | | | |
|---|---|---|---|
| table 1 | Synthesis | Structure/Name | Data |
| 48 | Intermediate 1 and morpholine (36% yield) | 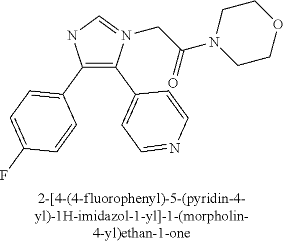 | 1H NMR (500 MHz, DMSO-d6) δ 8.65 (d, J = 6.0 Hz, 2H), 7.80 (s, 1H), 7.37 (dd, J = 8.9, 5.6 Hz, 2H), 7.27 (d, J = 6.0 Hz, 2H), 7.10 (t, J = 9.0 Hz, 2H), 4.92 (s, 2H), 3.49 − 3.44 (m, 4H), 3.37 − 3.35 (m, 4H). LCMS (Analytical Method A) Rt = 1.25 min, MS (ESIpos): m/z 367.2 [M + H]+, Purity = 100%. |
| 53 | Intermediate 3 and 1- methylpipera- zine (70% yield) | 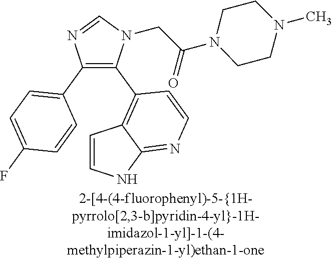 [SMILES: CN1CCN(C(=O)Cn2cnc(-c3ccc(F)cc3)c2-c2ccnc3[nH]ccc23)CC1] | 1H NMR (400 MHz, DMSO-d6) δ 8.26 (d, J = 4.8 Hz, 1H), 7.81 (s, 1H), 7.47 − 7.39 (m, 3H), 7.00 − 6.92 (m, 2H), 6.88 (d, J = 4.8 Hz, 1H), 5.97 (d, J = 3.2 Hz, 1H), 4.81 − 4.63 (m, 2H), 3.32 − 3.12 (m, 4H), 2.15 (s, 3H), 2.13 − 2.01 (m, 4H). LCMS (Analytical Method A) Rt = 0.99 min, MS (ESIpos): m/z 419.4 [M + H]+, Purity = 96%. |
| 5 | Intermediate 2a and 2- methyl- octahydropy- rrolo[3,4- c]pyrrole (37% yield) | 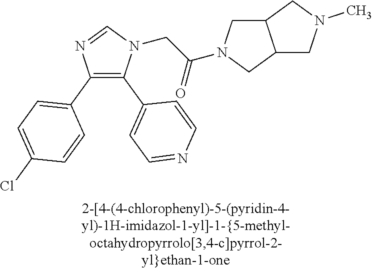 [SMILES: CN1CC2CN(C(=O)Cn3cnc(-c4ccc(Cl)cc4)c3-c3ccncc3)CC2C1] | 1H NMR (400 MHz, DMSO-d6) δ 8.68 − 8.62 (m, 2H), 7.83 (s, 1H), 7.38 − 7.28 (m, 6H), 4.79 (d, J = 3.0 Hz, 2H), 3.52 (dd, J = 10.8, 8.7 Hz, 1H), 3.45 (dd, J = 12.2, 8.8 Hz, 1H), 3.24 − 3.14 (m, 2H), 2.85 − 2.75 (m, 1H), 2.74 − 2.64 (m, 1H), 2.44 − 2.37 (m, 2H), 2.29 (dt, J = 8.6, 3.8 Hz, 2H), 2.20 (s, 3H). LCMS (Analytical Method B) Rt = 2.34 min, MS (ESIpos): m/z 422.3 [M + H]+, Purity = 97%. |
| 13 | Intermediate 2a and N,N- dimethylpyrro- lidin-3-amine (41% yield) | 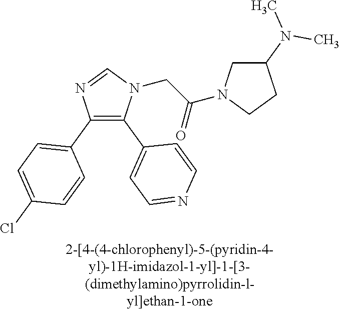 [SMILES: CN(C)C1CCN(C(=O)Cn2cnc(-c3ccc(Cl)cc3)c2-c2ccncc2)C1] | ¹H NMR (500 MHz, DMSO-d₆) δ 8.68 − 8.60 (m, 2H), 7.89 − 7.75 (m, 1H), 7.38 − 7.26 (m, 6H), 4.84 − 4.71 (m, 2H), 3.62 − 3.39 (m, 2H), 3.31 − 3.07 (m, 1H), 3.07 − 2.88 (m, 1H), 2.68 − 2.53 (m, 1H), 2.13 (s, 3H), 2.11 (s, 3H), 2.08 − 1.91 (m, 1H), 1.74 − 1.47 (m, 1H). LCMS (Analytical Method B) Rt = 2.29 min, MS (ESIpos): m/z 410.3 [M + H]+, Purity = 97%. |
| 8 | Intermediate 2a and 1- methylpipera- zine (57% yield) | 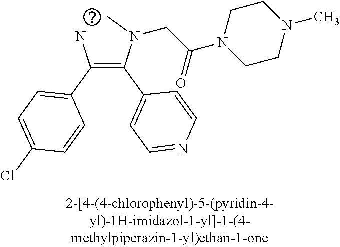 | 1H NMR (400 MHz, DMSO-d6) δ 8.70 − 8.63 (m, 2H), 7.83 (s, 1H), 7.40 − 7.34 (m, 2H), 7.34 − 7.30 (m, 2H), 7.30 − 7.25 (m, 2H), 4.92 (s, 2H), 3.40 − 3.32 (m, 4H), 2.20 − 2.11 (m, 4H). LCMS (Analytical Method B) Rt = 2.26 min, MS (ESIpos): m/z 396.2 [M + H]+, Purity = 100%. |
| 15 | Intermediate 2a and 1- methyl-1,4- diazepane (43% yield) | 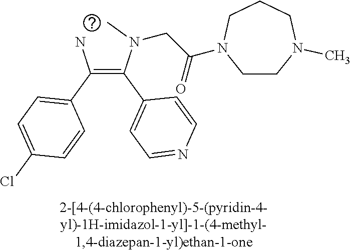 | 1H NMR (400 MHz, DMSO-d6) δ 8.69 − 8.60 (m, 2H), 7.79 (s, 1H), 7.43 − 7.36 (m, 2H), 7.33 − 7.25 (m, 4H), 4.85 (s, 2H), 3.51 − 3.32 (m, 4H), 2.45 − 2.40 (m, 2H), 2.40 − 2.35 (m, 2H), 2.24 (s, 3H), 1.77 − 1.61 (m, 2H). LCMS (Analytical Method B) Rt = 2.27 min, MS (ESIpos): m/z 410.3 [M + H]+, Purity = 99%. |
| 17 | Intermediate 2a and 2- oxa-6- azaspiro[3.3] heptane (47% yield) | 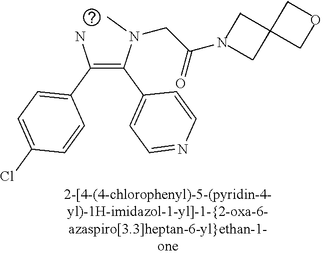 | 1H NMR (400 MHz, DMSO-d6) δ 8.71 − 8.64 (m, 2H), 7.83 (s, 1H), 7.38 − 7.27 (m, 6H), 4.62 (s, 4H), 4.59 (s, 2H), 4.13 (s, 2H), 3.97 (s, 2H). LCMS (Analytical Method B) Rt = 2.18 min, MS (ESIpos): m/z 395.2 [M + H]+, Purity = 99%. |
| 9 | Intermediate 2a and 1- methyl-1,6- diazaspiro[3. 4]octane (30% yield) | 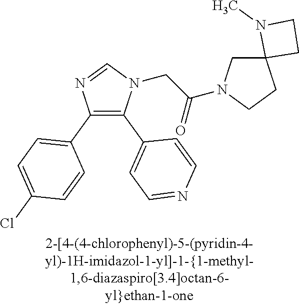 [SMILES: CN1CCC12CCN(C(=O)Cn1cnc(-c3ccc(Cl)cc3)c1-c1ccncc1)C2] | 1H NMR (500 MHz, DMSO-d6) δ 8.68 − 8.61 (m, 2H), 7.84 (s, 1H), 7.39 − 7.27 (m, 6H), 4.91 − 4.71 (m, 2H), 3.33 (s, 3H), 3.17 − 2.74 (m, 3H), 2.08 − 2.01 (m, 3H), 2.02 − 1.75 (m, 4H). LCMS (Analytical Method B) Rt = 2.28 min, MS (ESIpos): m/z 422.3 [M + H]+, Purity = 96%. |
| 16 | Intermediate 2a and 2- methyl-2,6- diazaspiro[3. 3]heptane (17% yield) | 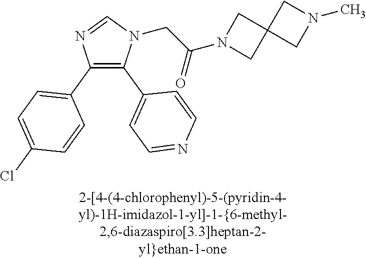 [SMILES: CN1CC2(C1)CN(C(=O)Cn1cnc(-c3ccc(Cl)cc3)c1-c1ccncc1)C2] | 1H NMR (500 MHz, DMSO-d6) δ 8.71 − 8.65 (m, 2H), 7.84 (s, 1H), 7.38 − 7.28 (m, 6H), 4.59 (s, 2H), 3.98 (s, 2H), 3.83 (s, 2H), 3.19 − 3.11 (m, 4H), 2.14 (s, 3H). LCMS (Analytical Method A) Rt = 1.13 min, MS (ESIpos): m/z 408.3 [M + H]+, Purity = 96%. |
| 1 | Intermediate 2b and piperidine (50% yield) | 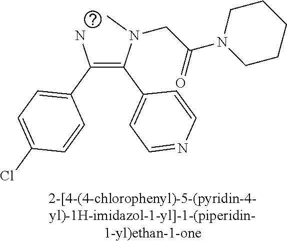 | 1H NMR (400 MHz, DMSO-d6) δ 8.69 − 8.61 (m, 2H), 7.82 (s, 1H), 7.39 − 7.30 (m, 4H), 7.30 − 7.26 (m, 2H), 4.89 (s, 2H), 3.37 − 3.26 (m, 4H), 1.60 − 1.49 (m, 2H), 1.40 − 1.27 (m, 4H). LCMS (Analytical Method A) Rt = 1.93 min, MS (ESIpos): m/z 381.3 [M + H]+, Purity = 95%. |
| 34 | Intermediate 2b and 4- methoxypiper- idine (16% yield) | 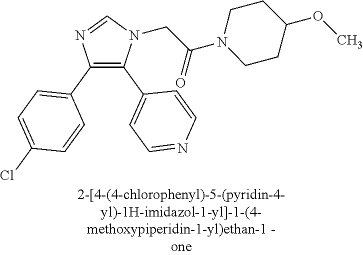 [SMILES: COC1CCN(C(=O)Cn2cnc(-c3ccc(Cl)cc3)c2-c2ccncc2)CC1] | 1H NMR (500 MHz, DMSO-d6) δ 8.69 − 8.61 (m, 2H), 7.83 (s, 1H), 7.39 − 7.34 (m, 2H), 7.34 − 7.30 (m, 2H), 7.30 7.26 (m, 2H), 4.92 (s, 2H), 3.75 − 3.65 (m, 1H), 3.57 − 3.47 (m, 1H), 3.41 − 3.34 (m, 1H), 3.24 (s, 3H), 3.12 (ddd, J = 13.2, 8.9, 2.6 Hz, 2H), 3.06 (ddd, J = 12.4, 9.0, 3.0 Hz, 2H), 1.75-1.64 (m, 2H), 1.34 − 1.16 (m, 2H). LCMS (Analytical Method B) Rt = 2.41 min, MS (ESIpos): m/z 411.3, 413.2 [M + H]+, Purity = 94%. |
| 19 | Intermediate 2b and 2- methyl-2,6- diazaspiro[3. 4]octane (9% yield) | 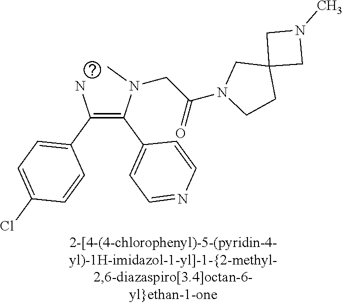 | 1H NMR (400 MHz, DMSO-d6) δ 8.66 − 8.63 (m, 2H), 7.80 (s, 1H), 7.42 − 7.36 (m, 2H), 7.32 − 7.24 (m, 4H), 4.73 (d, J = 4.4 Hz, 2H), 3.41 (s, 1H), 3.33 (s, 1H), 3.26 (q, J = 6.9 Hz, 2H), 3.06 − 2.97 (m, 5H), 2.85 (s, 1H), 2.22 (s, 3H), 2.00 (t, J = 6.5 Hz, 1H), 1.89 (t, J = 7.0 Hz, 1H). LCMS (Analytical Method B) Rt = 2.39 min, MS (ESIpos): m/z 422.3 [M + H]+, Purity = 87%. |
| 39 | Intermediate 2b and piperidin-4-ol (21% yield) | 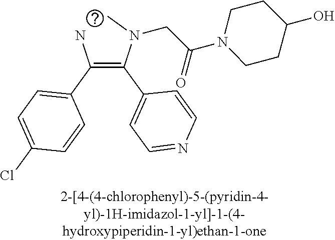 [SMILES: CN(CC(=O)N1CCC(O)CC1)/C(=C(\N)c1ccc(Cl)cc1)c1ccncc1] | 1H NMR (400 MHz, DMSO-d6) δ 8.69 − 8.61 (m, 2H), 8.46 (s, 1/2H) 7.82 (s, 1H), 7.39 − 7.34 (m, 2H), 7.34 − 7.30 (m, 2H), 7.29 − 7.24 (m, 2H), 4.91 (s, 2H), 4.77 (s, 1H), 3.81 − 3.72 (m, 1H), 3.72 − 3.61 (m, 1H), 3.58-3.51 (m, 1H), 3.14 − 2.93 (m, 2H), 1.68 − 1.55 (m, 2H), 1.27-1.11 (m, 2H). LCMS (Analytical Method B) Rt = 2.24 min, MS (ESIpos): m/z 397.3, 399.2 [M + H]+, Purity = 98%. |
| 35 | Intermediate 2a and 1- (oxolan-3- yl)methana- mine(15% yield) | 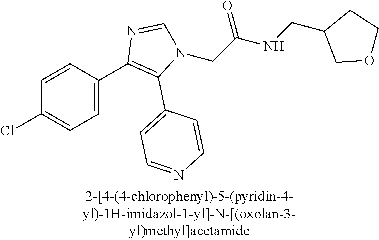 [SMILES: O=C(Cn1cnc(-c2ccc(Cl)cc2)c1-c1ccncc1)NCC1CCOC1] | 1H NMR (400 MHz, DMSO-d6) δ 8.69 − 8.62 (m, 2H), 8.12 (t, J = 5.7 Hz, 1H), 7.86 (s, 1H), 7.39 − 7.26 (m, 6H), 4.59 (s, 2H), 3.65 (td, J = 8.1, 5.7 Hz, 1H), 3.60 − 3.49 (m, 2H), 3.22 (dd, J = 8.6, 5.5 Hz, 1H), 3.01 − 2.89 (m, 2H), 2.19 − 2.10 (m, 1H), 1.83 − 1.74 (m, 1H), 1.41 − 1.32 (m, 1H). LCMS (Analytical Method A) Rt = 1.63 min, MS (ESIpos): m/z 397.3 [M + H]+, Purity = 100%. |
| 20 | Intermediate 2a and 1- methylpiperi- din-4-amine (15% yield) | 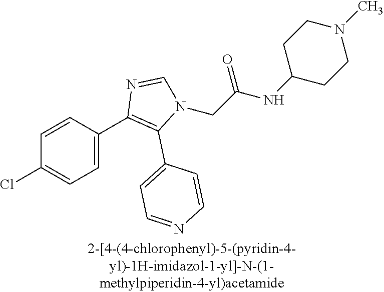 [SMILES: CN1CCC(NC(=O)Cn2cnc(-c3ccc(Cl)cc3)c2-c2ccncc2)CC1] | 1H NMR (400 MHz, DMSO-d6) δ 8.68 − 8.61 (m, 2H), 7.95 (d, J = 7.6 Hz, 1H), 7.85 (s, 1H), 7.39 − 7.24 (m, 6H), 4.55 (s, 2H), 3.46 − 3.35 (m, 1H), 2.65 − 2.56 (m, 2H), 2.11 (s, 3H), 1.93 − 1.82 (m, 2H), 1.60 − 1.48 (m, 2H), 1.33 − 1.17 (m, 2H). LCMS (Analytical Method B) Rt = 2.41 min, MS (ESIpos): m/z 410.4 [M + H]+, Purity = 100%. |
| 58 | Intermediate 3 and morpholine (70% yield) | 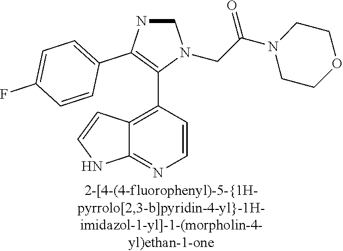 | 1H NMR (400 MHz, DMSO-d6) δ 11.82 (s, 1H), 8.31 (d, J = 4.8 Hz, 1H), 7.84 (s, 1H), 7.44 (d, J = 3.4 Hz, 1H), 7.36 − 7.30 (m, 2H), 7.02 − 6.93 (m, 3H), 5.92 (d, J = 3.4 Hz, 1H), 4.92 − 4.58 (m, 2H), 3.45 − 3.37 (m, 2H), 3.32 − 3.24 (m, 4H), 3.20 − 3.13 (m, 2H). LCMS (Analytical Method B) Rt = 2.12 min, MS (ESIpos): m/z 406.3 [M + H]+, Purity = 100%. |
| 24 | Intermediate 2a and 3- aminocyclo- pentan-1-ol hydrochloride (4% yield) | 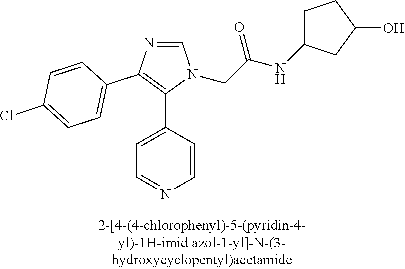 [SMILES: O=C(Cn1cnc(-c2ccc(Cl)cc2)c1-c1ccncc1)NC1CCC(O)C1] | 1H NMR (500 MHz, Chloroform-d) δ 8.76 − 8.61 (m, 2H), 7.67 (s, 1H), 7.40 − 7.33 (m, 2H), 7.29 − 7.26 (m, 2H), 7.24 − 7.18 (m, 2H), 6.56 (d, J = 8.5 Hz, 1H), 4.45(d, J = 0.9 Hz, 2H), 4.43 − 4.36 (m, 2H), 2.06 − 1.92 (m, 1H), 1.88 − 1.60 (m, 5H). LCMS (Analytical Method B) Rt = 2.25 min, MS (ESIpos): m/z 397.3 [M + H]+, Purity = 100%. |
| 46 | Intermediate 2a and 1- (pyridin-2- yl)methana- mine(31% yield) | 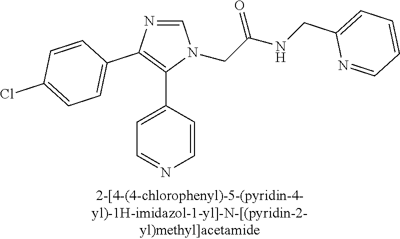 [SMILES: O=C(Cn1cnc(-c2ccc(Cl)cc2)c1-c1ccncc1)NCc1ccccn1] | 1H NMR (500 MHz, DMSO-d6) δ 8.72 − 8.61 (m, 3H), 8.49 (d, J = 4.1 Hz, 1H), 7.90 (s, 1H), 7.72 (td, J = 7.7, 1.8 Hz, 1H), 7.38 − 7.33 (m, 2H), 7.33 − 7.29 (m, 4H), 7.29 − 7.24 (m, 1H), 6.95 (d, J = 7.8 Hz, 1H), 4.72 (s, 2H), 4.30 (d, J = 5.9 Hz, 2H). LCMS (Analytical Method B) Rt = 2.42 min, MS (ESIpos): m/z 404.3 [M + H]+, Purity = 99%. |
| 41 | Intermediate 2a and 1- (pyridin-3- yl)methana- mine(16% yield) | 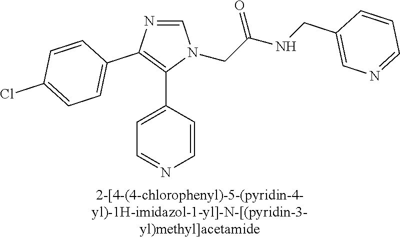 [SMILES: O=C(Cn1cnc(-c2ccc(Cl)cc2)c1-c1ccncc1)NCc1cccnc1] | 1H NMR (500 MHz, DMSO-d6) δ 8.64 − 8.56 (m, 3H), 8.46 (dd, J = 4.7, 1.6 Hz, 1H), 8.39 (d, J = 1.8 Hz, 1H), 7.89 (s, 1H), 7.43 − 7.39 (m, 1H), 7.37 − 7.29 (m, 5H), 7.28 − 7.24 (m, 2H), 4.67 (s, 2H), 4.22 (d, J = 5.8 Hz, 2H). LCMS (Analytical Method B) Rt = 2.33 min, MS (ESIpos): m/z 404.3 [M + H]+, Purity = 99%. |
| 42 | Intermediate 2a and 1- (pyridin-4- yl)methana mine(36% yield) | 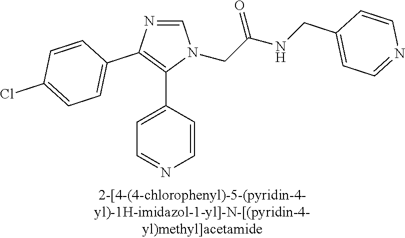 [SMILES: O=C(Cn1cnc(-c2ccc(Cl)cc2)c1-c1ccncc1)NCc1ccncc1] | 1H NMR (500 MHz, DMSO-d6) δ 8.71 − 8.58 (m, 3H), 8.48 − 8.42 (m, 2H), 7.91 (s, 1H), 7.37 − 7.33 (m, 2H), 7.33 − 7.29 (m, 4H), 7.01 − 6.96 (m, 2H), 4.75 (s, 2H), 4.22 (d, J = 6.0 Hz, 2H). LCMS (Analytical Method B) Rt = 2.29 min, MS (ESIpos): m/z 404.3 [M + H]+, Purity = 99%. |
| 4 | Intermediate 11 and 1- methylpipera- zine (13% yield) | 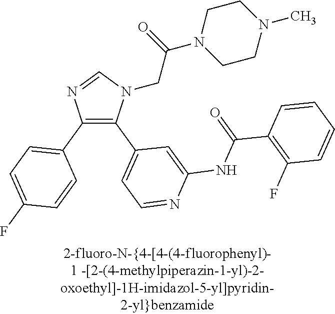 [SMILES: CN1CCN(C(=O)Cn2cnc(-c3ccc(F)cc3)c2-c2ccnc(NC(=O)c3ccccc3F)c2)CC1] | 1H NMR (500 MHz, MeOH-d4) δ 8.34 − 8.31 (m, 1H), 8.26 (s, 1H), 7.89 − 7.84 (m, 2H), 7.65 − 7.58 (m, 1H), 7.47 − 7.42 (m, 2H), 7.35 (d, J = 0.9 Hz, 1H), 7.32 − 7.26 (m, 1H), 7.06 − 7.00 (m, 2H), 6.97 (dd, J = 5.1, 1.5 Hz, 1H), 5.08 (s, 2H), 3.64 − 3.59 (m, 2H), 3.54 − 3.48 (m, 2H), 2.42 − 2.34 (m, 4H), 2.24 (s, 3H). LCMS (Analytical Method B) Rt = 2.68 min, MS (ESIpos): m/z 517.4 [M + H]+, Purity = 98%. |
| 7 | Intermediate 12 and 1- methylpipera zine- (60% yield) | 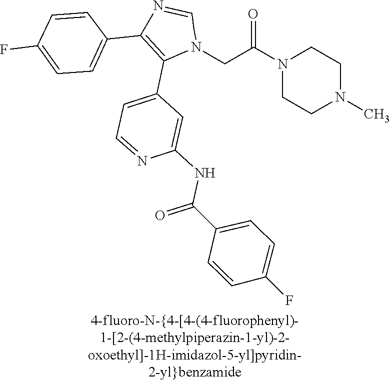 | 1H NMR (500 MHz, DMSO-d6) δ 11.01 (s, 1H), 8.43 (dd, J = 5.1, 0.6 Hz, 1H), 8.12 − 8.07 (m, 3H), 7.81 (s, 1H), 7.48 − 7.43 (m, 2H), 7.37 - 7.32 (m, 2H), 7.14 − 7.08 (m, 2H), 6.99 (dd, J = 5.1, 1.5 Hz, 1H), 4.91 (s, 2H), 3.43 − 3.39 (m, 2H), 3.36 − 3.34 (m, 2H), 2.20 − 2.12 (m, 4H), 2.07 (s, 3H). LCMS (Analytical Method B) Rt = 2.80 min, MS (ESIpos): m/z 517.4 [M + H]+, Purity = 99%. |
| 40 | Intermediate 39 and 1- methylpipera- zine (60% yield) | 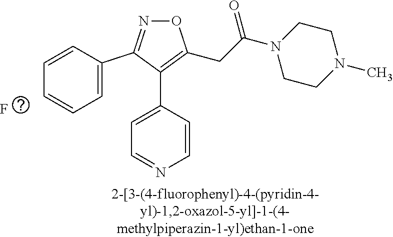 | 1H NMR (400 MHz, DMSO-d6) δ 8.64 − 8.58 (m, 2H), 7.46 − 7.38 (m, 2H), 7.33 − 7.26 (m, 2H), 7.23 − 7.18 (m, 2H), 4.09 (s, 2H), 3.53 − 3.41 (m, 4H), 2.32 − 2.22 (m, 4H), 2.19 (s, 3H). LCMS (Analytical Method B) Rt = 2.47 min, MS (ESIpos): m/z 381.3 [M + H]+, Purity = 100%. |
| 60 | Intermediate 41 and 1- methylpipera- zine (36% yield) | 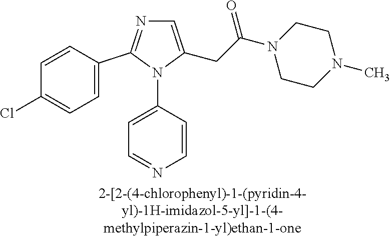 [SMILES: CN1CCN(C(=O)Cc2cnc(-c3ccc(Cl)cc3)n2-c2ccncc2)CC1] | 1H NMR (500 MHz, DMSO-d6) δ 8.72 − 8.65 (m, 2H), 7.41 − 7.29 (m, 4H), 7.29 − 7.21 (m, 2H), 7.05 (s, 1H), 3.70 (s, 2H), 3.34 − 3.26 (m, 4H), 2.19 − 2.14 (m, 2H), 2.13 (s, 3H), 2.11 − 2.06 (m, 2H). LCMS (Analytical Method B) Rt = 2.14 min, MS (ESIpos): m/z 396.3 [M + H]+, Purity = 99%. |
| 63 | Intermediate 45-4 and 1- methylpipera- zine (61% yield) | 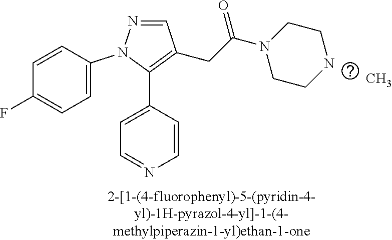 | 1H NMR (400 MHz, Chloroform-d) δ 8.65 − 8.57 (m, 2H), 7.72 (s, 1H), 7.23 − 7.16 (m, 2H), 7.16 − 7.11 (m, 2H), 7.05 − 6.96 (m, 2H), 3.69 − 3.62 (m, 2H), 3.53 (s, 2H), 3.47 − 3.41 (m, 2H), 2.42 − 2.36 (m, 2H), 2.36 − 2.31 (m, 2H), 2.30 (s, 3H). LCMS (Analytical Method B) Rt = 2.10 min, MS (ESIpos): m/z 380.3 [M + H]+, Purity = 100%. |

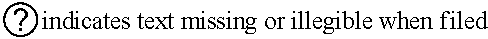

### 1.6—Synthesis of More Compounds of General Formula (I)

### 1.6.1—Synthesis of 2-[4-(4-fluorophenyl)-5-(pyridin-4-yl)-1H-imidazol-1-yl]-1-(piperazin-1-yl)ethan-1-one/Compound 30 of Table 1

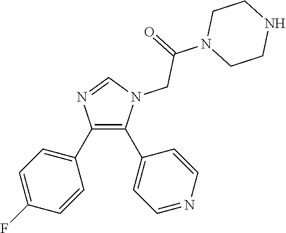 [SMILES: O=C(Cn1cnc(-c2ccc(F)cc2)c1-c1ccncc1)N1CCNCC1]

tert-Butyl 4-[2-[4-(4-fluorophenyl)-5-(4-pyridyl)imidazol-1-yl]acetyl]piperazine-1-carboxylate (14 mg, 0.0271 mmol) (Intermediate 9) was dissolved in 4 M HCl in dioxane (0.5 mL) and stirred at RT for 1 h. The reaction was concentrated in vacuo and the residue was purified by preparative HPLC (Method A1) to afford the title compound (3 mg, 29% yield). 1H NMR (500 MHz, Chloroform-d) δ 8.70-8.66 (m, 2H), 7.64 (s, 1H), 7.43-7.38 (m, 2H), 7.25-7.23 (m, 2H), 6.96-6.90 (m, 2H), 4.59 (s, 2H), 3.61-3.55 (m, 2H), 3.32-3.25 (m, 2H), 2.86-2.80 (m, 2H), 2.80-2.75 (m, 2H). LCMS (Analytical Method A) Rt=0.80 min, MS (ESIpos): m/z 366.2 [M+H]+, Purity=96%.

### 1.6.2—Synthesis of 2-[4-(4-chlorophenyl)-5-(pyridin-4-yl)-1H-imidazol-1-yl]-1-(piperazin-1-yl)ethan-1-one/Compound 18 of Table 1

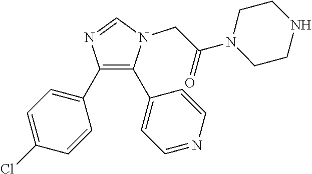 [SMILES: O=C(Cn1cnc(-c2ccc(Cl)cc2)c1-c1ccncc1)N1CCNCC1]

TFA (125 μL, 1.68 mmol) was added to a solution of tert-butyl 4-[2-[4-(4-chlorophenyl)-5-(4-pyridyl)imidazol-1-yl]acetyl]piperazine-1-carboxylate (Intermediate 4) (43 mg, 0.0839 mmol) in DCM (1 mL). The reaction was stirred for 1 h then concentrated in vacuo. The residue was purified by preparative HPLC (Method A1) to yield the title compound as a white solid (24 mg, 75% yield). 1H NMR (400 MHz, DMSO-d6) δ 8.65 (d, J=6.0 Hz, 2H), 7.82 (s, 1H), 7.38-7.33 (m, 2H), 7.33-7.29 (m, 2H), 7.29-7.25 (m, 2H), 4.88 (s, 2H), 3.27-3.22 (m, 4H), 2.55-2.52 (m, 4H). LCMS (Analytical Method B) Rt=2.20 min, MS (ESIpos): m/z 382.2, 384.2 [M+H]+, Purity=100%.

### Example 1.6.3—Further Synthesis of Further Compounds

Each of the Compounds listed in Table 3 were prepared according to the method of example 1.6.1 (4M HCl) or 1.6.2 (TEA), using the intermediates listed in the “Synthesis” column for such compounds. The final compounds were purified by preparative HPLC.

TABLE 3
synthesis of several compounds from table 1

| # in | | | |
|---|---|---|---|
| table 1 | Synthesis | Structure/Name | Data |
| 49 | Intermediate 5 (using 4M HCl) (60% yield) | 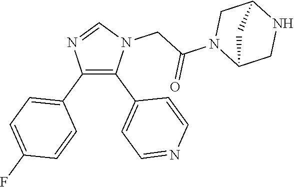 [SMILES: O=C(Cn1cnc(-c2ccc(F)cc2)c1-c1ccncc1)N1C[C@@H]2C[C@H]1CN2] 1-[(1S,4S)-2,5- diazabicyclo[2.2.1]heptan-2-yl]-2- [4-(4-fluorophenyl)-5-(pyridin-4- yl)-1H-imidazol-1-yl]ethan-1-one | 1H NMR (400 MHz, DMSO-d6) δ 8.70-8.57 (m, 2H), 7.82 (s, 1H), 7.42-7.33 (m, 2H), 7.33-7.23 (m, 2H), 7.12-7.06 (m, 2H), 5.06-4.61 (m, 2H), 4.55-4.43 (m, 1H), 3.95- 3.82 (m, 1H), 3.34-3.20 (m, 2H), 2.97-2.84 (m, 1H), 2.80-2.63 (m, 1H), 1.74-1.51 (m, 2H). LCMS (Analytical Method B) Rt = 1.87 min, MS (ESIpos): m/z 378.2 [M + H]+, Purity = 99%. |
| 14 | Intermediate 6 (using 4M HCl) (46% yield) | 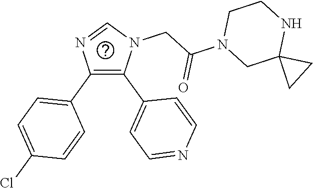 [SMILES: O=C(Cn1cnc(-c2ccc(Cl)cc2)c1-c1ccncc1)N1CCNC2(CC2)C1] 2-[4-(4-chlorophenyl)-5-(pyridin-4- yl)-1H-imidazol-1-yl]-1-{4,7- diazaspiro[2.5]octan-7-yl}ethan-1-one | 1H NMR (400 MHz, DMSO-d6, 357.9K) δ 8.69-8.60 (m, 2H), 7.78 (s, 1H), 7.46-7.37 (m, 2H), 7.32- 7.25 (m, 4H), 4.82 (s, 2H), 3.34 (s, 2H), 3.21 (s, 2H), 2.65 (q, J = 5.4, 5.0 Hz, 2H), 2.25 (t, J = 5.6 Hz, 1H), 0.42-0.33 (m, 4H). LCMS (Analytical Method B) Rt = 2.30 min, MS (ESIpos): m/z 409 [M + H]+, Purity = 98%. |
| 32 | Intermediate 7 (using TFA) (53% yield) | 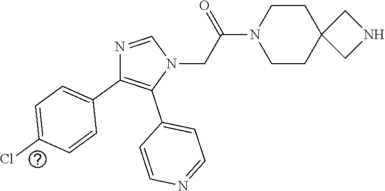 [SMILES: O=C(Cn1cnc(-c2ccc(Cl)cc2)c1-c1ccncc1)N1CCC2(CC1)CNC2] 2-[4-(4-chlorophenyl)-5-(pyridin-4- yl)-1H-imidazol-1-yl]-1-{2,7- diazaspiro[3.5]nonan-7-yl}ethan-1-one | 1H NMR (500 MHz, Chloroform-d) δ 8.72-8.67 (m, 2H), 7.66 (s, 1H), 7.42-7.36 (m, 2H), 7.28-7.25 (m, 2H), 7.25-7.20 (m, 2H), 4.61 (s, 2H), 3.56-3.49 (m, 2H), 3.46 (d, J = 7.7 Hz, 2H), 3.39 (d, J = 7.6 Hz, 2H), 3.28-3.20 (m, 2H), 1.74 (m, 4H). LCMS (Analytical Method B) Rt = 2.66 min, MS (ESIpos): m/z 422.3, 424.3 [M + H]+, Purity = 99%. |
| 29 | Intermediate 8 (using TFA) (69% yield) | 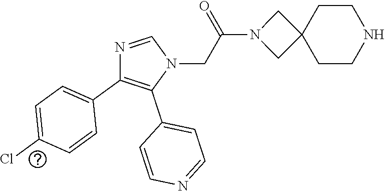 [SMILES: O=C(Cn1cnc(-c2ccc(Cl)cc2)c1-c1ccncc1)N1CC2(CCNCC2)C1] 2-[4-(4-chlorophenyl)-5-(pyridin-4- yl)-1H-imidazol-1-yl]-1-{2,7- diazaspiro[3.5]nonan-2-yl}ethan-1-one | 1H NMR (500 MHz, Chloroform-d) δ 8.76-8.72 (m, 2H), 7.70 (s, 1H), 7.41-7.36 (m, 2H), 7.32-7.29 (m, 2H), 7.25-7.21 (m, 2H), 4.40 (s, 2H), 3.71 (s, 2H), 3.60 (s, 2H), 2.76 (s, 4H), 1.74-1.64 (m, 4H). LCMS (Analytical Method B) Rt = 2.47 min, MS (ESIpos): m/z 422.3, 424.3 [M + H]+, Purity = 99%. |
| 38 | Intermediate 13 (using 4M HCl) (77% yield) | 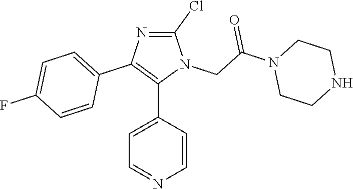 [SMILES: O=C(Cn1c(Cl)nc(-c2ccc(F)cc2)c1-c1ccncc1)N1CCNCC1] 2-[2-chloro-4-(4-fluorophenyl)-5- (pyridin-4-yl)-1H-imidazol-1-yl]-1- (piperazin-1-yl)ethan-1-one | 1H NMR (400 MHz, DMSO-d6) δ 8.70 (d, J = 5.8 Hz, 2H), 7.38-7.31 (m, 2H), 7.30-7.26 (m, 2H), 7.16- 7.06 (m, 2H), 4.79 (s, 2H), 3.43- 3.35 (m, 2H), 2.66-2.57 (m, 4H). LCMS (Analytical Method B) Rt = 2.23 min, MS (ESIpos): m/z 400.3 [M + H]+, Purity = 97%. |
| 6 | Intermediate 14 (using TFA) (52% yield) | 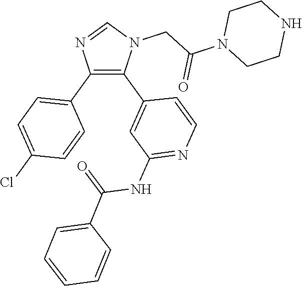 [SMILES: O=C(Nc1cc(-c2c(-c3ccc(Cl)cc3)ncn2CC(=O)N2CCNCC2)ccn1)c1ccccc1] N-{4-[4-(4-chlorophenyl)-1-[2-oxo- 2-(piperazin-1-yl)ethyl]-1H- imidazol-5-yl]pyridin-2-yl}benzamide | 1H NMR (500 MHz, DMSO-d6) δ 10.95 (s, 1H), 8.44 (d, J = 5.0 Hz, 1H), 8.10 (s, 1H), 8.02-8.01 (m, 1H), 8.01-7.99 (m, 1H), 7.62-7.58 (m, 1H), 7.54-7.49 (m, 2H), 7.47- 7.43 (m, 2H), 7.36-7.32 (m, 2H), 7.00 (dd, J = 5.1, 1.5 Hz, 1H), 4.90 (s, 2H), 3.31-3.24 (m, 4H), 2.56 (s, 4H). LCMS (Analytical Method B) Rt = 2.82 min, MS (ESIpos): m/z 501.3, 503.2 [M + H]+, Purity = 98%. |
| 10 | Intermediate 15 (using 4M HCl) (92% yield) | 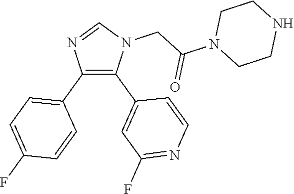 [SMILES: O=C(Cn1cnc(-c2ccc(F)cc2)c1-c1ccnc(F)c1)N1CCNCC1] 2-[4-(4-fluorophenyl)-5-(2- fluoropyridin-4-yl)-1H-imidazol-1- yl]-1-(piperazin-1-yl)ethan-1-one | 1H NMR (400 MHz, DMSO-d6) δ 8.29 (d, J = 5.1 Hz, 1H), 7.83 (s, 1H), 7.43-7.36 (m, 2H), 7.20-7.16 (m, 1H), 7.15-7.08 (m, 3H), 4.97 (s, 2H), 3.30-3.26 (m, 4H), 2.60-2.55 (m, 2H), 2.55-2.52 (m, 2H). LCMS (Analytical Method B) Rt = 2.18 min, MS (ESIpos): m/z 384.3 [M + H]+, Purity = 99%. |
| 66 | Intermediate 16 (using TFA) (53% yield) | 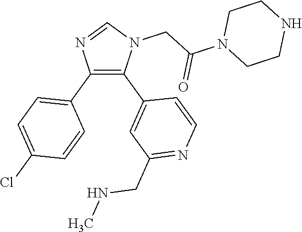 [SMILES: CNCc1cc(-c2c(-c3ccc(Cl)cc3)ncn2CC(=O)N2CCNCC2)ccn1] 2-[4-(4-chlorophenyl)-5-{2- [(methylamino)methyl]pyridin-4- yl}-1H-imidazol-1-yl]-1-(piperazin- 1-yl)ethan-1-one | 1H NMR (500 MHz, DMSO-d6) δ 8.56 (d, J = 5.0 Hz, 1H), 7.81 (s, 1H), 7.39-7.36 (m, 2H), 7.33-7.28 (m, 3H), 7.11 (dd, J = 5.0, 1.4 Hz, 1H), 4.86 (s, 2H), 3.76 (s, 2H), 3.27- 3.21 (m, 4H), 2.56-2.53 (m, 4H), 2.24 (s, 3H). LCMS (Analytical Method B) Rt = 2.08 min, MS (ESIpos): m/z 425.3 [M + H]+, Purity = 95%. |
| 50 | Intermediate 17 (using 4M HCl) (45% yield) | 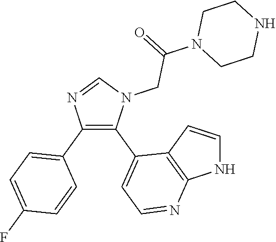 [SMILES: O=C(Cn1cnc(-c2ccc(F)cc2)c1-c1ccnc2[nH]ccc12)N1CCNCC1] 2-[4-(4-fluorophenyl)-5-{1H- pyrrolo[2,3-b]pyridin-4-yl}-1H- imidazol-1-yl]-1-(piperazin-1- yl)ethan-1-one | 1H NMR (400 MHz, MeOH-d4) δ 8.28 (d, J = 5.0 Hz, 1H), 7.91 (s, 1H), 7.40 (d, J = 3.5 Hz, 1H), 7.36-7.30 (m, 2H), 7.04 (d, J = 5.0 Hz, 1H), 6.92-6.85 (m, 2H), 6.12 (d, J = 3.5 Hz, 1H), 4.97-4.91 (m, 1H), 4.78- 4.70 (m, 1H), 3.43-3.36 (m, 2H), 3.21-3.14 (m, 2H), 2.65-2.57 (m, 2H), 2.50-2.42 (m, 2H). LCMS (Analytical Method B) Rt = 1.95 min, MS (ESIpos): m/z 405.3 [M + H]+, Purity = 100%. |
| 73 | Intermediate 18 (using 4M HCl) (59% yield) | 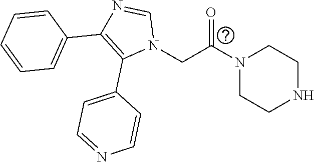 [SMILES: O=C(Cn1cnc(-c2ccccc2)c1-c1ccncc1)N1CCNCC1] 2-[4-phenyl-5-(pyridin-4-yl)-1H- imidazol-1-yl]-1-(piperazin-1- yl)ethan-1-one | 1H NMR (400 MHz, DMSO-d6) δ 8.69-8.59 (m, 2H), 7.80 (s, 1H), 7.39-7.34 (m, 2H), 7.30-7.21 (m, 4H), 7.20-7.13 (m, 1H), 4.88 (s, 2H), 3.29-3.19 (m, 4H), 2.60-2.52 (m, 4H). LCMS (Analytical Method B) Rt = 1.79 min, MS (ESIpos): m/z 348.3 [M + H]+, Purity = 99%. |
| 62 | Intermediate 20 (using 4M HCl) (67% yield) | 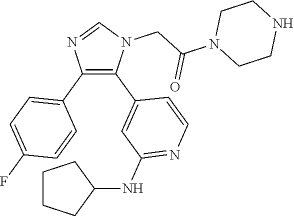 [SMILES: O=C(Cn1cnc(-c2ccc(F)cc2)c1-c1ccnc(NC2CCCC2)c1)N1CCNCC1] 2-{5-[2-(cyclopentylamino)pyridin- 4-yl]-4-(4-fluorophenyl)-1H- imidazol-1-yl}-1-(piperazin-1- yl)ethan-1-one | 1H NMR (400 MHz, DMSO-d6) δ 8.02 (d, J = 5.1 Hz, 1H), 7.72 (s, 1H), 7.50-7.43 (m, 2H), 7.14-7.07 (m, 2H), 6.62 (d, J = 6.7 Hz, 1H), 6.30 (dd, J = 1.4, 5.2 Hz, 1H), 6.27 (s, 1H), 4.78 (s, 2H), 4.07-3.95 (m, 1H), 3.33-3.27 (m, 4H), 2.64-2.56 (m, 4H), 1.91-1.82 (m, 2H), 1.69- 1.60 (m, 2H), 1.56-1.47 (m, 2H), 1.44-1.34 (m, 2H). LCMS (Analytical Method B) Rt = 2.65 min, MS (ESIpos): m/z 449.4 [M + H]+, Purity = 99%. |
| 33 | Intermediate 19 (using 4M HCl) (61% yield) | 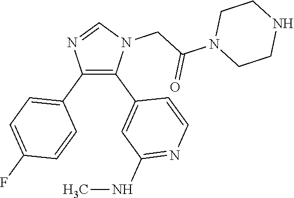 [SMILES: CNc1cc(-c2c(-c3ccc(F)cc3)ncn2CC(=O)N2CCNCC2)ccn1] 2-[4-(4-fluorophenyl)-5-[2- (methylamino)pyridin-4-yl]-1H- imidazol-1-yl]-1-(piperazin-1- yl)ethan-1-one | 1H NMR (500 MHz, DMSO-d6) δ 8.04 (d, J = 5.1 Hz, 1H), 7.72 (s, 1H), 7.48-7.43 (m, 2H), 7.13-7.07 (m, 2H), 6.61 (q, J = 4.8 Hz, 1H), 6.32 (dd, J = 5.2, 1.3 Hz, 1H), 6.28 (s, 1H), 4.79 (s, 2H), 3.36 (s, 2H), 3.32- 3.30 (m, 2H), 2.74 (d, J = 4.8 Hz, 3H), 2.67-2.60 (m, 4H). LCMS (Analytical Method B) Rt = 1.99 min, MS (ESIpos): m/z 395.3 [M + H]+, Purity = 100%. |
| 57 | Intermediate 32 (using 4M HCl) (64% yield) | 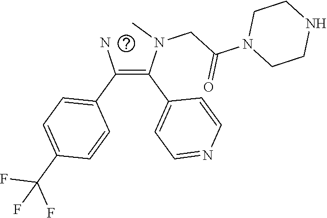 1-(piperazin-1-yl)-2-[5-(pyridin-4- yl)-4-[4-(trifluoromethyl)phenyl]- 1H-imidazol-1-yl]ethan-1-one | 1H NMR (400 MHz, DMSO-d6) δ 8.72-8.64 (m, 2H), 7.87 (s, 1H), 7.62 (d, J = 8.4 Hz, 2H), 7.56 (d, J = 8.3 Hz, 2H), 7.34-7.27 (m, 2H), 4.90 (s, 2H), 3.29-3.21 (m, 4H), 2.55-2.52 (m, 4H). LCMS (Analytical Method B) Rt = 2.39 min, MS (ESIpos): m/z 416.3 [M + H]+, Purity = 95%. |
| 56 | Intermediate 31 (using 4M HCl) (26% yield) | 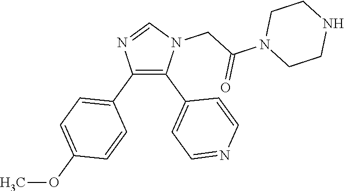 [SMILES: COc1ccc(-c2ncn(CC(=O)N3CCNCC3)c2-c2ccncc2)cc1] 2-[4-(4-methoxyphenyl)-5-(pyridin- 4-yl)-1H-imidazol-1-yl]-1- (piperazin-1-yl)ethan-1-one | 1H NMR (500 MHz, DMSO-d6) δ 8.66-8.60 (m, 2H), 7.76 (s, 1H), 7.30-7.27 (m, 2H), 7.26-7.24 (m, 2H), 6.86-6.80 (m, 2H), 4.88 (s, 2H), 3.72 (s, 3H), 3.30-3.23 (m, 4H), 2.57-2.53 (m, 4H). LCMS (Analytical Method B) Rt = 1.79 min, MS (ESIpos): m/z 378.3 [M + H]+, Purity = 97%. |
| 45 | Intermediate 30 (using 4M HCl) (60% yield) | 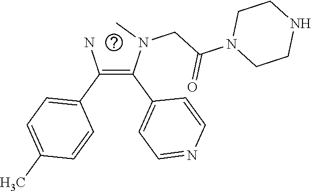 2-[4-(4-methylphenyl)-5-(pyridin- 4-yl)-1H-imidazol-1-yl]-1- (piperazin-1-yl)ethan-1-one | 1H NMR (500 MHz, DMSO-d6) δ 8.65-8.60 (m, 2H), 7.77 (s, 1H), 7.27-7.21 (m, 4H), 7.05 (d, J = 8.0 Hz, 2H), 4.88 (s, 2H), 3.31-3.22 (m, 4H), 2.55-2.52 (m, 4H), 2.25 (S, 3H). LCMS (Analytical Method B) Rt = 2.01 min, MS (ESIpos): m/z 362.3 [M + H]+, Purity = 97%. |
| 59 | Intermediate 34 (39% yield) (using 4M HCl) | 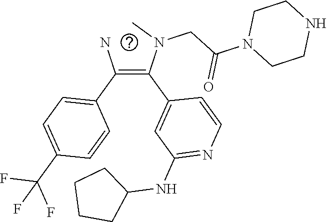 2-{5-[2-(cyclopentylamino)pyridin- 4-yl]-4-[4-(trifluoromethyl)phenyl]- 1H-imidazol-1-yl}-1-(piperazin-1- yl)ethan-1-one | 1H NMR (400 MHz, DMSO-d6) δ 8.06 (d, J = 5.1 Hz, 1H), 7.80 (s, 1H), 7.69-7.61 (m, 4H), 6.64 (d, J = 6.7 Hz, 1H), 6.34 (dd, J = 5.1, 1.4 Hz, 1H), 6.30 (S, 1H), 4.81 (s, 2H), 4.02 (h, J = 6.2, 5.8 Hz, 1H), 3.36-3.23 (m, 4H), 2.63-2.51 (m, 4H), 1.93- 1.81 (m, 2H), 1.73-1.59 (m, 2H), 1.59-1.46 (m, 2H), 1.48-1.34 (m, 2H). LCMS (Analytical Method B) Rt = 3.04 min, MS (ESIpos): m/z 499.4 [M + H]+, Purity = 91%. |
| 3 | Intermediate 33 (using 4M HCl) (59% yield) | 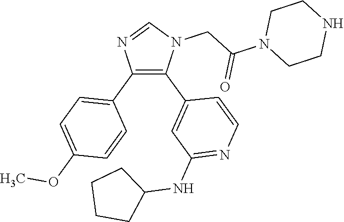 [SMILES: COc1ccc(-c2ncn(CC(=O)N3CCNCC3)c2-c2ccnc(NC3CCCC3)c2)cc1] 2-{5-[2-(cyclopentylamino)pyridin-4-yl]-4- (4-methoxyphenyl)-1H-imidazol-1-yl}-1- (piperazin-1-yl)ethan-1-one | 1H NMR (400 MHz, DMSO-d6) δ 8.01 (d, J = 5.1 Hz, 1H), 7.68 (s, 1H), 7.42-7.34 (m, 2H), 6.88-6.80 (m, 2H), 6.57 (d, J = 6.7 Hz, 1H), 6.34- 6.25 (m, 2H), 4.76 (s, 2H), 4.01 (h, J = 6.1 Hz, 1H), 3.72 (S, 3H), 3.37- 3.22 (m, 4H), 2.62-2.54 (m, 4H), 1.87 (dq, J = 12.0, 6.2, 5.5 Hz, 2H), 1.73-1.59 (m, 2H), 1.58-1.47 (m, 2H), 1.46-1.33 (m, 2H). LCMS (Analytical Method B) Rt = 2.47 min, MS (ESIpos): m/z 461.4 [M + H]+, Purity = 94%. |
| 52 | Intermediate 37 (using 4M HCl) (81% yield) | 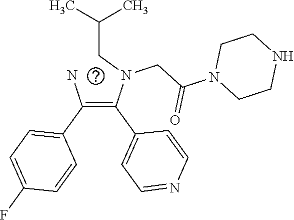 2-[4-(4-fluorophenyl)-2-(propan-2- yl)-5-(pyridin-4-yl)-1H-imidazol-1- yl]-1-(piperazin-1-yl)ethan-1-one | 1H NMR (400 MHz, DMSO-d6) δ 8.67-8.62 (m, 2H), 7.38-7.31 (m, 2H, H17), 7.26-7.21 (m, 2H), 7.10- 7.02 (m, 2H), 4.74 (s, 2H), 3.35- 3.30 (m, 4H), 3.29-3.26 (m, 2H), 3.03-2.94 (m, 1H), 2.59-2.53 (m, 2H), 1.26 (d, J = 6.8 Hz, 6H). LCMS (Analytical Method B) Rt = 2.29 min, MS (ESIpos): m/z 408.3 [M + H]+, Purity = 98%. |
| 51 | Intermediate 36 (using TFA) (87% yield) | 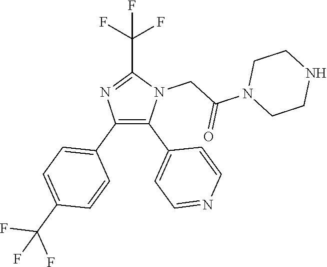 [SMILES: O=C(Cn1c(C(F)(F)F)nc(-c2ccc(C(F)(F)F)cc2)c1-c1ccncc1)N1CCNCC1] 2-[4-(4-fluorophenyl)-5-(pyridin-4- yl)-2-(trifluoromethyl)-1H-imidazol- 1-yl]-1-(piperazin-1-yl)ethan-1-one | 1H NMR (400 MHz, Chloroform-d) δ 8.76-8.70 (m, 2H, H24, H26), 7.41- 7.34 (m, 2H, H7, H11), 7.34-7.29 (m, 2H, H23, H27), 6.96-6.89 (m, 2H, H8, H10), 4.65 (s, 2H, H13), 3.64-3.52 (m, 2H), 3.38-3.26 (m, 2H), 2.89-2.72 (m, 4H). LCMS (Analytical Method E) Rt = 2.55 min, MS (ESIpos): m/z 434.2 [M + H]+, Purity = 100%. |
| 31 | Intermediate 35 (using 4M HCl) (67% yield) | 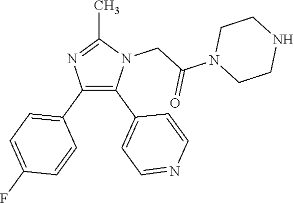 [SMILES: Cc1nc(-c2ccc(F)cc2)c(-c2ccncc2)n1CC(=O)N1CCNCC1] 2-[4-(4-fluorophenyl)-2-methyl-5- (pyridin-4-yl)-1H-imidazol-1-yl]-1- (piperazin-1-yl)ethan-1-one | 1H NMR (400 MHz, Chloroform-d) δ 8.68-8.64 (m, 2H), 7.40-7.35 (m, 2H), 7.23-7.19 (m, 2H), 6.94-6.88 (m, 2H), 4.47 (s, 2H), 3.65-3.58 (m, 2H), 3.36-3.28 (m, 2H), 2.89-2.82 (m, 2H), 2.81-2.75 (m, 2H), 2.43 (s, 3H). LCMS (Analytical Method B) Rt = 1.90 min, MS (ESIpos): m/z 380.3 [M + H]+, Purity = 100%. |
| 55 | Intermediate 38 (using 4M HCl) (47% yield) | 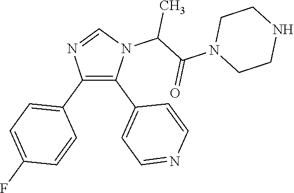 [SMILES: CC(C(=O)N1CCNCC1)n1cnc(-c2ccc(F)cc2)c1-c1ccncc1] 2-[4-(4-fluorophenyl)-5-(pyridin-4- yl)-1H-imidazol-1-yl]-1-(piperazin- 1-yl)propan-1-one | 1H NMR (500 MHz, Chloroform-d) δ 8.74-8.71 (m, 2H), 7.90 (s, 1H), 7.39-7.34 (m, 2H), 7.22-7.20 (m, 2H), 6.94-6.89 (m, 2H), 4.86 (q, J = 7.1 Hz, 1H), 3.71-3.65 (m, 1H), 3.44-3.37 (m, 1H), 3.15-3.08 (m, 1H), 3.07-3.00 (m, 1H), 2.83-2.78 (m, 1H), 2.73-2.66 (m, 2H), 2.43- 2.35 (m, 1H), 1.73 (d, J = 7.1 Hz, 3H). LCMS (Analytical Method B) Rt = 1.94 min, MS (ESIpos): m/z 380.3 [M + H1+, Purity = 100%. |
| 44 | Intermediate 40 (using 4M HCl) (62% yield) | 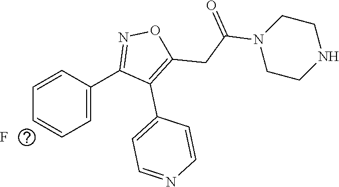 2-[3-(4-fluorophenyl)-4-(pyridin-4- yl)-1,2-oxazol-5-yl]-1-(piperazin-1- yl)ethan-1-one | 1H NMR (400 MHz, DMSO-d6) δ 8.64-8.57 (m, 2H), 7.46-7.38 (m, 2H), 7.34-7.26 (m, 2H), 7.24-7.17 (m, 2H), 4.06 (s, 2H), 3.44-3.34 (m, 4H), 2.71-2.59 (m, 4H). LCMS (Analytical Method B) Rt = 2.26 min, MS (ESIpos): m/z 367.2 [M + H]+, Purity = 100%. |
| 54 | Intermediate 42 (using TFA) (38% yield) | 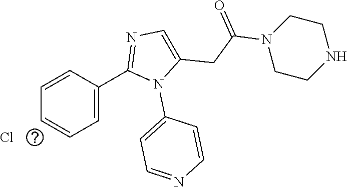 [SMILES: Cl ; O=C(Cc1cnc(-c2ccccc2)n1-c1ccncc1)N1CCNCC1] 2-[2-(4-chlorophenyl)-1-(pyridin-4- yl)-1H-imidazol-5-yl]-1-(piperazin- 1-yl)ethan-1-one | 1H NMR (500 MHz, DMSO-d6) δ 8.71-8.67 (m, 2H), 7.39-7.32 (m, 4H), 7.28-7.23 (m, 2H), 7.05 (s, 1H), 3.67 (s, 2H), 3.27-3.20 (m, 4H), 2.56-2.52 (m, 2H), 2.50-2.46 (m, 2H). LCMS (Analytical Method B) Rt = 1.98 min, MS (ESIpos): m/z 382.2 [M + H]+, Purity = 100%. |
| 23 | Intermediate 44 (using TFA) (13% yield) | 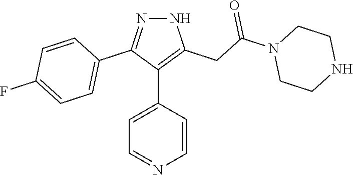 [SMILES: O=C(Cc1[nH]nc(-c2ccc(F)cc2)c1-c1ccncc1)N1CCNCC1] 2-[3-(4-fluorophenyl)-4-(pyridin-4- yl)-1H-pyrazol-5-yl]-1-(piperazin- 1-yl)ethan-1-one | 1H NMR (400 MHz, Chloroform-d) δ 8.59-8.58 (m, 2H), 7.33 (dd, J = 8.7, 5.4 Hz, 2H), 7.14-7.12 (m, 2H), 7.00 (t, J = 8.7 Hz, 2H), 3.75 (s, 2H), 3.65-3.62 (m, 2H), 3.41-3.38 (m, 2H), 2.87-2.80 (m, 4H). LCMS (Analytical Method B) Rt = 1.96 min, MS (ESIpos): m/z 366.3 [M + H]+, Purity = 100%. |
| 43 | Intermediate 45 (using TFA) (67% yield) | 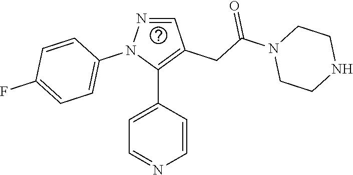 [SMILES: O=C(Cc1cnn(-c2ccc(F)cc2)c1-c1ccncc1)N1CCNCC1] 2-[1-(4-fluorophenyl)-5-(pyridin-4- yl)-1H-pyrazol-4-yl]-1-(piperazin- 1-yl)ethan-1-one | 1H NMR (500 MHz, Chloroform-d) δ 8.66-8.57 (m, 2H), 7.73 (s, 1H), 7.22-7.17 (m, 2H), 7.16-7.13 (m, 2H), 7.04-6.98 (m, 2H), 3.67-3.60 (m, 2H), 3.53 (s, 2H), 3.45-3.40 (m, 2H), 2.90-2.85 (m, 2H), 2.85-2.79 (m, 2H). LCMS (Analytical Method B) Rt = 1.91 min, MS (ESIpos): m/z 366.3 [M + H]+, Purity = 100%. |

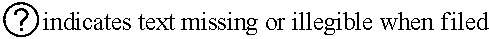

### Example 1.7—Further Synthetic Procedures

### Synthesis of 2-[4-(4-chlorophenyl)-5-[2-(difluoromethyl)pyridin-4-yl]-1H-imidazol-1-yl]-1-{octahydropyrrolo[3,4-c]pyrrol-2-yl}ethan-1-one/Compound 37 of Table 1

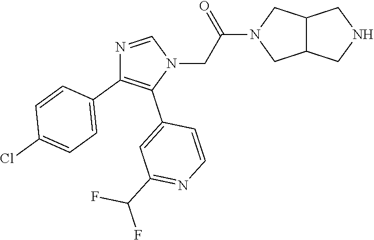 [SMILES: O=C(Cn1cnc(-c2ccc(Cl)cc2)c1-c1ccnc(C(F)F)c1)N1CC2CNCC2C1]

DIPEA (130 μL, 0.744 mmol) was added to a solution of 2-[4-(4-chlorophenyl)-5-[2-(difluoromethyl)-4-pyridyl]imidazol-1-yl]acetic acid (Intermediate 10) (120 mg, 0.330 mmol) and tert-butyl hexahydropyrrolo[3,4-c]pyrrole-2(1H)-carboxylate (120 mg, 0.565 mmol) in EtOAc (2 mL), followed by T3P (50%, 250 μL, 0.420 mmol), and the reaction stirred at RT for 1.5 h. Additional tert-butyl hexahydropyrrolo[3,4-c]pyrrole-2(1H)-carboxylate (70 mg, 0.330 mmol), DIPEA (110 μL, 0.630 mmol) and T3P (50%, 200 μL, 0.330 mmol) were added and the reaction was stirred for 1.5 h. The reaction was quenched into water and the aqueous layer was extracted into EtOAc, dried over MgSO₄ and concentrated in vacuo. The residue was dissolved in 4 M HCl in 1,4-dioxane (2 mL) and stirred at RT for 1 h. The mixture was concentrated in vacuo and the residue was purified by preparative HPLC (Method A1) to afford the title compound as a white solid (80 mg, 52% yield). 1H NMR (500 MHz, DMSO-d6) δ 8.74 (d, J=5.0 Hz, 1H), 7.86 (s, 1H), 7.56 (s, 1H), 7.49-7.45 (m, 1H), 7.38-7.30 (m, 4H), 6.97 (t, J=54.8 Hz, 1H), 4.83 (d, J=17.4 Hz, 1H), 4.81 (d, J=17.3 Hz, 1H), 3.52 (dd, J=10.7, 8.4 Hz, 1H), 3.44 (dd, J=12.2, 8.4 Hz, 1H), 3.17 (dd, J=10.8, 4.5 Hz, 1H), 3.12 (dd, J=12.3, 4.5 Hz, 1H), 2.89-2.84 (m, 2H), 2.77-2.69 (m, 1H), 2.65-2.56 (m, 1H), 2.53-2.51 (m, 1H), 2.49-2.45 (m, 1H). LCMS (Analytical Method A) Rt=1.61 min, MS (ESIpos): m/z 458.3, 460.3 [M+H]+, Purity=100%.

### 2-[4-(4-chlorophenyl)-5-(pyridin-4-yl)-1H-imidazol-1-yl]-1-{7-methyl-2,7-diazaspiro[3.5]nonan-2-yl}ethan-1-one/Compound 36 of Table 1

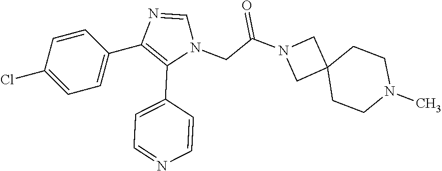 [SMILES: CN1CCC2(CC1)CN(C(=O)Cn1cnc(-c3ccc(Cl)cc3)c1-c1ccncc1)C2]

2-[4-(4-chlorophenyl)-5-(pyridin-4-yl)-1H-imidazol-1-yl]-1-{2,7-diazaspiro[3.5]nonan-2-yl}ethan-1-one (compound 29 of table 1) (25 mg, 0.059 mmol) and 13 M formaldehyde (5.2 μL, 0.066 mmol) were dissolved in THF (1.7 mL) and stirred at RT for 30 min. NaBH(OAc)₃ (26 mg, 0.122 mmol) was added and the reaction was stirred for 2 h. Addition of 13 M formaldehyde (5.2 μL, 0.066 mmol) and NaBH(OAc)₃ (26 mg, 0.122 mmol) was repeated daily for the next 2 days. The reaction was concentrated in vacuo. The residue was partitioned between DCM and 1 M NaOH. The organic layer was separated using a Telos phase separator and the filtrate was concentrated in vacuo. The residue was purified by preparative HPLC (Instrument pump: Gilson 331 & 332; auto injector: Gilson GX281; UV detector: Gilson 159; collector: Gilson GX281; Column: Waters X-Bridge C18 19×100 mm, 5 μm; eluent A: water+0.2 vol % ammonium hydroxide, eluent B: acetonitrile+0.2 vol % ammonium hydroxide; gradient: 0-30% B; flow 20 mL/min; temperature: 25° C.; UV scan: 215 nm) to afford the title compound as a white solid (12 mg, 47% yield). 1H NMR (500 MHz, Chloroform-d) δ 8.74-8.69 (m, 2H), 7.68 (s, 1H), 7.39-7.34 (m, 2H), 7.29-7.27 (m, 2H), 7.23-7.17 (m, 2H), 4.37 (s, 2H), 3.67 (s, 2H), 3.57 (s, 2H), 2.24 (s, 7H), 1.79-1.65 (m, 4H). LCMS (Analytical Method A) Rt=1.19 min, MS (ESIpos): m/z 436.3 [M+H]+, Purity=100%.

### 2-[4-(4-chlorophenyl)-5-[2-(difluoromethyl)pyridin-4-yl]-1H-imidazol-1-yl]-1-{5-methyl-octahydropyrrolo[3,4-c]pyrrol-2-yl}ethan-1-one/Compound 11 of Table 1

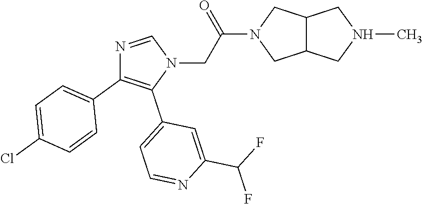 [SMILES: CN1CC2CN(C(=O)Cn3cnc(-c4ccc(Cl)cc4)c3-c3ccnc(C(F)F)c3)CC2C1]

2-[4-(4-chlorophenyl)-5-[2-(difluoromethyl)pyridin-4-yl]-1H-imidazol-1-yl]-1-{octahydropyrrolo[3,4-c]pyrrol-2-yl}ethan-1-one (compound 37 of table 1) (30 mg, 0.0584 mmol) and 13 M formaldehyde (5.4 μL, 0.0701 mmol) were dissolved in DCM (0.6231 mL) and stirred at RT for 10 min. Then NaBH(OAc)₃ (22 mg, 0.105 mmol) was added, and the reaction was stirred at RT for 1 h. The reaction was quenched with sat. NaHCO₃solution and extracted with DCM. The organic layers were filtered through a Telos phase separator and concentrated in vacuo. The residue was purified by flash chromatography (10 g, silica) eluting with 0-7% MeOH/DCM to afford the title compound (6 mg, 19% yield). 1H NMR (400 MHz, DMSO-d6) δ 8.74 (d, J=5.0 Hz, 1H), 7.87 (s, 1H), 7.58-7.55 (m, 1H), 7.49-7.45 (m, 1H), 7.38-7.30 (m, 4H), 6.96 (t, J=54.8 Hz, 1H), 4.90-4.78 (m, 2H), 3.57-3.49 (m, 1H), 3.49-3.42 (m, 1H), 3.23-3.12 (m, 2H), 2.84-2.75 (m, 1H), 2.73-2.64 (m, 1H), 2.44-2.38 (m, 2H), 2.31-2.25 (m, 2H), 2.18 (s, 3H). LCMS (Analytical Method A) Rt=1.64 min, MS (ESIpos): m/z 472.3, 474.3 [M+H]+, Purity=100%.

### 2-[4-(4-chlorophenyl)-5-(pyridin-4-yl)-1H-imidazol-1-yl]-1-{2-methyl-2,7-diazaspiro[3.5]nonan-7-yl}ethan-1-one/Compound 12 of Table 1

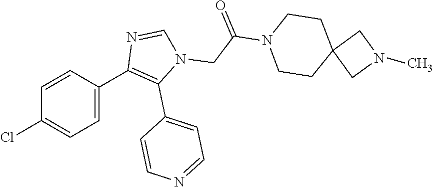 [SMILES: CN1CC2(CCN(C(=O)Cn3cnc(-c4ccc(Cl)cc4)c3-c3ccncc3)CC2)C1]

2-[4-(4-chlorophenyl)-5-(pyridin-4-yl)-1H-imidazol-1-yl]-1-{2,7-diazaspiro[3.5]nonan-7-yl}ethan-1-one (Compound 32 in Table 1) (13 mg, 0.030 mmol) and 13 M formaldehyde (2.7 μL, 0.034 mmol) were dissolved in THF (0.9 mL) and stirred at RT for 30 min. NaBH(OAc)₃ (13 mg, 0.064 mmol) was added and the reaction was stirred for 2 h. Addition of 13 M formaldehyde (2.7 μL, 0.034 mmol) and NaBH(OAc)₃ (13 mg, 0.064 mmol) was repeated daily for the next 2 days. The reaction was concentrated in vacuo. The residue was partitioned between DCM and 1 M NaOH. The organic layer was separated using a Telos phase separator and the filtrate was concentrated in vacuo and triturated with Et₂O to afford the title compound as a white solid (8 mg, 59% yield). 1H NMR (400 MHz, Chloroform-d) δ 8.70-8.65 (m, 2H), 7.63 (s, 1H), 7.40-7.34 (m, 2H), 7.25-7.22 (m, 2H), 7.22-7.18 (m, 2H), 4.58 (s, 2H), 3.53-3.43 (m, 2H), 3.29-3.15 (m, 4H), 3.15-3.00 (m, 2H), 2.41 (s, 3H), 1.71-1.66 (m, 4H). LCMS (Analytical Method B) Rt=2.61 min, MS (ESIpos): m/z 436.3 [M+H]+, Purity=98%.

### N-{4-[4-(4-chlorophenyl)-1-[2-(4-methylpiperazin-1-yl)-2-oxoethyl]-1H-imidazol-5-yl]pyridin-2-yl}benzamide/compound 27 of Table 1

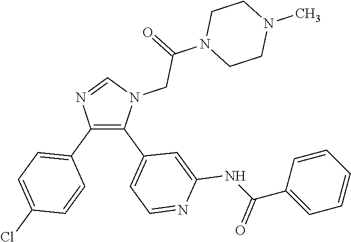 [SMILES: CN1CCN(C(=O)Cn2cnc(-c3ccc(Cl)cc3)c2-c2ccnc(NC(=O)c3ccccc3)c2)CC1]

N-[4-[5-(4-Chlorophenyl)-3-(2-oxo-2-piperazin-1-yl-ethyl)imidazol-4-yl]-2-pyridyl]benzamide (Compound 6 in Table 1) (17 mg, 0.034 mmol) and 13 M formaldehyde (3.0 μL, 0.038 mmol) were dissolved in THF (1 mL) and stirred at RT for 30 min. NaBH(OAc)₃ (15 mg, 0.071 mmol) was added and the reaction was stirred for 30 min. The reaction was concentrated in vacuo. The residue was partitioned between DCM and 1 M NaOH. The organic layer was separated using a Telos phase separator and the filtrate was concentrated in vacuo to afford the title compound (17 g, 91% yield). 1H NMR (500 MHz, DMSO-d6) δ 10.99 (s, 1H), 8.45 (d, J=5.0 Hz, 1H), 8.11 (s, 1H), 8.04-8.03 (m, 1H), 8.02-8.01 (m, 1H), 7.84 (s, 1H), 7.63-7.59 (m, 1H), 7.55-7.50 (m, 2H), 7.47-7.44 (m, 2H), 7.37-7.33 (m, 2H), 7.00 (dd, J=5.1, 1.5 Hz, 1H), 4.92 (s, 2H), 3.43-3.40 (m, 4H), 2.20-2.14 (m, 4H), 2.08 (s, 3H). LCMS (Analytical Method A) Rt=1.81 min, MS (ESIpos): m/z 515.4, 517.4 [M+H]+, Purity=94%.

### 2-[4-(4-chlorophenyl)-5-{1H-pyrrolo[2,3-b]pyridin-4-yl}-1H-imidazol-1-yl]-1-(4-methylpiperazin-1-yl)ethan-1-one/Compound 28 of Table 1

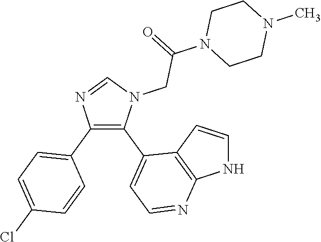 [SMILES: CN1CCN(C(=O)Cn2cnc(-c3ccc(Cl)cc3)c2-c2ccnc3[nH]ccc23)CC1]

2-[4-(4-Chlorophenyl)-5-(1H-pyrrolo[2,3-b]pyridin-4-yl)imidazol-1-yl]-1-piperazin-1-yl-ethanone (Intermediate 22) (80 mg, 0.190 mmol) and 12.7 M formaldehyde (16 μL, 0.203 mmol) were dissolved in THF (1 mL). The mixture was stirred for 30 min then NaBH(OAc)₃ (80 mg, 0.377 mmol) was added. The reaction was stirred for 30 min. The reaction was concentrated in vacuo. The crude product was purified by preparative HPLC (Method A2) then preparative HPLC (Method B1) to afford the title compound (44 mg, 53% yield). 1H NMR (500 MHz, DMSO-d6) δ 11.84 (s, 1H), 8.30 (d, J=4.8 Hz, 1H), 7.86 (s, 1H), 7.45 (d, J=3.4 Hz, 1H), 7.34-7.29 (m, 2H), 7.22-7.18 (m, 2H), 6.94 (d, J=4.8 Hz, 1H), 5.92 (d, J=3.4 Hz, 1H), 4.87 (d, J=17.0 Hz, 1H), 4.62 (d, J=17.0 Hz, 1H), 3.30-3.25 (m, 2H), 3.17-3.11 (m, 2H), 2.09 (s, 3H), 2.08-2.05 (m, 2H), 1.97-1.86 (m, 2H). LCMS (Analytical Method A) Rt=1.28 min, MS (ESIpos): m/z 435.3, 437.3 [M+H]+, Purity=100%.

### 2-[4-(4-chlorophenyl)-5-{2-[(cyclopentylamino)methyl]pyridin-4-yl}-1H-imidazol-1-yl]-1-(piperazin-1-yl)ethan-1-one/Compound 65 of Table 1

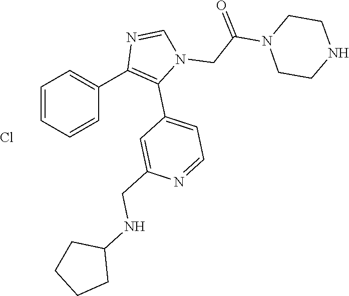 [SMILES: Cl ; O=C(Cn1cnc(-c2ccccc2)c1-c1ccnc(CNC2CCCC2)c1)N1CCNCC1]

Benzyl 4-[2-[4-(4-chlorophenyl)-5-[2-[(cyclopentylamino)methyl]-4-pyridyl]imidazol-1-yl]acetyl]piperazine-1-carboxylate (Intermediate 25) (81 mg, 0.10 mmol) was dissolved in 12 M HCl (aq., 1.5 mL, 18.0 mmol) and heated at 60° C. for 30 min. The mixture was then diluted with MeCN (20 mL) and concentrated in vacuo. The residue was dissolved in MeOH and loaded onto an SCX cartridge (1 g). The column was flushed with MeOH (6 CV) followed by 3M NH₃ in MeOH (6 CV) and the solvent was removed in vacuo. The residue was purified by preparative HPLC (Method A1) to afford the title compound as an off white solid (5.4 mg, 10% yield). 1H NMR (500 MHz, DMSO-d6) δ 8.57 (d, J=5.0 Hz, 1H), 7.82 (s, 1H), 7.38-7.35 (m, 2H), 7.33-7.28 (m, 3H), 7.13 (dd, J=5.0, 1.5 Hz, 1H), 4.88 (s, 2H), 3.78 (s, 2H), 3.30-3.24 (m, 4H), 2.91 (p, J=6.1 Hz, 1H), 2.56-2.54 (m, 4H), 1.66-1.53 (m, 4H), 1.49-1.39 (m, 2H), 1.34-1.20 (m, 2H). LCMS (Analytical Method B) Rt=2.66 min, MS (ESIpos): m/z 479.4 [M+H]+, Purity=97%.

### 2-[4-(4-chlorophenyl)-5-{2-[(dimethylamino)methyl]pyridin-4-yl}-1H-imidazol-1-yl]-1-(piperazin-1-yl)ethan-1-one/Compound 68 of Table 1

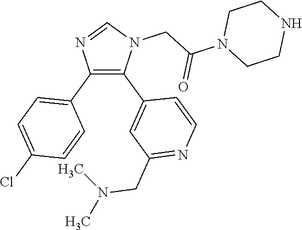 [SMILES: CN(C)Cc1cc(-c2c(-c3ccc(Cl)cc3)ncn2CC(=O)N2CCNCC2)ccn1]

Benzyl 4-[2-[4-(4-chlorophenyl)-5-[2-[(dimethylamino)methyl]-4-pyridyl]imidazol-1-yl]acetyl]piperazine-1-carboxylate (Intermediate 24) (18 mg, 0.016 mmol) was dissolved in 12 M HCl (aq., 0.25 mL, 3.00 mmol) and heated at 60° C. for 15 min. The mixture was then diluted with MeCN (20 mL) and concentrated in vacuo. The residue was dissolved in MeOH and loaded onto an SCX cartridge (1 g). The column was flushed with MeOH (6 CV) followed by 3M NH₃ in MeOH (6 CV) to afford the title compound as an off white solid (3.6 mg, 49% yield). 1H NMR (500 MHz, DMSO-d6) δ 8.56 (d, J=5.0 Hz, 1H), 7.81 (s, 1H), 7.38-7.35 (m, 2H), 7.33-7.28 (m, 2H), 7.26 (s, 1H), 7.16 (dd, J=5.0, 1.5 Hz, 1H), 4.88 (s, 2H), 3.54 (s, 2H), 3.29-3.24 (m, 4H), 2.61-2.57 (m, 4H), 2.15 (s, 6H). LCMS (Analytical Method B) Rt=2.26 min, MS (ESIpos): m/z 439.4 [M+H]+, Purity=93%.

### 2-{5-[2-(aminomethyl)pyridin-4-yl]-4-(4-chlorophenyl)-1H-imidazol-1-yl}-1-(piperazin-1-yl)ethan-1-one/Compound 67 of Table 1

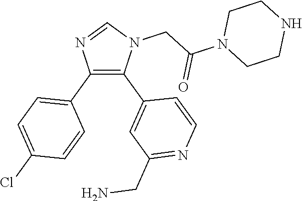 [SMILES: NCc1cc(-c2c(-c3ccc(Cl)cc3)ncn2CC(=O)N2CCNCC2)ccn1]

Benzyl 4-[2-[4-(4-chlorophenyl)-5-[2-(formamidomethyl)-4-pyridyl]imidazol-1-yl]acetyl]piperazine-1-carboxylate (Intermediate 23) (7.0 mg, 0.012 mmol) was dissolved in 12 M HCl (aq., 0.25 mL, 3.00 mmol) and heated at 60° C. for 15 min. The mixture was then diluted with MeCN (20 mL) and concentrated in vacuo. The residue was dissolved in MeOH and loaded onto an SCX cartridge (1 g). The column was flushed with MeOH (6 CV) followed by 3M NH₃ in MeOH (6 CV) to afford the title compound as an off white solid (4.5 mg, 83% yield). 1H NMR (500 MHz, DMSO-d6) δ 8.57 (d, J=4.9 Hz, 1H), 7.82 (s, 1H), 7.42-7.36 (m, 3H), 7.32 (d, J=8.6 Hz, 2H), 7.10 (d, J=4.4 Hz, 1H), 4.89 (s, 2H), 3.92 (s, 2H), 3.30-3.24 (m, 4H), 2.60-2.53 (m, 4H). LCMS (Analytical Method B) Rt=1.94 min, MS (ESIpos): m/z 411.3 [M+H]+, Purity=93%.

### N-({4-[4-(4-chlorophenyl)-1-[2-oxo-2-(piperazin-1-yl)ethyl]-1H-imidazol-5-yl]pyridin-2-yl}methyl)acetamide/compound 70 of Table 1

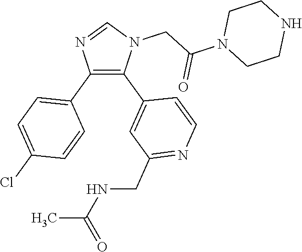 [SMILES: CC(=O)NCc1cc(-c2c(-c3ccc(Cl)cc3)ncn2CC(=O)N2CCNCC2)ccn1]

Benzyl 4-[2-[5-[2-(acetamidomethyl)-4-pyridyl]-4-(4-chlorophenyl)imidazol-1-yl]acetyl]piperazine-1-carboxylate/(Intermediate 29) (80 mg, 0.14 mmol) was dissolved in 12 M HCl (aq., 2.0 mL, 24.0 mmol) and heated at 60° C. for 30 min. The mixture was then diluted with MeCN (20 mL) and concentrated in vacuo. The residue was dissolved in MeOH and loaded onto an SCX cartridge (1 g). The column was flushed with MeOH (6 CV) followed by 3M NH₃ in MeOH (6 CV) to afford the title compound as an off white solid (17.5 mg, 27% yield). 1H NMR (500 MHz, DMSO-d6) δ 8.56 (d, J=5.0 Hz, 1H), 8.39 (t, J=5.8 Hz, 1H), 7.81 (s, 1H), 7.40-7.35 (m, 2H), 7.34-7.28 (m, 2H), 7.17 (s, 1H), 7.12 (dd, J=5.0, 1.4 Hz, 1H), 4.86 (s, 2H), 4.34 (d, J=5.9 Hz, 2H), 3.31-3.23 (m, 4H), 2.62-2.53 (m, 4H), 1.81 (s, 3H). LCMS (Analytical Method B) Rt=1.93 min, MS (ESIpos): m/z 453.3 [M+H]+, Purity=96%.

### 2-[5-(2-aminopyridin-4-yl)-4-(4-fluorophenyl)-1H-imidazol-1-yl]-1-(piperazin-1-yl)ethan-1-one/compound 61 of Table 1

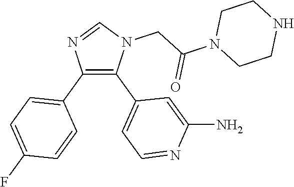 [SMILES: Nc1cc(-c2c(-c3ccc(F)cc3)ncn2CC(=O)N2CCNCC2)ccn1]

A mixture of Pd/C (10%, 8.2 mg, 7.77 μmol) and benzyl 4-[2-[5-(2-amino-4-pyridyl)-4-(4-fluorophenyl)imidazol-1-yl]acetyl]piperazine-1-carboxylate (Intermediate 21) (20 mg, 0.0389 mmol) in a mixture of EtOH (2.00 mL) and MeOH (0.40 mL) was stirred under an atmosphere of hydrogen for 20 h. The mixture was filtered through celite, washing with MeOH and concentrated in vacuo. The residue was purified by preparative HPLC (Method A1) to yield the title compound (2 mg, 14% yield). 1H NMR (500 MHz, MeOH-d4) δ 7.97 (d, J=5.3 Hz, 1H), 7.80 (s, 1H), 7.44 (dd, J=8.9, 5.4 Hz, 2H), 7.01 (t, J=8.9 Hz, 2H), 6.50 (dd, J=5.3, 1.4 Hz, 1H), 6.47 (s, 1H), 4.92 (s, 2H), 4.59 (s, 2H), 3.54-3.48 (m, 2H), 3.44-3.39 (m, 2H), 2.78-2.69 (m, 4H). LCMS (Analytical Method C) Rt=2.70 min, MS (ESIpos): m/z 381.2 [M+H]+, Purity=98%.

### N-{4-[4-(4-fluorophenyl)-1-[2-oxo-2-(piperazin-1-yl)ethyl]-1H-imidazol-5-yl]pyridin-2-yl}benzamide/Compound 2 of Table 1

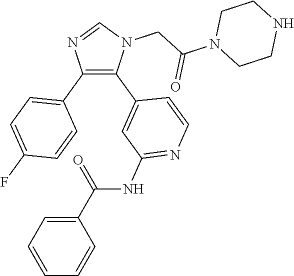 [SMILES: O=C(Nc1cc(-c2c(-c3ccc(F)cc3)ncn2CC(=O)N2CCNCC2)ccn1)c1ccccc1]

EtOH (1 mL) and MeOH (0.2 mL) were added to a mixture of benzyl 4-[2-[5-(2-benzamido-4-pyridyl)-4-(4-fluorophenyl)imidazol-1-yl]acetyl]piperazine-1-carboxylate (Intermediate 26) (12 mg, 0.0194 mmol) and Pd/C (10%, 4.1 mg, 3.88 μmol). The reaction was stirred under an atmosphere of hydrogen for 24 h. Additional Pd/C (10%, 4.1-mg, 3.88 μmol) was added, and the reaction stirred under an atmosphere of hydrogen for 24 h. The mixture was filtered through a pad of celite and the filtrate was concentrated in vacuo. The residue was purified by preparative HPLC (Method A1) to yield the title compound (4 mg, 43% yield). 1H NMR (400 MHz, Chloroform-d) δ 8.64 (s, 1H), 8.31-8.24 (m, 2H), 7.95-7.87 (m, 2H), 7.68 (s, 1H), 7.61 (t, J=7.4 Hz, 1H), 7.53 (t, J=7.4 Hz, 2H), 7.50-7.44 (m, 2H), 7.01-6.92 (m, 3H), 4.84 (s, 2H), 3.61-3.55 (m, 2H), 3.40-3.33 (m, 2H), 2.85-2.73 (m, 4H). LCMS (Analytical Method A) Rt=1.54 min, MS (ESIpos): m/z 485.2 [M+H]+, Purity=100%.

### N-{4-[4-(4-fluorophenyl)-1-[2-oxo-2-(piperazin-1-yl)ethyl]-1H-imidazol-5-yl]pyridin-2-yl}cyclopropanecarboxamide/compound 21 of Table 1

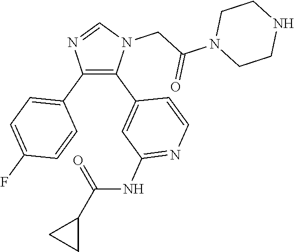 [SMILES: O=C(Nc1cc(-c2c(-c3ccc(F)cc3)ncn2CC(=O)N2CCNCC2)ccn1)C1CC1]

Benzyl 4-[2-[5-[2-(cyclopropanecarbonylamino)-4-pyridyl]-4-(4-fluorophenyl)imidazol-1-yl]acetyl]piperazine-1-carboxylate (Intermediate 27) (35 mg, 0.0565 mmol) was dissolved in MeOH (2 mL). The solution was passed through an H-Cube Pro hydrogenation system charged with a 10% Pd/C CatCart cartridge (1 mL/min, 50° C.). The solution was concentrated in vacuo and purified by preparative HPLC (Method A2) to afford the title compound (7 mg, 27% yield). 1H NMR (500 MHz, Chloroform-d) δ 8.25 (s, 1H), 8.22 (d, J=5.1 Hz, 1H), 8.09 (s, 1H), 7.64 (s, 1H), 7.44 (dd, J=8.8, 5.5 Hz, 2H), 6.96-6.91 (m, 3H), 4.78-4.74 (m, 2H), 3.55-3.51 (m, 2H), 3.33-3.28 (m, 2H), 2.80-2.77 (m, 2H), 2.76-2.72 (m, 2H), 1.59-1.55 (m, 1H), 1.09-1.05 (m, 2H), 0.95-0.90 (m, 2H). LCMS (Analytical Method B) Rt=2.19 min, MS (ESIpos): m/z 449.3 [M+H]+, Purity=98%.

### N-{4-[4-(4-fluorophenyl)-1-[2-oxo-2-(piperazin-1-yl)ethyl]-1H-imidazol-5-yl]pyridin-2-yl}acetamide/compound 26 of Table 1

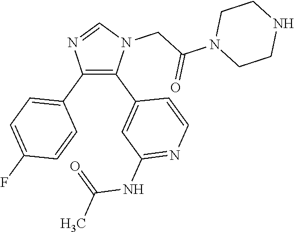 [SMILES: CC(=O)Nc1cc(-c2c(-c3ccc(F)cc3)ncn2CC(=O)N2CCNCC2)ccn1]

Benzyl 4-[2-[5-(2-acetamido-4-pyridyl)-4-(4-fluorophenyl)imidazol-1-yl]acetyl]piperazine-1-carboxylate (Intermediate 28) (55 mg, 0.0791 mmol) was dissolved in 1:1 MeOH/dioxane (3 mL) and the solution was passed through a H-Cube Pro hydrogenation system charged with a 10% Pd/C CatCart cartridge (1 mL/min, 50° C.). The solution was concentrated in vacuo, and the residue was purified by preparative HPLC (Method B1). The product containing fractions were combined, and the MeCN was removed in vacuo. The solution was basified with 1 M NaOH and extracted with DCM (2×). The organics were combined, concentrated in vacuo and lyophilised overnight to afford the title compound (13 mg, 37% yield). 1H NMR (500 MHz, MeOH-d4) δ 8.45 (s, 1H), 8.25 (d, J=5.1 Hz, 1H), 8.01 (s, 1H), 7.82 (s, 1H), 7.43-7.38 (m, 2H), 7.05-6.99 (m, 2H), 6.87 (dd, J=5.1, 1.5 Hz, 1H), 5.07 (s, 2H), 3.78 (s, 2H), 3.68 (s, 2H), 3.19 (s, 2H), 3.12 (s, 2H), 2.18 (s, 3H). LCMS (Analytical Method B) Rt=1.88 min, MS (ESIpos): m/z 423.3 [M+H]+, Purity=95%.

### Synthesis of N-{4-[4-(4-fluorophenyl)-1-[2-(morpholin-4-yl)-2-oxoethyl]-1H-imidazol-5-yl]pyridin-2-yl}benzamide/Compound 25 of Table 1

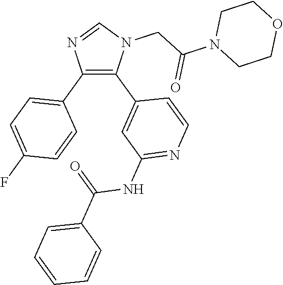 [SMILES: O=C(Nc1cc(-c2c(-c3ccc(F)cc3)ncn2CC(=O)N2CCOCC2)ccn1)c1ccccc1]

A mixture of 2-[5-bromo-4-(4-fluorophenyl)-1H-imidazol-1-yl]-1-(morpholin-4-yl)ethan-1-one (Intermediate 43) (130 mg, 0.300 mmol), (2-benzamido-4-pyridyl)boronic acid (Intermediate 14-5) (455 mg, 0.564 mmol), Pd(PPh₃)₄ (17 mg, 0.015 mmol) and 2 M Na₂CO₃ (0.75 mL, 1.50 mmol) in DME (2.2 mL) was degassed by sparging with nitrogen. The reaction was heated to 125° C. for 1 h under microwave/irradiation. The mixture was diluted with EtOAc, washed with water, dried, filtered and concentrated in vacuo. The residue was purified by preparative HPLC (Method A1) to yield the title compound (75 mg, 50% yield). 1H NMR (500 MHz, DMSO-d6) δ 10.97 (s, 1H), 8.44 (dd, J=5.0, 0.6 Hz, 1H), 8.12-8.08 (m, 1H), 8.04-7.99 (m, 2H), 7.82 (s, 1H), 7.64-7.59 (m, 1H), 7.57-7.49 (m, 2H), 7.51-7.43 (m, 2H), 7.17-7.09 (m, 2H), 6.99 (dd, J=5.1, 1.5 Hz, 1H), 4.95 (s, 2H), 3.56-3.47 (m, 4H), 3.46-3.38 (m, 4H). LCMS (Analytical Method B) Rt=2.75 min, MS (ESIpos): m/z 486.3 [M+H]+, Purity=98%.

### Synthesis of tert-butyl 2-[4-(4-fluorophenyl)-5-(pyridin-3-yl)-1H-imidazol-1-yl]acetate/Intermediate X-1

Tert-butyl 2-[5-bromo-4-(4-fluorophenyl)imidazol-1-yl]acetate (Intermediate 1-2) (350 mg, 0.985 mmol), 3-pyridylboronic acid (140 mg, 1.14 mmol) and sodium carbonate (308 mg, 2.91 mmol) were suspended in DME (4 mL) and Water (1 mL) and degassed with nitrogen for 5 min. tetrakis(triphenylphosphine)palladium (58 mg, 0.0505 mmol) was added and the mixture was sealed under nitrogen and stirred at 100° C. for 2 hours. Additional 3-pyridylboronic acid (61 mg, 0.493 mmol) and tetrakis(triphenylphosphine)palladium (57 mg, 0.0493 mmol) were added and the reaction was stirred for 16 hours at 100° C. The mixture was diluted with water and extracted with DCM. The organics were combined and concentrated in vacuo and the residue was purified by flash chromatography (25 g, silica) eluting with 0-10% MeOH/DCM. The resulting product was further purified by preparative HPLC to yield the title compound (110 mg, 30% yield). 1H NMR (400 MHz, DMSO-d6) δ 8.65 (dd, J=1.7, 4.8 Hz, 1H), 8.46 (dd, J=0.8, 2.2 Hz, 1H), 7.88 (s, 1H), 7.77 (dt, J=1.8, 7.8 Hz, 1H), 7.52 (ddd, J=0.8, 4.9, 7.8 Hz, 1H), 7.40-7.34 (m, 2H), 7.11-7.04 (m, 2H), 4.76 (s, 2H), 1.24 (s, 9H). MS (ESIpos): m/z 354.2 [M+H]+, Purity=97%.

### Synthesis of 2-[4-(4-fluorophenyl)-5-(pyridin-3-yl)-1H-imidazol-1-yl]acetic acid; bis(trifluoroacetic acid)/Intermediate X

Tert-butyl 2-[4-(4-fluorophenyl)-5-(3-pyridyl)imidazol-1-yl]acetate (Intermediate X-1) (100 mg, 0.283 mmol) was dissolved in DCM (2 mL) and TFA (0.5 mL). The mixture was stirred at room temperature for 2 days, then concentrated in vacuo to yield the title compound (104 mg, 98% yield), which was used in the next step without further purification. 1H NMR (400 MHz, DMSO-d6) δ 8.75-8.71 (m, 2H), 8.55 (d, J=1.6 Hz, 1H), 7.89 (dt, J=1.8, 7.9 Hz, 1H), 7.60 (ddd, J=0.7, 4.9, 7.9 Hz, 1H), 7.42-7.35 (m, 2H), 7.23-7.15 (m, 2H), 4.90 (s, 2H). MS (ESIpos): m/z 298.1 [M+H]+, Purity=83%.

### Synthesis of tert-butyl 4-{2-[4-(4-fluorophenyl)-5-(pyridin-3-yl)-1H-imidazol-1-yl]acetyl}piperazine-1-carboxylate/Intermediate Y

To a stirred solution of 2-[4-(4-fluorophenyl)-5-(pyridin-3-yl)-1H-imidazol-1-yl]acetic acid bis(trifluoroacetic acid (Intermediate X) (80%, 50 mg, 0.135 mmol) and tert-butyl piperazine-1-carboxylate (45 mg, 0.242 mmol) in EtOAc (1.5 mL), DIPEA (70 uL, 0.401 mmol) was added followed by T3P (50%, 100 uL, 0.168 mmol), and the reaction was stirred for 2.5 hours at room temperature. Water was added and the mixture was extracted with EtOAc, dried over MgSO₄, filtered and concentrated in vacuo to afford the title compound (30 mg, 58% yield), which was used in the next step without further purification. MS (ESIpos): m/z 466.3 [M+H]+, Purity=86%.

### Reference Compound 107 from Table 1/2-[4-(4-fluorophenyl)-5-(pyridin-3-yl)-1H-imidazol-1-yl]-1-(4-methylpiperazin-1-yl)ethan-1-one

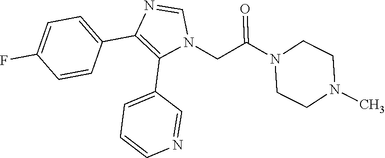 [SMILES: CN1CCN(C(=O)Cn2cnc(-c3ccc(F)cc3)c2-c2cccnc2)CC1]

Intermediate X and 1-methylpiperazine (39% yield)₁H NMR (500 MHz, DMSO-d6) δ 8.65 (dd, J=1.7, 4.8 Hz, 1H), 8.43 (dd, J=0.8, 2.2 Hz, 1H), 7.80 (s, 1H), 7.69 (dt, J=1.9, 7.8 Hz, 1H), 7.51 (ddd, J=0.8, 4.9, 7.8 Hz, 1H), 7.38-7.33 (m, 2H), 7.10-7.04 (m, 2H), 4.86 (s, 2H), 3.33-3.28 (m, 4H), 2.15-2.09 (m, 7H). LCMS Rt=2.09 min, MS (ESIpos): m/z 380.3 [M+H]+, Purity=100%.

### Reference Compound 108 from Table 1/2-[4-(4-fluorophenyl)-5-(pyridin-3-yl)-1H-imidazol-1-yl]-1-(piperazin-1-yl)ethan-1-one

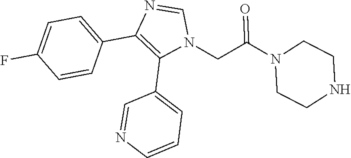 [SMILES: O=C(Cn1cnc(-c2ccc(F)cc2)c1-c1cccnc1)N1CCNCC1]

Intermediate Y (using 4M HCl) (36% yield)₁H NMR (400 MHz, Methanol-d4) δ 8.62 (dd, J=1.5, 4.9 Hz, 1H), 8.45 (d, J=1.5 Hz, 1H), 7.85 (s, 1H), 7.81 (dt, J=1.8, 7.9 Hz, 1H), 7.54 (dd, J=5.0, 7.6 Hz, 1H), 7.39-7.31 (m, 2H), 7.03-6.94 (m, 2H), 4.94 (s, 2H), 3.50-3.43 (m, 2H), 3.41-3.34 (m, 2H), 2.73-2.65 (m, 4H). LCMS Rt=1.92 min, MS (ESIpos): m/z 366.3 [M+H]+, Purity=97%.

### Example 1.8—More Intermediates

### Synthesis of tert-butyl 2-[4-(4-chlorophenyl)-5-(3-chloropyridin-4-yl)-1H-imidazol-1-yl]acetate/Intermediate 46-1

A mixture of tert-butyl 2-[5-bromo-4-(4-chlorophenyl)imidazol-1-yl]acetate (Intermediate 2-2) (660 mg, 1.78 mmol), 3-chloro-4-(4,4,5,5-tetramethyl-1,3,2-dioxaborolan-2-yl)pyridine (553 mg, 2.31 mmol), and Pd(PPh₃)₄ (164 mg, 0.142 mmol) in DME (9 mL) was degassed by sparging with nitrogen whilst stirring. 2 M Na₂CO₃ (3.0 mL, 6.00 mmol) was added and the mixture stirred for 3 more min. The mixture was heated to 110° C. under microwave irradiation for 3 h. The mixture was partitioned between water (50 mL) and EtOAc (50 mL). The organic layer was separated and the aqueous layer was extracted with EtOAc (50 mL). The combined organics were dried over MgSO₄ and concentrated in vacuo. The residue was purified by flash chromatography (silica, 25 g) eluting with 0-2% MeOH/DCM. A second flash chromatography was carried out (silica, 25 g) eluting with 0-60% EtOAc/heptane to yield the title compound (212 mg, 25% yield) as a pale-yellow oil containing the product impure with the dehalogenated product. 1H NMR (400 MHz, Chloroform-d) δ 8.79 (s, 1H), 8.56 (d, J=4.9 Hz, 1H), 7.70 (s, 1H), 7.35-7.30 (m, 2H), 7.27-7.25 (m, 1H), 7.23-7.17 (m, 2H), 4.51 (d, J=17.7 Hz, 1H), 4.28 (d, J=17.7 Hz, 1H), 1.36 (s, 9H). LCMS (Analytical Method J) Rt=1.01 min, MS (ESIpos): m/z 404.2, 406.2 [M+H]+, Purity=87%.

### Synthesis of 2-[4-(4-chlorophenyl)-5-(3-chloropyridin-4-yl)-1H-imidazol-1-yl]acetic acid/Intermediate 46

TFA (0.51 mL, 6.82 mmol) was added to a solution of tert-butyl 2-[4-(4-chlorophenyl)-5-(3-chloro-4-pyridyl)imidazol-1-yl]acetate (Intermediate 46-1) (83% purity, 220 mg, 0.452 mmol) in DCM (2 mL) and the resulting mixture stirred at RT for 24 h. The solvent was evaporated under reduced pressure and Et₂O was added and evaporated multiple times. The product was dried in the vacuum oven overnight to yield the title compound as TFA salt (201 mg, 63% yield) as a pale-brown solid, which was used in the next step without further purification. 1H NMR (400 MHz, Methanol-d4) δ 8.81 (s, 1H), 8.70 (s, 1H), 8.64 (d, J=4.9 Hz, 1H), 7.53-7.50 (m, 1H), 7.39-7.34 (m, 2H), 7.34-7.28 (m, 2H), 5.00 (d, J=18.0 Hz, 1H), 4.72 (d, J=18.0 Hz, 1H). LCMS (Analytical Method J) Rt=0.68 min, MS (ESIpos): m/z 348.1, 350.0 [M+H]+, Purity=84%.

### Synthesis of 2-chloro-N-(pyridin-4-yl)acetamide/Intermediate 47-1

A solution of pyridin-4-amine (500 mg, 5.31 mmol) in DCM (5 mL) was added dropwise to a solution of 2-chloroacetyl chloride (634 uL, 7.97 mmol) in DCM (10 mL) that was cooled in an ice bath. triethylamine (1.5 mL, 10.6 mmol) was added and the reaction was left to warm to RT and stirred for 3 h. The reaction was concentrated under reduced pressure and diluted in EtOAc, then washed with water followed by brine. Organic layer was dried with MgSO₄ and concentrated under reduced pressure to give the crude product. Crude product was purified by flash chromatography (25 g, silica) eluting with 0-100% EtOAc/Heptane to afford the title compound (75 mg, 8.3% yield) as a colourless oil. 1H NMR (400 MHz, DMSO-d6) δ 10.65 (s, 1H), 8.46 (d, J=6.2 Hz, 2H), 7.68-7.43 (m, 2H), 4.31 (s, 2H). LCMS (Analytical Method H) Rt=0.31 min, MS (ESIpos): m/z 170.7 [M+H]+, Purity=98%.

### Synthesis of 2-[4-(4-chlorophenyl)-1H-imidazol-1-yl]-N-(pyridin-4-yl)acetamide/Intermediate 47-2

4-(4-Chlorophenyl)-1H-imidazole (73 mg, 0.410 mmol) was dissolved in THF (1.3 mL) and cooled to 0° C., then NaH (60%, 16 mg, 0.410 mmol) was added. The mixture was stirred for 5 min then 2-chloro-N-(4-pyridyl)acetamide (Intermediate 47-1) (70 mg, 0.410 mmol) was added. The reaction was stirred for 2 h. The reaction was quenched with water and extracted with DCM. Organic phase was washed with brine and concentrated under reduced pressure. Crude product was purified via flash chromatography (10 g, silica) eluting with 0-100% MeOH in DCM and fractions were collected to afford the title compound (45 mg, 32% yield) as a brown oil. 1H NMR (500 MHz, DMSO-d6) δ 10.82 (s, 1H), 8.49-8.44 (m, 2H), 7.79-7.75 (m, 2H), 7.72 (d, J=1.2 Hz, 1H), 7.68 (d, J=1.2 Hz, 1H), 7.59-7.55 (m, 2H), 7.43-7.38 (m, 2H), 5.01 (s, 2H). LCMS (Analytical Method E) Rt=0.48 min, MS (ESIpos): m/z 313.2 [M+H]+, Purity=92%.

### Synthesis of 2-[5-bromo-4-(4-chlorophenyl)-1H-imidazol-1-yl]-N-(pyridin-4-yl)acetamide/Intermediate 47

2-[4-(4-Chlorophenyl)imidazol-1-yl]-N-(4-pyridyl)acetamide (Intermediate 47-2) (45 mg, 0.144 mmol) was dissolved in THF (1 mL) and cooled to 0° C., then N-bromosuccinimide (26 mg, 0.144 mmol) was added. The reaction was allowed to warm to RT and stirred for 1 h. The reaction was quenched with water and extracted with DCM. The organics were combined and concentrated in vacuo and the crude product was purified via flash chromatography (11 g, KP-NH) eluting with 0-100% DCM/MeOH. Fractions collected and concentrated under reduced pressure to afford the title compound (35 mg, 45% yield) as a brown oil. LCMS (Analytical Method H) Rt=0.53 min, MS (ESIpos): m/z 391.2/393.1 [M+H]+, Purity=72%.

### Synthesis of ethyl 2-[5-(2-aminopyridin-4-yl)-4-(4-fluorophenyl)-1H-imidazol-1-yl]acetate/Intermediate 48-1

tert-Butyl 2-[5-[2-(tert-butoxycarbonylamino)-4-pyridyl]-4-(4-fluorophenyl)imidazol-1-yl]acetate (Intermediate 11-1) (73% purity, 480 mg, 0.748 mmol) was dissolved in 4M HCl in dioxane (5 mL) and EtOH (3 mL) and stirred at RT for 4 h. The reaction was left standing over the weekend. Additional 4M HCl in dioxane (6 mL) was added and the reaction was stirred at RT for 2 h. The reaction was stirred at 50° C. for 8 h. The mixture was concentrated in vacuo then purified via HPLC (Method A1). The product containing fractions were combined and the MeCN removed in vacuo then extracted with DCM. The organics were concentrated in vacuo to afford the title compound (48 mg, 17% yield) as a white solid. 1H NMR (500 MHz, Chloroform-d) δ 8.12 (d, J=5.2 Hz, 1H), 7.62 (s, 1H), 7.49-7.45 (m, 2H), 6.97-6.91 (m, 2H), 6.55 (dd, J=1.4, 5.2 Hz, 1H), 6.41 (s, 1H), 4.73 (s, 2H), 4.55 (s, 2H), 4.19 (q, J=7.1 Hz, 2H), 1.24 (t, J=7.1 Hz, 3H). LCMS (Analytical Method J) Rt=0.51 min, MS (ESIpos): m/z 341.2 [M+H]+, Purity=92%.

### Synthesis of 2-{5-[2-(2,2-dimethylpropanamido)pyridin-4-yl]-4-(4-fluorophenyl)-1H-imidazol-1-yl}acetic acid/Intermediate 48

Ethyl 2-[5-(2-amino-4-pyridyl)-4-(4-fluorophenyl)imidazol-1-yl]acetate (Intermediate 48-1) (24 mg, 0.0670 mmol) and DIPEA (36 uL, 0.206 mmol) were dissolved in THF-Anhydrous (2 mL), then pivaloyl chloride (18 uL, 0.148 mmol) was added. The reaction was stirred for 2 h at RT. 2 M aq. NaOH (1.0 mL, 2.00 mmol) was added and the reaction was stirred for 1 h. The reaction was diluted with water and extracted with DCM. The organics were combined and concentrated in vacuo to afford the title compound (15 mg, 44% yield) as a white solid without further purification. LCMS (Analytical Method J) Rt=0.66 min, MS (ESIpos): m/z 397.3 [M+H]+, Purity=78%.

### Synthesis of 2-[5-(2-cyclopentaneamidopyridin-4-yl)-4-(4-fluorophenyl)-1H-imidazol-1-yl]acetic acid/Intermediate 49

Ethyl 2-[5-(2-amino-4-pyridyl)-4-(4-fluorophenyl)imidazol-1-yl]acetate (Intermediate 48-1) (24 mg, 0.0670 mmol) and DIPEA (35 uL, 0.200 mmol) were dissolved in THF-Anhydrous (2 mL), then cyclopentanecarbonyl chloride (20 uL, 0.165 mmol) was added. The mixture was stirred at RT for 2 h. Additional cyclopentanecarbonyl chloride (10 uL, 0.0670 mmol) and DIPEA (35 uL, 0.200 mmol) were added and the reaction was stirred for 15 min. 2 M aq. NaOH (1.0 mL, 2.00 mmol) was added and the mixture was stirred for 1 h. The reaction was diluted with water and extracted with DCM. The organics were combined and concentrated in vacuo to afford the title compound (9 mg, 29% yield). The material was used directly in the next step. LCMS (Analytical Method J) Rt=0.68 min, MS (ESIpos): m/z 409.3 [M+H]+, Purity=89%.

### Synthesis of tert-butyl 2-[2-chloro-4-(4-fluorophenyl)-5-(pyridin-4-yl)-1H-imidazol-1-yl]acetate/Intermediate 50-1

N-chlorosuccinimide (76 mg, 0.570 mmol) was added to an ice-cold solution of tert-butyl 2-[4-(4-fluorophenyl)-5-(4-pyridyl)imidazol-1-yl]acetate (Intermediate 1-3) (175 mg, 0.475 mmol) in DCM (4 mL) and the resulting mixture was allowed to stir at RT for 3 h, then at 50° C. for 2 h. Reaction quenched with 1M aq. NaOH. Organic layer isolated using a Telos phase separator and evaporated under reduced pressure. The residue was purified by flash chromatography (25 g, silica) eluting with 0-50% EtOAc/heptane to yield the title compound (112 mg, 57% yield) as a pale yellow solid. 1H NMR (400 MHz, Chloroform-d) δ 8.72 (d, J=5.7 Hz, 2H), 7.41-7.33 (m, 2H), 7.25-7.21 (m, 2H), 6.97-6.88 (m, 2H), 4.42 (s, 2H), 1.44 (s, 9H). LCMS (Analytical Method J) Rt=0.96 min, MS (ESIpos): m/z 388.2, 390.2 [M+H]+, Purity=94%.

### Synthesis of 2-[2-chloro-4-(4-fluorophenyl)-5-(pyridin-4-yl)-1H-imidazol-1-yl]acetic acid/Intermediate 50

TFA (0.99 mL, 13.3 mmol) was added to a solution of tert-butyl 2-[2-chloro-4-(4-fluorophenyl)-5-(4-pyridyl)imidazol-1-yl]acetate (Intermediate 50-1) (110 mg, 0.267 mmol) in DCM 2 mL) and the resulting mixture stirred at RT for 6 h. The solvent was evaporated under reduced pressure and Et₂O was added and evaporated multiple times. The product was dissolved in MeCN/water and freeze-dried overnight to afford the title compound as TFA salt (125 mg, 75% yield). 1H NMR (400 MHz, DMSO-d6) δ 8.78 (d, J=6.1 Hz, 2H), 7.50-7.44 (m, 2H), 7.39-7.32 (m, 2H), 7.16-7.09 (m, 2H), 4.68 (s, 2H). LCMS (Analytical Method H) Rt=0.30 min, MS (ESIpos): m/z 332.2, 334.2 [M+H]+.

### Synthesis of tert-butyl 7-{2-[2-chloro-4-(4-fluorophenyl)-5-(pyridin-4-yl)-1H-imidazol-1-yl]acetyl}-2,7-diazaspiro[3.5]nonane-2-carboxylate/Intermediate 51-1

To a stirred solution of 2-[2-chloro-4-(4-fluorophenyl)-5-(4-pyridyl)imidazol-1-yl]acetic acid; 2,2,2-trifluoroacetic acid (Intermediate 50) (75 mg, 0.134 mmol), tert-butyl 2,7-diazaspiro[3.5]nonane-2-carboxylate (38 mg, 0.161 mmol) and DIPEA (0.094 mL, 0.536 mmol) in EtOAc (2 mL), T3P (50%, 0.16 mL, 0.268 mmol) was added and the resulting mixture was allowed to stir at 60° C. overnight. The reaction was diluted with EtOAc (5 mL) and washed with sat. aq NaHCO₃ (5 mL). The aq layer was extracted with EtOAc (2×5 mL). The combined organics were dried over MgSO₄, filtered, and evaporated under reduced pressure. The residue was purified by flash chromatography (10 g, silica) eluting with 0-4% MeOH/DCM to yield the title compound (55 mg, 71% yield) as an off-white solid. 1H NMR (500 MHz, Chloroform-d) δ 8.72-8.63 (m, 2H), 7.41-7.33 (m, 2H), 7.28-7.24 (m, 2H), 6.96-6.87 (m, 2H), 4.56 (s, 2H), 3.69 (d, J=8.1 Hz, 2H), 3.66 (d, J=8.2 Hz, 2H), 3.55 (s, 2H), 3.29 (t, J=5.3 Hz, 2H), 1.79-1.65 (m, 4H), 1.45 (s, 9H). LCMS (Analytical Method J) Rt=0.90 min, MS (ESIpos): m/z 540.3, 542.2 [M+H]+, Purity=93%.

### Synthesis of 2-[2-chloro-4-(4-fluorophenyl)-5-(pyridin-4-yl)-1H-imidazol-1-yl]-1-{2,7-diazaspiro[3.5]nonan-7-yl}ethan-1-one/Intermediate 51

TFA (0.15 mL, 2.04 mmol) was added to a stirred solution of tert-butyl 7-[2-[2-chloro-4-(4-fluorophenyl)-5-(4-pyridyl)imidazol-1-yl]acetyl]-2,7-diazaspiro[3.5]nonane-2-carboxylate (Intermediate 51-1) (55 mg, 0.102 mmol) in DCM (1.6 mL), and the resulting mixture was stirred at RT overnight. Reaction was diluted with DCM (2 mL) and carefully quenched with 1 M aq. NaOH (3 mL). The organic layer was separated and the aqueous layer extracted with DCM (2×3 mL). The combined organics were collected using a Telos phase separator and evaporated in vacuo to yield the title compound (47 mg, 93% yield) as a white solid. 1H NMR (500 MHz, Chloroform-d) δ 8.69-8.66 (m, 2H), 7.39-7.34 (m, 2H), 7.27-7.24 (m, 2H), 6.95-6.88 (m, 2H), 4.55 (s, 2H), 3.55-3.50 (m, 2H), 3.49 (d, J=8.0 Hz, 2H), 3.42 (d, J=7.9 Hz, 2H), 3.31-3.23 (m, 2H), 1.79-1.73 (m, 4H). LCMS (Analytical Method J) Rt=0.59 min, MS (ESIpos): m/z 440.2 [M+H]+, Purity=89%.

### Synthesis of tert-butyl (3S)-3-{2-[4-(4-chlorophenyl)-5-[2-(difluoromethyl)pyridin-4-yl]-1H-imidazol-1-yl]acetamido}pyrrolidine-1-carboxylate/Intermediate 52-1

T3P (50% in EtOAc) (50%, 189 uL, 0.317 mmol) was added to a solution of 2-[4-(4-chlorophenyl)-5-[2-(difluoromethyl)-4-pyridyl]imidazol-1-yl]acetic acid; 2,2,2-trifluoroacetic acid (Intermediate 10) (75 mg, 0.127 mmol), DIPEA (155 uL, 0.887 mmol) and tert-butyl (3S)-3-aminopyrrolidine-1-carboxylate (59 mg, 0.317 mmol) in EtOAc (3 mL). The reaction was stirred for 1 hour then quenched into water. The aqueous layer was extracted into EtOAc three times, the combined organics washed with brine, dried over MgSO₄ and concentrated in vacuo. The residue was purified by flash chromatography (10 g, silica), eluting with 0-10% MeOH/DCM. The relevant fractions were combined and concentrated in vacuo to yield the title compound (55 mg, 69% yield) as a white solid. 1H NMR (500 MHz, Chloroform-d) δ 8.70 (d, J=5.0 Hz, 1H), 7.72 (s, 1H), 7.58 (s, 1H), 7.37-7.31 (m, 3H), 7.24 (d, J=8.6 Hz, 2H), 6.68 (t, J=55.3 Hz, 1H), 4.54-4.45 (m, 2H), 4.45-4.38 (m, 1H), 3.57 (d, J=5.0 Hz, 2H), 3.39 (s, 2H), 2.16-2.01 (m, 2H), 1.44 (s, 9H). LCMS (Analytical Method J) Rt=0.93 min, MS (ESIpos): m/z 532.2, 534.2 [M+H]+, Purity=85%.

### Synthesis of tert-butyl (3S)-3-{2-[4-(4-chlorophenyl)-5-[2-(difluoromethyl)pyridin-4-yl]-1H-imidazol-1-yl]acetamido}pyrrolidine-1-carboxylate/Intermediate 52

TFA (0.25 mL, 3.26 mmol) was added to a solution of tert-butyl (3S)-3-[[2-[4-(4-chlorophenyl)-5-[2-(difluoromethyl)-4-pyridyl]imidazol-1-yl]acetyl]amino]pyrrolidine-1-carboxylate (Intermediate 52-1) (85% purity, 55 mg, 0.088 mmol) in DCM (1 mL). The reaction was stirred for 1 hour then concentrated in vacuo. The residue was repeatedly taken up in toluene and concentrated in vacuo. The residue was taken up in DCM/MeOH and loaded onto an SCX-2 ion exchange cartridge, primed with MeOH. The cartridge was washed sequentially with MeOH then 2M ammonia in MeOH. The basic fraction was concentrated in vacuo to yield the title compound (42 mg, 100% yield) as a colourless oil. 1H NMR (500 MHz, Chloroform-d) δ 8.68 (d, J=5.0 Hz, 1H), 7.70 (s, 1H), 7.59 (s, 1H), 7.37-7.31 (m, 3H), 7.22 (d, J=8.6 Hz, 2H), 6.66 (t, J=55.3 Hz, 1H), 4.45 (s, 2H), 4.36 (ddt, J=10.5, 7.4, 3.8 Hz, 1H), 3.05-2.95 (m, 2H), 2.87 (td, J=10.4, 9.6, 6.3 Hz, 1H), 2.71 (dd, J=11.0, 2.6 Hz, 1H), 2.10 (dq, J=10.4, 4.3, 2.6 Hz, 2H). LCMS (Analytical Method J) Rt=0.7 min, MS (ESIpos): m/z 432.2, 434.2 [M+H]+, Purity=86%.

### Synthesis of tert-butyl (3R)-3-{2-[4-(4-chlorophenyl)-5-[2-(difluoromethyl)pyridin-4-yl]-1H-imidazol-1-yl]acetamido}pyrrolidine-1-carboxylate/Intermediate 53-1

T3P (50% in EtOAc) (50%, 189 uL, 0.317 mmol) was added to a solution of 2-[4-(4-chlorophenyl)-5-[2-(difluoromethyl)-4-pyridyl]imidazol-1-yl]acetic acid; 2,2,2-trifluoroacetic acid (Intermediate 10) (75 mg, 0.127 mmol), DIPEA (155 uL, 0.887 mmol) and tert-butyl (3R)-3-aminopyrrolidine-1-carboxylate (24 mg, 0.127 mmol) in EtOAc (3 mL). The reaction was stirred for 1 hour then quenched into water. The aqueous layer was extracted into EtOAc (10 mL) three times, the combined organics washed with brine, dried over MgSO₄ and concentrated in vacuo. The residue was purified by flash chromatography (10 g, silica), eluting with 0-10% MeOH/DCM. The relevant fractions were combined and concentrated in vacuo to yield the title compound (41 mg, 44% yield) as a white solid. 1H NMR (500 MHz, Chloroform-d) δ 8.71 (d, J=5.0 Hz, 1H), 7.72 (s, 1H), 7.58 (s, 1H), 7.36 (d, J=8.6 Hz, 2H), 7.33 (d, J=4.8 Hz, 1H), 7.24 (d, J=8.6 Hz, 2H), 6.68 (t, J=55.3 Hz, 1H), 4.53-4.45 (m, 2H), 4.42-4.38 (m, 1H), 3.63-3.54 (m, 2H), 3.47-3.36 (m, 2H), 2.17-2.08 (m, 2H), 1.44 (s, 9H). LCMS (Analytical Method J) Rt=0.93 min, MS (ESIpos): m/z 532.2, 534.2 [M+H]+, Purity=73%.

### Synthesis of 2-[4-(4-chlorophenyl)-5-[2-(difluoromethyl)pyridin-4-yl]-1H-imidazol-1-yl]-N-[(3R)-pyrrolidin-3-yl]acetamide/Intermediate 53

TFA (0.25 mL, 3.26 mmol) was added to a solution of tert-butyl (3R)-3-[[2-[4-(4-chlorophenyl)-5-[2-(difluoromethyl)-4-pyridyl]imidazol-1-yl]acetyl]amino]pyrrolidine-1-carboxylate (Intermediate 53-1) (73% purity, 56 mg, 0.0771 mmol) in DCM (1 mL). The reaction was stirred for 1 hour then concentrated in vacuo. The residue was repeatedly taken up in toluene and concentrated in vacuo. The residue was taken up in DCM/MeOH and loaded onto an SCX-2 ion exchange cartridge, primed with MeOH. The cartridge was washed sequentially with MeOH then 2M ammonia in MeOH. The basic fraction was concentrated in vacuo to yield the title compound (24 mg, 72% yield) as a pale yellow oil. LCMS (Analytical Method J) Rt=0.65 min, MS (ESIpos): m/z 432.2, 434.2 [M+H]+, Purity=98%.

### Synthesis of tert-butyl (3R)-3-{2-[4-(4-chlorophenyl)-5-[2-(difluoromethyl)pyridin-4-yl]-1H-imidazol-1-yl]acetamido}pyrrolidine-1-carboxylate/Intermediate 54-1

tert-Butyl 4-amino-3,3-difluoropyrrolidine-1-carboxylate (50 mg, 0.225 mmol) and 2-[4-(4-chlorophenyl)-5-(4-pyridyl)imidazol-1-yl]acetic acid; 2,2,2-trifluoroacetic acid (Intermediate 2a) (100 mg, 0.183 mmol) were suspended in EtOAc (1.5 mL) then DIPEA (162 uL, 0.928 mmol) and T3P (50%, 220 uL, 0.370 mmol) were added. The mixture was stirred at RT for 1 h. The reaction mixture was partitioned between water and EtOAc. The organic phase was separated and the aqueous was extracted with EtOAc. The combined organics were concentrated in vacuo. The crude product was purified via flash chromatography (10 g, silica) eluting with 0-10% MeOH in DCM to afford the title compound (67 mg, 70% yield) as a yellow gum. LCMS (Analytical Method H) Rt=0.58 min, MS (ESIpos): m/z 518.3, 520.2 [M+H]+, Purity=99%.

### Synthesis of 2-[4-(4-chlorophenyl)-5-(pyridin-4-yl)-1H-imidazol-1-yl]-N-(4,4-difluoropyrrolidin-3-yl)acetamide/Intermediate 54

tert-Butyl 4-[[2-[4-(4-chlorophenyl)-5-(4-pyridyl)imidazol-1-yl]acetyl]amino]-3,3-difluoro-pyrrolidine-1-carboxylate (Intermediate 54-1) (67 mg, 0.129 mmol) was dissolved in 4M HCl in dioxane (1.5 mL) and MeOH (0.5 mL) and the mixture was stirred at RT for 45 min. The reaction was concentrated in vacuo to afford the title compound as an HCl salt (67 mg, 93% yield) (yellow gum). 1H NMR (400 MHz, DMSO-d6) δ 10.33 (s, 1H), 9.29 (d, J=8.3 Hz, 1H), 8.98 (s, 1H), 8.90-8.86 (m, 2H), 7.81-7.76 (m, 2H), 7.48-7.40 (m, 4H), 5.10-5.00 (m, 2H), 4.69-4.67 (m, 1H), 3.77-3.60 (m, 3H), 3.50-3.45 (m, 1H) LCMS (Analytical Method H) Rt=0.44 min, MS (ESIpos): m/z 418.3, 420.2 [M+H]+, Purity=100%.

### Synthesis of tert-butyl (3R)-3-{2-[4-(4-chlorophenyl)-5-(pyridin-4-yl)-1H-imidazol-1-yl]acetamido}piperidine-1-carboxylate/Intermediate 55-1

tert-Butyl (3R)-3-aminopiperidine-1-carboxylate (32 mg, 0.158 mmol) and 2-[4-(4-chlorophenyl)-5-(4-pyridyl)imidazol-1-yl]acetic acid; 2,2,2-trifluoroacetic acid (Intermediate 2a) (75 mg, 0.137 mmol) were dissolved in EtOAc (1 mL) then T3P (50%, 132 uL, 0.222 mmol) and DIPEA (79 uL, 0.453 mmol) were added. The mixture was stirred at RT for 16 h. Additional T3P (50%, 80 uL, 0.134 mmol) was added and the reaction was stirred for 4 h. The reaction was diluted with EtOAc and partitioned with water. The organic layer was separated and the aqueous extracted with EtOAc. The organics were combined and concentrated in vacuo. The crude product was purified via flash chromatography (10 g, silica) eluting with 0-50% MeOH in DCM to afford the title compound (67 mg, 99% yield). LCMS (Analytical Method H) Rt=0.56 min, MS (ESIpos): m/z 496.4, 498.3 [M+H]+, Purity=100%.

### Synthesis of 2-[4-(4-chlorophenyl)-5-(pyridin-4-yl)-1H-imidazol-1-yl]-N-[(3R)-piperidin-3-yl]acetamide/Intermediate 55

tert-Butyl (3R)-3-[[2-[4-(4-chlorophenyl)-5-(4-pyridyl)imidazol-1-yl]acetyl]amino]piperidine-1-carboxylate (Intermediate 55-1) (67 mg, 0.135 mmol) was dissolved in 4M HCl in dioxane (1.5 mL) and MeOH (0.5 mL) and stirred at RT for 45 min. The reaction was concentrated in vacuo to afford the title compound as an HCl salt (75 mg, 98% yield) (yellow solid). 1H NMR (400 MHz, DMSO-d6) δ 9.36 (s, 1H), 9.27 (s, 1H), 9.02 (s, 1H), 8.93 (d, J=7.4 Hz, 1H), 8.88 (d, J=6.3 Hz, 2H), 7.75 (d, J=6.3 Hz, 2H), 7.48-7.39 (m, 4H), 4.97-4.88 (m, 2H), 3.89-3.80 (m, 1H), 3.08-2.98 (m, 2H), 2.87-2.79 (m, 1H), 2.69-2.59 (m, 1H), 1.82-1.70 (m, 1H), 1.69-1.58 (m, 2H), 1.54-1.44 (m, 1H). LCMS (Analytical Method H) Rt=0.46 min, MS (ESIpos): m/z 396.3, 398.2 [M+H]+, Purity=100%.

### Synthesis of tert-butyl (3S)-3-{2-[4-(4-chlorophenyl)-5-(pyridin-4-yl)-1H-imidazol-1-yl]acetamido}piperidine-1-carboxylate/Intermediate 56-1

2-[4-(4-Chlorophenyl)-5-(4-pyridyl)imidazol-1-yl]acetic acid; 2,2,2-trifluoroacetic acid (Intermediate 2a) (75 mg, 0.137 mmol) and tert-butyl (3S)-3-aminopiperidine-1-carboxylate (32 mg, 0.158 mmol) were dissolved in EtOAc (1 mL) then T3P (50%, 132 uL, 0.222 mmol) and DIPEA (79 uL, 0.453 mmol) were added. The mixture was stirred at RT for 2 h. Additional T3P (50%, 80 uL, 0.134 mmol) was added and the reaction was stirred for 4 h. Water was added and the mixture was extracted with EtOAc. The organics were combined and concentrated in vacuo and the crude product was purified via flash chromatography (10 g, silica) eluting with 0-10% MeOH in DCM to afford the title compound (67 mg, 93% yield) as a yellow gum. LCMS (Analytical Method B) Rt=0.56 min, MS (ESIpos): m/z 496.4, 498.2 [M+H]+, Purity=94%.

### Synthesis of 2-[4-(4-chlorophenyl)-5-(pyridin-4-yl)-1H-imidazol-1-yl]-N-[(3S)-piperidin-3-yl]acetamide/Intermediate 56

tert-Butyl (3S)-3-[[2-[4-(4-chlorophenyl)-5-(4-pyridyl)imidazol-1-yl]acetyl]amino]piperidine-1-carboxylate (Intermediate 56-1) (67 mg, 0.127 mmol) was dissolved in 4M HCl in dioxane (1.5 mL) and MeOH (0.5 mL) and stirred at RT for 45 min. The reaction was stirred for 30 min then concentrated in vacuo to afford the title compound as an HCl salt (65 mg, 96% yield) (yellow solid). 1H NMR (400 MHz, DMSO-d6) δ 9.39-9.18 (m, 2H), 9.05 (s, 1H), 8.92 (d, J=7.4 Hz, 1H), 8.90-8.85 (m, 2H), 7.79-7.73 (m, 2H), 7.48-7.39 (m, 4H), 5.00-4.92 (m, 2H), 3.89-3.79 (m, 1H), 3.08-2.97 (m, 2H), 2.89-2.76 (m, 1H), 2.71-2.60 (m, 1H), 1.83-1.73 (m, 1H), 1.71-1.57 (m, 2H), 1.38-1.29 (m, 1H). LCMS (Analytical Method H) Rt=0.46 min, MS (ESIpos): m/z 396.3, 398.2 [M+H]+, Purity=95%.

### Synthesis of tert-butyl 7-{2-[4-(4-chlorophenyl)-5-(pyridin-4-yl)-1H-imidazol-1-yl]acetamido}-5-oxa-2-azaspiro[3.4]octane-2-carboxylate/Intermediate 57-1

T3P (50% in EtOAc) (50%, 417 uL, 0.701 mmol) was added to a solution of 2-[4-(4-chlorophenyl)-5-(4-pyridyl)imidazol-1-yl]acetic acid; 2,2,2-trifluoroacetic acid (Intermediate 2a) (323 mg, 0.596 mmol), tert-butyl 7-amino-5-oxa-2-azaspiro[3.4]octane-2-carboxylate (160 mg, 0.701 mmol) and DIPEA (612 uL, 3.50 mmol) in EtOAc (6.4 mL). The reaction was stirred for 3 hours then quenched into water. The aqueous layer was extracted into EtOAc (15 mL) three times, the combined organics washed with brine, dried over MgSO₄ and concentrated in vacuo. The residue was purified by flash chromatography (10 g, silica), eluting with 0-10% MeOH/DCM. The relevant fractions were combined and concentrated in vacuo to yield the title compound (252 mg, 63% yield) as a colourless foam. 1H NMR (400 MHz, Chloroform-d) δ 8.70 (d, J=6.0 Hz, 2H), 7.69 (s, 1H), 7.37 (d, J=8.6 Hz, 2H), 7.25-7.20 (m, 4H), 4.49 (s, 2H), 4.47-4.41 (m, 1H), 3.98 (d, J=9.4 Hz, 2H), 3.90 (dd, J=9.7, 5.6 Hz, 1H), 3.86-3.81 (m, 2H), 3.58 (dd, J=9.7, 3.3 Hz, 1H), 2.44 (dd, J=13.8, 7.3 Hz, 1H), 1.95 (dd, J=13.8, 4.1 Hz, 1H), 1.43 (s, 9H). LCMS (Analytical Method J) Rt=0.84 min, MS (ESIpos): m/z 524.3, 526.2 [M+H]+, Purity=92%.

### Synthesis of 2-[4-(4-chlorophenyl)-5-(pyridin-4-yl)-1H-imidazol-1-yl]-N-{5-oxa-2-azaspiro[3.4]octan-7-yl}acetamide/Intermediate 57

TFA (0.17 mL, 2.20 mmol) was added to a solution of tert-butyl 7-[[2-[4-(4-chlorophenyl)-5-(4-pyridyl)imidazol-1-yl]acetyl]amino]-5-oxa-2-azaspiro[3.4]octane-2-carboxylate (Intermediate 57-1) (50 mg, 0.0878 mmol) in DCM (1 mL). The reaction was stirred for 2 hours then concentrated in vacuo. The residue was repeatedly taken up in toluene and concentrated in vacuo. The residue was purified by preparative HPLC (Method A1). The relevant fractions were combined and concentrated in vacuo to yield the title compound (26 mg, 66% yield) as a colourless oil. 1H NMR (400 MHz, Chloroform-d) δ 8.72 (d, J=6.0 Hz, 2H), 7.68 (s, 1H), 7.37 (d, J=8.7 Hz, 2H), 7.25-7.20 (m, 3H), 5.50 (d, J=7.5 Hz, 1H), 4.49 (s, 2H), 4.47-4.40 (m, 1H), 3.87 (dd, J=9.6, 5.3 Hz, 1H), 3.82-3.77 (m, 2H), 3.53 (dd, J=9.5, 2.7 Hz, 1H), 3.44 (d, J=8.2 Hz, 1H), 3.38 (d, J=8.1 Hz, 1H), 2.49 (dd, J=13.7, 7.0 Hz, 1H), 2.01 (dd, J=13.6, 4.1 Hz, 1H). LCMS (Analytical Method J) Rt=0.67 min, MS (ESIpos): m/z 424.3, 426.3 [M+H]+, Purity=94%.

### Synthesis of tert-butyl 8-{2-[4-(4-chlorophenyl)-5-(pyridin-4-yl)-1H-imidazol-1-yl]acetyl}-5-oxa-2,8-diazaspiro[3.5]nonane-2-carboxylate/Intermediate 58

tert-Butyl 5-oxa-2,8-diazaspiro[3.5]nonane-2-carboxylate (45 mg, 0.197 mmol) and 2-[4-(4-chlorophenyl)-5-(4-pyridyl)imidazol-1-yl]acetic acid; 2,2,2-trifluoroacetic acid (Intermediate 2a) (100 mg, 0.183 mmol) were dissolved in a solution of EtOAc (2 mL) and DIPEA (160 uL, 0.916 mmol) then T3P (50%, 220 uL, 0.370 mmol) was added. The reaction was stirred at RT for 1 h. The reaction was concentrated in vacuo. The crude product was purified via preparative HPLC (Method A1) to afford the title compound (36 mg, 37% yield) as a brown solid. 1H NMR (500 MHz, Chloroform-d) δ 8.72-8.69 (m, 2H), 7.65 (s, 1H), 7.39-7.35 (m, 2H), 7.25-7.23 (m, 2H), 7.23-7.20 (m, 2H), 4.67-4.58 (m, 2H), 3.85-3.76 (m, 2H), 3.73-3.59 (m, 3H), 3.58-3.48 (m, 3H), 3.41-3.37 (m, 1H), 3.31-3.26 (m, 1H), 1.45 (s, 9H). LCMS (Analytical Method H) Rt=0.56 min, MS (ESIpos): m/z 524.4, 526.2 [M+H]+, Purity=100%.

### Synthesis of tert-butyl (3R)-3-{2-[4-(4-chlorophenyl)-5-(pyridin-4-yl)-1H-imidazol-1-yl]-N-methylacetamido}pyrrolidine-1-carboxylate/Intermediate 59

tert-Butyl (3R)-3-(methylamino)pyrrolidine-1-carboxylate (40 uL, 0.206 mmol) and 2-[4-(4-chlorophenyl)-5-(4-pyridyl)imidazol-1-yl]acetic acid; 2,2,2-trifluoroacetic acid (Intermediate 2a) (100 mg, 0.183 mmol) were dissolved in a solution of DIPEA (160 uL, 0.916 mmol) and EtOAc (2 mL) then T3P (50%, 220 uL, 0.370 mmol) was added. The mixture was stirred at RT for 16 h. The reaction mixture was partitioned between water and EtOAc. The organic phase was separated and the aqueous was extracted with EtOAc. The combined organics were concentrated in vacuo. The crude product was purified via flash chromatography (10 g, silica) eluting with 0-100% MeOH in DCM) followed by preparative HPLC (Method A1) to afford the title compound (45 mg, 49% yield) as a brown solid. 1H NMR (400 MHz, Chloroform-d) δ 8.71-8.65 (m, 2H), 7.62 (s, 1H), 7.40-7.35 (m, 2H), 7.24-7.15 (m, 4H), 5.13-4.96 (m, 1H), 4.57 (s, 2H), 3.58-3.46 (m, 2H), 3.35-3.26 (m, 1H), 3.24-3.10 (m, 1H), 2.80 (s, 3H), 2.08-1.94 (m, 1H), 1.94-1.80 (m, 1H), 1.46 (s, 9H). LCMS (Analytical Method H) Rt=0.58 min, MS (ESIpos): m/z 496.4, 498.2 [M+H]+, Purity=99%.

### Synthesis of tert-butyl (3S)-3-{2-[4-(4-chlorophenyl)-5-(pyridin-4-yl)-1H-imidazol-1-yl]-N-methylacetamido}pyrrolidine-1-carboxylate/Intermediate 60

tert-Butyl (3S)-3-(methylamino)pyrrolidine-1-carboxylate (40 uL, 0.206 mmol) and 2-[4-(4-chlorophenyl)-5-(4-pyridyl)imidazol-1-yl]acetic acid; 2,2,2-trifluoroacetic acid (Intermediate 2a) (100 mg, 0.183 mmol) were dissolved in a solution of DIPEA (160 uL, 0.916 mmol) and EtOAc (2 mL) then T3P (50%, 220 uL, 0.370 mmol) was added. The reaction was stirred for 16 h at RT. The reaction mixture was partitioned between water and EtOAc. The organic phase was separated and the aqueous was extracted with EtOAc. The combined organics were concentrated in vacuo. The crude product was purified via flash chromatography (10 g, silica) eluting with 0-100% MeOH in DCM followed by preparative HPLC (Method A1) to afford the title compound (47 mg, 51% yield) as a brown solid. 1H NMR (400 MHz, Chloroform-d) δ 7.63 (s, 1H), 7.40-7.36 (m, 2H), 7.25-7.18 (m, 4H), 5.13-4.96 (m, 1H), 4.58 (s, 2H), 3.59-3.44 (m, 2H), 3.36-3.26 (m, 1H), 3.26-3.10 (m, 1H), 2.80 (s, 3H), 2.08-1.95 (m, 1H), 1.94-1.79 (m, 1H), 1.47 (s, 9H). LCMS (Analytical Method H) Rt=0.58 min, MS (ESIpos): m/z 496.4, 498.3 [M+H]+, Purity=99%.

### Synthesis of tert-butyl 9-{2-[4-(4-chlorophenyl)-5-(pyridin-4-yl)-1H-imidazol-1-yl]acetyl}-1-oxa-4,9-diazaspiro[5.5]undecane-4-carboxylate/Intermediate 61

tert-Butyl 1-oxa-4,9-diazaspiro[5.5]undecane-4-carboxylate (50 mg, 0.195 mmol) and 2-[4-(4-chlorophenyl)-5-(4-pyridyl)imidazol-1-yl]acetic acid; 2,2,2-trifluoroacetic acid (Intermediate 2a) (100 mg, 0.183 mmol) were dissolved in a solution of EtOAc (2 mL) and DIPEA (160 uL, 0.916 mmol) then T3P (50%, 220 uL, 0.370 mmol) was added. The mixture was stirred at RT for 1 h. The reaction was concentrated in vacuo. The crude product was purified via preparative HPLC (Method A1) to afford the title compound (85 mg, 83% yield) as a brown solid. 1H NMR (400 MHz, DMSO-d6) δ 8.67-8.61 (m, 2H), 7.78 (s, 1H), 7.41-7.36 (m, 2H), 7.31-7.25 (m, 4H), 4.86 (s, 2H), 3.83-3.69 (m, 1H), 3.63-3.57 (m, 2H), 3.51-3.39 (m, 1H), 3.36-3.32 (m, 2H), 3.20 (s, 4H), 1.70-1.62 (m, 2H), 1.44 (s, 9H), 1.32-1.23 (m, 2H). LCMS (Analytical Method H) Rt=0.57 min, MS (ESIpos): m/z 552.5, 554.3 [M+H]+, Purity=100%.

### Synthesis of tert-butyl 8-{2-[4-(4-chlorophenyl)-5-(pyridin-4-yl)-1H-imidazol-1-yl]acetyl}-1-oxa-4,8-diazaspiro[5.5]undecane-4-carboxylate/Intermediate 62

tert-Butyl 1-oxa-4,8-diazaspiro[5.5]undecane-4-carboxylate (50 mg, 0.195 mmol) and 2-[4-(4-chlorophenyl)-5-(4-pyridyl)imidazol-1-yl]acetic acid; 2,2,2-trifluoroacetic acid (Intermediate 2a) (100 mg, 0.183 mmol) were dissolved in a solution of EtOAc (2 mL) and DIPEA (160 uL, 0.916 mmol) then T3P (50%, 220 uL, 0.370 mmol) was added. The reaction was stirred at RT for 1 h. The reaction was concentrated in vacuo. The crude product was purified via preparative HPLC (Method A1) to afford the title compound (56 mg, 53% yield) as a brown solid. 1H NMR (400 MHz, Chloroform-d) δ 8.69-8.62 (m, 2H), 7.63 (s, 1H), 7.43-7.35 (m, 2H), 7.25-7.16 (m, 4H), 4.77-4.68 (m, 1H), 4.68-4.56 (m, 2H), 3.92-3.83 (m, 1H), 3.76-3.66 (m, 1H), 3.63-3.54 (m, 2H), 3.51-3.37 (m, 1H), 3.24-2.98 (m, 3H), 2.95-2.59 (m, 1H), 1.86-1.62 (m, 2H), 1.54-1.39 (m, 11H). LCMS (Analytical Method H) Rt=0.58 min, MS (ESIpos): m/z 552.5, 554.3 [M+H]+, Purity=100%.

### Synthesis of tert-butyl 8-{2-[4-(4-chlorophenyl)-5-(pyridin-4-yl)-1H-imidazol-1-yl]acetyl}-3,8-diazabicyclo[3.2.1]octane-3-carboxylate/Intermediate 63

2-[4-(4-Chlorophenyl)-5-(4-pyridyl)imidazol-1-yl]acetic acid; 2,2,2-trifluoroacetic acid (Intermediate 2a) (75 mg, 0.137 mmol) and tert-butyl 3,8-diazabicyclo[3.2.1]octane-3-carboxylate (35 mg, 0.165 mmol) was dissolved in a solution of EtOAc (1 mL) and DIPEA (120 uL, 0.687 mmol) then T3P (50%, 165 uL, 0.277 mmol) was added. The reaction was stirred at RT for 1 h. The reaction mixture was partitioned between water and EtOAc. The organic phase was separated and the aqueous was extracted with EtOAc. The combined organics were concentrated in vacuo. The crude product was purified via flash chromatography (silica) eluting with 0-10% 7N ammonia MeOH/DCM to afford the title compound (69 mg, 89% yield) as a white solid. 1H NMR (400 MHz, DMSO-d6) δ 8.65-8.62 (m, 2H), 7.81 (s, 1H), 7.42-7.37 (m, 2H), 7.31-7.26 (m, 4H), 4.93-4.77 (m, 2H), 4.51-4.16 (m, 2H), 3.72-3.61 (m, 2H), 2.78-2.64 (m, 2H), 1.79-1.68 (m, 2H), 1.64-1.55 (m, 2H), 1.43 (s, 9H). LCMS (Analytical Method J) Rt=0.74 min, MS (ESIpos): m/z 508.3, 510.2 [M+H]+, Purity=88%.

### Synthesis of tert-butyl (1R,4R)-5-{2-[4-(4-chlorophenyl)-5-(pyridin-4-yl)-1H-imidazol-1-yl]acetyl}-2,5-diazabicyclo[2.2.2]octane-2-carboxylate/Intermediate 64

2-[4-(4-Chlorophenyl)-5-(4-pyridyl)imidazol-1-yl]acetic acid; 2,2,2-trifluoroacetic acid (Intermediate 2a) (50 mg, 0.0914 mmol) and tert-butyl (1R,4R)-2,5-diazabicyclo[2.2.2]octane-2-carboxylate (20 mg, 0.0942 mmol) were suspended in a solution of EtOAc (1 mL) and DIPEA (81 uL, 0.464 mmol) then T3P (50%, 110 uL, 0.185 mmol) was added. The mixture was stirred for 1 h at RT. The reaction was stirred for 1 h then concentrated in vacuo. The crude product was purified via preparative HPLC (Method A1) to afford the title compound (28 mg, 60% yield) as a white solid. LCMS (Analytical Method H) Rt=0.56 min, MS (ESIpos): m/z 508.4, 510.5 [M+H]+, Purity=100%.

### Synthesis of tert-butyl 2-{2-[4-(4-chlorophenyl)-5-(pyridin-4-yl)-1H-imidazol-1-yl]acetyl}-5-oxa-2,8-diazaspiro[3.5]nonane-8-carboxylate/Intermediate 65

2-[4-(4-Chlorophenyl)-5-(4-pyridyl)imidazol-1-yl]acetic acid; 2,2,2-trifluoroacetic acid (Intermediate 2a) (60 mg, 0.110 mmol) and tert-butyl 5-oxa-2,8-diazaspiro[3.5]nonane-8-carboxylate (26 mg, 0.114 mmol) were dissolved in a solution of EtOAc (1.2 mL) and DIPEA (100 uL, 0.573 mmol), then T3P (50%, 130 uL, 0.218 mmol) was added. The mixture was stirred for 1 h at RT. Additional tert-butyl 5-oxa-2,8-diazaspiro[3.5]nonane-8-carboxylate (6.0 mg, 0.0263 mmol) was added and the reaction was stirred for 30 min. The reaction mixture was partitioned between water and EtOAc. The organic phase was separated and the aqueous was extracted with EtOAc. The combined organics were concentrated in vacuo. The crude product was purified via flash chromatography (10 g, silica) eluting with 0-10% MeOH in DCM to afford the title compound (56 mg, 83% yield) as a yellow solid. 1H NMR (400 MHz, DMSO-d6) δ 8.70-8.65 (m, 2H), 7.86 (s, 1H), 7.38-7.29 (m, 6H), 5.76 (s, 1H), 4.75-4.64 (m, 2H), 3.89-3.83 (m, 2H), 3.69-3.62 (m, 2H), 3.62-3.47 (m, 3H), 3.43-3.38 (m, 2H), 1.42 (s, 9H). LCMS (Analytical Method B) Rt=min, MS (ESIpos): m/z 524.4, 526.4 [M+H]+, Purity=98%.

### Synthesis of tert-butyl (1S,4S)-5-{2-[4-(4-chlorophenyl)-5-(pyridin-4-yl)-1H-imidazol-1-yl]acetyl}-2,5-diazabicyclo[2.2.2]octane-2-carboxylate/Intermediate 66

2-[4-(4-Chlorophenyl)-5-(4-pyridyl)imidazol-1-yl]acetic acid; 2,2,2-trifluoroacetic acid (Intermediate 2a) (75 mg, 0.137 mmol) and tert-butyl (1S,4S)-2,5-diazabicyclo[2.2.2]octane-2-carboxylate (30 mg, 0.141 mmol) were dissolved in a solution of EtOAc (1.5 mL) and DIPEA (120 uL, 0.687 mmol), then T3P (50%, 165 uL, 0.277 mmol) was added. The reaction was stirred at RT for 1 h. The reaction mixture was diluted with EtOAc and partitioned with water. The organic phase was separated and the aqueous was extracted with DCM/MeOH (9:1). The combined organics were concentrated in vacuo. The crude product was purified via flash chromatography (10 g, silica) eluting with 0-10% 2M ammonia MeOH/DCM to afford the title compound (70 mg, 95% yield) as a yellow solid. 1H NMR (400 MHz, DMSO-d6) δ 8.66-8.61 (m, 2H), 7.81-7.79 (m, 1H), 7.41-7.37 (m, 2H), 7.31-7.25 (m, 4H), 4.95-4.79 (m, 1H), 4.77-4.72 (m, 1H), 4.46-3.99 (m, 2H), 3.58-3.38 (m, 2H), 3.38-3.25 (m, 2H), 1.89-1.78 (m, 1H), 1.74-1.64 (m, 3H), 1.43 (s, 9H). LCMS (Analytical Method H) Rt=0.55 min, MS (ESIpos): m/z 508.3, 510.3 [M+H]+, Purity=95%.

### Synthesis of tert-butyl (1R,4R)-5-{2-[4-(4-chlorophenyl)-5-(pyridin-4-yl)-1H-imidazol-1-yl]acetyl}-2,5-diazabicyclo[2.2.1]heptane-2-carboxylate/Intermediate 67-1

T3P in EtOAc (50%, 270 uL, 0.454 mmol) was added to a stirred solution of 2-[4-(4-chlorophenyl)-5-(4-pyridyl)imidazol-1-yl]acetic acid; 2,2,2-trifluoroacetic acid (Intermediate 2a) (100 mg, 0.183 mmol) and DIPEA (160 uL, 0.916 mmol) in EtOAc (2 mL). After stirring for 5 min tert-butyl (1R,4R)-2,5-diazabicyclo[2.2.1]heptane-2-carboxylate; hydrochloride (60 mg, 0.256 mmol) was incorporated to the reaction, and the mixture was stirred at RT for 3.5 h. 1 M aq. NaOH was added (3 mL) and the organic layer was separated. The aqueous layer was extracted with DCM (2×3 mL), and the organic layer were combined and dried using a hydrophobic Telos phase separator and evaporated under reduced pressure to afford the title compound (139 mg, 94% yield) as a brown solid. LCMS (Analytical Method H) Rt=0.53 min, MS (ESIpos): m/z 494.4 [M+H]+, Purity=61%.

### Synthesis of 2-[4-(4-chlorophenyl)-5-(pyridin-4-yl)-1H-imidazol-1-yl]-1-[(1R,4R)-2,5-diazabicyclo[2.2.1]heptan-2-yl]ethan-1-one/Intermediate 67

TFA (255 uL, 3.43 mmol) was added to a stirred solution of tert-butyl (1R,4R)-5-[2-[4-(4-chlorophenyl)-5-(4-pyridyl)imidazol-1-yl]acetyl]-2,5-diazabicyclo[2.2.1]heptane-2-carboxylate (Intermediate 67-1) (61% purity, 139 mg, 0.172 mmol) in DCM (2.75 mL), and the resulting mixture was stirred at RT for 2 h. Reaction was diluted with DCM (2 mL) and carefully quenched with 1M aq. NaOH (12 mL). The organic layer was separated and the aq layer extracted with DCM (2×6 mL). The combined organics were collected using a Telos phase separator and evaporated in vacuo to yield the title compound (64 mg, 75% yield) as an off-white solid. LCMS (Analytical Method H) Rt=0.41 min, MS (ESIpos): m/z 394.3 [M+H]+, Purity=79%.

### Synthesis of tert-butyl 2-[2-chloro-4-(4-chlorophenyl)-5-[2-(difluoromethyl)pyridin-4-yl]-1H-imidazol-1-yl]acetate/Intermediate 68-1

N-chlorosuccinimide (80 mg, 0.600 mmol) was added to an ice-cold solution of tert-butyl 2-[4-(4-chlorophenyl)-5-[2-(difluoromethyl)-4-pyridyl]imidazol-1-yl]acetate (Intermediate 10-2) (214 mg, 0.500 mmol) in DCM (4 mL) and the resulting mixture was allowed to stir at 50° C. for 3 h. Reaction quenched with 1 M aq. NaOH (15 mL). The mixture was extracted with DCM (2×10 mL), the organic layer isolated using a Telos phase separator and evaporated under reduced pressure. The residue was purified by flash chromatography (10 g, silica) eluting with 0-100% EtOAc/heptane to yield the title compound (137 mg, 60% yield) as a pale-yellow oil. 1H NMR (400 MHz, Chloroform-d) δ 8.72 (d, J=5.0 Hz, 1H), 7.61 (s, 1H), 7.32 (dt, J=9.1, 2.6 Hz, 3H), 7.25-7.19 (m, 2H), 6.68 (t, J=55.3 Hz, 1H), 4.43 (s, 2H), 1.45 (s, 9H). LCMS (Analytical Method H) Rt=0.73 min, MS (ESIpos): m/z 454.2, 456.2, 458.2 [M+H]+, Purity=100%.

### Synthesis of 2-[2-chloro-4-(4-chlorophenyl)-5-[2-(difluoromethyl)pyridin-4-yl]-1H-imidazol-1-yl]acetic acid/Intermediate 68-2

TFA (0.44 mL, 5.94 mmol) was added to a solution of tert-butyl 2-[2-chloro-4-(4-chlorophenyl)-5-[2-(difluoromethyl)-4-pyridyl]imidazol-1-yl]acetate (Intermediate 68-1) (135 mg, 0.297 mmol) in DCM (2.1637 mL) and the resulting mixture stirred at RT for 8 h. The solvent was evaporated under reduced pressure and the residue was dried in the vacuum oven to yield the title compound as a TFA salt (153 mg, 38% yield) (yellow solid). LCMS (Analytical Method H) Rt=0.37 min, MS (ESIpos): m/z 398.1, 400.1 [M+H]+, Purity=46%.

### Synthesis of tert-butyl 7-{2-[2-chloro-4-(4-chlorophenyl)-5-[2-(difluoromethyl)pyridin-4-yl]-1H-imidazol-1-yl]acetyl}-2,7-diazaspiro[3.5]nonane-2-carboxylate and tert-butyl 7-{2-[4-(4-chlorophenyl)-5-[2-(difluoromethyl)pyridin-4-yl]-2-hydroxy-1H-imidazol-1-yl]acetyl}-2,7-diazaspiro[3.5]nonane-2-carboxylate/Intermediate 68-3 and Intermediate 68-4

To a stirred solution of 2-[2-chloro-4-(4-chlorophenyl)-5-[2-(difluoromethyl)-4-pyridyl]imidazol-1-yl]acetic acid; 2,2,2-trifluoroacetic acid (Intermediate 68-2) (81% purity, 153 mg, 0.198 mmol) and DIPEA (0.14 mL, 0.792 mmol) in EtOAc (2.9541 mL), T3P (50%, 0.24 mL, 0.396 mmol) was added followed by tert-butyl 2,7-diazaspiro[3.5]nonane-2-carboxylate (57 mg, 0.237 mmol) and the resulting mixture was allowed to stir at 60° C. overnight. The reaction was diluted with EtOAc (5 mL) and washed with saturated NaHCO₃ (5 mL). The aqueous layer was extracted with EtOAc (2×5 mL). The combined organics were dried over MgSO₄, filtered, and evaporated under reduced pressure. The residue was purified by preparative HPLC (Method A1) to yield the title compounds (Intermediate 68-3) (40 mg, 0.0651 mmol, 33% yield) and (Intermediate 68-4) (43 mg, 37% yield) as white solids. (Intermediate 68-3) 1H NMR (500 MHz, Methanol-d4) δ 8.71 (d, J=5.0 Hz, 1H), 7.62 (s, 1H), 7.47-7.42 (m, 1H), 7.33-7.30 (m, 2H), 7.30-7.27 (m, 2H), 6.77 (t, J=55.1 Hz, 1H), 4.91 (s, 2H), 3.66 (s, 4H), 3.56-3.50 (m, 2H), 3.44-3.37 (m, 2H), 1.73-1.63 (m, 4H), 1.44 (s, 9H). LCMS (Analytical Method J) Rt=1.16 min, MS (ESIpos): m/z 606.2, 608.22, 610 [M+H]+, Purity=100%. (Intermediate 68-4) 1H NMR (400 MHz, Methanol-d4) δ 8.62 (d, J=5.1 Hz, 1H), 7.61 (s, 1H), 7.41 (d, J=5.1 Hz, 1H), 7.36-7.28 (m, 2H), 7.27-7.16 (m, 2H), 6.71 (t, J=55.1 Hz, 1H), 4.57 (s, 2H), 3.65 (s, 4H), 3.56-3.45 (m, 2H), 3.45-3.37 (m, 2H), 1.76-1.62 (m, 4H), 1.44 (s, 9H). LCMS (Analytical Method J) Rt=1.01 min, MS (ESIpos): m/z 588.2, 590.2 [M+H]+, Purity=100%.

### Synthesis of 2-[2-chloro-4-(4-chlorophenyl)-5-[2-(difluoromethyl)pyridin-4-yl]-1H-imidazol-1-yl]-1-{2,7-diazaspiro[3.5]nonan-7-yl}ethan-1-one/Intermediate 68

TFA (0.096 mL, 1.29 mmol) was added to a stirred solution of tert-butyl 7-[2-[2-chloro-4-(4-chlorophenyl)-5-[2-(difluoromethyl)-4-pyridyl]imidazol-1-yl]acetyl]-2,7-diazaspiro[3.5]nonane-2-carboxylate (Intermediate 68-3) (39 mg, 0.0643 mmol) in DCM (1.0418 mL), and the resulting mixture was stirred at RT for 6 h. Reaction was diluted with DCM (2 mL) and carefully quenched with 1 M aq. NaOH (3 mL). The organic layer was separated and the aq. layer extracted with DCM (2×3 mL). The combined organics were collected using a Telos phase separator and evaporated in vacuo to yield the title compound (30 mg, 87% yield) as a white solid. 1H NMR (500 MHz, Chloroform-d) δ 8.67 (d, J=5.0 Hz, 1H), 7.59 (s, 1H), 7.37-7.33 (m, 1H), 7.33-7.29 (m, 2H), 7.24-7.18 (m, 2H), 6.66 (t, J=55.3 Hz, 1H), 4.57 (s, 2H), 3.59-3.51 (m, 2H), 3.46 (d, J=7.7 Hz, 2H), 3.38 (d, J=7.7 Hz, 2H), 3.30-3.23 (m, 2H), 1.83-1.74 (m, 4H). LCMS (Analytical Method J) Rt=0.66 min, MS (ESIpos): m/z 506.2, 508.2, 510.2 [M+H]+, Purity=94%.

### Synthesis of 2-[5-bromo-4-(4-fluorophenyl)-1H-imidazol-1-yl]acetic acid/Intermediate 69-1

tert-Butyl 2-[5-bromo-4-(4-fluorophenyl)imidazol-1-yl]acetate (Intermediate 1-2) (500 mg, 1.41 mmol) was dissolved in DCM (5 mL) and TFA (1 mL) and stirred at RT for 21 h then concentrated in vacuo to afford the title compound as TFA salt (580 mg, 82% yield). ¹H NMR (500 MHz, DMSO-d6) δ 8.31 (s, 1H), 7.94-7.88 (m, 2H), 7.35-7.29 (m, 2H), 6.09-5.66 (m, 2H), 4.96 (s, 2H). LCMS (Analytical Method J) Rt=0.61 min, MS (ESIpos): m/z 299.0, 301.0 [M+H]+, Purity=82%.

### Synthesis of N-[4-(4,4,5,5-tetramethyl-1,3,2-dioxaborolan-2-yl)pyridin-2-yl]benzamide/Intermediate 69-2

N-(4-bromo-2-pyridyl)benzamide (405 mg, 1.46 mmol), 4,4,5,5-tetramethyl-2-(4,4,5,5-tetramethyl-1,3,2-dioxaborolan-2-yl)-1,3,2-dioxaborolane (Intermediate 14-3) (372 mg, 1.47 mmol) and potassium; acetate (292 mg, 2.95 mmol) were suspended in 1,4-Dioxane (4 mL) and the mixture was degassed for 5 min with nitrogen then cyclopentyl(diphenyl)phosphane; dichloromethane; dichloropalladium; iron (120 mg, 0.147 mmol) was added. The reaction was sealed and stirred at 100° C. for 1 h. The reaction was cooled to room temperature and diluted with water, then extracted with EtOAc. The organics were combined and concentrated in vacuo. The crude product was purified via flash chromatography (25 g, silica) eluting with 0-10% MeOH in DCM. The product was purified again via flash chromatography (25 g, silica) eluting with 50-100% EtOAc in heptane then 0-40% MeOH in EtOAc. The relevant fractions were isolated to afford the title compound (50 mg, 10% yield) as a white solid. 1H NMR (400 MHz, Chloroform-d) δ 8.72 (s, 1H), 8.64 (s, 1H), 8.30 (dd, J=0.8, 4.8 Hz, 1H), 7.96-7.92 (m, 2H), 7.60-7.54 (m, 1H), 7.53-7.48 (m, 2H), 7.42-7.39 (m, 1H), 1.35 (s, 12H) LCMS (Analytical Method J) Rt=0.51 min, MS (ESIpos): m/z 243.1 [M+H]+, Purity=58%.

### Synthesis of 2-[5-bromo-4-(4-fluorophenyl)-1H-imidazol-1-yl]-1-{2-oxa-6-azaspiro[3.3]heptan-6-yl}ethan-1-one/Intermediate 69

2-[5-Bromo-4-(4-fluorophenyl)imidazol-1-yl]acetic acid; 2,2,2-trifluoroacetic acid (Intermediate 69-1) (100 mg, 0.242 mmol) was dissolved in a solution of DIPEA (169 uL, 0.968 mmol), EtOAc (1 mL) and DMF (0.3 mL), then T3P (50%, 218 uL, 0.367 mmol) was added, followed by 2-oxa-6-azaspiro[3.3]heptane ethanedioate (2:1) (70 mg, 0.242 mmol). The mixture was stirred at RT for 30 min. The reaction was left standing for 72 hours. Additional 2-oxa-6-azaspiro[3.3]heptane ethanedioate (2:1) (35 mg, 0.121 mmol), DIPEA (42 uL, 0.242 mmol) and T3P (50%, 72 uL, 0.121 mmol) were added and the mixture was stirred for 2 h. The mixture was partitioned with water and extracted with EtOAc. The organics were combined and concentrated in vacuo and the crude product was purified via flash chromatography (10 g, silica) eluting with 50-100% EtOAc in heptane then 0-80% MeOH in EtOAc to afford the title compound (98 mg, 96% yield) as an off white solid. 1H NMR (400 MHz, DMSO-d6) δ 7.95-7.88 (m, 2H), 7.87 (s, 1H), 7.30-7.23 (m, 2H), 4.75 (s, 2H), 4.72-4.67 (m, 4H), 4.42 (s, 2H), 4.11 (s, 2H). LCMS (Analytical Method J) Rt=0.68 min, MS (ESIpos): m/z 380.1, 382.1 [M+H]+, Purity=90%.

### Synthesis of 2-[5-bromo-4-(4-fluorophenyl)-1H-imidazol-1-yl]-1-{2-oxa-6-azaspiro[3.4]octan-6-yl}ethan-1-one/Intermediate 70

2-[5-Bromo-4-(4-fluorophenyl)imidazol-1-yl]acetic acid (Intermediate 69-1) (88% purity, 100 mg, 0.294 mmol) and 2-oxa-6-azaspiro[3.4]octane (35 mg, 0.309 mmol) were suspended in a solution of EtOAc (2 mL), DMF (0.2 mL) and DIPEA (150 uL, 0.859 mmol) then T3P (50%, 250 uL, 0.420 mmol) was added. The mixture was stirred at RT for 2.5 h. Additional T3P (50%, 80 uL, 0.134 mmol) was added and the reaction was stirred for 3 h. The mixture was concentrated in vacuo and the residue was purified via preparative HPLC (Method B1) to afford the title compound (73 mg, 63% yield) as a white solid. 1H NMR (400 MHz, DMSO-d6) δ 7.98-7.91 (m, 2H), 7.85 (s, 1H), 7.27-7.19 (m, 2H), 4.96-4.87 (m, 2H), 4.62-4.49 (m, 4H), 3.89-3.82 (m, 1H), 3.65-3.58 (m, 2H), 3.42-3.37 (m, 1H), 2.32-2.26 (m, 1H), 2.17-2.11 (m, 1H). LCMS (Analytical Method J) Rt=0.70 min, MS (ESIpos): m/z 394.1, 396.1 [M+H]+, Purity=100%.

### Synthesis of 2-[5-bromo-4-(4-fluorophenyl)-1H-imidazol-1-yl]-1-{6-oxa-2-azaspiro[3.4]octan-2-yl}ethan-1-one/Intermediate 71

6-Oxa-2-azaspiro[3.4]octane; oxalic acid (50 mg, 0.158 mmol) and 2-[5-bromo-4-(4-fluorophenyl)imidazol-1-yl]acetic acid (Intermediate 69-1) (88% purity, 50 mg, 0.147 mmol) were suspended in EtOAc (1 mL) and DIPEA (105 uL, 0.601 mmol), then T3P (50%, 120 uL, 0.202 mmol) was added. The reaction was stirred at RT for 1 h. DMF (0.4 ml) was added to aid solubility and the reaction was stirred for 16 h. Additional T3P (50%, 20 uL, 0.0336 mmol) was added and the reaction was stirred for 1 h. The reaction was concentrated in vacuo then purified via preparative HPLC (Method A1) to afford the title compound (57 mg, 96% yield) as a white solid. 1H NMR (400 MHz, DMSO-d6) δ 7.95-7.90 (m, 2H), 7.89 (s, 1H), 7.30-7.22 (m, 2H), 4.79 (s, 2H), 4.22 (s, 2H), 3.95-3.89 (m, 2H), 3.83-3.75 (m, 2H), 3.72 (t, J=7.0 Hz, 2H), 2.16-2.11 (m, 2H). LCMS (Analytical Method H) Rt=0.47 min, MS (ESIpos): m/z 394.2, 396.2 [M+H]+, Purity=98%.

### Synthesis of 4-(4,4,5,5-tetramethyl-1,3,2-dioxaborolan-2-yl)-2-(trifluoromethyl)pyridine/Intermediate 72-1

A mixture of 4-bromo-2-(trifluoromethyl)pyridine (750 mg, 3.32 mmol), 4,4,5,5-tetramethyl-2-(4,4,5,5-tetramethyl-1,3,2-dioxaborolan-2-yl)-1,3,2-dioxaborolane (927 mg, 3.65 mmol), potassium acetate (691 mg, 6.97 mmol) and PdCl2(dppf). CH2Cl2 (272 mg, 0.332 mmol) in 1,4-Dioxane (10 mL) was degassed by sparging with nitrogen. The reaction was heated to 80° C. for 20 hours. The reaction was cooled and quenched into water. The aqueous layer was extracted into EtOAc three times, the combined organics washed with brine, dried over MgSO₄ and concentrated in vacuo. The residue was purified by chromatography (10 g, silica), eluting with 40-100% EtOAc/heptane. The relevant fractions were combined and concentrated in vacuo to yield the title compound as a pale brown oil (862 mg, 90% yield). 1H NMR (500 MHz, DMSO-d6) δ 8.83 (d, J=4.6 Hz, 1H), 7.91 (s, 1H), 7.88 (d, J=4.6 Hz, 1H), 1.33 (s, 12H). LCMS (Analytical Method E) Rt=0.87 min, MS (ESIpos): m/z 192.1 [M+H]+, Purity=55%.

### Synthesis of tert-butyl 2-[4-(4-chlorophenyl)-5-[2-(trifluoromethyl)pyridin-4-yl]-1H-imidazol-1-yl]acetate/Intermediate 72-2

tert-Butyl 2-[5-bromo-4-(4-chlorophenyl)imidazol-1-yl]acetate (Intermediate 2-2) (463 mg, 1.25 mmol), 4-(4,4,5,5-tetramethyl-1,3,2-dioxaborolan-2-yl)-2-(trifluoromethyl)pyridine (Intermediate 72-1) (340 mg, 1.25 mmol) and Na₂CO₃ (396 mg, 3.74 mmol) were suspended in Water (1.2 mL) and DME (4.5 mL). The mixture was degassed with nitrogen for 5 min then Tetrakistriphenylphosphine palladium (72 mg, 0.0623 mmol) was added. The mixture was degassed for 5 min then sealed and stirred at 100° C. for 2 h with heating via microwave irradiation. The reaction was cooled and quenched into water. The aqueous layer was extracted into EtOAc three times, the combined organics washed with brine, dried over MgSO₄ and concentrated in vacuo. The residue was purified by flash chromatography (25 g, silica), eluting with 0-80% EtOAc/heptane. The relevant fractions were combined and concentrated in vacuo to yield the title compound as a colourless oil (406 mg, 68% yield). 1H NMR (400 MHz, Chloroform-d) δ 8.77 (d, J=5.0 Hz, 1H), 7.68 (s, 1H), 7.65 (s, 1H), 7.44-7.38 (m, 1H), 7.34 (d, J=8.6 Hz, 2H), 7.24 (d, J=8.6 Hz, 2H), 4.48 (s, 2H), 1.40 (s, 9H). LCMS (Analytical Method J) Rt=1.08 min, MS (ESIpos): m/z 438.2, 440.2 [M+H]+, Purity=91%.

### Synthesis of 2-[4-(4-chlorophenyl)-5-[2-(trifluoromethyl)pyridin-4-yl]-1H-imidazol-1-yl]acetic acid/Intermediate 72-3

TFA (1.8 mL, 24.2 mmol) was added to a solution of tert-butyl 2-[4-(4-chlorophenyl)-5-[2-(trifluoromethyl)-4-pyridyl]imidazol-1-yl]acetate (Intermediate 72-2) (406 mg, 0.927 mmol) in DCM (7.1859 mL). The reaction was stirred for 20 hours then concentrated in vacuo. The residue was repeatedly taken up in toluene and concentrated in vacuo to yield the title compound as a TFA salt (colourless oil) (560 mg, 61% yield). 1H NMR (500 MHz, DMSO-d6) δ 8.86 (d, J=5.0 Hz, 1H), 8.25 (s, 1H), 7.84 (s, 1H), 7.67 (dd, J=5.0, 1.3 Hz, 1H), 7.40-7.37 (m, 4H), 4.94 (s, 2H). LCMS (Analytical Method E) Rt=1.03 min, MS (ESIpos): m/z 382.0, 384.0 [M+H]+, Purity=62%.

### Synthesis of tert-butyl 7-{2-[4-(4-chlorophenyl)-5-[2-(trifluoromethyl)pyridin-4-yl]-1H-imidazol-1-yl]acetyl}-2,7-diazaspiro[3.5]nonane-2-carboxylate/Intermediate 72-4

tert-Butyl 2,7-diazaspiro[3.5]nonane-2-carboxylate (29 mg, 0.128 mmol) was added to a solution of 2-[4-(4-chlorophenyl)-5-[2-(trifluoromethyl)-4-pyridyl]imidazol-1-yl]acetic acid; 2,2,2-trifluoroacetic acid (Intermediate 72-3) (78 mg, 0.128 mmol) and DIPEA (89 uL, 0.512 mmol) in EtOAc (1.344 mL). T3P (50% in EtOAc) (50%, 95 uL, 0.160 mmol) was added and the reaction stirred for 20 hours. Additional tert-butyl 2,7-diazaspiro[3.5]nonane-2-carboxylate (29 mg, 0.128 mmol) and DIPEA (89 uL, 0.512 mmol) were added followed by T3P (50% in EtOAc) (50%, 95 uL, 0.160 mmol) and the reaction stirred for 18 hours. The reaction was quenched into water and the aqueous layer was extracted into EtOAc (20 mL) three times, the combined organics washed with brine (15 mL), dried over MgSO₄ and concentrated in vacuo. The residue was purified by flash chromatography (10 g, silica), eluting with 0-10% MeOH/DCM. The relevant fractions were combined and concentrated in vacuo to yield the title compound as a colourless gum (48 mg, 51% yield). LCMS (Analytical Method J) Rt=1.08 min, MS (ESIpos): m/z 590.3, 592.3 [M+H]+, Purity=100%.

### Synthesis of 2-[4-(4-chlorophenyl)-5-[2-(trifluoromethyl)pyridin-4-yl]-1H-imidazol-1-yl]-1-{2,7-diazaspiro[3.5]nonan-7-yl}ethan-1-one/Intermediate 72

TFA (0.25 mL, 3.26 mmol) was added to a solution of tert-butyl 7-[2-[4-(4-chlorophenyl)-5-[2-(trifluoromethyl)-4-pyridyl]imidazol-1-yl]acetyl]-2,7-diazaspiro[3.5]nonane-2-carboxylate (Intermediate 72-4) (52 mg, 0.0881 mmol) in DCM (1 mL). The reaction was stirred for 90 minutes then concentrated in vacuo. The residue was repeatedly taken up in toluene and concentrated in vacuo to yield the title compound as a TFA salt (65 mg, 89% yield) (colourless oil). The crude product was progressed directly to the next step with no purification or analysis.

### Synthesis of tert-butyl 7-{2-[4-(4-chlorophenyl)-5-[2-(difluoromethyl)pyridin-4-yl]-1H-imidazol-1-yl]acetyl}-2,7-diazaspiro[3.5]nonane-2-carboxylate/Intermediate 73-3

tert-Butyl 2,7-diazaspiro[3.5]nonane-2-carboxylate (29 mg, 0.127 mmol) was added to a solution of 2-[4-(4-chlorophenyl)-5-[2-(difluoromethyl)-4-pyridyl]imidazol-1-yl]acetic acid; 2,2,2-trifluoroacetic acid (Intermediate 10) (75 mg, 0.127 mmol) and DIPEA (89 uL, 0.507 mmol) in EtOAc (1.3315 mL). T3P (50% in EtOAc) (50%, 94 uL, 0.158 mmol) was added and the reaction stirred for 20 hours. Additional tert-butyl 2,7-diazaspiro[3.5]nonane-2-carboxylate (29 mg, 0.127 mmol) and DIPEA (89 uL, 0.507 mmol) were added followed by T3P (50% in EtOAc) (50%, 94 uL, 0.158 mmol) and the reaction stirred for 18 hours. The reaction was quenched into water and the aqueous layer was extracted into EtOAc (20 mL) three times, the combined organics washed with brine (15 ml), dried over MgSO₄ and concentrated in vacuo. The residue was purified by chromatography (10 g, silica), eluting with 0-10% MeOH/DCM. The relevant fractions were combined and concentrated in vacuo to yield the title compound as a colourless gum (52 mg, 72% yield) LCMS (Analytical Method J) Rt=1.00 min, MS (ESIpos): m/z 572.3, 574.3 [M+H]+, Purity=100%.

### Synthesis of 2-[4-(4-chlorophenyl)-5-[2-(difluoromethyl)pyridin-4-yl]-1H-imidazol-1-yl]-1-{2,7-diazaspiro[3.5]nonan-7-yl}ethan-1-one/Intermediate 73

TFA (0.25 mL, 3.26 mmol) was added to a solution of tert-butyl 7-[2-[4-(4-chlorophenyl)-5-[2-(difluoromethyl)-4-pyridyl]imidazol-1-yl]acetyl]-2,7-diazaspiro[3.5]nonane-2-carboxylate (Intermediate 73-3) (48 mg, 0.084 mmol) in DCM (1 mL). The reaction was stirred for 90 minutes then concentrated in vacuo. The residue was repeatedly taken up in toluene and concentrated in vacuo to yield the title compound as a TFA salt (60 mg, 88% yield). The crude product was progressed directly to the next step with no purification or analysis.

### Synthesis of tert-butyl 2-(4-bromo-1H-imidazol-1-yl)acetate/Intermediate 74-1

To an ice-cold solution of 4-bromo-1H-imidazole (5.00 g, 34.0 mmol) in THF-Anhydrous (100 mL), NaH (60%, 1497 mg, 37.4 mmol) was added portionwise and the mixture was allowed to stir at 0° C. for 15 minutes. tert-butyl bromoacetate (5.5 mL, 37.4 mmol) was then added, and the mixture stirred at 0° C. for 90 minutes. The reaction was slowly quenched with water. The aqueous layer was extracted into EtOAc (2×), the combined organics washed with brine, dried over MgSO₄, filtered and concentrated in vacuo. The residue was purified by flash chromatography (100 g, silica), eluting with 0-70% EtOAc/heptane to yield the title compound (4.70 g, 53% yield) as an off-white solid. 1H NMR (400 MHz, Chloroform-d) δ 7.34 (d, J=1.3 Hz, 1H), 6.91 (d, J=1.5 Hz, 1H), 4.53 (s, 2H), 1.46 (s, 9H). LCMS (Analytical Method J) Rt=0.76 min, MS (ESIpos): m/z 261.0, 263.0 [M+H]+, Purity=88%.

### Synthesis of tert-butyl 2-(4-bromo-5-iodo-1H-imidazol-1-yl)acetate/Intermediate 74-2

N-Iodosuccinimide (10.12 g, 45.0 mmol) was added to a stirred solution of tert-butyl 2-(4-bromoimidazol-1-yl)acetate (Intermediate 74-1) (4.70 g, 18.0 mmol) in MeCN-Anhydrous (94 mL) and the resulting mixture was heated at 85° C. for 7 h. The mixture was diluted with EtOAc and washed with 1 M aq. Na₂S2O₃ (3×). The combined aq. layers were extracted with EtOAc (2×), and the combined organics were washed with brine, dried over MgSO₄ and evaporated under reduced pressure. The residue was purified by flash chromatography (100 g, silica) eluting with 0-70% EtOAc/heptane to yield the title compound (2.56 g, 31% yield) as an off-white solid. 1H NMR (400 MHz, Chloroform-d) δ 7.62 (s, 1H), 4.57 (s, 2H), 1.48 (s, 9H). LCMS (Analytical Method B) Rt=3.18 min, MS (ESIpos): m/z 387.0, 389.0 [M+H]+, Purity=83%.

### Synthesis of tert-butyl 2-[4-bromo-5-(pyridin-4-yl)-1H-imidazol-1-yl]acetate/Intermediate 74-3

A mixture of tert-butyl 2-(4-bromo-5-iodo-imidazol-1-yl)acetate (Intermediate 74-2) (1.70 g, 4.39 mmol), pyridin-4-ylboronic acid (594 mg, 4.83 mmol), Tetrakis(triphenylphosphine)palladium (254 mg, 0.220 mmol), and 2 M Na₂CO₃ (6.6 mL, 13.2 mmol) in DME (32 mL) was degassed by sparging with nitrogen. The reaction was heated to 100° C. for 5 h under microwave irradiation. The reaction mixture was diluted with EtOAc (50 mL) and washed with water (60 mL). The organic layer was dried over MgSO₄, filtered and evaporated under reduced pressure. The residue was purified by flash chromatography (50 g, silica) eluting with 0-100% EtOAc/heptane to yield the impure product. The residue was dissolved in MeOH and loaded onto an SCX column, which was flushed with MeOH (3CV) followed by 7N NH₃ in MeOH to recover the product. The solvent was evaporated in vacuo to yield the title compound (517 mg, 1.36 mmol, 31% yield). 1H NMR (500 MHz, Chloroform-d) δ 8.74-8.69 (m, 2H), 7.55 (s, 1H), 7.33-7.30 (m, 2H), 4.54 (s, 2H), 1.39 (s, 9H). LCMS (Analytical Method J) Rt=0.65 min, MS (ESIpos): m/z 338.1, 340.1 [M+H]+, Purity=89%.

### Synthesis of 2-[4-bromo-5-(pyridin-4-yl)-1H-imidazol-1-yl]acetic acid/Intermediate 74-4

TFA (5.0 mL, 67.8 mmol) was added to a solution of tert-butyl 2-[4-bromo-5-(4-pyridyl)imidazol-1-yl]acetate (Intermediate 74-3) (89% purity, 515 mg, 1.36 mmol) in DCM (10 mL) and the resulting mixture stirred at RT overnight. The solvent was evaporated under reduced pressure and Et2O was added and evaporated multiple times to provide the title compound as a TFA salt (736 mg, 90% yield) 1H NMR (400 MHz, DMSO-d6) δ 8.77 (d, J=6.0 Hz, 2H), 7.93 (s, 1H), 7.69-7.52 (m, 2H), 4.97 (s, 2H). LCMS (Analytical Method J) Rt=0.23 min, MS (ESIpos): m/z 282.0, 284.0 [M+H]+.

### Synthesis of -[4-bromo-5-(pyridin-4-yl)-1H-imidazol-1-yl]-1-(4-methylpiperazin-1-yl)ethan-1-one/Intermediate 74

To a stirred suspension of 2-[4-bromo-5-(4-pyridyl)imidazol-1-yl]acetic acid, 2,2,2-trifluoroacetic acid (Intermediate 74-4) (85% purity, 736 mg, 1.23 mmol) in EtOAc (20 mL), DIPEA (0.64 mL, 3.68 mmol) was added and the mixture stirred until it became a clear solution. T3P (50%, 2.9 mL, 4.91 mmol) was then added followed by 1-methylpiperazine (0.27 mL, 2.45 mmol) and the resulting mixture was allowed to stir at 60° C. overnight. The reaction was diluted with EtOAc (10 mL) and extracted with water (30 mL). The organic layer was discarded and the aq layer was extracted with 10% MeOH in DCM (3×30 mL). The combined organics were dried over MgSO₄, filtered, and evaporated under reduced pressure to yield crude product. The aq layer was also evaporated and the solid was triturated with EtOAc, filtered off and evaporated in vacuo to provide more crude product. Both fractions were combined and purified by flash chromatography (11 g, KP-NH) eluting with 0-2% MeOH/DCM to yield the title compound (310 mg, 64% yield) as a yellow sticky oil. 1H NMR (500 MHz, Chloroform-d) δ 8.74-8.69 (m, 2H), 7.55 (s, 1H), 7.34-7.29 (m, 2H), 4.66 (s, 2H), 3.67-3.59 (m, 2H), 3.38-3.31 (m, 2H), 2.42-2.32 (m, 4H), 2.30 (s, 3H). LCMS (Analytical Method J) Rt=0.23 min, MS (ESIpos): m/z 364.1, 366.1 [M+H]+.

### Synthesis of tert-butyl 6-{2-[4-(4-chlorophenyl)-5-(pyridin-4-yl)-1H-imidazol-1-yl]acetyl}-2,6-diazaspiro[3.4]octane-2-carboxylate/Intermediate 75-1

DIPEA (75 uL, 0.428 mmol) followed by tert-butyl 2,7-diazaspiro[3.4]octane-2-carboxylate; hydrochloride (33 uL, 0.142 mmol) were added to a solution of 2-[4-(4-chlorophenyl)-5-(4-pyridyl)imidazol-1-yl]acetic acid; 2,2,2-trifluoroacetic acid (Intermediate 2a) (58% purity, 100 mg, 0.107 mmol) in EtOAc (1.1248 mL). T3P (50% in EtOAc) (50%, 80 uL, 0.134 mmol) was added and the reaction was heated to 60° C. for 24 hours. The reaction was cooled and quenched into water. The aqueous layer was extracted into EtOAc (30 mL) three times, the combined organics washed with brine (20 mL), dried over MgSO₄ and concentrated in vacuo. The residue was purified by flash chromatography (10 g, silica), eluting with 0-10% MeOH/DCM. The relevant fractions were combined and concentrated in vacuo to yield the title compound as a white solid (57 mg, 100% yield). LCMS (Analytical Method J) Rt=0.78 min, MS (ESIpos): m/z 508.3 [M+H]+, Purity=95%.

### Synthesis of 2-[4-(4-chlorophenyl)-5-(pyridin-4-yl)-1H-imidazol-1-yl]-1-{2,6-diazaspiro[3.4]octan-6-yl}ethan-1-one/Intermediate 75

TFA (0.48 mL, 6.22 mmol) was added to a solution of tert-butyl 7-[2-[4-(4-chlorophenyl)-5-(4-pyridyl)imidazol-1-yl]acetyl]-2,7-diazaspiro[3.4]octane-2-carboxylate (Intermediate 75-1) (95 mg, 0.178 mmol) in DCM (2 mL). The reaction was stirred for 90 minutes then concentrated in vacuo. The residue was repeatedly taken up in toluene and concentrated in vacuo. The material was purified by preparative HPLC (Method A1). The relevant fractions were combined and concentrated in vacuo to yield the title compound as a white solid (45 mg, 62% yield). LCMS (Analytical Method J) Rt=0.52 min, MS (ESIpos): m/z 408.3, 410.3 [M+H]+, Purity=100%.

### Synthesis of tert-butyl (3S)-3-{2-[4-(4-chlorophenyl)-5-(pyridin-4-yl)-1H-imidazol-1-yl]acetamido}pyrrolidine-1-carboxylate/Intermediate 76-1

2-[4-(4-Chlorophenyl)-5-(4-pyridyl)imidazol-1-yl]acetic acid; 2,2,2-trifluoroacetic acid (Intermediate 2a) (75 mg, 0.137 mmol) and tert-butyl (3S)-3-aminopyrrolidine-1-carboxylate (30 mg, 0.161 mmol) were dissolved in EtOAc (1 mL) then T3P (50%, 130 uL, 0.218 mmol) and DIPEA (80 uL, 0.458 mmol) were added. The reaction was stirred at RT for 16 h. Additional T3P (50%, 40 uL, 0.0672 mmol) was added. Additional T3P (50%, 40 uL, 0.0672 mmol) and DIPEA (80 uL, 0.458 mmol) was added and the reaction was stirred for 3 h. Additional tert-butyl (3S)-3-aminopyrrolidine-1-carboxylate (30 mg, 0.161 mmol) was added and the reaction was stirred for 6 h. The reaction was diluted with EtOAc and partitioned with water. The organic layer was separated and the aqueous extracted with EtOAc. The organics were combined and concentrated in vacuo. The crude product was purified via flash chromatography (10 g, silica) eluting with 0-100% MeOH in DCM to afford the title compound (65 mg, 77% yield) as a yellow gum. LCMS (Analytical Method H) Rt=0.54 min, MS (ESIpos): m/z 482.3, 484.2 [M+H]+, Purity=78%.

### Synthesis of 2-[4-(4-chlorophenyl)-5-(pyridin-4-yl)-1H-imidazol-1-yl]-N-[(3S)-pyrrolidin-3-yl]acetamide/Intermediate 76

tert-Butyl (3S)-3-[[2-[4-(4-chlorophenyl)-5-(4-pyridyl)imidazol-1-yl]acetyl]amino]pyrrolidine-1-carboxylate (Intermediate 76-1) (78% purity, 65 mg, 0.105 mmol) was dissolved in 4M HCl in dioxane (1 mL) and MeOH (0.5 ml). The mixture was stirred at RT for 1 h. The reaction was stirred for 30 min then concentrated in vacuo to afford the title compound as a HCl salt (50 mg, 97% yield), yellow solid. LCMS (Analytical Method H) Rt=0.48 min, MS (ESIpos): m/z 382.3, 384.2 [M+H]+, Purity=100%.

### Synthesis of tert-butyl (3R)-3-{2-[4-(4-chlorophenyl)-5-(pyridin-4-yl)-1H-imidazol-1-yl]acetamido}pyrrolidine-1-carboxylate/Intermediate 77-1

2-[4-(4-Chlorophenyl)-5-(4-pyridyl)imidazol-1-yl]acetic acid; 2,2,2-trifluoroacetic acid (Intermediate 2a) (75 mg, 0.137 mmol) and tert-butyl (3R)-3-aminopyrrolidine-1-carboxylate (30 mg, 0.161 mmol) were dissolved in EtOAc (1 mL) then T3P (50%, 130 uL, 0.218 mmol) and DIPEA (80 uL, 0.458 mmol) were added. The reaction was stirred at RT for 16 h. Additional T3P (50%, 40 uL, 0.0672 mmol) was added and the reaction was stirred for 4 h. The reaction was diluted with EtOAc and partitioned with water. The organic layer was separated and the aqueous extracted with EtOAc. The organics were combined and concentrated in vacuo. The crude product was purified via flash chromatography (10 g, silica) eluting with 0-100% MeOH in DCM to afford the title compound (70 mg, 94% yield) as a yellow gum. LCMS (Analytical Method H) Rt=0.54 min, MS (ESIpos): m/z 482.2, 484.3 [M+H]+, Purity=89%.

### Synthesis of 2-[4-(4-chlorophenyl)-5-(pyridin-4-yl)-1H-imidazol-1-yl]-N-[(3R)-pyrrolidin-3-yl]acetamide/Intermediate 77

tert-Butyl (3R)-3-[[2-[4-(4-chlorophenyl)-5-(4-pyridyl)imidazol-1-yl]acetyl]amino]pyrrolidine-1-carboxylate (Intermediate 77-1) (89% purity, 70 mg, 0.129 mmol) was dissolved in 4M HCl in dioxane (1 mL) and MeOH (0.5 mL) and the mixture was stirred at RT for 1 h. The reaction was stirred for 30 min then concentrated in vacuo to afford the title compound as a HCl salt (60 mg, 90% yield), a yellow solid. LCMS (Analytical Method H) Rt=0.48 min, MS (ESIpos): m/z 382.3, 384.2 [M+H]+, Purity=95%.

### Synthesis of tert-butyl 2-[2-bromo-4-(4-chlorophenyl)-5-(pyridin-4-yl)-1H-imidazol-1-yl]acetate/Intermediate 78-1

tert-Butyl 2-[4-(4-chlorophenyl)-5-(4-pyridyl)imidazol-1-yl]acetate (Intermediate 2-3) (76% purity, 344 mg, 0.707 mmol) was dissolved in MeCN (6 mL) and NBS (130 mg, 0.730 mmol) was added. The mixture was stirred at RT for 1 h. The reaction was stirred at 50° C. for 2 h. The reaction was stirred at 80° C. for 2 h. Additional NBS (130 mg, 0.730 mmol) was added and the reaction was stirred at 80° C. for 2 h. The reaction was concentrated in vacuo and the residue was partitioned between water and DCM. The organic phase was separated and the aqueous extracted with DCM. The organics were combined and concentrated in vacuo and the crude product was purified via flash chromatography (25 g, silica) eluting with 0-100% EtOAc in heptane then 0-80% MeOH in EtOAc. Product containing fractions were combined and concentrated to give the title compound (54 mg, 12% yield). 1H NMR (500 MHz, DMSO-d6) δ 8.73-8.71 (m, 2H), 7.34-7.31 (m, 6H), 4.66-4.57 (m, 2H), 1.33 (s, 9H). LCMS (Analytical Method H) Rt=0.68 min, MS (ESIpos): m/z 448.2, 450.1 [M+H]+, Purity=73%.

### Synthesis of tert-butyl 2-[4-(4-chlorophenyl)-2-(1-methyl-1H-pyrazol-4-yl)-5-(pyridin-4-yl)-1H-imidazol-1-yl]acetate/Intermediate 78

tert-Butyl 2-[2-bromo-4-(4-chlorophenyl)-5-(4-pyridyl)imidazol-1-yl]acetate (Intermediate 78-1) (73% purity, 54 mg, 0.0878 mmol), (1-methyl-1H-pyrazol-4-yl)boronic acid (17 mg, 0.135 mmol) and 2 M Na₂CO₃ (135 uL, 0.270 mmol) were dissolved in DME (0.5 mL) and the mixture was degassed with nitrogen for 5 min, then Pd(PPh₃)₄ (9.0 mg, 7.79 μmol) was added. The reaction was stirred for 1 h at 100° C. (microwave). The reaction mixture was partitioned between water and DCM. The organic phase was separated and the aqueous was extracted with DCM. The combined organics were concentrated in vacuo. The crude product was purified via preparative HPLC (Method A1) to afford the title compound (16 mg, 41% yield) as an off white solid. 1H NMR (500 MHz, Methanol-d4) δ 8.65 (d, J=5.5 Hz, 2H), 8.02 (s, 1H), 7.80 (d, J=0.6 Hz, 1H), 7.40-7.35 (m, 4H), 7.30-7.27 (m, 2H), 4.69 (s, 2H), 3.99 (s, 3H), 1.37 (s, 9H). LCMS (Analytical Method H) Rt=0.59 min, MS (ESIpos): m/z 450.3, 452.3 [M+H]+, Purity=100%.

### Synthesis of N-[2-(4-chlorophenyl)-2-oxoethyl]-4-methoxybenzamide/Intermediate 79-1

2-Amino-1-(4-chlorophenyl)ethanone hydrochloride (300 mg, 1.46 mmol) was dissolved in a solution of DCM (5 mL) and DIPEA (550 uL, 3.15 mmol), then 4-methoxybenzoyl chloride (200 uL, 1.48 mmol) was added at 0° C. The mixture was stirred for 15 min at this temperature then at RT for 45 min. The reaction mixture was partitioned between water and DCM. The organic phase was separated and the aqueous was extracted with DCM. The combined organics were concentrated in vacuo. The crude product was purified via flash chromatography (25 g, silica) eluting with 0-100% EtOAc in heptane to afford the title compound (440 mg, 98% yield) as a cream coloured solid. 1H NMR (500 MHz, DMSO-d6) δ 8.73 (t, J=5.6 Hz, 1H), 8.07-8.03 (m, 2H), 7.90-7.85 (m, 2H), 7.65-7.60 (m, 2H), 7.05-6.99 (m, 2H), 4.73 (d, J=5.6 Hz, 2H), 3.82 (s, 3H) LCMS (Analytical Method H) Rt=0.56 min, MS (ESIpos): m/z 304.1, 306.0 [M+H]+, Purity=99%.

### Synthesis of 4-(4-chlorophenyl)-2-(4-methoxyphenyl)-1H-imidazole/Intermediate 79-2

N-[2-(4-Chlorophenyl)-2-oxo-ethyl]-4-methoxy-benzamide (Intermediate 79-1) (430 mg, 1.40 mmol) and ammonium acetate (1000 mg, 13.0 mmol) were suspended in acetic acid (6 mL) and the mixture was sealed and stirred at 120° C. for 18 h. The reaction was stirred at 140° C. for 24 h then concentrated in vacuo. The crude product was purified via flash chromatography (25 g, silica) eluting with 0-10% MeOH in DCM. The product still contained a significant impurity and was further purified via preparative HPLC (Method A1) to afford the title compound (135 mg, 34% yield) as an off-white solid. 1H NMR (400 MHz, DMSO-d6) δ 12.52 (s, 1H), 7.95-7.89 (m, 2H), 7.88-7.83 (m, 2H), 7.75 (s, 1H), 7.43-7.38 (m, 2H), 7.06-7.01 (m, 2H), 3.81 (s, 3H). LCMS (Analytical Method H) Rt=0.61 min, MS (ESIpos): m/z 285.1, 287.0 [M+H]+, Purity=99%.

### Synthesis of tert-butyl 4-{2-[4-(4-chlorophenyl)-2-(4-methoxyphenyl)-1H-imidazol-1-yl]acetyl}piperazine-1-carboxylate/Intermediate 79-3

4-(4-Chlorophenyl)-2-(4-methoxyphenyl)-1H-imidazole (Intermediate 79-2) (125 mg, 0.439 mmol) was dissolved in THF (4 mL) then at 0° C. NaH (60%, 18 mg, 0.450 mmol) was added. The mixture was stirred for 10 min then tert-butyl 4-(2-chloroacetyl)piperazine-1-carboxylate (Intermediate 14-1) (125 mg, 0.476 mmol) was added. The reaction was stirred for 1 h. The reaction was left standing overnight. Additional NaH (60%, 18 mg, 0.450 mmol) was added and the reaction was stirred for 1 h. The reaction was quenched with water and extracted with EtOAc. The organics were combined and concentrated in vacuo and the crude product was purified via flash chromatography (10 g, silica) eluting with 0-100% EtOAc in heptane to afford the title compound (181 mg, 75% yield) as an off white solid. 1H NMR (400 MHz, DMSO-d6) δ 7.81-7.77 (m, 2H), 7.65 (s, 1H), 7.50-7.45 (m, 2H), 7.44-7.39 (m, 2H), 7.07-7.02 (m, 2H), 5.02 (s, 2H), 3.81 (s, 3H), 3.51-3.44 (m, 4H), 3.38-3.33 (m, 4H), 1.42 (s, 9H). LCMS (Analytical Method H) Rt=0.66 min, MS (ESIpos): m/z 511.4, 513.3 [M+H]+, Purity=93%.

### Synthesis of tert-butyl 4-{2-[5-bromo-4-(4-chlorophenyl)-2-(4-methoxyphenyl)-1H-imidazol-1-yl]acetyl}piperazine-1-carboxylate/Intermediate 79-4

tert-Butyl 4-[2-[4-(4-chlorophenyl)-2-(4-methoxyphenyl)imidazol-1-yl]acetyl]piperazine-1-carboxylate (Intermediate 79-3) (170 mg, 0.309 mmol) was dissolved in DCM (2 mL) and cooled to 0° C. N-bromosuccinimide (60 mg, 0.337 mmol) was added and the mixture was stirred for 2 h. The reaction was diluted with DCM and quenched with 1M aq. NaOH. The organic layer was separated and the aqueous extracted with DCM. The organics were combined and concentrated in vacuo and the crude product was purified via flash chromatography (10 g silica) eluting with 0-100% EtOAc in heptane to afford the title compound (156 mg, 83% yield) as a white solid. 1H NMR (500 MHz, DMSO-d6) δ 8.00-7.97 (m, 2H, H7, H11), 7.53-7.49 (m, 2H, H8, H10), 7.49-7.45 (m, 2H, H18, H22), 7.10-7.06 (m, 2H, H19, H21), 4.99 (s, 2H, H13), 3.81 (s, 3H, H24), 3.56-3.49 (m, 4H, H26, H30), 3.39-3.34 (m, 4H, H27, H29), 1.42 (s, 9H, H35, H36, H37). LCMS (Analytical Method G) Rt=1.96 min, MS (ESIpos): m/z 591.3 [M+H]+, Purity=97%.

### Synthesis of tert-butyl 4-{2-[4-(4-chlorophenyl)-2-(4-methoxyphenyl)-5-(pyridin-4-yl)-1H-imidazol-1-yl]acetyl}piperazine-1-carboxylate/Intermediate 79

tert-Butyl 4-[2-[5-bromo-4-(4-chlorophenyl)-2-(4-methoxyphenyl)imidazol-1-yl]acetyl]piperazine-1-carboxylate (Intermediate 79-4) (145 mg, 0.246 mmol), pyridin-4-ylboronic acid (35 mg, 0.285 mmol) and Na₂CO₃ (60 mg, 0.566 mmol) were suspended in DME (1.6 mL) and Water (0.4 mL). The mixture was degassed with nitrogen for 10 min then palladium; triphenylphosphane (30 mg, 0.0260 mmol) was added. The mixture was sealed under nitrogen and stirred at 100° C. (microwave) for 2 h. The mixture was retreated with pyridin-4-ylboronic acid (15 mg, 0.122 mmol), palladium triphenylphosphane (14 mg, 0.0121 mmol) and Na₂CO₃ (26 mg, 0.245 mmol) then stirred at 100° C. (microwave) for 1 h. The reaction mixture was partitioned between water and EtOAc. The organic phase was separated and the aqueous was extracted with EtOAc. The combined organics were concentrated in vacuo and the crude product was purified via flash chromatography (10 g, silica) eluting with 0-80% MeOH in EtOAc to afford the title compound (85 mg, 56% yield) as a pale yellow solid. 1H NMR (500 MHz, DMSO-d6) δ 8.72-8.69 (m, 2H), 7.57-7.52 (m, 2H), 7.44-7.40 (m, 2H), 7.37-7.31 (m, 4H), 7.12-7.08 (m, 2H), 4.75 (s, 2H), 3.83 (s, 3H), 3.43-3.38 (m, 2H), 3.27-3.23 (m, 2H), 3.23-3.19 (m, 2H), 3.08-3.02 (m, 2H), 1.41 (s, 9H). LCMS (Analytical Method G) Rt=1.75 min, MS (ESIpos): m/z 588.4, 590.4 [M+H]+, Purity=97%.

### Synthesis of N-[2-(4-chlorophenyl)-2-oxoethyl]-6-methoxypyridine-3-carboxamide/Intermediate 80-1

6-Methoxypyridine-3-carboxylic acid (350 mg, 2.29 mmol) and HATU (950 mg, 2.50 mmol) were dissolved in a solution of DMF (7 mL) and DIPEA (1230 uL, 7.04 mmol) and stirred for 10 min at RT, then 2-amino-1-(4-chlorophenyl)ethanone hydrochloride (500 mg, 2.43 mmol) was added. The reaction was stirred for 1 h. The reaction was diluted with water causing a precipitate to form. The solid was collected via filtration and dried via vacuum oven to give the title compound (471 mg, 68% yield) as an off-white solid. 1H NMR (500 MHz, DMSO-d6) δ 8.92 (t, J=5.5 Hz, 1H), 8.72 (d, J=2.2 Hz, 1H), 8.16 (dd, J=8.7, 2.5 Hz, 1H), 8.07-8.03 (m, 2H), 7.66-7.61 (m, 2H), 6.92 (d, J=8.7 Hz, 1H), 4.77 (d, J=5.6 Hz, 2H), 3.92 (s, 3H). LCMS (Analytical Method G) Rt=1.51 min, MS (ESIpos): m/z 305.1, 307.1 [M+H]+, Purity=100%.

### Synthesis of 5-[4-(4-chlorophenyl)-1H-imidazol-2-yl]-2-methoxypyridine/Intermediate 80-2

N-[2-(4-Chlorophenyl)-2-oxo-ethyl]-6-methoxy-pyridine-3-carboxamide (Intermediate 80-1) (460 mg, 1.51 mmol) and ammonium acetate (1000 mg, 13.0 mmol) were suspended in acetic acid (6 mL) and the mixture was sealed and stirred at 120° C. for 18 h. The reaction was stirred at 140° C. for 24 h then concentrated in vacuo. The crude product was purified via flash chromatography (25 g silica) eluting with 0-10% MeOH in DCM. The product was purified again via preparative HPLC (Method A1) to afford the title compound (110 mg, 25% yield) as an off-white solid. 1H NMR (400 MHz, DMSO-d6) δ 12.69 (s, 1H), 8.76 (d, J=2.1 Hz, 1H), 8.25 (dd, J=8.7, 2.4 Hz, 1H), 7.88-7.83 (m, 2H), 7.81 (s, 1H), 7.47-7.37 (m, 2H), 6.94 (d, J=8.6 Hz, 1H), 3.90 (s, 3H). LCMS (Analytical Method H) Rt=0.58 min, MS (ESIpos): m/z 286.1, 288.1 [M+H]+, Purity=98%.

### Synthesis of tert-butyl 4-{2-[4-(4-chlorophenyl)-2-(6-methoxypyridin-3-yl)-1H-imidazol-1-yl]acetyl}piperazine-1-carboxylate/Intermediate 80-3

5-[4-(4-Chlorophenyl)-1H-imidazol-2-yl]-2-methoxy-pyridine (Intermediate 80-2) (100 mg, 0.350 mmol) was dissolved in THF (3 mL) then at 0° C. NaH (60%, 17 mg, 0.425 mmol) was added. The mixture was stirred for 30 min then tert-butyl 4-(2-chloroacetyl)piperazine-1-carboxylate (Intermediate 14-1) (110 mg, 0.419 mmol) was added. The reaction was stirred for 20 h. Additional NaH (60%, 4.0 mg, 0.100 mmol) and tert-butyl 4-(2-chloroacetyl)piperazine-1-carboxylate (Intermediate 14-1) (10 mg, 0.0381 mmol) were added and the mixture was stirred for 2 h. The reaction was quenched with water and extracted with EtOAc. The organics were combined and concentrated in vacuo and the crude product was purified via flash chromatography (10 g, silica) eluting with 0-100% EtOAc in heptane to afford the title compound (150 mg, 77% yield) as a white solid. 1H NMR (500 MHz, DMSO-d6) δ 8.35 (dd, J=2.5, 0.5 Hz, 1H, H34), 7.87 (dd, J=8.6, 2.5 Hz, 1H, H30), 7.82-7.78 (m, 2H, H7, H11), 7.70 (s, 1H, H4), 7.45-7.40 (m, 2H, H8, H10), 6.95 (dd, J=8.6, 0.6 Hz, 1H, H31), 5.10 (s, 2H, H13), 3.90 (s, 3H, H36), 3.49-3.44 (m, 4H, H17, H21), 3.38-3.35 (m, 2H), 3.31-3.29 (m, 2H), 1.42 (s, 9H, H26, H27, H28). LCMS (Analytical Method H) Rt=0.64 min, MS (ESIpos): m/z 512.3, 514.3 [M+H]+, Purity=92%.

### Synthesis of tert-butyl 4-{2-[5-bromo-4-(4-chlorophenyl)-2-(6-methoxypyridin-3-yl)-1H-imidazol-1-yl]acetyl}piperazine-1-carboxylate/Intermediate 80-4

tert-Butyl 4-[2-[4-(4-chlorophenyl)-2-(6-methoxy-3-pyridyl)imidazol-1-yl]acetyl]piperazine-1-carboxylate (Intermediate 80-3) (140 mg, 0.273 mmol) was dissolved in DCM (3 mL) then cooled to 0° C. NBS (55 mg, 0.309 mmol) was added and the mixture was stirred for 3 h. The reaction was quenched 1 M aq. NaOH and extracted with DCM. The organics were combined and concentrated in vacuo and the crude product was purified via flash chromatography (10 g silica) eluting with 0-10% MeOH in DCM to afford the title compound (99 mg, 51% yield) as a pale yellow solid. 1H NMR (500 MHz, DMSO-d6) δ 8.37-8.32 (m, 1H), 8.00-7.96 (m, 2H), 7.84 (dd, J=8.6, 2.5 Hz, 1H), 7.54-7.50 (m, 2H), 6.98 (d, J=8.6 Hz, 1H), 5.06 (s, 2H), 3.91 (s, 3H), 3.56-3.52 (m, 2H), 3.51-3.48 (m, 2H), 3.40-3.36 (m, 2H), 3.35-3.33 (m, 2H), 1.42 (s, 9H). LCMS (Analytical Method G) Rt=1.89 min, MS (ESIpos): m/z 590.3, 592.3, 594.2 [M+H]+, Purity=83%.

### Synthesis of tert-butyl 4-{2-[4-(4-chlorophenyl)-2-(6-methoxypyridin-3-yl)-5-(pyridin-4-yl)-1H-imidazol-1-yl]acetyl}piperazine-1-carboxylate/Intermediate 80

tert-Butyl 4-[2-[5-bromo-4-(4-chlorophenyl)-2-(6-methoxy-3-pyridyl)imidazol-1-yl]acetyl]piperazine-1-carboxylate (Intermediate 80-4) (83% purity, 90 mg, 0.126 mmol), pyridin-4-ylboronic acid (25 mg, 0.203 mmol) and Na₂CO₃ (30 mg, 0.283 mmol) were suspended in DME (1.6 mL) and Water (0.4 mL) and the mixture was degassed with nitrogen for 5 min, then palladium; triphenylphosphane (15 mg, 0.0130 mmol) was added. The mixture was degassed for a further 5 min then sealed and stirred at 100° C. (microwave) for 3 h then cooled to room temperature and partitioned between water and EtOAc. The organic phase was separated and the aqueous was extracted with EtOAc. The combined organics were concentrated in vacuo. The crude product was purified via flash chromatography (10 g, silica) eluting with 0-10% MeOH in DCM. The product was purified again via preparative HPLC (Method A1) to afford the title compound (36 mg, 47% yield) as an off white solid. 1H NMR (500 MHz, DMSO-d6) δ 8.73-8.69 (m, 2H), 8.40 (d, J=1.9 Hz, 1H), 7.91 (dd, J=8.6, 2.4 Hz, 1H), 7.43-7.39 (m, 2H), 7.36-7.31 (m, 4H), 7.00 (d, J=8.6 Hz, 1H), 4.80 (s, 2H), 3.91 (s, 3H), 3.40-3.37 (m, 2H), 3.27-3.21 (m, 2H), 3.21-3.16 (m, 2H), 3.07-3.00 (m, 2H), 1.40 (s, 9H). LCMS (Analytical Method H) Rt=0.63 min, MS (ESIpos): m/z 589.4, 591.3 [M+H]+, Purity=97%.

### Synthesis of tert-butyl 3-[(methanesulfonyloxy)methyl]azetidine-1-carboxylate/Intermediate 81-1

To a stirring solution of tert-butyl 3-(hydroxymethyl)azetidine-1-carboxylate (750 mg, 4.01 mmol), N-ethyl-N-isopropyl-propan-2-amine (2.0 mL, 11.5 mmol), and N,N-dimethylpyridin-4-amine (50 mg, 0.409 mmol) in DCM-Anhydrous (10 mL) was added methanesulfonyl chloride (345 uL, 4.46 mmol) dropwise at 0° C. The reaction was then warmed to RT and stirred for 3 hours. The organic layer was diluted with NaHCO₃(20 mL), and the organic layer extracted with DCM (2×20 mL). The combined organic layers were washed with brine (2×15 mL), dried over Na₂SO₄, and concentrated under vacuum to afford the title compound (1.18 g, quantitative yield) as a brown oil. 1H NMR (400 MHz, Chloroform-d) δ 4.35 (d, J=6.8 Hz, 2H), 4.05 (t, J=8.7 Hz, 2H), 3.75-3.69 (m, 2H), 3.04 (s, 3H), 2.97-2.87 (m, 1H), 1.44 (s, 9H).

### Synthesis of tert-butyl 3-[(1H-imidazol-1-yl)methyl]azetidine-1-carboxylate/Intermediate 81-2

To a solution of 1H-imidazole (901 mg, 13.2 mmol) in DMF-Anhydrous (8 mL) was added NaH (311 mg, 12.9 mmol) at 0° C. and stirred for 1 hour. tert-butyl 3-(methylsulfonyloxymethyl)azetidine-1-carboxylate (Intermediate 81-1) (1.18 g, 4.45 mmol) was then added to the reaction and stirred at 60° C. for 3 hours. Reaction mixture was cooled to 0° C. and slowly quenched with water (10 mL). The organic layer was extracted with DCM (2×15 mL), before being concentrated in vacuo. The resulting residue was purified by flash chromatography (50 g, silica) eluting with 0-50% MeOH in DCM. Product containing fractions were combined and concentrated under vacuum to afford the title compound (0.87 g, 81% yield) as a yellow oil. 1H NMR (400 MHz, Chloroform-d) δ 7.47 (s, 1H), 7.07 (t, J=1.0 Hz, 1H), 6.89 (t, J=1.2 Hz, 1H), 4.16 (d, J=7.8 Hz, 2H), 4.03 (t, J=8.6 Hz, 2H), 3.65 (dd, J=9.0, 5.1 Hz, 2H), 2.94-2.89 (m, 1H), 1.43 (s, 9H). LCMS (Analytical Method J) Rt=0.42 min, MS (ESIpos): m/z 238.2 [M+H]+, Purity=99%.

### Synthesis of 1-[(azetidin-3-yl)methyl]-1H-imidazole/Intermediate 81

TFA (5.4 mL, 72.3 mmol) was added to a stirred solution of tert-butyl 3-(imidazol-1-ylmethyl)azetidine-1-carboxylate (Intermediate 81-2) (867 mg, 3.62 mmol) in DCM (20 mL), and the resulting mixture was stirred at RT for 2 h. Reaction mixture was concentrated in vacuo and the residue taken up in MeOH (˜5 ml) and passed down an ISOLUTE Flash SCX-2 column (20 g). The column was eluted with MeOH (3×20 ml) and then with 3 M NH₃ solution in MeOH (5×20 ml). The basic eluate was concentrated to give the title compound (505 mg, 71% yield) as a yellow oil. 1H NMR (400 MHz, DMSO-d6) δ 7.58-7.54 (m, 1H), 7.10-7.06 (m, 1H), 6.90-6.86 (m, 1H), 4.19 (d, J=7.4 Hz, 2H), 3.65 (t, J=8.4 Hz, 2H), 3.43-3.37 (m, 2H), 3.09-3.00 (m, 1H).

### Synthesis of tert-butyl 4-{2-[4-(4-fluorophenyl)-5-(pyrimidin-4-yl)-1H-imidazol-1-yl]acetyl}piperazine-1-carboxylate/Intermediate 82-1

tert-Butyl 4-(2-aminoacetyl)piperazine-1-carboxylate (210 mg, 0.863 mmol), pyrimidine-4-carbaldehyde (80% purity, 82 uL, 0.867 mmol) and MgSO₄ (104 mg, 0.863 mmol) were dissolved in DCM (3 mL) and the mixture was stirred at RT for 2 h. The reaction was stirred for 1 h then filtered. The filtrate was concentrated in vacuo then taken up in DMF (3 mL). (4-fluorophenyl)(isocyano)methyl 4-methylphenyl sulfone (250 mg, 0.864 mmol) and potassium carbonate (163 mg, 1.18 mmol) were added and the reaction was stirred for 17 h. The reaction was quenched with water and extracted with DCM. The organics were combined and concentrated and the crude product was purified via flash chromatography (10 g, silica) eluting with 0-10% MeOH in DCM, then by preparative HPLC (Method A1) to afford the title compound (150 mg, 37% yield) as a white solid. 1H NMR (400 MHz, DMSO-d6) δ 9.18 (d, J=1.3 Hz, 1H), 8.62 (d, J=5.4 Hz, 1H), 7.88 (s, 1H), 7.52-7.45 (m, 2H), 7.25-7.17 (m, 3H), 5.35 (s, 2H), 3.54-3.47 (m, 2H), 3.41 (d, J=1.1 Hz, 2H), 3.30-3.20 (m, 4H), 1.42 (s, 9H). LCMS (Analytical Method J) Rt=0.76 min, MS (ESIpos): m/z 467.3 [M+H]+, Purity=90%.

### Synthesis of 2-[4-(4-fluorophenyl)-5-(pyrimidin-4-yl)-1H-imidazol-1-yl]-1-(piperazin-1-yl)ethan-1-one/Intermediate 82

tert-Butyl 4-[2-[4-(4-fluorophenyl)-5-pyrimidin-4-yl-imidazol-1-yl]acetyl]piperazine-1-carboxylate (Intermediate 82-1) (30 mg, 0.0643 mmol) was dissolved in 4M HCl in dioxane (1.5 mL) and the mixture was stirred at RT for 2 h. The mixture was stirred for 1 h then concentrated in vacuo to afford the title compound as a HCl salt (42 mg, 96% yield) 1H NMR (500 MHz, DMSO-d6) δ 9.61 (s, 1H), 9.36 (d, J=1.0 Hz, 1H), 9.19 (s, 1H), 8.78 (d, J=5.3 Hz, 1H), 7.62-7.55 (m, 2H), 7.40-7.33 (m, 2H), 7.28 (dd, J=1.1, 5.3 Hz, 1H), 5.57 (s, 2H), 3.69-3.63 (m, 4H), 3.21-3.17 (m, 2H), 3.07-3.01 (m, 2H). LCMS (Analytical Method J) Rt=0.46 min, MS (ESIpos): m/z 367.3 [M+H]+, Purity=93%.

### Synthesis of ethyl (3Z)-5-(4-fluorophenyl)-3-hydroxy-5-oxopent-3-enoate and methyl (3Z)-5-(4-fluorophenyl)-3-hydroxy-5-oxopent-3-enoate/Intermediate 83-1

To a stirred solution of ethyl 3-oxobutanoate (2.0 mL, 15.8 mmol) in THF-Anhydrous (30.87 mL) at −78° C. and under N2, 2 M LDA (24 mL, 47.4 mmol) was added followed by TMEDA (2.4 mL, 15.8 mmol), and the mixture was allowed to stir at 0° C. for 3 h. Then methyl 4-fluorobenzoate (2.74 g, 17.4 mmol) was added, and the reaction was allowed to warm up to RT and stirred at RT overnight. The reaction mixture was cooled down to 0° C. and acetic acid was then added. After stirring for 10 min, the mixture was diluted with 1 N HCl and extracted with EtOAc (2×). The combined organics were washed with brine, dried over MgSO₄, filtered, and evaporated under reduced pressure. The residue was purified by flash chromatography (100 g, silica) eluting with 0-100% DCM/heptane to afford the title compounds (1.40 g, 25% yield) as a ˜1:1 mixture of the methyl and ethyl esters. LCMS (Analytical Method J) Rt=0.92, 0.99 min, MS (ESIpos): m/z 239.1; 253.1 [M+H]+, Purity=70%.

### Synthesis of ethyl 2-[3-(4-fluorophenyl)-1-methyl-1H-pyrazol-5-yl]acetate, methyl 2-[3-(4-fluorophenyl)-1-methyl-1H-pyrazol-5-yl]acetate, ethyl 2-[5-(4-fluorophenyl)-1-methyl-1H-pyrazol-3-yl]acetate and methyl 2-[5-(4-fluorophenyl)-1-methyl-1H-pyrazol-3-yl]acetate/Intermediate 83-2

Methylhydrazine (0.18 mL, 3.36 mmol) was added to a solution of the two isomers from the previous step (Intermediate 83-1) (770 mg, 3.05 mmol) in MeOH (15.4 mL) and acetic acid (0.462 mL) and the reaction mixture was heated at 70° C. overnight. The mixture was diluted with EtOAc (30 mL) and washed with 1 M aq. NaOH (25 mL). The aq layer was extracted with EtOAc (2×30 mL). The combined organics were dried over MgSO₄, filtered and evaporated under reduced pressure to yield the title compound (715 mg, 83% yield) as mixture of methyl and ethyl esters with the corresponding two possible regioisomers from the cyclisation which was used in the next step without further purification. LCMS (Analytical Method J) Rt=0.83, 0.85, 0.90, 0.91 min, MS (ESIpos): m/z 249.1, 263.1 [M+H]+, Purity=93%.

### Synthesis of ethyl 2-[4-bromo-3-(4-fluorophenyl)-1-methyl-1H-pyrazol-5-yl]acetate, methyl 2-[4-bromo-3-(4-fluorophenyl)-1-methyl-1H-pyrazol-5-yl]acetate, ethyl 2-[4-bromo-5-(4-fluorophenyl)-1-methyl-1H-pyrazol-3-yl]acetate and methyl 2-[4-bromo-5-(4-fluorophenyl)-1-methyl-1H-pyrazol-3-yl]acetate/Intermediate 83-3

N-bromosuccinimide (541 mg, 3.04 mmol) was added to an ice cold solution of the four isomers from the previous step (Intermediate 83-2) (715 mg, 2.54 mmol) in DCM (19.696 mL), and the reaction was stirred for 60 minutes. Water was added (20 mL), and the organic layer was separated. The aqueous layer was extracted into DCM (2×20 mL), and the organic extracts were combined, dried over MgSO₄, filtered and concentrated in vacuo to yield the title compounds (966 mg, quantitative yield) as a mixture of four products. LCMS (Analytical Method J) Rt=0.93, 0.97, 1.00, 1.03 min, MS (ESIpos): m/z 327.0, 329.0; 341.0, 343.0 [M+H]+, Purity=97%.

### Synthesis of ethyl 2-[3-(4-fluorophenyl)-1-methyl-4-(pyridin-4-yl)-1H-pyrazol-5-yl]acetate, methyl 2-[3-(4-fluorophenyl)-1-methyl-4-(pyridin-4-yl)-1H-pyrazol-5-yl]acetate, ethyl 2-[5-(4-fluorophenyl)-1-methyl-4-(pyridin-4-yl)-1H-pyrazol-3-yl]acetate and methyl 2-[5-(4-fluorophenyl)-1-methyl-4-(pyridin-4-yl)-1H-pyrazol-3-yl]acetate/Intermediate 83-4

A mixture of the four isomers from the previous step (Intermediate 83-3) (966 mg, 2.75 mmol), pyridin-4-ylboronic acid (506 mg, 4.12 mmol), palladium; triphenylphosphane (159 mg, 0.137 mmol) and 2 M Na₂CO₃ (3.9 mL, 7.76 mmol) in DME (14.55 mL) was degassed by sparging with nitrogen. The reaction was heated at 125° C. for 2 hours under microwave irradiation. The reaction mixture was diluted with EtOAc (20 mL), washed with water (15 mL), dried over MgSO₄, filtered and evaporated under reduced pressure. The residue was loaded onto an SCX column. The column was washed with MeOH (3 CV), then the product was eluted with 7N NH₃ in MeOH and the solvent evaporated in vacuo to yield the title compounds (499 mg, 50% yield) as a mixture of four products. LCMS (Analytical Method J) Rt=0.58, 0.63, 0.67 min, MS (ESIpos): m/z 326.2, 340.2 [M+H]+, Purity=93%.

### Synthesis of lithium(1+) 2-[3-(4-fluorophenyl)-1-methyl-4-(pyridin-4-yl)-1H-pyrazol-5-yl]acetate and lithium(1+) 2-[5-(4-fluorophenyl)-1-methyl-4-(pyridin-4-yl)-1H-pyrazol-3-yl]acetate/Intermediate 83

To a stirred solution of the four isomers from the previous step (Intermediate 83-4) (499 mg, 1.37 mmol) in MeOH (14.85 mL) and Water (7.4251 mL), lithium hydroxide hydrate (1:1:1) (287 mg, 6.84 mmol) was added, and the resulting mixture was allowed to stir at RT for overnight. Solvent was evaporated under reduced pressure and the residue was dried in the vacuum oven to provide the title compound (590 mg, quantitative yield) as a pale-brown solid. Product was isolated as a mixture of two regioisomers and was used in the next step without further purification. LCMS (Analytical Method B) Rt=1.37 min, MS (ESIpos): m/z 312.2 [M+H]+, Purity=78%.

### Synthesis of methyl (3Z)-5-(4-fluorophenyl)-3-hydroxy-5-oxopent-3-enoate/Intermediate 84-1

To a stirred solution of methyl 3-oxobutanoate (2.0 mL, 18.5 mmol) in THF-Anhydrous (38 mL) at −78° C. and under N2, 2 M LDA (28 mL, 56.0 mmol) was added over 15 min using an addition funnel, followed by TMEDA (2.8 mL, 18.7 mmol), and the mixture was allowed to stir at 0° C. for 3 h. Then a solution of methyl 4-fluorobenzoate (2.7 mL, 20.5 mmol) in THF-Anhydrous (20 mL) was added over 10 min using an addition funnel, and the reaction was allowed to warm up to RT and stirred at RT overnight. The reaction mixture was cooled down to 0° C. and acetic acid (5 mL) was then added. After stirring for 10 min, the mixture was diluted with 1 N HCl (50 mL) and extracted with EtOAc (2×80 mL). The combined organics were washed with brine (150 mL), dried over MgSO₄, filtered, and evaporated under reduced pressure. The residue was purified by flash chromatography (100 g, silica) eluting with 0-100% DCM/heptane to yield the title compound (2.45 g, 50% yield) as a brown oil. 1H NMR (400 MHz, Chloroform-d) δ 15.79 (s, 1H), 7.93-7.88 (m, 2H), 7.17-7.10 (m, 2H), 6.24 (s, 1H), 3.77 (s, 3H), 3.48 (s, 2H). LCMS (Analytical Method J) Rt=0.92 min, MS (ESIpos): m/z 239.1 [M+H]+, Purity=82%.

### Synthesis of methyl 2-[1-tert-butyl-5-(4-fluorophenyl)-1H-pyrazol-3-yl]acetate/Intermediate 84-2

tert-Butylhydrazine hydrochloride (0.52 g, 4.16 mmol) was added to a solution of methyl 5-(4-fluorophenyl)-3,5-dioxo-pentanoate (Intermediate 84-1) (1.00 g, 3.78 mmol) in MeOH (18 mL) and acetic acid (0.54 mL) and the reaction mixture was heated at 70° C. for 1.5 hours. The mixture was diluted with EtOAc (50 mL) and washed with 1 M aq. NaOH (30 mL). The aq layer was extracted with EtOAc (2×30 mL). The combined organics were dried over MgSO₄, filtered and evaporated under reduced pressure. The residue was purified by flash chromatography (50 g, silica), eluting with 0-20% EtOAc/heptane to yield the title compound (770 mg, 70% yield). 1H NMR (500 MHz, Chloroform-d) δ 7.34-7.28 (m, 2H), 7.06 (t, J=8.6 Hz, 2H), 6.10 (s, 1H), 3.73 (s, 3H), 3.70 (s, 2H), 1.42 (s, 9H). LCMS (Analytical Method J) Rt=1.05 min, MS (ESIpos): m/z 291.1 [M+H]+, Purity=84%.

### Synthesis of methyl 2-[4-bromo-1-tert-butyl-5-(4-fluorophenyl)-1H-pyrazol-3-yl]acetate/Intermediate 84-3

N-bromosuccinimide (566 mg, 3.18 mmol) was added to an ice cold solution of methyl 2-[1-tert-butyl-5-(4-fluorophenyl)pyrazol-3-yl]acetate (Intermediate 84-2) (770 mg, 2.65 mmol) in DCM (15 mL), and the reaction was stirred for 4 h. Water was added (10 mL), and the organic layer was separated. The aqueous layer was extracted into DCM (2×10 mL), and the organic extracts were combined, dried over MgSO₄, filtered and concentrated in vacuo to provide the title compound (1.07 g, 96% yield), which was used in the next step without further purification. 1H NMR (500 MHz, Chloroform-d) δ 7.32-7.26 (m, 2H), 7.17-7.10 (m, 2H), 3.74 (s, 3H), 3.71 (s, 2H), 1.41 (s, 9H). LCMS (Analytical Method J) Rt=1.12 min, MS (ESIpos): m/z 369.1, 371.1 [M+H]+, Purity=88%.

### Synthesis of methyl 2-[1-tert-butyl-5-(4-fluorophenyl)-4-(pyridin-4-yl)-1H-pyrazol-3-yl]acetate/Intermediate 84-4

A mixture of methyl 2-[4-bromo-1-tert-butyl-5-(4-fluorophenyl)pyrazol-3-yl]acetate (Intermediate 84-3) (0.88 g, 2.38 mmol), pyridin-4-ylboronic acid (439 mg, 3.57 mmol), palladium; triphenylphosphane (140 mg, 0.121 mmol) and 2 M Na₂CO₃ (3.5 mL, 7.00 mmol) in DME (13 mL) was degassed by sparging with nitrogen. The reaction was heated at 125° C. for 2 hours under microwave irradiation. The reaction mixture was diluted with EtOAc (20 mL), washed with water (30 mL), dried over MgSO₄, filtered and evaporated under reduced pressure. The residue was purified by flash chromatography (50 g, silica) eluting with 0-5% MeOH/DCM to yield the title compound (695 mg, 75% yield). 1H NMR (500 MHz, Chloroform-d) δ 8.41-8.34 (m, 2H), 7.29-7.22 (m, 2H), 7.08-7.00 (m, 2H), 6.94-6.89 (m, 2H), 3.72 (s, 2H), 3.65 (s, 3H), 1.46 (s, 9H). LCMS (Analytical Method J) Rt=0.75 min, MS (ESIpos): m/z 368.2 [M+H]+, Purity=94%.

### Synthesis of lithium(1+) 2-[1-tert-butyl-5-(4-fluorophenyl)-4-(pyridin-4-yl)-1H-pyrazol-3-yl]acetate/Intermediate 84-5

To a stirred solution of methyl 2-[1-tert-butyl-5-(4-fluorophenyl)-4-(4-pyridyl)pyrazol-3-yl]acetate (Intermediate 84-4) (649 mg, 1.77 mmol) in MeOH (15 mL) and Water (8 mL), lithium hydroxide hydrate (1:1:1) (370 mg, 8.83 mmol) was added, and the resulting mixture was allowed to stir at RT for 6 h. Solvent was evaporated under reduced pressure and the residue was triturated with acetone, filtered off, and dried in the vacuum oven to yield the title compound (703 mg, quantitative yield) as an off-white solid. 1H NMR (400 MHz, DMSO-d6) δ 8.28-8.23 (m, 2H), 7.44-7.37 (m, 2H), 7.28-7.21 (m, 4H), 3.15 (s, 2H), 1.38 (s, 9H). LCMS (Analytical Method J) Rt=0.68 min, MS (ESIpos): m/z 354.2 [M+H]+, Purity=93%.

### Synthesis of 2-[1-tert-butyl-5-(4-fluorophenyl)-4-(pyridin-4-yl)-1H-pyrazol-3-yl]-1-(4-methylpiperazin-1-yl)ethan-1-one/Intermediate 84 and Reference Compound 106 from Table 1

To a stirred solution of lithium; 2-[1-tert-butyl-5-(4-fluorophenyl)-4-(4-pyridyl)pyrazol-3-yl]acetate (Intermediate 84-5) (180 mg, 0.501 mmol) and DIPEA (0.26 mL, 1.50 mmol) in EtOAc (3.6 mL) at RT and under nitrogen, T3P (50%, 0.60 mL, 1.00 mmol) was added followed by 1-methylpiperazine (112 uL, 1.00 mmol), and the resulting mixture was allowed to stir at 60° C. overnight. The reaction was diluted with EtOAc (5 mL) and washed with water (10 mL). The aq layer was extracted with EtOAc (2×10 mL). The combined organics were dried over MgSO₄, filtered, and evaporated under reduced pressure to yield the title compound (226 mg, 98% yield) as a pale yellow solid. 1H NMR (500 MHz, Methanol-d4) δ 8.32-8.27 (m, 2H), 7.42-7.35 (m, 2H), 7.19-7.11 (m, 4H), 3.82 (s, 2H), 3.71-3.65 (m, 2H), 3.63-3.57 (m, 2H), 2.49-2.43 (m, 2H), 2.44-2.38 (m, 2H), 2.32 (s, 3H), 1.47 (s, 9H). LCMS (Analytical Method B) Rt=2.96 min, MS (ESIpos): m/z 436.4 [M+H]+, Purity=95%.

### Synthesis of tert-butyl N-{4-[4-(4-fluorophenyl)-1-[2-(morpholin-4-yl)-2-oxoethyl]-1H-imidazol-5-yl]pyridin-2-yl}carbamate/Intermediate 85-1

2-[5-Bromo-4-(4-fluorophenyl)imidazol-1-yl]-1-morpholino-ethanone (Intermediate 43) (430 mg, 1.07 mmol), tert-butyl [4-(4,4,5,5-tetramethyl-1,3,2-dioxaborolan-2-yl)pyridin-2-yl]carbamate (400 mg, 1.22 mmol) and Na₂CO₃ (300 mg, 2.83 mmol) were suspended in DME (5 mL) and Water (1.5 mL) then degassed with nitrogen for 5 min. palladium; triphenylphosphane (125 mg, 0.108 mmol) was added and the reaction was stirred at 100° C. for 1 h (microwave). The reaction mixture was cooled to room temperature and diluted with water, then extracted with DCM, The organics were combined, filtered and the filtrate was concentrated in vacuo. The crude product was purified via flash chromatography (25 g, silica) eluting with 0-10% MeOH in DCM to afford the title compound (439 mg, 46% yield). 1H NMR (500 MHz, DMSO-d6) δ 9.92 (s, 1H), 8.28 (d, J=5.1 Hz, 1H), 7.78 (s, 1H), 7.65 (s, 1H), 7.44-7.39 (m, 2H), 7.13-7.08 (m, 2H), 6.84 (dd, J=5.1, 1.4 Hz, 1H), 4.87 (s, 2H), 3.54-3.51 (m, 2H), 3.49-3.46 (m, 2H), 3.44-3.42 (m, 2H), 3.38-3.36 (m, 2H), 1.44 (s, 9H). LCMS (Analytical Method J) Rt=0.75 min, MS (ESIpos): m/z 482.3 [M+H]+, Purity=54%.

### Synthesis of 2-[5-(2-aminopyridin-4-yl)-4-(4-fluorophenyl)-1H-imidazol-1-yl]-1-(morpholin-4-yl)ethan-1-one/Intermediate 85

tert-Butyl N-[4-[5-(4-fluorophenyl)-3-(2-morpholino-2-oxo-ethyl)imidazol-4-yl]-2-pyridyl]carbamate (Intermediate 85-1) (56% purity, 430 mg, 0.500 mmol) was dissolved in 4M HCl in dioxane (5 mL) and stirred at RT for 20 h. The reaction was concentrated in vacuo. The crude product was loaded to an SCX-2 column then washed with DCM/MeOH. The product was eluted with 7N ammonia in MeOH. The eluate was concentrated in vacuo to afford the title compound (225 mg, 85% yield) as a beige solid. 1H NMR (400 MHz, DMSO-d6) δ 7.99 (d, J=5.2 Hz, 1H), 7.73 (s, 1H), 7.49-7.43 (m, 2H), 7.14-7.07 (m, 2H), 6.35 (dd, J=5.2, 1.4 Hz, 1H), 6.28 (s, 1H), 6.06 (s, 2H), 4.80 (s, 2H), 3.54-3.50 (m, 2H), 3.50-3.46 (m, 2H), 3.43-3.36 (m, 4H) LCMS (Analytical Method H) Rt=0.54 min, MS (ESIpos): m/z 382.3 [M+H]+, Purity=72%.

### Synthesis of tert-butyl 7-{2-[4-(4-chlorophenyl)-5-(pyridin-4-yl)-1H-imidazol-1-yl]-N-methylacetamido}-5-oxa-2-azaspiro[3.4]octane-2-carboxylate/Intermediate 86-1

NaH (60%, 10 mg, 0.252 mmol) was added to an ice cold solution of tert-butyl 7-[[2-[4-(4-chlorophenyl)-5-(4-pyridyl)imidazol-1-yl]acetyl]amino]-5-oxa-2-azaspiro[3.4]octane-2-carboxylate (Intermediate 57-1) (90 mg, 0.168 mmol) in DMF (2 mL). The reaction was stirred for 5 minutes then 2 M iodomethane (2M TBME) (252 uL, 0.505 mmol) was added and stirring continued for 1 hour. The reaction was quenched by dropwise addition of water, then allowed to warm to room temperature. The aqueous layer was extracted into EtOAc (10 mL) three times, the combined organics washed with brine, dried over MgSO₄ and concentrated in vacuo. The residue was purified by flash chromatography (10 g, silica), eluting with 0-10% MeOH/DCM. The relevant fractions were combined and concentrated in vacuo to yield the title compound (62 mg, 63% yield) as a pale yellow solid. 1H NMR (500 MHz, Chloroform-d) δ 8.69 (d, J=5.9 Hz, 2H), 7.63 (s, 1H), 7.37 (d, J=8.7 Hz, 2H), 7.23 (d, J=5.9 Hz, 2H), 7.22-7.18 (m, 2H), 5.18 (s, 1H), 4.57 (s, 2H), 4.12-4.08 (m, 1H), 4.00 (d, J=9.4 Hz, 1H), 3.93 (d, J=9.1 Hz, 1H), 3.84 (dt, J=12.9, 8.2 Hz, 2H), 3.72 (dd, J=10.1, 3.9 Hz, 1H), 2.81 (s, 3H), 2.47 (dd, J=13.9, 8.7 Hz, 1H), 1.93 (dd, J=13.8, 5.6 Hz, 1H), 1.44 (s, 9H). LCMS (Analytical Method J) Rt=0.89 min, MS (ESIpos): m/z 538.3, 540.3 [M+H]+, Purity=92%.

### Synthesis of 2-[4-(4-chlorophenyl)-5-(pyridin-4-yl)-1H-imidazol-1-yl]-N-methyl-N-{5-oxa-2-azaspiro[3.4]octan-7-yl}acetamide/Intermediate 86

TFA (0.25 mL, 3.26 mmol) was added to a solution of tert-butyl 7-[[2-[4-(4-chlorophenyl)-5-(4-pyridyl)imidazol-1-yl]acetyl]-methyl-amino]-5-oxa-2-azaspiro[3.4]octane-2-carboxylate (Intermediate 86-1) (67 mg, 0.115 mmol) in DCM (1 mL). The reaction was stirred for 3 hours then concentrated in vacuo. The residue was repeatedly taken up in toluene and concentrated in vacuo. The residue was purified by preparative HPLC (Method B1). The relevant fractions were combined and concentrated in vacuo to yield the title compound (21 mg, 42% yield) as a pale yellow solid. LCMS (Analytical Method E) Rt=0.82 min, MS (ESIpos): m/z 438.2, 440.2 [M+H]+, Purity=98%.

### Synthesis of tert-butyl 4-{2-[2-chloro-4-(4-chlorophenyl)-5-(pyridin-4-yl)-1H-imidazol-1-yl]acetyl}piperazine-1-carboxylate/Intermediate 87-1

tert-Butyl 4-[2-[4-(4-chlorophenyl)-5-(4-pyridyl)imidazol-1-yl]acetyl]piperazine-1-carboxylate (Intermediate 4) (503 mg, 0.981 mmol) was dissolved in DCM (8 mL) then N-chlorosuccinimide (155 mg, 1.16 mmol) was added. The mixture was stirred at 40° C. for 2 h. The reaction mixture was concentrated in vacuo then taken up in MeCN (8 ml). The reaction was stirred at 70° C. for 3 h. The reaction mixture was partitioned between water and DCM. The organic phase was separated and the aqueous was extracted with DCM. The combined organics were concentrated in vacuo and the crude product was purified via flash chromatography (25 g, silica) eluting with 0-10% MeOH in DCM to afford the title compound (209 mg, 29% yield) as a colourless gum. 1H NMR (500 MHz, Methanol-d4) δ 8.67-8.64 (m, 2H), 7.39-7.36 (m, 2H), 7.33-7.30 (m, 2H), 7.29-7.25 (m, 2H), 4.92 (s, 2H), 3.58-3.49 (m, 4H), 3.46-3.42 (m, 4H), 1.47 (s, 9H). LCMS (Analytical Method H) Rt=0.62 min, MS (ESIpos): m/z 516.3, 518.3, 520.3 [M+H]+, Purity=71%.

### Synthesis of tert-butyl 4-{2-[4-(4-chlorophenyl)-2-cyclopropyl-5-(pyridin-4-yl)-1H-imidazol-1-yl]acetyl}piperazine-1-carboxylate/Intermediate 87

tert-Butyl 4-[2-[2-chloro-4-(4-chlorophenyl)-5-(4-pyridyl)imidazol-1-yl]acetyl]piperazine-1-carboxylate (Intermediate 87-1) (66 mg, 0.128 mmol), 2-cyclopropyl-4,4,5,5-tetramethyl-1,3,2-dioxaborolane (25 uL, 0.137 mmol) and Na₂CO₃ (30 mg, 0.283 mmol) were suspended in a solution of DME (1.2 mL) and Water (0.3 mL). The mixture was degassed with nitrogen for 5 min then palladium; triphenylphosphane (15 mg, 0.0130 mmol) was added. The reaction was stirred at 100° C. (microwave) for 1 h. Additional 2-cyclopropyl-4,4,5,5-tetramethyl-1,3,2-dioxaborolane (25 uL, 0.137 mmol) was added and the reaction was stirred at 110° C. (microwave) for 1 h. The reaction was stirred at 110° C. for 13 h. The reaction mixture was partitioned between water and EtOAc. The organic phase was separated and the aqueous was extracted with EtOAc. The organics were combined and concentrated in vacuo and the crude product was purified via flash chromatography (silica) eluting with 0-10% 7N ammonia MeOH/DCM. The resulting material was loaded onto a Biotage SCX-2 column. The column was washed with 9:1 DCM/MeOH then the product was eluted with 7N ammonia MeOH. The eluate was concentrated in vacuo to afford the title compound (35 mg, 25% yield). 1H NMR (500 MHz, Methanol-d4) δ 8.6-8.6 (m, 2H), 7.3-7.3 (m, 2H), 7.3-7.2 (m, 4H), 5.0 (s, 2H), 3.6-3.5 (m, 2H), 3.5-3.5 (m, 2H), 3.4-3.4 (m, 4H), 1.9-1.9 (m, 1H), 1.5 (s, 9H), 1.1-1.0 (m, 4H). LCMS (Analytical Method H) Rt=0.61 min, MS (ESIpos): m/z 522.3, 524.4 [M+H]+, Purity=62%.

### Synthesis of 2-[4-(4-chlorophenyl)-5-[2-(difluoromethyl)pyridin-4-yl]-2-hydroxy-1H-imidazol-1-yl]-1-{2,7-diazaspiro[3.5]nonan-7-yl}ethan-1-one/Intermediate 88

TFA (0.11 mL, 1.43 mmol) was added to a stirred solution of tert-butyl 7-[2-[4-(4-chlorophenyl)-5-[2-(difluoromethyl)-4-pyridyl]-2-hydroxy-imidazol-1-yl]acetyl]-2,7-diazaspiro[3.5]nonane-2-carboxylate (Intermediate 68-4) (42 mg, 0.0714 mmol) in DCM (1.1571 mL), and the resulting mixture was stirred at RT for 1.5 h. Reaction was diluted with DCM (2 mL) and carefully quenched with 1 M aq. NaOH (3 mL). The organic layer was separated and the aq. layer extracted with DCM (2×3 mL). The combined organics were collected using a Telos phase separator and evaporated in vacuo to yield the title compound (14 mg, 39% yield) as a white solid. 1H NMR (400 MHz, Chloroform-d) δ 8.60 (d, J=5.0 Hz, 1H), 7.59 (s, 1H), 7.32 (d, J=4.5 Hz, 1H), 7.24 (d, J=8.6 Hz, 2H), 7.13 (d, J=8.6 Hz, 2H), 6.62 (t, J=55.3 Hz, 1H), 4.39 (s, 2H), 3.55-3.35 (m, 6H), 3.33-3.24 (m, 2H), 1.79-1.69 (m, 4H). LCMS (Analytical Method J) Rt=0.60 min, MS (ESIpos): m/z 488.2, 490.2 [M+H]+, Purity=96%.

### Synthesis of 2-[2-chloro-4-(4-chlorophenyl)-5-(pyridin-4-yl)-1H-imidazol-1-yl]-1-{2-methyl-2,7-diazaspiro[3.5]nonan-7-yl}ethan-1-one/Intermediate 89

N-chlorosuccinimide (30 mg, 0.225 mmol) was added to a solution of 2-[4-(4-chlorophenyl)-5-(4-pyridyl)imidazol-1-yl]-1-(2-methyl-2,7-diazaspiro[3.5]nonan-7-yl)ethanone (compound 12 of Table 1) (89 mg, 0.184 mmol) in MeCN (1.5 mL) and the resulting mixture was allowed to stir at 60° C. for 2.5 h. The reaction mixture was quenched with 1M aq. NaOH (2 mL). Organic layer isolated using a Telos phase separator and evaporated under reduced pressure. The resulting residue was purified by preparative HPLC (Method A1) to yield the title compound (24 mg, 28% yield) as an off-white solid. 1H NMR (400 MHz, Chloroform-d) δ 8.71-8.67 (m, 2H), 7.35-7.31 (m, 2H), 7.28-7.25 (m, 2H), 7.22-7.16 (m, 2H), 4.54 (s, 2H), 3.55-3.47 (m, 2H), 3.30-3.23 (m, 2H), 3.08 (d, J=7.0 Hz, 2H), 3.00 (d, J=7.1 Hz, 2H), 2.34 (s, 3H), 1.74-1.67 (m, 4H). LCMS (Analytical Method H) Rt=0.53 min, MS (ESIpos): m/z 470.3 [M+H]+, Purity=100%.

### Example 1.9—More Compounds

### Synthesis of 2-[4-(4-chlorophenyl)-5-(pyridin-4-yl)-1H-imidazol-1-yl]-N-(1-methylpyrrolidin-3-yl)acetamide/Compound 109 from Table 1

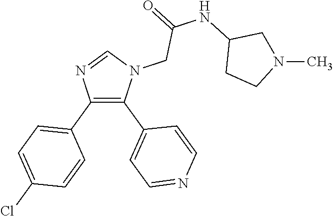 [SMILES: CN1CCC(NC(=O)Cn2cnc(-c3ccc(Cl)cc3)c2-c2ccncc2)C1]

DIPEA (169 uL, 0.969 mmol) and T3P (50%, 231 uL, 0.388 mmol) were dissolved in DMF (1.5 mL) then 2-[4-(4-chlorophenyl)-5-(4-pyridyl)imidazol-1-yl]acetic acid; 2,2,2-trifluoroacetic acid (Intermediate 2a) (105 mg, 0.194 mmol) and (3R)-1-methylpyrrolidin-3-amine (39 mg, 0.388 mmol) were added. The reaction was stirred for 1 h. Reaction was dissolved in EtOAc and washed with water followed by brine. Organic phase was dried (MgSO₄) and concentrated under reduced pressure. Crude product was purified by preparative HPLC (Method A2) and freeze-dried to afford product as a brown solid. Product was loaded onto a 1 g SCX-2 cartridge and flushed with MeOH. Product was eluted with 2.3 M NH₃/MeOH solution and concentrated under reduced pressure to afford the product as a brown solid. Product was purified by preparative HPLC (instrument pump: Gilson 331 & 332; auto injector: Gilson GX281; UV detector: Gilson 159; collector: Gilson GX281; Column: Waters X-Bridge CSH 30×100 mm, 5 μm; eluent A: water+0.2 vol % ammonium hydroxide, eluent B: acetonitrile+0.2 vol % ammonium hydroxide; gradient: 0-2 min 5% B, 2-2.5 min 5-15% B, 2.5-16.5 min 15-30% B, flow 20 mL/min; temperature: 25° C.; UV scan: 215 nm) to receive the title compound (14 mg, 18% yield) as a white solid. 1H NMR (500 MHz, DMSO-d6) δ 8.69-8.61 (m, 2H), 8.23 (d, J=7.3 Hz, 1H), 7.84 (s, 1H), 7.37-7.27 (m, 6H), 4.63-4.49 (m, 2H), 4.08-3.98 (m, 1H), 2.56-2.52 (m, 1H), 2.41 (dd, J=9.4, 6.8 Hz, 1H), 2.19 (s, 4H), 2.11 (dd, J=9.4, 4.0 Hz, 1H), 2.04-1.94 (m, 1H), 1.39-1.30 (m, 1H). LCMS (Analytical Method A) Rt=2.39 min, MS (ESIpos): m/z 396.3 [M+H]+, Purity=98%.

### Synthesis of 2-[4-(4-chlorophenyl)-5-(3-chloropyridin-4-yl)-1H-imidazol-1-yl]-N-[(3R)-1-methylpyrrolidin-3-yl]acetamide/Compound 140-R from Table 1

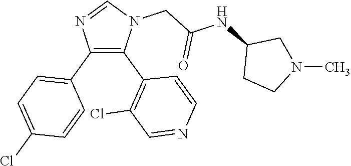 [SMILES: CN1CC[C@@H](NC(=O)Cn2cnc(-c3ccc(Cl)cc3)c2-c2ccncc2Cl)C1]

T3P in EtOAc (50%, 0.21 mL, 0.351 mmol) was added to a stirred solution of 2-[4-(4-chlorophenyl)-5-(3-chloro-4-pyridyl)imidazol-1-yl]acetic acid; 2,2,2-trifluoroacetic acid (Intermediate 46) (81% purity, 100 mg, 0.141 mmol) and DIPEA (0.12 mL, 0.703 mmol) in EtOAc (1.4 mL). After stirring for 5 min (3R)-1-methylpyrrolidin-3-amine (21 mg, 0.211 mmol) was incorporated to the reaction, and the mixture was stirred at 60° C. overnight. 1M aq. NaOH was added (3 mL) and the organic layer was separated. The aqueous layer was extracted with DCM (2×3 mL), and the organic layer were combined and dried using a hydrophobic Telos phase separator and evaporated under reduced pressure. The residue was purified by preparative HPLC (Method A1) to yield the title compound (30 mg, 49% yield) as a pale-yellow solid. 1H NMR (400 MHz, Methanol-d4) δ 8.77 (d, J=0.3 Hz, 1H), 8.58 (dd, J=4.9, 1.9 Hz, 1H), 8.02-7.92 (m, 1H), 7.42 (ddd, J=5.0, 2.0, 0.5 Hz, 1H), 7.35-7.18 (m, 4H), 4.76 (dd, J=16.7, 5.7 Hz, 1H), 4.45 (dd, J=16.6, 1.5 Hz, 1H), 4.23-4.08 (m, 1H), 2.70 (td, J=10.0, 7.2 Hz, 1H), 2.65-2.56 (m, 1H), 2.50-2.41 (m, 1H), 2.33-2.30 (m, 3H), 2.29-2.09 (m, 2H), 1.58-1.37 (m, 1H). LCMS (Analytical Method B) Rt=2.60 min, MS (ESIpos): m/z 430.3, 432.3 [M+H]+, Purity=98%.

### Synthesis of 2-[4-(4-chlorophenyl)-5-(3-chloropyridin-4-yl)-1H-imidazol-1-yl]-N-[(3S)-1-methylpyrrolidin-3-yl]acetamide/Compound 140-S from Table 1

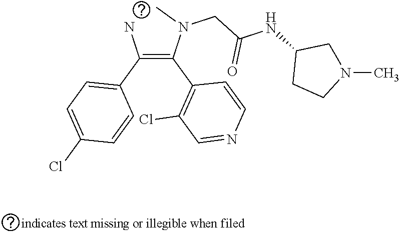 [SMILES: CN1CC[C@H](NC(=O)CN(C)/C(=C(\N)c2ccc(Cl)cc2)c2ccncc2Cl)C1]

T3P in EtOAc (50%, 0.21 mL, 0.351 mmol) was added to a stirred solution of 2-[4-(4-chlorophenyl)-5-(3-chloro-4-pyridyl)imidazol-1-yl]acetic acid; 2,2,2-trifluoroacetic acid (Intermediate 46) (81% purity, 100 mg, 0.141 mmol) and DIPEA (0.12 mL, 0.703 mmol) in EtOAc (1.4 mL). After stirring for 5 min (3S)-1-methylpyrrolidin-3-amine (21 mg, 0.211 mmol) was incorporated to the reaction, and the mixture was stirred at 60° C. overnight. 1 M aq. NaOH was added (3 mL) and the organic layer was separated. The aqueous layer was extracted with DCM (2×3 mL), and the organic layer were combined and dried using a hydrophobic Telos phase separator and evaporated under reduced pressure. The residue was purified by preparative HPLC (Method A1) to yield the title compound (33 mg, 52% yield) as a pale-yellow solid. 1H NMR (400 MHz, Methanol-d4) δ 8.77 (d, J=0.5 Hz, 1H), 8.58 (dd, J=4.9, 1.9 Hz, 1H), 7.98-7.92 (m, 1H), 7.42 (ddd, J=4.9, 2.0, 0.6 Hz, 1H), 7.32-7.23 (m, 4H), 4.76 (dd, J=16.7, 5.6 Hz, 1H), 4.45 (dd, J=16.6, 1.5 Hz, 1H), 4.22-4.10 (m, 1H), 2.69 (td, J=9.9, 7.2 Hz, 1H), 2.65-2.57 (m, 1H), 2.51-2.41 (m, 1H), 2.34-2.30 (m, 3H), 2.29-2.09 (m, 2H), 1.59-1.36 (m, 1H). LCMS (Analytical Method B) Rt=2.60 min, MS (ESIpos): m/z 430.3, 432.3 [M+H]+, Purity=97%.

### Synthesis of 2-[4-(4-chlorophenyl)-5-(pyridin-4-yl)-1H-imidazol-1-yl]-N-(pyridin-4-yl)acetamide/Compound 113 of Table 1

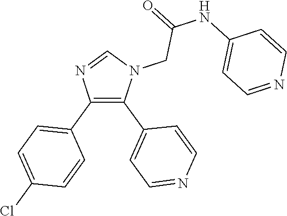 [SMILES: O=C(Cn1cnc(-c2ccc(Cl)cc2)c1-c1ccncc1)Nc1ccncc1]

2-[5-Bromo-4-(4-chlorophenyl)imidazol-1-yl]-N-(4-pyridyl)acetamide (Intermediate 47) (72% purity, 35 mg, 0.0643 mmol), Na₂CO₃ (41 mg, 0.386 mmol) and pyridin-4-ylboronic acid (19 mg, 0.154 mmol) were suspended in water (0.3 mL) and DME (0.7 mL) and degassed with nitrogen for 5 min. palladium; triphenylphosphane (7.4 mg, 6.43 μmol) was added and the reaction was heated at 100° C. for 2 h via microwave radiation. The reaction was diluted with water and extracted with DCM. Organic phase was passed through a phase separator and concentrated under reduced pressure before purifying by preparative HPLC (Method A2) and freeze dried to afford the title compound (2.1 mg, 6.8% yield) as a white solid. 1H NMR (400 MHz, Chloroform-d) δ 8.71-8.64 (m, 2H), 8.55-8.49 (m, 2H), 8.13 (s, 1H), 7.75 (s, 1H), 7.44-7.39 (m, 2H), 7.39-7.34 (m, 2H), 7.25-7.21 (m, 4H), 4.65 (s, 2H). LCMS (Analytical Method A) Rt=1.34 min, MS (ESIpos): m/z 390.2 [M+H]+, Purity=81%.

### Synthesis of N-{4-[4-(4-fluorophenyl)-1-[2-(morpholin-4-yl)-2-oxoethyl]-1H-imidazol-5-yl]pyridin-2-yl}-2,2-dimethylpropanamide/Compound 110 of Table 1

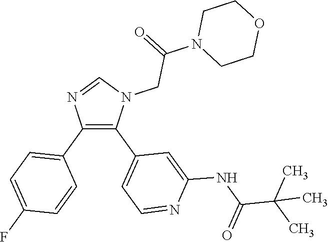 [SMILES: CC(C)(C)C(=O)Nc1cc(-c2c(-c3ccc(F)cc3)ncn2CC(=O)N2CCOCC2)ccn1]

2-[5-[2-(2,2-Dimethylpropanoylamino)-4-pyridyl]-4-(4-fluorophenyl)imidazol-1-yl]acetic acid (Intermediate 48) (79% purity, 15 mg, 0.0299 mmol), morpholine (10 uL, 0.0823 mmol), DIPEA (26 uL, 0.149 mmol) and T3P (50%, 35 uL, 0.0588 mmol) were dissolved in EtOAc (1 mL) and the mixture was stirred at RT for 1 h. The reaction was quenched with water and extracted with EtOAc. The organics were combined and concentrated in vacuo and the crude product was purified via preparative HPLC (Method A1) and lyophilised overnight to afford the title compound (2 mg, 14% yield) as a white solid. 1H NMR (500 MHz, Chloroform-d) δ 8.21 (dd, J=0.6, 5.1 Hz, 1H), 8.12-8.11 (m, 1H), 8.08 (s, 1H), 7.65 (s, 1H), 7.48-7.43 (m, 2H), 6.98-6.92 (m, 2H), 6.91 (dd, J=1.5, 5.2 Hz, 1H), 4.82 (s, 2H), 3.69-3.64 (m, 2H), 3.63-3.58 (m, 4H), 3.41-3.37 (m, 2H), 1.34 (s, 9H). LCMS (Analytical Method B) Rt=2.82 min, MS (ESIpos): m/z 466.3 [M+H]+, Purity=100%.

### Synthesis of N-{4-[4-(4-fluorophenyl)-1-[2-(morpholin-4-yl)-2-oxoethyl]-1H-imidazol-5-yl]pyridin-2-yl}cyclopentanecarboxamide/Compound 79 of Table 1

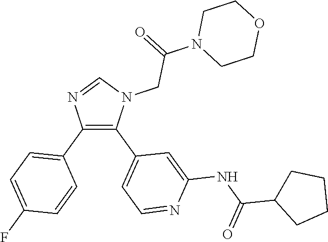 [SMILES: O=C(Nc1cc(-c2c(-c3ccc(F)cc3)ncn2CC(=O)N2CCOCC2)ccn1)C1CCCC1]

2-[5-[2-(Cyclopentanecarbonylamino)-4-pyridyl]-4-(4-fluorophenyl)imidazol-1-yl]acetic acid (Intermediate 49) (32 mg, 0.0721 mmol), morpholine (20 uL, 0.165 mmol) and DIPEA (40-uL, 0.229 mmol) were dissolved in EtOAc (1 mL) then T3P (50%, 65 uL, 0.109 mmol) was added. The reaction was stirred for 1 h. The reaction was stirred for 30 min then concentrated in vacuo. The crude product was purified via preparative HPLC (Method B1) and lyophilised overnight to afford the title compound (19 mg, 55% yield) as a white solid. 1H NMR (500 MHz, DMSO-d6) δ 10.58 (s, 1H), 8.33 (d, J=5.1 Hz, 1H), 7.99 (s, 1H), 7.79 (s, 1H), 7.45-7.39 (m, 2H), 7.13-7.08 (m, 2H), 6.88 (dd, J=1.4, 5.1 Hz, 1H), 4.88 (s, 2H), 3.56-3.53 (m, 2H), 3.46-3.45 (m, 2H), 2.93 (p, J=7.9 Hz, 1H), 1.88-1.80 (m, 2H), 1.71-1.60 (m, 5H), 1.56-1.48 (m, 3H), 0.99-0.90 (m, 2H). LCMS (Analytical Method A) Rt=2.17 min, MS (ESIpos): m/z 478.4 [M+H]+, Purity=99%.

### Synthesis of 2-[2-chloro-4-(4-fluorophenyl)-5-(pyridin-4-yl)-1H-imidazol-1-yl]-1-(4-methylpiperazin-1-yl)ethan-1-one/Compound 112 of Table 1

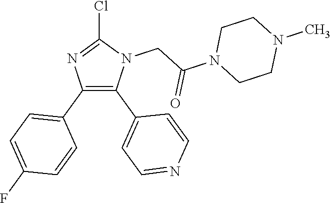 [SMILES: CN1CCN(C(=O)Cn2c(Cl)nc(-c3ccc(F)cc3)c2-c2ccncc2)CC1]

To a stirred solution of 2-[2-chloro-4-(4-fluorophenyl)-5-(4-pyridyl)imidazol-1-yl]acetic acid; 2,2,2-trifluoroacetic acid (Intermediate 50) (50 mg, 0.0893 mmol) and DIPEA (0.062 mL, 0.357 mmol) in EtOAc (1.5 mL) at RT and under nitrogen, T3P (50%, 0.11 mL, 0.179 mmol) was added followed by 1-methylpiperazine (15 uL, 0.134 mmol), and the resulting mixture was allowed to stir at 60° C. overnight. The reaction was diluted with EtOAc (5 mL) and washed with sat. NaHCO₃(5 mL). The aqueous layer was extracted with EtOAc (2×5 mL). The combined organics were dried over MgSO₄, filtered, and evaporated under reduced pressure. The residue was purified preparative HPLC (Method A1) to yield the title compound (28 mg, 71% yield) as a white solid. 1H NMR (500 MHz, Chloroform-d) δ 8.76-8.62 (m, 2H), 7.42-7.32 (m, 2H), 7.28-7.26 (m, 2H), 6.97-6.86 (m, 2H), 4.56 (s, 2H), 3.65 (app t, J=4.7 Hz, 2H), 3.39 (app t, J=4.9 Hz, 2H), 2.39 (app t, J=4.9 Hz, 2H), 2.34 (app t, J=4.8 Hz, 2H), 2.31 (s, 3H). LCMS (Analytical Method B) Rt=2.43 min, MS (ESIpos): m/z 414.3, 416.3 [M+H]+, Purity=96%.

### Synthesis of 2-[2-chloro-4-(4-fluorophenyl)-5-(pyridin-4-yl)-1H-imidazol-1-yl]-1-{2-methyl-2,7-diazaspiro[3.5]nonan-7-yl}ethan-1-one/Compound 115 of Table 1

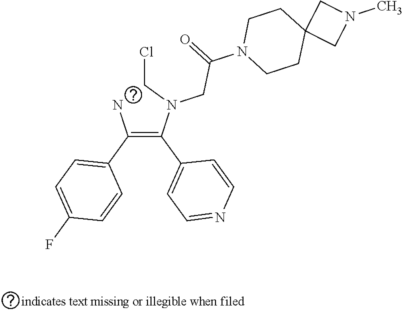 [SMILES: CN1CC2(CCN(C(=O)CN(CCl)/C(=C(\N)c3ccc(F)cc3)c3ccncc3)CC2)C1]

12.7 M Formaldehyde (0.20 mL, 2.54 mmol) was added to a solution of 2-[2-chloro-4-(4-fluorophenyl)-5-(4-pyridyl)imidazol-1-yl]-1-(2,7-diazaspiro[3.5]nonan-7-yl)ethanone (Intermediate 51) (39 mg, 0.0878 mmol) in THF (2 mL) and the mixture was stirred at RT for 30 min. STAB (39 mg, 0.183 mmol) was then added and the reaction was stirred at RT overnight. The reaction was concentrated in vacuo and the residue was partitioned between DCM (3 mL) and 1 M aq. NaOH (3 mL). The aqueous layer was extracted with DCM (2×3 mL). The combined organics were separated using a Telos phase separator. The filtrate was concentrated in vacuo and the residue was purified by preparative HPLC (Method A1) to afford the title compound (23 mg, 57% yield) as a white solid. 1H NMR (400 MHz, Chloroform-d) δ 8.70-8.65 (m, 2H), 7.40-7.33 (m, 2H), 7.27-7.24 (m, 2H), 6.95-6.88 (m, 2H), 4.55 (s, 2H), 3.56-3.47 (m, 2H), 3.32-3.21 (m, 2H), 3.09 (d, J=7.1 Hz, 2H), 3.00 (d, J=7.1 Hz, 2H), 2.35 (s, 3H), 1.76-1.67 (n, 4H). LCMS (Analytical Method A) Rt=1.36 min, MS (ESIpos): m/z 454.3, 456.3 [M+H]+, Purity=97%.

Each of the Compounds listed in Table 1.9.3 were prepared according to the method of Compound 115 of table 1 using the intermediates listed in the “Synthesis” column for such compounds. The final compounds were purified by preparative HPLC Methods, A1.

TABLE 1.9.3

| Example | Synthesis | Structure/Name | Data |
|---|---|---|---|
| Compound 136-S | Intermediate 52(40% yield) | 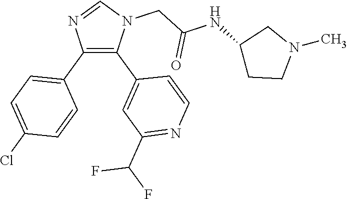 [SMILES: CN1CC[C@H](NC(=O)Cn2cnc(-c3ccc(Cl)cc3)c2-c2ccnc(C(F)F)c2)C1] 2-[4-(4-chlorophenyl)-5-[2- (difluoromethyl)pyridin-4-yl]-1H- imidazol-1-yl]-N-[(3S)-1- methylpyrrolidin-3-yl]acetamide | 1H NMR (500 MHz, DMSO- d6) δ 8.75 (d, J = 5.0 Hz, 1H), 8.28 (d, J = 7.2 Hz, 1H), 7.88 (s, 1H), 7.54 (s, 1H), 7.49 (d, J = 5.0 Hz, 1H), 7.35-7.30 (m, 4H), 6.97 (t, J = 54.7 Hz, 1H), 4.61 (d, J = 16.9 Hz, 1H), 4.57 (d, J = 16.9 Hz, 1H), 4.05-3.98 (m, 1H), 2.53-2.51 (m, 1H), 2.42 (dd, J = 9.4, 6.8 Hz, 1H), 2.24-2.19 (m, 1H), 2.09 (dd, J = 9.5, 4.1 Hz, 1H), 2.03-1.95 (m, 1H), 1.38-1.30 (m, 1H). LCMS (Analytical Method B) Rt = 2.72 min, MS (ESIpos): m/z 446.3, 448.2 [M + H]+, Purity = 97%. |
| Compound 136-R | Intermediate 53 (44% yield) | 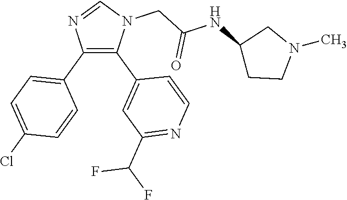 [SMILES: CN1CC[C@@H](NC(=O)Cn2cnc(-c3ccc(Cl)cc3)c2-c2ccnc(C(F)F)c2)C1] 2-[4-(4-chlorophenyl)-5-[2- (difluoromethyl)pyridin-4-yl]-1H- imidazol-1-yl]-N-[(3R)-1- methylpyrrolidin-3-yl]acetamide | 1H NMR (500 MHz, DMSO- d6) δ 8.75 (d, J = 5.0 Hz, 1H), 8.28 (d, J = 7.2 Hz, 1H), 7.88 (s, 1H), 7.54 (s, 1H), 7.49 (d, J = 5.0 Hz, 1H), 7.36-7.30 (m, 4H), 6.97 (t, J = 54.8 Hz, 1H), 4.61 (d, J = 16.8 Hz, 1H), 4.57 (d, J= 16.9 Hz, 1H), 4.05- 3.98 (m, 1H), 2.53-2.51 (m, 1H), 2.42 (dd, J = 9.4, 6.8 Hz, 1H), 2.24-2.19 (m, 1H), 2.09 (dd, J = 9.5, 4.1 Hz, 1H), 2.00 (ddd, J = 16.8, 8.3, 4.1 Hz, 1H), 1.33 (ddd, J = 19.4, 8.1, 4.4 Hz, 1H). LCMS (Analytical Method B) Rt = 2.72 min, MS (ESIpos): m/z 446.3, 448.3 [M + H]+, |
| | | | Purity = 96%. |
| Compound 137 | Intermediate 54, DIPEA (44% yield) | 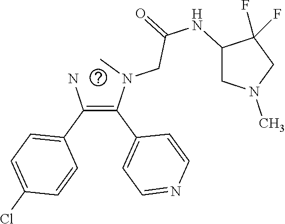 [SMILES: CN1CC(NC(=O)CN(C)/C(=C(\N)c2ccc(Cl)cc2)c2ccncc2)C(F)(F)C1] 2-[4-(4-chlorophenyl)-5-(pyridin-4-yl)- 1H-imidazol-1-yl]-N-(4,4-difluoro-1- methylpyrrolidin-3-yl)acetamide | 1HNMR(400 MHz, DMSO-d6) δ 8.66-8.61 (m, 2H), 8.47 (d, J = 8.3 Hz, 1H), 7.87 (s, 1H), 7.37-7.29 (m, 4H), 7.28- 7.25 (m, 2H), 4.71-4.58 (m, 2H), 4.41-4.27 (m, 1H), 3.10-2.99 (m, 1H), 2.98- 2.91 (m, 1H), 2.73-2.60 (m, 1H), 2.24 (s, 3H), 2.21-2.18 (m, 1H). LCMS (Analytical Method B) Rt = 2.49 min, MS (ESIpos): m/z 432.3, 434.2 [M + H]+, Purity = 100%. |
| Compound 138R | Intermediate 55, DIPEA (53% yield) | 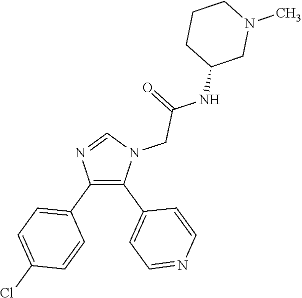 [SMILES: CN1CCC[C@@H](NC(=O)Cn2cnc(-c3ccc(Cl)cc3)c2-c2ccncc2)C1] 2-[4-(4-chlorophenyl)-5-(pyridin- 4-yl)-1H-imidazol-1-yl]-N-[(3R)- 1-methylpiperidin-3-yl]acetamide | 1HNMR(400 MHz, DMSO-d6) δ 8.67-8.62 (m, 2H), 7.96 (d, J = 8.0 Hz, 1H), 7.84 (s, 1H), 7.37-7.27 (m, 6H), 4.64- 4.53 (m, 2H), 3.65-3.56 (m, 1H), 2.42-2.33 (m, 2H), 2.10 (s, 3H), 2.00-1.89 (m, 1H), 1.73-1.62 (m, 1H), 1.59- 1.44 (m, 2H), 1.44-1.33 (m, 1H), 1.10-0.97 (m, 1H). LCMS (Analytical Method B) Rt = 2.40 min, MS (ESIpos): m/z 410.3, 412.2 [M + H]+, Purity = 100%. |
| Compound 138S | Intermediate 56, DIPEA (55% yield) | 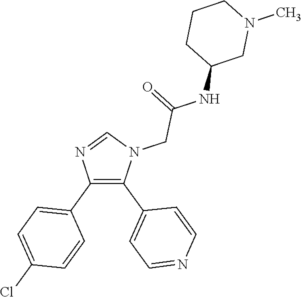 [SMILES: CN1CCC[C@H](NC(=O)Cn2cnc(-c3ccc(Cl)cc3)c2-c2ccncc2)C1] 2-[4-(4-chlorophenyl)-5-(pyridin- 4-yl)-1H-imidazol-1-yl]-N-[(3S)- 1-methylpiperidin-3-yl]acetamide | 1HNMR(400 MHz, DMSO-d6) δ 8.67-8.62 (m, 2H), 7.96 (d, J = 8.0 Hz, 1H), 7.85 (s, 1H), 7.38-7.26 (m, 6H), 4.65- 4.52 (m, 2H), 3.65-3.55 (m, 1H), 2.42-2.32 (m, 2H), 2.09 (d, J = 12.9 Hz, 3H), 1.99-1.89 (m, 1H), 1.73-1.63 (m, 1H), 1.59-1.45 (m, 2H), 1.44-1.33 (m, 1H), 1.09- 0.97 (m, 1H). LCMS (Analytical Method B) Rt = 2.40 min, MS (ESIpos): m/z 410.3, 412.2 [M + H]+, Purity = 100%. |
| Compound 143 | Intermediate 57 (51% yield) | 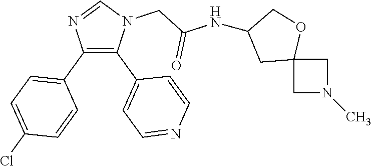 [SMILES: CN1CC2(CC(NC(=O)Cn3cnc(-c4ccc(Cl)cc4)c3-c3ccncc3)CO2)C1] 2-[4-(4-chlorophenyl)-5-(pyridin-4-yl)-1H-imidazol-1-yl]-N- {2-methyl-5-oxa-2-azaspiro[3.4]octan-7-yl]acetamide | 1H NMR (400 MHz, DMSO- d6) δ 8.65 (d, J = 6.0 Hz, 2H), 8.25 (d, J = 6.3 Hz, 1H), 7.85 (s, 1H), 7.35 (d, J = 8.8 Hz, 2H), 7.32-7.26 (m, 4H), 4.57 (s, 2H), 4.12-4.03 (m, 1H), 3.71 (dd, J = 9.0, 6.2 Hz, 1H), 3.28 (dd, J = 9.0, 4.3 Hz, 1H), 3.25-3.19 (m, 2H), 2.91 (dd, J = 7.9, 2.9 Hz, 2H), 2.23- 2.16 (m, 4H), 1.82 (dd, J = 13.1, 4.8 Hz, 1H). LCMS (Analytical Method B) Rt = 2.30 min, MS (ESIpos): m/z 438.3, 440.2 [M + H]+, Purity = 98%. |
| Compound 166-RR | Intermediate 67 (43% yield) | 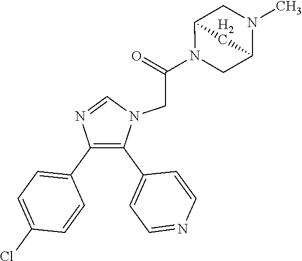 [SMILES: CN1C[C@H]2C[C@@H]1CN2C(=O)Cn1cnc(-c2ccc(Cl)cc2)c1-c1ccncc1] 2-[4-(4-chlorophenyl)-5-(pyridin-4-yl)-1H- imidazol-1-yl]-1-[(1R,4R)-5-methyl-2,5- diazabicyclo[2.2.1]heptan-2-yl]ethan-1-one | 1H NMR (400 MHz, DMSO- d6) δ 8.69-8.62 (m, 2H), 7.80 (s, 1H), 7.43-7.35 (m, 2H), 7.32-7.25 (m, 4H), 4.96- 4.62 (m, 2H), 4.45-4.25 (m, 1H), 3.43-3.31 (m, 2H), 3.14-3.10 (m, 1H), 2.73- 2.69 (m, 1H), 2.28-2.11 (m, 4H), 1.82-1.71 (m, 1H), 1.57- 1.42 (m, 1H). LCMS (Analytical Method B) Rt = 2.22 min, MS (ESIpos): m/z 408.3 [M + H1+, Purity = 99%. |

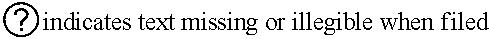

### Synthesis of 2-[4-(4-chlorophenyl)-5-(pyridin-4-yl)-1H-imidazol-1-yl]-1-{2-methyl-5-oxa-2,8-diazaspiro[3.5]nonan-8-yl}ethan-1-one/Compound 145 of Table 1

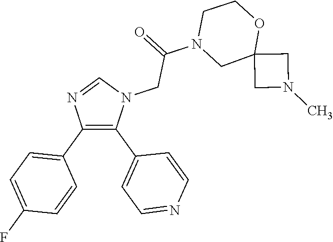 [SMILES: CN1CC2(C1)CN(C(=O)Cn1cnc(-c3ccc(F)cc3)c1-c1ccncc1)CCO2]

tert-Butyl 8-[2-[4-(4-chlorophenyl)-5-(4-pyridyl)imidazol-1-yl]acetyl]-5-oxa-2,8-diazaspiro[3.5]nonane-2-carboxylate (Intermediate 58) (36 mg, 0.0680 mmol) was dissolved in DCM (2 mL) and TFA (0.5 mL) and the mixture was stirred at RT for 16 h. The reaction was concentrated in vacuo. The residue was taken up in DCM (2 mL) and DIPEA (80 uL, 0.459 mmol) then 12 M formaldehyde (40 uL, 0.480 mmol) and STAB (29 mg, 0.136 mmol) were added. The reaction was stirred for 1 h. The reaction was concentrated in vacuo. The crude product was purified via preparative HPLC (Method A) and lyophilised overnight to afford the title compound (23 mg, 77% yield) as a white solid. 1H NMR (400 MHz, DMSO-d6) δ 8.67-8.63 (m, 2H), 7.79 (s, 1H), 7.42-7.37 (m, 2H), 7.31-7.26 (m, 4H), 4.90 (s, 2H), 3.48 (s, 2H), 3.46-3.42 (m, 2H), 3.36-3.32 (m, 2H), 3.09-3.07 (m, 2H), 2.72-2.68 (m, 2H), 2.26 (s, 3H). LCMS (Analytical Method B) Rt=2.32 min, MS (ESIpos): m/z 438.3, 440.2 [M+H]+, Purity=100%.

### Synthesis of 2-[4-(4-chlorophenyl)-5-(pyridin-4-yl)-1H-imidazol-1-yl]-1-{4-methyl-1-oxa-4,9-diazaspiro[5.5]undecan-9-yl}ethan-1-one/Compound 149 of Table 1

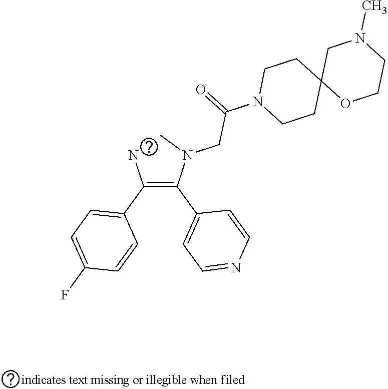 [SMILES: CN1CCOC2(CCN(C(=O)CN(C)/C(=C(\N)c3ccc(F)cc3)c3ccncc3)CC2)C1]

tert-Butyl 9-[2-[4-(4-chlorophenyl)-5-(4-pyridyl)imidazol-1-yl]acetyl]-1-oxa-4,9-diazaspiro[5.5]undecane-4-carboxylate (Intermediate 61) (80 mg, 0.142 mmol) was dissolved in 4M HCl in dioxane (1.5 mL) and stirred at RT for 16 h. The reaction was concentrated in vacuo. The residue was taken up in DCM (1.5 mL) and DIPEA (177 uL, 1.02 mmol) then 12 M formaldehyde (88 uL, 1.06 mmol) and STAB (60 mg, 0.284 mmol) were added. The mixture was stirred for 1 h. The reaction was concentrated in vacuo. The crude product was purified via preparative HPLC (Method A1) and lyophilised overnight to afford the title compound (57 mg, 86% yield) as a white solid. 1H NMR (400 MHz, DMSO-d6) δ 8.67-8.62 (m, 2H), 7.81 (s, 1H), 7.38-7.24 (m, 6H), 4.90 (s, 2H), 3.87-3.79 (m, 1H), 3.63-3.57 (m, 2H), 3.47-3.39 (m, 1H), 3.20-3.10 (m, 1H), 2.94-2.83 (m, 1H), 2.26-2.19 (m, 2H), 2.12 (s, 3H), 2.09 (s, 2H), 1.81-1.71 (m, 2H), 1.27-1.12 (m, 2H). LCMS (Analytical Method B) Rt=2.47 min, MS (ESIpos): m/z 466.4, 468.3 [M+H]+, Purity=100%.

Each of the compounds as listed in Table 1.9.4 were prepared according to the method of Compound 145 (using TFA) or Compound 149 (using 4 M HCl), using the intermediates listed in the “Synthesis” column for such compounds. The final compounds were purified by preparative HPLC Methods, A1 or via trituration with DMSO.

TABLE 1.9.4

| Example | Synthesis | Structure/Name | Data |
|---|---|---|---|
| Compound 148-R | Intermediate 59 (using 4M HCl) (97% yield) | 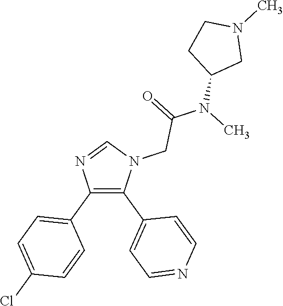 [SMILES: CN1CC[C@@H](N(C)C(=O)Cn2cnc(-c3ccc(Cl)cc3)c2-c2ccncc2)C1] 2-[4-(4-chlorophenyl)-5-(pyridin- 4-yl)-1H-imidazol-1-yl]-N-methyl- N-[(3R)-1-methylpyrrolidin-3- yl]acetamide | 1HNMR(400 MHz, DMSO-d6) δ 8.66-8.62 (m, 2H), 7.77 (s, 1H), 7.41-7.37 (m, 2H), 7.31- 7.23 (m, 4H), 4.83 (s, 2H), 4.68-4.14 (m, 1H), 2.79 (s, 3H), 2.73-2.66 (m, 1H), 2.49- 2.43 (m, 1H), 2.39-2.30 (m, 1H), 2.22 (s, 3H), 2.22- 2.15 (m, 1H), 2.02-1.91 (m, 1H), 1.63-1.52 (m, 1H). LCMS (Analytical Method B) Rt = 2.43 min, MS (ESIpos): m/z 410.3, 412.2 [M + H]+, Purity = 100%. |
| Compound 148-S | Intermediate 60 (using 4M HCl) (96% yield) | 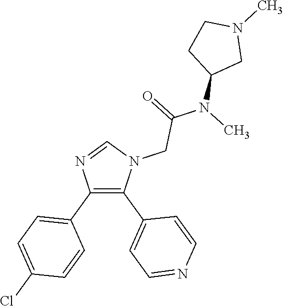 [SMILES: CN1CC[C@H](N(C)C(=O)Cn2cnc(-c3ccc(Cl)cc3)c2-c2ccncc2)C1] 2-[4-(4-chlorophenyl)-5-(pyridin- 4-yl)-1H-imidazol-1-yl]-N-methyl- N-[(3S)-1-methylpyrrolidin-3- yl]acetamide | 1HNMR(400 MHz, DMSO-d6) δ 8.66-8.62 (m, 2H), 7.77 (s, 1H), 7.41-7.36 (m, 2H), 7.31- 7.24 (m, 4H), 4.83 (s, 2H), 4.67-4.15 (m, 1H), 2.79 (s, 3H), 2.73-2.65 (m, 1H), 2.48- 2.42 (m, 1H), 2.38-2.29 (m, 1H), 2.22 (s, 3H), 2.21- 2.15 (m, 1H), 2.01-1.92 (m, 1H), 1.64-1.51 (m, 1H). LCMS (Analytical Method B) Rt = 2.43 min, MS (ESIpos): m/z 410.3, 412.2 [M + H]+, Purity = 100%. |
| Compound 150 | Intermediate 62 (using 4M HCl) (77% yield) | 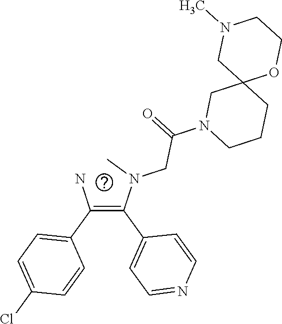 [SMILES: CN1CCOC2(CCCN(C(=O)CN(C)/C(=C(\N)c3ccc(Cl)cc3)c3ccncc3)C2)C1] 2-[4-(4-chlorophenyl)-5-(pyridin- 4-yl)-1H-imidazol-1-yl]-1-{4- methyl-1-oxa-4,8- diazaspiro[5.5]undecan-8- yl}ethan-1-one | 1H NMR (400 MHz, DMSO- d6) δ 8.66-8.59 (m, 2H), 7.76 (s, 1H), 7.42-7.36 (m, 2H), 7.31-7.24 (m, 4H), 4.97- 4.71 (m, 2H), 4.52-4.31 (m, 1H), 3.84-3.63 (m, 1H), 3.62-3.23 (m, 4H), 2.89- 2.66 (m, 1H), 2.33-1.97 (m, 6H), 1.92-1.79 (m, 1H), 1.58- 1.44 (m, 2H), 1.41-1.30 (m, 1H). LCMS (Analytical Method B) Rt = 2.56 min, MS (ESIpos): m/z 466.3, 468.3 [M + H]+, Purity = 100%. |
| Compound 153 | Intermediate 63 (using 4M HCl) (38% yield) | 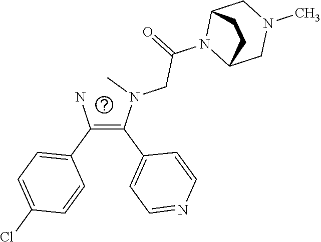 2-[4-(4-chlorophenyl)-5-(pyridin-4-yl)- 1H-imidazol-1-yl]-1-[(1R,5S)-3-methyl- 3,8-diazabicyclo[3.2.1]octan-8-yl]ethan-1-one | 1HNMR(500 MHz, Methanol- d4) δ 8.65-8.61 (m, 2H), 7.89 (s, 1H), 7.42-7.39 (m, 2H), 7.37-7.32 (m, 2H), 7.29- 7.25 (m, 2H), 5.04 (m, 1H), 4.90 (m, 1H), 4.44-4.40 (m, 1H), 4.18-4.13 (m, 1H), 2.71- 2.62 (m, 2H), 2.17 (s, 3H), 1.98-1.92 (m, 2H), 1.89- 1.80 (m, 3H), 1.75-1.66 (m, 1H). LCMS (Analytical Method B) Rt = 2.42 min, MS (ESIpos): m/z 422.3, 424.3 [M + H]+, Purity = 100%. |
| Compound 157-RR | Intermediate 64 (75% yield) | 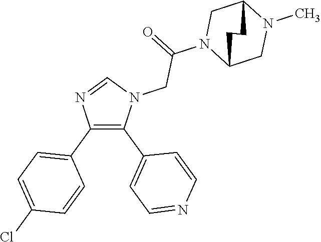 [SMILES: CN1C[C@H]2CC[C@@H]1CN2C(=O)Cn1cnc(-c2ccc(Cl)cc2)c1-c1ccncc1] 2-[4-(4-chlorophenyl)-5-(pyridin-4-yl)-1H- imidazol-1-yl]-1-[(1R,4R)-5-methyl-2,5- diazabicyclo[2.2.2]octan-2-yl]ethan-1-one | 1HNMR(400 MHz, DMSO-d6) δ 8.67-8.63 (m, 2H), 7.82- 7.77 (m, 1H), 7.42-7.37 (m, 2H), 7.32-7.25 (m, 4H), 4.87- 4.71 (m, 2H), 4.18-3.69 (m, 1H), 3.67-3.49 (m, 1H), 3.25-3.14 (m, 1H), 2.80- 2.73 (m, 1H), 2.72-2.66 (m, 1H), 2.65-2.57 (m, 1H), 2.27 (s, 3H), 1.99-1.89 (m, 1H), 1.69-1.60 (m, 1H), 1.60- 1.51 (m, 1H), 1.50-1.41 (m, 1H). LCMS (Analytical Method B) Rt = 2.33 min, MS (ESIpos): m/z 422.4, 424.3 [M + H]+, Purity = 100%. |
| Compound 160 | Intermediate 65 (using TFA) (52% yield) | 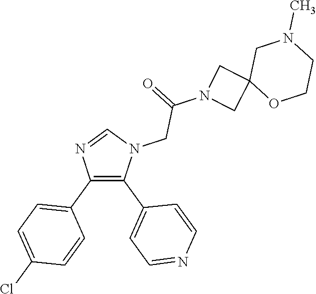 [SMILES: CN1CCOC2(C1)CN(C(=O)Cn1cnc(-c3ccc(Cl)cc3)c1-c1ccncc1)C2] 2-[4-(4-chlorophenyl)-5-(pyridin- 4-yl)-1H-imidazol-1-yl]-1-{8- methyl-5-oxa-2,8-diazaspiro[3.5] nonan-2-yl}ethan-1-one | 1HNMR(500 MHz, DMSO-d6) δ 8.69-8.66 (m, 2H), 7.85 (s, 1H), 7.36-7.29 (m, 6H), 4.67 (s, 2H), 3.84 (s, 2H), 3.67- 3.56 (m, 3H), 3.54-3.48 (m, 1H), 2.39-2.34 (m, 1H), 2.31- 2.25 (m, 2H), 2.22-2.16 (m, 4H). LCMS (Analytical Method B) Rt = 2.34 min, MS (ESIpos): m/z 438.3, 440.2 [M + H]+, Purity = 100%. |
| Compound 157-SS | Intermediate 66 (using 4M HCl) (51% yield) | 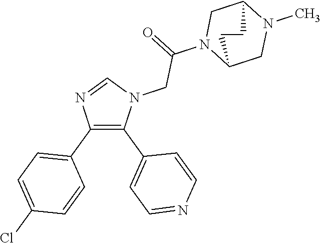 [SMILES: CN1C[C@@H]2CC[C@H]1CN2C(=O)Cn1cnc(-c2ccc(Cl)cc2)c1-c1ccncc1] 2-[4-(4-chlorophenyl)-5-(pyridin-4-yl)-1H- imidazol-1-yl]-1-[(1S,4S)-5-methyl-2,5- diazabicyclo[2.2.2]octan-2-yl]ethan-1-one | 1HNMR(500 MHz, DMSO-d6) δ 8.67-8.63 (m, 2H), 7.86- 7.81 (m, 1H), 7.39-7.25 (m, 6H), 4.93-4.77 (m, 2H), 4.13- 3.72 (m, 1H), 3.67-3.45 (m, 1H), 3.25-3.11 (m, 1H), 2.78-2.71 (m, 1H), 2.71- 2.67 (m, lH), 2.59-2.52 (m, 1H), 2.25-2.22 (m, 3H), 1.94- 1.86 (m, 1H), 1.66-1.56 (m, 1H), 1.56-1.47 (m, 1H), 1.47-1.38 (m, 1H). LCMS (Analytical Method B) Rt = 2.34 min, MS (ESIpos): m/z 422.4, 424.3 [M + H]+, Purity = 100%. |

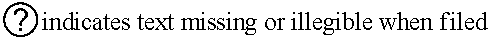

### Synthesis of 2-[2-chloro-4-(4-chlorophenyl)-5-[2-(difluoromethyl)pyridin-4-yl]-1H-imidazol-1-yl]-1-{2-methyl-2,7-diazaspiro[3.5]nonan-7-yl}ethan-1-one/Compound 152 of Table 1

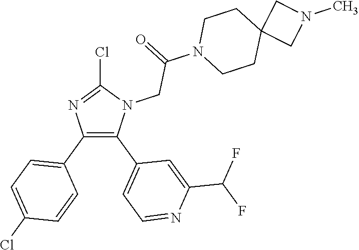 [SMILES: CN1CC2(CCN(C(=O)Cn3c(Cl)nc(-c4ccc(Cl)cc4)c3-c3ccnc(C(F)F)c3)CC2)C1]

12.7 M Formaldehyde (0.15 mL, 1.90 mmol) was added to a solution of 2-[2-chloro-4-(4-chlorophenyl)-5-[2-(difluoromethyl)-4-pyridyl]imidazol-1-yl]-1-(2,7-diazaspiro[3.5]nonan-7-yl)ethanone (Intermediate 68) (28 mg, 0.0520 mmol) in THF (0.4 mL) and the mixture was stirred at RT for 2 h. STAB (23 mg, 0.108 mmol) was then added and the reaction was stirred at RT for 5 h. The reaction was concentrated in vacuo and the residue was partitioned between DCM (3 mL) and 1M aq. NaOH (3 mL). The aqueous layer was extracted with DCM (2×3 mL). The combined organics were separated using a Telos phase separator. The filtrate was concentrated in vacuo. The residue was purified by preparative HPLC (Method B2). The product-containing fractions were combined, basified with 1M aq. NaOH, and extracted with DCM (2×5 mL). The combined organics were dried (hydrophobic frit) and evaporate under reduced pressure to yield the title compound (17 mg, 62% yield) as a white solid. 1H NMR (500 MHz, Chloroform-d) δ 8.67 (d, J=5.0 Hz, 1H), 7.59 (s, 1H), 7.35 (d, J=5.0 Hz, 1H), 7.34-7.30 (m, 2H), 7.23-7.19 (m, 2H), 6.66 (t, J=55.3 Hz, 1H), 4.57 (s, 2H), 3.59-3.50 (m, 2H), 3.31-3.22 (m, 2H), 3.09 (d, J=7.1 Hz, 2H), 3.00 (d, J=7.2 Hz, 2H), 2.35 (s, 3H), 1.76-1.68 (m, 4H). LCMS (Analytical Method A) Rt=2.08 min, MS (ESIpos): m/z 520.2, 522.2, 524.2 [M+H]+, Purity=99%.

### Synthesis of N-{4-[4-(4-fluorophenyl)-1-(2-{2-oxa-6-azaspiro[3.3]heptan-6-yl}-2-oxoethyl)-1H-imidazol-5-yl]pyridin-2-yl}benzamide/Compound 116 of Table 1

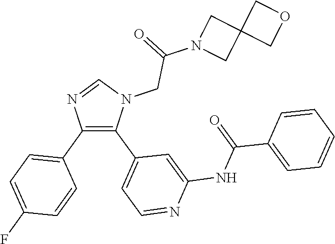 [SMILES: O=C(Nc1cc(-c2c(-c3ccc(F)cc3)ncn2CC(=O)N2CC3(COC3)C2)ccn1)c1ccccc1]

2-[5-Bromo-4-(4-fluorophenyl)imidazol-1-yl]-1-(2-oxa-6-azaspiro[3.3]heptan-6-yl)ethanone (Intermediate 69) (90 mg, 0.213 mmol), N-[4-(4,4,5,5-tetramethyl-1,3,2-dioxaborolan-2-yl)-2-pyridyl]benzamide (Intermediate 69-2) (80% purity, 100-mg, 0.247 mmol), Na₂CO₃ (60 mg, 0.566 mmol) were suspended in DME (1 mL) and water (0.3 mL) and degassed with nitrogen for 5 min. palladium; triphenylphosphane (25 mg, 0.0216 mmol) was added and the mixture was sealed under nitrogen and stirred at 100° C. (microwave) for 1 h. The reaction was quenched with water and extracted with DCM. The organics were combined and concentrated in vacuo. The crude product was taken up in DMSO/MeOH then water was added, causing a precipitate to form. The solid was removed by filtration and the filtrate was concentrated in vacuo. The crude product was purified via preparative HPLC (Method A1) and lyophilised overnight to afford the title compound (8 mg, 8% yield). 1H NMR (500 MHz, DMSO-d6) δ 10.98 (s, 1H), 8.45 (dd, J=5.1, 0.7 Hz, 1H), 8.10-8.08 (m, 1H), 8.04-8.00 (m, 2H), 7.82 (s, 1H), 7.63-7.59 (m, 1H), 7.55-7.50 (m, 2H), 7.46-7.41 (m, 2H), 7.14-7.08 (m2H), 7.03 (dd, J=5.1, 1.5 Hz, 1H), 4.61 (s, 2H), 4.58-4.52 (m, 4H), 4.12 (s, 2H), 4.02 (s, 2H). LCMS (Analytical Method B) Rt=2.69 min, MS (ESIpos): m/z 498.3 [M+H]+, Purity=94%.

### Synthesis of N-{4-[4-(4-fluorophenyl)-1-(2-{2-oxa-6-azaspiro[3.4]octan-6-yl}-2-oxoethyl)-1H-imidazol-5-yl]pyridin-2-yl}benzamide/Compound 130 of Table 1

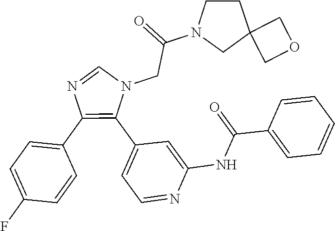 [SMILES: O=C(Nc1cc(-c2c(-c3ccc(F)cc3)ncn2CC(=O)N2CCC3(COC3)C2)ccn1)c1ccccc1]

2-[5-Bromo-4-(4-fluorophenyl)imidazol-1-yl]-1-(2-oxa-7-azaspiro[3.4]octan-7-yl)ethanone (Intermediate 70) (65 mg, 0.165 mmol), N-[4-(4,4,5,5-tetramethyl-1,3,2-dioxaborolan-2-yl)-2-pyridyl]benzamide (Intermediate 69-2) (60 mg, 0.185 mmol) and Na₂CO₃ (45 mg, 0.425 mmol) were suspended in water (0.2 mL) and DME (0.8 mL) and the mixture was degassed with nitrogen for 10 min, then palladium; triphenylphosphane (20 mg, 0.0173 mmol) was added. The mixture was sealed and stirred at 100° C. (microwave) for 2.5 h. The reaction was filtered and the filtrate was partitioned with water and EtOAc. The organic phase was separated and concentrated in vacuo. The crude product was purified via preparative HPLC (Method A1) and lyophilised overnight to afford the title compound (29 mg, 33% yield) as an off-white solid. 1H NMR (500 MHz, DMSO-d6) δ 10.94 (s, 1H), 8.41 (d, J=5.1 Hz, 1H), 8.09 (s, 1H), 8.02-7.97 (m, 2H), 7.82 (s, 1H), 7.63-7.58 (m, 1H), 7.55-7.49 (m, 2H), 7.48-7.42 (m, 2H), 7.15-7.08 (m, 2H), 7.01-6.97 (m, 1H), 4.81 (m, 2H), 4.46-4.33 (m, 4H), 3.60 (s, 1H), 3.52 (s, 1H), 3.35-3.31 (m, 2H), 2.14-2.09 (m, 1H), 2.04-1.99 (m, 1H). LCMS (Analytical Method B) Rt=2.73 min, MS (ESIpos): m/z 512.4 [M+H]+, Purity=96%.

### Synthesis of N-{4-[4-(4-fluorophenyl)-1-(2-{6-oxa-2-azaspiro[3.4]octan-2-yl}-2-oxoethyl)-1H-imidazol-5-yl]pyridin-2-yl}benzamide/Compound 131 of Table 1

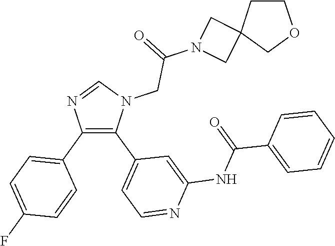 [SMILES: O=C(Nc1cc(-c2c(-c3ccc(F)cc3)ncn2CC(=O)N2CC3(CCOC3)C2)ccn1)c1ccccc1]

2-[5-Bromo-4-(4-fluorophenyl)imidazol-1-yl]-1-(6-oxa-2-azaspiro[3.4]octan-2-yl)ethanone (Intermediate 71) (48 mg, 0.119 mmol), N-[4-(4,4,5,5-tetramethyl-1,3,2-dioxaborolan-2-yl)-2-pyridyl]benzamide (Intermediate 69-2) (45 mg, 0.139 mmol) and Na₂CO₃ (30 mg, 0.283 mmol) were suspended in water (0.2 mL) and DME (0.8 mL) and the mixture was degassed with nitrogen for 10 min, then palladium; triphenylphosphane (15 mg, 0.0130 mmol) was added. The mixture was sealed and stirred at 100° C. (microwave) for 1 h. The reaction was retreated with N-[4-(4,4,5,5-tetramethyl-1,3,2-dioxaborolan-2-yl)-2-pyridyl]benzamide (20 mg, 0.0617 mmol) and stirred at 100° C. (microwave) for 1 h. The reaction was filtered and the filtrate was partitioned between water and EtOAc. The organic layer was separated and concentrated in vacuo. The crude product was purified via preparative HPLC (Method A1) and lyophilised overnight to afford the title compound (27 mg, 43% yield) as a pale pink solid. 1H NMR (500 MHz, DMSO-d6) δ 11.00 (s, 1H), 8.47 (dd, 1H), 8.14 (s, 1H), 8.04-8.00 (m, 2H), 7.84 (s, 1H), 7.63-7.58 (m, 1H), 7.55-7.49 (m, 2H), 7.47-7.42 (m, 2H), 7.15-7.08 (m, 2H), 7.04 (dd, J=5.1, 1.5 Hz, 1H), 4.69-4.60 (m, 2H), 3.96-3.89 (m, 2H), 3.81 (s, 2H), 3.65-3.64 (m, 2H), 3.64-3.59 (m, 2H), 2.04-1.95 (m, 2H). LCMS (Analytical Method B) Rt=2.77 min, MS (ESIpos): m/z 512.4 [M+H]+, Purity=97%.

### Synthesis of 2-[4-(4-chlorophenyl)-5-[2-(trifluoromethyl)pyridin-4-yl]-1H-imidazol-1-yl]-1-{2-methyl-2,7-diazaspiro[3.5]nonan-7-yl}ethan-1-one/Compound 120 of Table 1

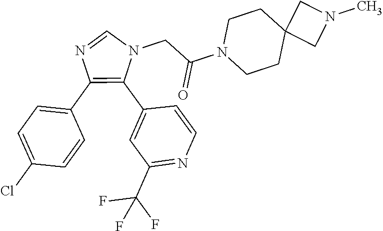 [SMILES: CN1CC2(CCN(C(=O)Cn3cnc(-c4ccc(Cl)cc4)c3-c3ccnc(C(F)(F)F)c3)CC2)C1]

Formaldehyde (37% in water) (37%, 63 mg, 0.781 mmol) was added to a solution of 2-[4-(4-chlorophenyl)-5-[2-(trifluoromethyl)-4-pyridyl]imidazol-1-yl]-1-(2,7-diazaspiro[3.5]nonan-7-yl)ethanone; 2,2,2-trifluoroacetic acid (Intermediate 72) (65 mg, 0.0781 mmol) in DCM (1 mL) and MeOH (0.2 mL). The reaction was stirred for 5 minutes then STAB (50 mg, 0.234 mmol) was added and the reaction stirred for 2 hours. The reaction was quenched into water. The aqueous layer was extracted into EtOAc (15 mL) three times, the combined organics washed with brine, dried over MgSO₄ and concentrated in vacuo. The residue was purified by preparative HPLC (Method A1) The relevant fractions were combined, the solvent volume reduced in vacuo and freeze dried to yield the title compound as a white solid (20 mg, 48% yield). 1H NMR (500 MHz, DMSO-d6) δ 8.79 (d, J=5.0 Hz, 1H), 7.86 (s, 1H), 7.74 (s, 1H), 7.54 (dd, J=5.0, 1.2 Hz, 1H), 7.38-7.32 (m, 4H), 5.00 (s, 2H), 3.30-3.26 (m, 2H), 3.26-3.21 (m, 2H), 2.89 (d, J=6.7 Hz, 2H), 2.86 (d, J=6.3 Hz, 2H), 2.20 (s, 3H), 1.52-1.47 (m, 2H), 1.47-1.40 (m, 2H). LCMS (Analytical Method B) Rt=3.19 min, MS (ESIpos): m/z 504.4, 506.4 [M+H]+, Purity=95%.

### Synthesis of 2-[4-(4-chlorophenyl)-5-[2-(difluoromethyl)pyridin-4-yl]-1H-imidazol-1-yl]-1-{2-methyl-2,7-diazaspiro[3.5]nonan-7-yl}ethan-1-one/Compound 121 of Table 1

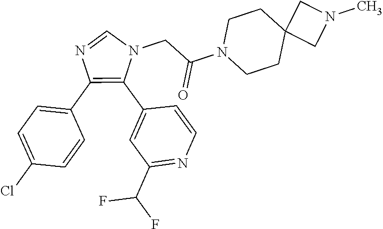 [SMILES: CN1CC2(CCN(C(=O)Cn3cnc(-c4ccc(Cl)cc4)c3-c3ccnc(C(F)F)c3)CC2)C1]

Formaldehyde (37% in water) (37%, 63 uL, 0.737 mmol) was added to a solution of 2-[4-(4-chlorophenyl)-5-[2-(difluoromethyl)-4-pyridyl]imidazol-1-yl]-1-(2,7-diazaspiro[3.5]nonan-7-yl)ethanone; 2,2,2-trifluoroacetic acid (Intermediate 73) (60 mg, 0.0737 mmol) in 0CM (1 mL) and MeOH (0.2 mL). The reaction was stirred for 5 minutes then STAB (47 mg, 0.221 mmol) was added and the reaction stirred for 1 hour. The reaction was quenched into water. The aqueous layer was extracted into EtOAc (15 mL) three times, the combined organics washed with brine, dried over MgSO₄ and concentrated in vacuo. The residue was purified by preparative HPLC (Method A1). The relevant fractions were combined, the solvent volume reduced in vacuo and freeze dried to yield the title compound as a white solid (20 mg, 54% yield). 1H NMR (500 MHz, DMSO-d6) δ 8.72 (d, J=5.0 Hz, 1H), 7.84 (s, 1H), 7.53 (s, 1H), 7.42 (d, J=4.5 Hz, 1H), 7.37-7.30 (m, 4H), 6.96 (t, J=54.8 Hz, 1H), 4.94 (s, 2H), 3.30-3.27 (m, 2H), 3.25-3.21 (m, 2H), 2.89 (d, J=6.7 Hz, 2H), 2.86 (d, J=6.7 Hz, 2H), 2.19 (s, 3H), 1.52-1.48 (m, 2H), 1.48-1.44 (m, 2H). LCMS (Analytical Method B) Rt=2.87 min, MS (ESIpos): m/z 486.4, 488.4 [M+H]+, Purity=97%.

### Synthesis of 2-[4-(4-chlorophenyl)-5-[2-(difluoromethyl)pyridin-4-yl]-1H-imidazol-1-yl]-1-{2-methyl-2,5-diazaspiro[3.4]octan-5-yl}ethan-1-one/Compound 122 of Table 1

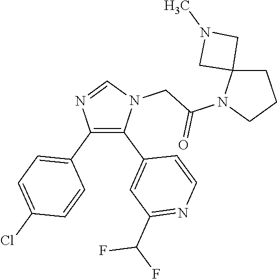 [SMILES: CN1CC2(CCCN2C(=O)Cn2cnc(-c3ccc(Cl)cc3)c2-c2ccnc(C(F)F)c2)C1]

T3P (50% in EtOAc) (50%, 126 uL, 0.211 mmol) was added to a solution of 2-[4-(4-chlorophenyl)-5-[2-(difluoromethyl)-4-pyridyl]imidazol-1-yl]acetic acid; 2,2,2-trifluoroacetic acid (Intermediate 10) (25 mg, 0.0422 mmol), DIPEA (74 uL, 0.422 mmol) and 1-methyl-1,6-diazaspiro[3.4]octane (27 mg, 0.211 mmol) in EtOAc (1 mL). The reaction was stirred for 1 hour then quenched into water. The aqueous layer was extracted into EtOAc (15 mL) three times, the combined organics washed with brine, dried over MgSO₄ and concentrated in vacuo. The residue was purified by preparative HPLC (Method A1). The relevant fractions were combined, the solvent volume reduced in vacuo and freeze dried to afford the title compound (8.0 mg, 40% yield) as a white solid. 1H NMR (400 MHz, DMSO-d6) δ 8.72 (d, J=4.3 Hz, 1H), 7.83 (s, 1H), 7.56 (s, 1H), 7.46 (d, J=4.7 Hz, 1H), 7.37 (d, J=8.6 Hz, 2H), 7.29 (d, J=8.5 Hz, 2H), 6.90 (t, J=54.9 Hz, 2H), 4.84-4.73 (m, 2H), 3.42-3.32 (m, 3H), 3.28 (d, J=10.4 Hz, 2H), 2.06 (d, J=10.6 Hz, 4H), 1.95 (dt, J=17.1, 9.3 Hz, 3H), 1.81 (d, J=5.3 Hz, 1H). LCMS (Analytical Method B) Rt=2.73 min, MS (ESIpos): m/z 472.3, 474.3 [M+H]+, Purity=100%.

Each of the compounds as listed in Table 1.9.5 were prepared according to the method of Compound 122, using the intermediates listed in the “Synthesis” column for such compounds. The final compounds were purified by preparative HPLC Methods, A1, B1 or B2 or for 168-R: custom method (instrument pump: Gilson 331 & 332; auto injector: Gilson GX281; UV detector: Gilson 159; collector: Gilson GX281; Column: Waters X-Bridge CSH 30×100 mm, 5 μm; eluent A: water+0.1 vol % formic acid, eluent B: acetonitrile+0.1 vol % formic acid; gradient: 0-2 min 1% B, 2-16 min 1-55%, flow 20 mL/min; temperature: 25° C.; UV scan: 215 nm).

TABLE 1.9.5

| Example | Synthesis | Structure/Name | Data |
|---|---|---|---|
| Compound 124 | Intermediate 10 and 2-oxa-6-azaspiro [3.3]heptane (77% yield) | 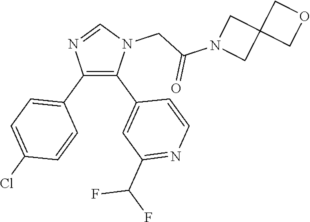 [SMILES: O=C(Cn1cnc(-c2ccc(Cl)cc2)c1-c1ccnc(C(F)F)c1)N1CC2(COC2)C1] 2-[4-(4-chlorophenyl)-5-[2- (difluoromethyl)pyridin-4-yl]-1H- imidazol-1-yl]-1-{2-oxa-6- azaspiro[3.3]heptan-6-yl}ethan- 1-one | 1H NMR (500 MHz, DMSO- d6) δ 8.76 (d, J = 5.0 Hz, 1H), 7.86 (s, 1H), 7.57 (s, 1H), 7.48 (d, J = 4.4 Hz, 1H), 7.36- 7.30 (m, 4H), 6.99 (t, J = 54.8 Hz, 1H), 4.64 (s, 2H), 4.61 (s, 4H), 4.16 (s, 2H), 3.95 (s, 2H). LCMS (Analytical Method A) Rt = 2.24 min, MS (ESIpos): m/z 445.3, 447.3 [M + H]+, Purity = 99%. |
| Compound 125 | Intermediate 72-3 and 1-methyl-1, 6- diazaspiro[3.4] octane (47% yield) | 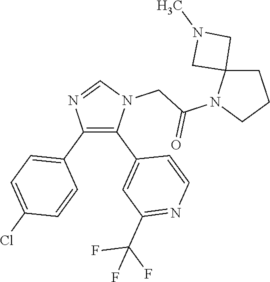 [SMILES: CN1CC2(CCCN2C(=O)Cn2cnc(-c3ccc(Cl)cc3)c2-c2ccnc(C(F)(F)F)c2)C1] 2-[4-(4-chlorophenyl)-5-[2-(trifluoromethyl) pyridin-4-yl]-1H-imidazol-1-yl]-1-{2- methyl-2,5-diazaspiro[3.4]octan-5-yl}ethan- 1-one | 1H NMR (400 MHz, DMSO- d6) δ 8.79 (d, J = 4.7 Hz, 1H), 7.85 (s, 1H), 7.73 (s, 1H), 7.60 (d, J = 4.9 Hz, 1H), 7.37 (d, J = 8.6 Hz, 2H), 7.31 (d, J = 8.7 Hz, 2H), 4.88-4.80 (m, 2H), 3.42 (s, 1H), 3.33 (dd, J = 22.1, 10.8 Hz, 3H), 2.06 (d, J = 12.6 Hz, 3H), 1.95 (td, J = 17.2, 16.4, 7.2 Hz, 3H), 1.85-1.77 (m, 1H). LCMS (Analytical Method B) Rt = 3.05 min, MS (ESIpos): m/z 490.3, 492.3 [M + H]+, Purity = 99%. |
| Compound 126 | Intermediate 72-3 and 2-oxa-6- azaspiro[3.3] heptane (49% yield) | 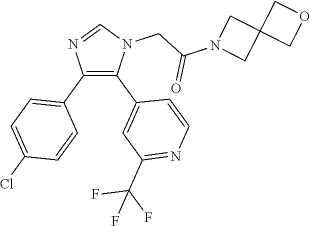 [SMILES: O=C(Cn1cnc(-c2ccc(Cl)cc2)c1-c1ccnc(C(F)(F)F)c1)N1CC2(COC2)C1] 2-[4-(4-chlorophenyl)-5-[2- (trifluoromethyl)pyridin-4-yl]-1H- imidazol-1-yl]-1-{2-oxa-6- azaspiro[3.3]heptan-6-yl}ethan-1-one | 1H NMR (500 MHz, DMSO- d6) δ 8.83 (d, J = 5.0 Hz, 1H), 7.88 (s, 1H), 7.79 (s, 1H), 7.61 (dd, J = 5.0, 1.2 Hz, 1H), 7.36- 7.32 (m, 4H), 4.69 (s, 2H), 4.62 (s, 4H), 4.20 (s, 2H), 3.96 (s, 2H). LCMS (Analytical Method A) Rt = 2.61 min, MS (ESIpos): m/z 463.3, 465.3 [M + H]+, Purity = 94%. |
| Compound 132 | Intermediate 2a and 4H, 5H, 6H, 7H- imidazo[1, 5-a] pyrazine (41% yield) | 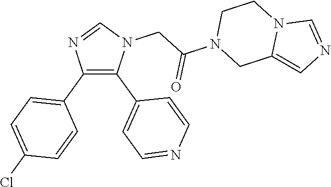 [SMILES: O=C(Cn1cnc(-c2ccc(Cl)cc2)c1-c1ccncc1)N1CCn2cncc2C1] 2-[4-(4-chlorophenyl)-5-(pyridin-4-yl)-1H-imidazol- 1-yl]-1-{5H,6H,7H,8H-imidazo[1,5-a] pyrazin-7-yl}ethan-1-one | 1H NMR (400 MHz, DMSO- d6) δ 8.58 (d, J = 5.9 Hz, 2H), 7.80 (s, 1H), 7.55 (s, 1H), 7.41- 7.35 (m, 2H), 7.31-7.24 (m, 4H), 6.74 (s, 1H), 4.97 (s, 2H), 4.63 (s, 2H), 4.00 (s, 2H), 3.77-3.72 (m, 2H). LCMS (Analytical Method B) Rt = 2.25 min, MS (ESIpos): m/z 419.3, 421.3 [M + H]+, Purity = 94%. |
| Compound 134-R | Intermediate 2a and (3R)-1- methylpyrrolidin-3- amine (64% yield) | 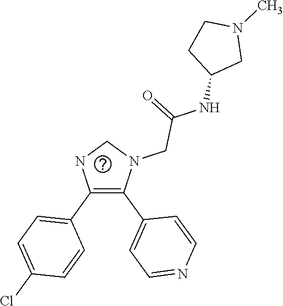 [SMILES: CN1CC[C@@H](NC(=O)CN2CNC(c3ccc(Cl)cc3)=C2c2ccncc2)C1] 2-[4-(4-chlorophenyl)-5-(pyridin- 4-yl)-1H-imidazol-1-yl]-N-[(3R)- 1-methylpyrrolidin-3-yl]acetamide | 1H NMR (400 MHz, DMSO- d6) δ 8.66-8.61 (m, 2H), 8.23 (d, J = 7.3 Hz, 1H), 7.84 (s, 1H), 7.37-7.27 (m, 6H), 4.61-4.50 (m, 2H), 4.07- 3.98 (m, 1H), 2.56-2.51 (m, 1H), 2.44-2.38 (m, 1H), 2.24- 2.16 (m, 4H), 2.14-2.08 (m, 1H), 2.05-1.95 (m, 1H), 1.39-1.29 (m, 1H). LCMS (Analytical Method B) Rt = 2.31 min, MS (ESIpos): m/z 396.3, 398.2 [M + H]+, Purity = 98%. |
| Compound 134-S | Intermediate 2a and (3S)-1- methylpyrrolidin-3- amine (91% yield) | 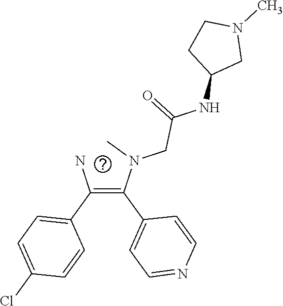 [SMILES: CN1CC[C@H](NC(=O)CN(C)/C(=C(\N)c2ccc(Cl)cc2)c2ccncc2)C1] 2-[4-(4-chlorophenyl)-5-(pyridin- 4-yl)-1H-imidazol-1-yl]-N-[(3S)- 1-methylpynrolidin-3-yl]acetamide | 1HNMR(400 MHz, DMSO-d6) δ 8.67-8.62 (m, 2H), 8.23 (d, J = 7.3 Hz, 1H), 7.84 (s, 1H), 7.37-7.26 (m, 6H), 4.62- 4.48 (m, 2H), 4.08-3.98 (m, 1H), 2.56-2.51 (m, 1H), 2.44-2.38 (m, 1H), 2.25- 2.15 (m, 4H), 2.14-2.08 (m, 1H), 2.06-1.95 (m, 1H), 1.39- 1.29 (m, 1H). LCMS (Analytical Method B) Rt = 2.31 min, MS (ESIpos): m/z 396.3, 398.2 [M + H]+, Purity = 99%. |
| Compound 135-S | Intermediate 2a and (3S)- tetrahydrofuran-3- amine (67% yield) | 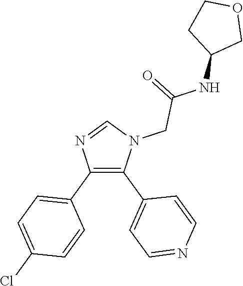 [SMILES: O=C(Cn1cnc(-c2ccc(Cl)cc2)c1-c1ccncc1)N[C@H]1CCOC1] 2-[4-(4-chlorophenyl)-5-(pyridin- 4-yl)-1H-imidazol-1-yl]-N-[(3S)- oxolan-3-yl]acetamide | 1HNMR(400 MHz, DMSO-d6) δ 8.67-8.62 (m, 2H), 8.30 (d, J = 6.7 Hz, 1H), 7.86 (s, 1H), 7.38-7.27 (m, 6H), 4.58 (s, 2H), 4.16-4.06 (m, 1H), 3.70-3.58 (m, 3H), 3.29- 3.25 (m, 1H), 2.03-1.93 (m, 1H), 1.56-1.46 (m, 1H). LCMS (Analytical Method A) Rt = 1.58 min, MS (ESIpos): m/z 383.3, 385.2 [M + H]+, Purity = 100%. |
| Compound 135-R | Intermediate 2a and (3R)-tetrahydrofuran- 3-amine (83% yield) | 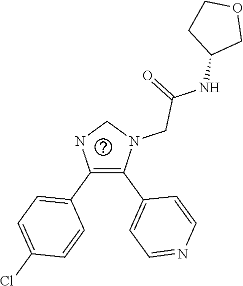 [SMILES: O=C(CN1CNC(c2ccc(Cl)cc2)=C1c1ccncc1)N[C@@H]1CCOC1] 2-[4-(4-chlorophenyl)-5-(pyridin- 4-yl)-1H-imidazol-1-yl]-N-[(3R)- oxolan-3-yl]acetamide | 1HNMR(500 MHz, DMSO-d6) δ 8.67-8.63 (m, 2H), 8.31 (d, J = 6.7 Hz, 1H), 7.86 (s, 1H), 7.37-7.28 (m, 6H), 4.58 (s, 2H), 4.14-4.08 (m, 1H), 3.69-3.59 (m, 3H), 3.27 (dd, J = 9.0, 3.5 Hz, 1H), 2.03- 1.94 (m, 1H), 1.55-1.47 (m, 1H). LCMS (Analytical Method A) Rt = 1.58 min, MS (ESIpos): m/z 383.3, 385.2 [M + H]+, Purity = 100%. |
| Compound 154 | Intermediate 2a and 4-(azetidin-3- yl)morpholine (31% yield) | 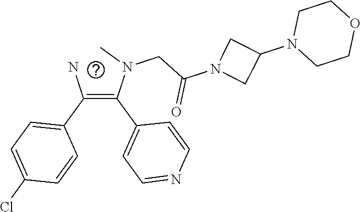 2-[4-(4-chlorophenyl)-5-(pyridin- 4-yl)-1H-imidazol-1-yl]-1-[3- (morpholin-4-yl)azetidin-1- yl]ethan-1-one | 1H NMR (400 MHz, Chloroform-d) δ 8.74-8.70 (m, 2H), 7.67 (s, 1H), 7.39- 7.34 (m, 2H), 7.29-7.26 (m, 2H), 7.23-7.18 (m, 2H), 4.38 (s, 2H), 4.06-3.98 (m, 1H), 3.94-3.84 (m, 2H), 3.82- 3.76 (m, 1H), 3.73 (t, J = 4.6 Hz, 4H), 3.19-3.11 (m, 1H), 2.39-2.25 (m, 4H). LCMS (Analytical Method B) Rt = 2.25 min, MS (ESIpos): m/z 438.3 [M + H]+, Purity = 100%. |
| Compound 162 | Intermediate 2a and 1-(1-ethyl-1H- imidazol-2-yl)- N-methylmethan amine (30% yield) | 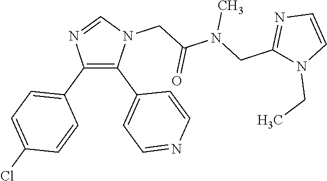 [SMILES: CCn1ccnc1CN(C)C(=O)Cn1cnc(-c2ccc(Cl)cc2)c1-c1ccncc1] 2-[4-(4-chlorophenyl)-5-(pyridin- 4-yl)-1H-imidazol-1-yl]-N-[(1- ethyl-1H-imidazol-2-yl)methyl]- N-methylacetamide | 1HNMR(500 MHz, Chloroform-d) δ 8.67-8.63 (m, 2H), 7.66 (s, 1H), 7.38- 7.34 (m, 2H), 7.23-7.18 (m, 4H), 7.03-7.01 (m, 1H), 6.93- 6.91 (m, 1H), 4.66 (d, J = 4.6 Hz, 4H), 3.97 (q, J = 7.3 Hz, 2H), 3.04 (s, 3H), 1.32 (t, J = 7.3 Hz, 3H). LCMS (Analytical Method A) Rt = 1.33 min, MS (ESIpos): m/z 435.2 [M + H]+, Purity = 100%. |
| Compound 163 | Intermediate 2a 1-(pyrrolidin-3- yl)-1H-imidazole hydrochloride (17% yield) | 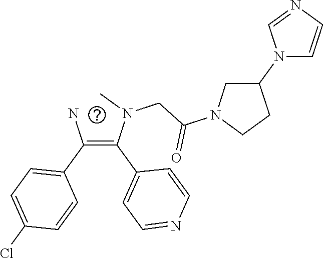 2-[4-(4-chlorophenyl)-5-(pyridin- 4-yl)-1H-imidazol-1-yl]-1-[3-(1H- imidazol-1-yl)pyrrolidin-1-yl]ethan-1-one | 1HNMR(400 MHz, DMSO-d6) δ 8.68-8.62 (m, 2H), 7.81 (s, 1H), 7.64 (s, 1H), 7.41-7.36 (m, 2H), 7.32-7.26 (m, 4H), 7.13-7.06 (m, 1H), 6.93 (s, 1H), 4.95-4.75 (m, 3H), 3.93- 3.72 (m, 1H), 3.58-3.35 (m, 3H), 2.44-2.27 (m, 1H), 2.26- 2.05 (m, 1H). LCMS (Analytical Method A) Rt = 1.18 min, MS (ESIpos): m/z 433.2 [M + H]+, Purity = 99%. |
| Compound 168-R | Intermediate 83 and (3S)-N, 1- dimethylpyrrolidin- 3-amine (10% yield) | 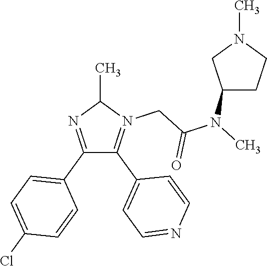 2-[3-(4-fluorophenyl)-1-methyl-4-(pyridin- 4-yl)-1H-pyrazol-5-yl]-N-methyl-N-[(3R)- 1-methylpyrrolidin-3-yl]acetamide | 1HNMR(400 MHz, DMSO-d6) δ 8.57-8.47 (m, 2H), 7.38- 7.27 (m, 2H), 7.14-7.03 (m, 4H), 5.09-4.36 (m, 1H), 3.80 (s, 3H), 2.88 (s, 3H), 2.76- 2.69 (m, 1H), 2.58-2.52 (m, 1H), 2.41-2.35 (m, 1H), 2.24 (s, 3H), 2.22-2.15 (m, 1H), 2.07-1.96 (m, 1H), 1.73- 1.61 (m, 1H). LCMS (Analytical Method A) Rt = 0.99 min, MS (ESIpos): m/z 408.2 [M + H]+, Purity = 96%. |

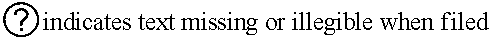

### Synthesis of 2-[4-(2-fluorophenyl)-5-(pyridin-4-yl)-1H-imidazol-1-yl]-1-(4-methylpiperazin-1-yl)ethan-1-one/Compound 127 of Table 1

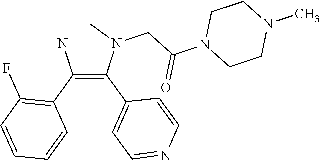 [SMILES: CN1CCN(C(=O)CN(C)/C(=C(\N)c2ccccc2F)c2ccncc2)CC1]

A mixture of 2-[4-bromo-5-(4-pyridyl)imidazol-1-yl]-1-(4-methylpiperazin-1-yl)ethanone (Intermediate 74) (100 mg, 0.253 mmol), (2-fluorophenyl)boronic acid (42 mg, 0.303 mmol), Pd(PPh₃)₄ (15 mg, 0.0126 mmol) and 2 M Na₂CO₃ (0.63 mL, 1.26 mmol) in DME (2.5 mL) was degassed by sparging with nitrogen. The reaction was heated to 125° C. under microwave irradiation for 2 h. The reaction mixture was diluted with EtOAc (5 mL), washed with water (5 mL), dried over MgSO₄, filtered and evaporated under reduced pressure. The residue was purified by flash chromatography (10 g, silica), eluting with 0-25% MeOH/DCM. After evaporating the solvent, the residue was freeze dried overnight to afford the title compound (35 mg, 37% yield) as a pale-yellow solid. 1H NMR (500 MHz, Chloroform-d) δ 8.63-8.58 (m, 2H), 7.72 (s, 1H), 7.58 (td, J=7.6, 1.8 Hz, 1H), 7.27-7.22 (m, 1H), 7.16-7.10 (m, 3H), 6.98-6.90 (m, 1H), 4.68 (s, 2H), 3.69-3.58 (m, 2H), 3.40-3.30 (m, 2H), 2.40-2.35 (m, 2H), 2.35-2.31 (m, 2H), 2.30 (s, 3H). LCMS (Analytical Method B) Rt=1.88 min, MS (ESIpos): m/z 380.3 [M+H]+, Purity=100%.

Each of the compounds as listed in Table 1.9.6 were prepared according to the method of Compound 127, using the intermediates listed in the “Synthesis” column for such compounds. The final compounds were purified by flash chromatography (10 g, silica) eluting with 0-25% MeOH/DCM and/or preparative HPLC Method A1.

TABLE 1.9.6

| Example | Synthesis | Structure/Name | Data |
|---|---|---|---|
| Compound 128 | Intermediate 74 and (3- fluorophenyl) boronic acid (41% yield) | 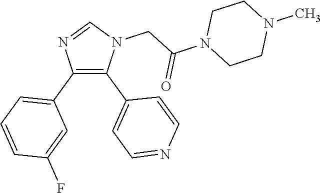 [SMILES: CN1CCN(C(=O)Cn2cnc(-c3cccc(F)c3)c2-c2ccncc2)CC1] 2-[4-(3-fluorophenyl)-5-(pyridin- 4-yl)-1H-imidazol-1-yl]-1-(4- methylpiperazin-1-yl)ethan-1-one | 1H NMR (400 MHz, Chloroform-d) δ 8.75-8.67 (m, 2H), 7.65 (s, 1H), 7.28- 7.25 (m, 2H), 7.24-7.19 (m, 1H), 7.19-7.14 (m, 2H), 6.92- 6.85 (m, 1H), 4.58 (s, 2H), 3.68-3.55 (m, 2H), 3.37- 3.26 (m, 2H), 2.41-2.33 (m, 2H), 2.32-2.26 (m, 5H). LCMS (Analytical Method B) Rt = 2.05 min, MS (ESIpos): m/z 380.3 [M + H]+, Purity = 100%. |
| Compound 129 | Intermediate 74 and 2-(2- chlorophenyl)- 4, 4, 5, 5- tetramethyl- 1, 3, 2- dioxaborolane (22% yield) | 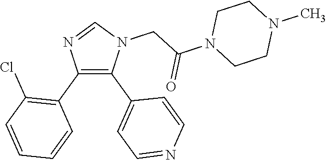 [SMILES: CN1CCN(C(=O)Cn2cnc(-c3ccccc3Cl)c2-c2ccncc2)CC1] 2-[4-(2-chlorophenyl)-5-(pyridin- 4-yl)-1H-imidazol-1-yl]-1-(4- methylpiperazin-1-yl)ethan-1-one | 1H NMR (400 MHz, Chloroform-d) δ 8.60-8.51 (m, 2H), 7.71 (s, 1H), 7.38- 7.32 (m, 2H), 7.25-7.17 (m, 2H), 7.09-7.04 (m, 2H), 4.74 (s, 2H), 3.71-3.60 (m, 2H), 3.43-3.33 (m, 2H), 2.43- 2.33 (m, 4H), 2.31 (s, 3H). LCMS (Analytical Method B) Rt = 1.98 min, MS (ESIpos): m/z 396.3, 398.2 [M + H]+, Purity = 97%. |
| Compound 156 | Intermediate 74 and (4- chloro-3- fluorophenyl) boronic acid (38% yield) | 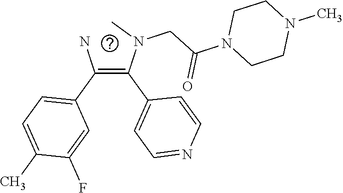 2-[4-(4-chloro-3-fluorophenyl)-5- (pyridin-4-yl)-1H-imidazol-1-yl]- 1-(4-methylpiperazin-1-yl)ethan-1-one | 1HNMR(500 MHz, DMSO-d6) δ 8.70-8.67 (m, 2H), 7.85 (s, 1H), 7.45 (t, J = 8.2 Hz, 1H), 7.33-7.29 (m, 3H), 7.11 (dd, J = 8.3, 1.8 Hz, 1H), 4.91 (s, 2H), 3.37-3.34 (m, 2H), 3.32-3.30 (m, 2H), 2.16- 2.11 (m, 7H). LCMS (Analytical Method B) Rt = 2.42 min, MS (ESIpos): m/z 414.3, 416.2 [M + H]+, Purity = 100%. |
| Compound 159 | Intermediate 74 and (4- chloro-2- fluorophenyl) boronic acid (26% yield) | 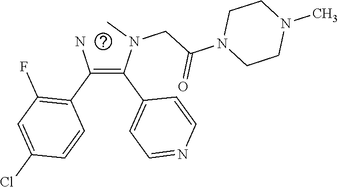 [SMILES: CN1CCN(C(=O)CN(C)/C(=C(\N)c2ccc(Cl)cc2F)c2ccncc2)CC1] 2-[4-(4-chloro-2-fluorophenyl)-5- (pyridin-4-yl)-1H-imidazol-1-yl]- 1-(4-methylpiperazin-1-yl)ethan-1-one | 1HNMR(500 MHz, DMSO-d6) δ 8.58-8.54 (m, 2H), 7.88 (s, 1H), 7.58 (t, J = 8.3 Hz, 1H), 7.33-7.29 (m, 2H), 7.15- 7.12 (m, 2H), 5.04 (s, 2H), 3.40-3.35 (m, 4H), 2.24- 2.19 (m, 2H), 2.18-2.13 (m, 5H) LCMS (Analytical Method B) Rt = 2.27 min, MS (ESIpos): m/z 414.3, 416.2 [M + H]+, Purity = 100%. |
| Compound 159 | Intermediate 74 and 2, 4- difluorophenyl boronic acid (19% yield) | 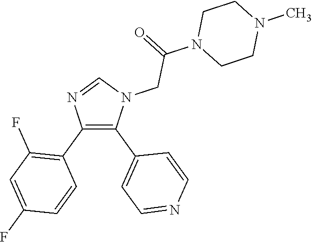 [SMILES: CN1CCN(C(=O)Cn2cnc(-c3ccc(F)cc3F)c2-c2ccncc2)CC1] 2-[4-(2,4-difluorophenyl)-5- (pyridin-4-yl)-1H-imidazol-1-yl]- 1-(4-methylpiperazin-1-yl)ethan-1-one | 1HNMR(500 MHz, DMSO-d6) 5 8.57-8.53 (m, 2H), 7.86 (s, 1H), 7.61-7.55 (m, 1H), 7.15- 7.09 (m, 4H), 5.04 (s, 2H), 3.40-3.35 (m, 4H), 2.25- 2.20 (m, 2H), 2.18-2.14 (m, 5H). LCMS (Analytical Method B) Rt = 2.01 min, MS (ESIpos): m/z 398.3 [M + H]+, Purity = 100%. |

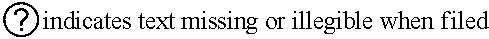

### Synthesis of 2-[4-(4-chlorophenyl)-5-(pyridin-4-yl)-1H-imidazol-1-yl]-1-[2-(2,2-difluoroethyl)-2,6-diazaspiro[3.4]octan-6-yl]ethan-1-one/Compound 119 of Table 1

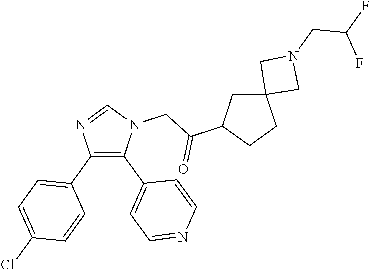 [SMILES: O=C(Cn1cnc(-c2ccc(Cl)cc2)c1-c1ccncc1)C1CCC2(C1)CN(CC(F)F)C2]

2,2-Difluoroethyl trifluoromethanesulfonate (12 uL, 0.0919 mmol) was added to a solution of 2-[4-(4-chlorophenyl)-5-(4-pyridyl)imidazol-1-yl]-1-(2,6-diazaspiro[3.4]octan-6-yl)ethanone (Intermediate 75) (25 mg, 0.0613 mmol) and Diisopropylethylamine (32 uL, 0.184 mmol) in THF (2.3 mL). The reaction was stirred for 1 hour then quenched into water. The aqueous layer was extracted into EtOAc (15 mL) three times, the combined organics washed with brine, dried over MgSO₄ and concentrated in vacuo. The residue was purified by preparative HPLC (Method A1). The relevant fractions were combined, the solvent volume reduced in vacuo and freeze dried to yield the title compound as a pale yellow solid (8 mg, 27% yield). 1H NMR (400 MHz, DMSO-d6) δ 8.64 (d, J=6.0 Hz, 2H), 7.79 (s, 1H), 7.38 (d, J=8.7 Hz, 2H), 7.31-7.26 (m, 4H), 5.89 (tt, J=55.9, 4.2 Hz, 1H), 4.75-4.70 (m, 2H), 3.43-3.35 (m, 2H), 3.28-3.25 (m, 2H), 3.19 (d, J=6.0 Hz, 4H), 2.81 (td, J=16.1, 4.2 Hz, 3H), 2.02-1.90 (m, 2H). LCMS (Analytical Method B) Rt=2.57 min, MS (ESIpos): m/z 472.3, 474.3 [M+H]+, Purity=98%.

### Synthesis of 2-[4-(4-chlorophenyl)-5-(pyridin-4-yl)-1H-imidazol-1-yl]-N-[(3S)-1-(2,2-difluoroethyl)pyrrolidin-3-yl]acetamide/Compound 142-S of Table 1

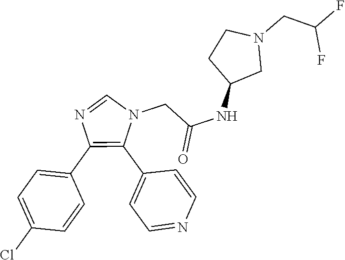 [SMILES: O=C(Cn1cnc(-c2ccc(Cl)cc2)c1-c1ccncc1)N[C@H]1CCN(CC(F)F)C1]

2-[4-(4-Chlorophenyl)-5-(4-pyridyl)imidazol-1-yl]-N-[(3S)-pyrrolidin-3-yl]acetamide; hydrochloride (Intermediate 76) (50 mg, 0.102 mmol) was dissolved in a solution of DIPEA (75 uL, 0.431 mmol) and THF (1.5 mL) then 2,2-difluoroethyl trifluoromethanesulfonate (14 uL, 0.106 mmol) was added. The reaction was stirred for 2 h. The reaction was stirred at 50° C. for 1 h. Additional DIPEA (75 uL, 0.431 mmol) was added and the reaction was stirred at 50° C. for 3 h. Additional

2,2-difluoroethyl trifluoromethanesulfonate (14 uL, 0.106 mmol) was added and the reaction was stirred for 6 h at 50° C. The reaction was concentrated in vacuo. The crude product was purified via preparative HPLC (Method A1) and lyophilised overnight to afford the title compound (8 mg, 18% yield) as a white solid. 1H NMR (500 MHz, DMSO-d6) δ 8.67-8.63 (m, 2H), 8.24 (d, J=7.0 Hz, 1H), 7.85 (s, 1H), 7.37-7.27 (m, 6H), 6.19-5.93 (m, 1H), 4.63-4.50 (m, 2H), 4.07-3.98 (m, 1H), 2.79 (td, J=16.0, 4.3 Hz, 2H), 2.72-2.62 (m, 2H), 2.47-2.41 (m, 1H), 2.27 (dd, J=9.6, 4.3 Hz, 1H), 2.03-1.94 (m, 1H), 1.42-1.32 (m, 1H). LCMS (Analytical Method B) Rt=2.57 min, MS (ESIpos): m/z 446.3, 448.2 [M+H]+, Purity=99%.

### Synthesis of 2-[4-(4-chlorophenyl)-5-(pyridin-4-yl)-1H-imidazol-1-yl]-N-[(3R)-1-(2,2-difluoroethyl)pyrrolidin-3-yl]acetamide/Compound 142-R of Table 1

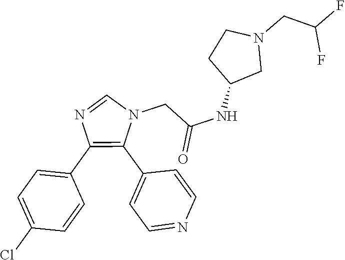 [SMILES: O=C(Cn1cnc(-c2ccc(Cl)cc2)c1-c1ccncc1)N[C@@H]1CCN(CC(F)F)C1]

2-[4-(4-Chlorophenyl)-5-(4-pyridyl)imidazol-1-yl]-N-[(3R)-pyrrolidin-3-yl]acetamide, hydrochloride (Intermediate 77) (60 mg, 0.116 mmol) was dissolved in DIPEA (85 uL, 0.488 mmol) and THF (1.5 mL) then 2,2-difluoroethyl trifluoromethanesulfonate (16 uL, 0.121 mmol) was added. The reaction was stirred for 1.5 h. The reaction was stirred at 50° C. for 2 h. Additional DIPEA (85 uL, 0.488 mmol) and 2,2-difluoroethyl trifluoromethanesulfonate (16 uL, 0.121 mmol) was added. Additional 2,2-difluoroethyl trifluoromethanesulfonate (16 uL, 0.121 mmol) was added. The reaction was concentrated in vacuo. The crude product was purified via preparative HPLC (Method A1) and lyophilised overnight to afford the title compound (15 mg, 28% yield) as a white solid. 1H NMR (500 MHz, DMSO-d6) δ 8.69-8.61 (m, 2H), 8.31-8.20 (m, 1H), 7.86 (s, 1H), 7.40-7.24 (m, 6H), 6.24-5.93 (m, 1H), 4.63-4.51 (m, 2H), 4.08-3.99 (m, 1H), 2.94-2.76 (m, 2H), 2.75-2.64 (m, 2H), 2.48-2.43 (m, 1H), 2.33-2.24 (m, 1H), 2.06-1.94 (m, 1H), 1.46-1.36 (m, 1H). LCMS (Analytical Method B) Rt=2.57 min, MS (ESIpos): m/z 446.3, 448.2 [M+H]+, Purity=100%.

### Synthesis of 2-[4-(4-chlorophenyl)-5-(pyridin-4-yl)-1H-imidazol-1-yl]-1-[2-(2,2-difluoroethyl)-2,7-diazaspiro[3.5]nonan-7-yl]ethan-1-one/Compound 139 of Table 1

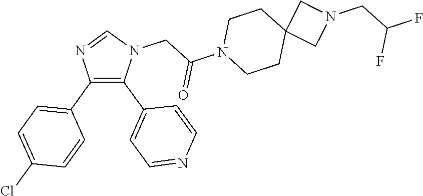 [SMILES: O=C(Cn1cnc(-c2ccc(Cl)cc2)c1-c1ccncc1)N1CCC2(CC1)CN(CC(F)F)C2]

2,2-Difluoroethyl trifluoromethanesulfonate (17 uL, 0.128 mmol) was added to a solution of 2-[4-(4-chlorophenyl)-5-(4-pyridyl)imidazol-1-yl]-1-(2,7-diazaspiro[3.5]nonan-7-yl)ethanone (compound 32 of table 1) (45 mg, 0.107 mmol) and DIPEA (37 uL, 0.213 mmol) in THF (1.5 mL). The reaction was stirred for 1 hour then quenched into water. The aqueous layer was extracted into EtOAc (15 mL) three times, the combined organics washed with brine, dried over MgSO₄ and concentrated in vacuo. The residue was purified by HPLC (Method A1). The relevant fractions were combined, the solvent volume reduced in vacuo and freeze dried to yield the title compound (33 mg, 62% yield) as a White solid. 1H NMR (400 MHz, DMSO-d6) δ 8.63 (d, J=6.0 Hz, 2H), 7.80 (s, 1H), 7.35 (d, J=8.8 Hz, 2H), 7.30 (d, J=8.8 Hz, 2H), 7.26 (d, J=6.0 Hz, 2H), 5.91 (tt, J=55.8, 4.2 Hz, 2H), 4.88 (s, 2H), 3.29-3.20 (m, 5H), 3.04 (s, 4H), 2.80 (td, J=16.1, 4.2 Hz, 2H), 1.48 (d, J=4.5 Hz, 4H). LCMS (Analytical Method B) Rt=2.70 min, MS (ESIpos): m/z 486.3, 488.2 [M+H]+, Purity=98%.

### Synthesis of 2-[4-(4-chlorophenyl)-2-(1-methyl-1H-pyrazol-4-yl)-5-(pyridin-4-yl)-1H-imidazol-1-yl]-1-(4-methylpiperazin-1-yl)ethan-1-one/Compound 141 of Table 1

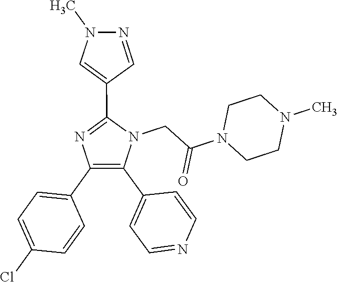 [SMILES: CN1CCN(C(=O)Cn2c(-c3cnn(C)c3)nc(-c3ccc(Cl)cc3)c2-c2ccncc2)CC1]

tert-Butyl 2-[4-(4-chlorophenyl)-2-(1-methylpyrazol-4-yl)-5-(4-pyridyl)imidazol-1-yl]acetate (Intermediate 78) (16 mg, 0.0356 mmol) was dissolved in DCM (0.75 mL) and TFA (0.25 mL) and the mixture was stirred at RT for 2 h. Additional TFA (0.25 mL) was added and the reaction was stirred for 18 h. The mixture was concentrated in vacuo. The residue was taken up in EtOAc (1 mL) then DIPEA (18 uL, 0.103 mmol), 1-methylpiperazine (6.0 uL, 0.0541 mmol) and T3P (50%, 30 uL, 0.0504 mmol) were added. The reaction was stirred for 1 h. Additional DIPEA (18 uL, 0.103 mmol), 1-methylpiperazine (6.0 uL, 0.0541 mmol) and T3P (50%, 30 uL, 0.0504 mmol) were added and the reaction was stirred for 2 h. The reaction was concentrated in vacuo. The crude product was purified via preparative HPLC (Method A1) and lyophilised overnight to afford the title compound (11 mg, 65% yield) as a white solid. 1H NMR (500 MHz, DMSO-d6) δ 8.71-8.68 (m, 2H), 8.01 (s, 1H), 7.68 (s, 1H), 7.40-7.36 (m, 2H), 7.34-7.31 (m, 2H), 7.30-7.27 (m, 2H), 4.77 (s, 2H), 3.92 (s, 3H), 3.47-3.41 (m, 2H), 3.36-3.33 (m, 2H), 2.23-2.18 (m, 2H), 2.15 (s, 3H), 2.13-2.08 (m, 2H). LCMS (Analytical Method B) Rt=2.39 min, MS (ESIpos): m/z 476.3, 478.2 [M+H]+, Purity=100%.

### Synthesis of 2-[4-(4-chlorophenyl)-2-(4-methoxyphenyl)-5-(pyridin-4-yl)-1H-imidazol-1-yl]-1-(4-methylpiperazin-1-yl)ethan-1-one/Compound 144 of Table 1

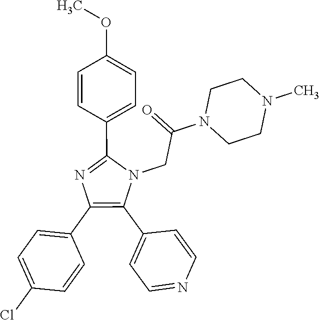 [SMILES: COc1ccc(-c2nc(-c3ccc(Cl)cc3)c(-c3ccncc3)n2CC(=O)N2CCN(C)CC2)cc1]

tert-Butyl 4-[2-[4-(4-chlorophenyl)-2-(4-methoxyphenyl)-5-(4-pyridyl)imidazol-1-yl]acetyl]piperazine-1-carboxylate (Intermediate N H, 79) (75 mg, 0.128 mmol) was dissolved 4M HCl in dioxane (1 mL) and stirred at RT for 16 h. The reaction was concentrated in vacuo. The residue was taken up in a solution of DCM (1 mL) and DIPEA (90 uL, 0.517 mmol) then 12 M formaldehyde (15 uL, 0.180 mmol) was added. The mixture was stirred for 1 h then STAB (54 mg, 0.255 mmol) was added. The reaction was stirred for 1 h at RT. The reaction was quenched with water and concentrated in vacuo. The crude product was purified via flash chromatography (10 g, silica) eluting with 0-10% MeOH in DCM and lyophilised overnight to afford the title compound (26 mg, 40% yield) as a beige coloured solid. 1H NMR (400 MHz, DMSO-d6) δ 8.78-8.74 (m, 2H), 7.62-7.57 (m, 2H), 7.50-7.45 (m, 2H), 7.42-7.37 (m, 4H), 7.17-7.10 (m, 2H), 4.76 (s, 2H), 3.88 (s, 3H), 3.49-3.43 (m, 2H), 3.32-3.25 (m, 2H), 2.23-2.19 (m, 2H), 2.17 (s, 3H), 2.08-2.02 (m, 2H). LCMS (Analytical Method B) Rt=2.97 min, MS (ESIpos): m/z 502.3, 504.2 [M+H]+, Purity=100%.

### Synthesis of 2-[4-(4-chlorophenyl)-2-(6-methoxypyridin-3-yl)-5-(pyridin-4-yl)-1H-imidazol-1-yl]-1-(4-methylpiperazin-1-yl)ethan-1-one/Compound 147 of Table 1

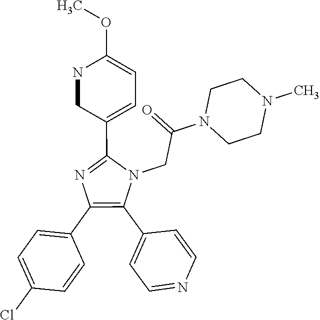

tert-Butyl 4-[2-[4-(4-chlorophenyl)-2-(6-methoxy-3-pyridyl)-5-(4-pyridyl)imidazol-1-yl]acetyl]piperazine-1-carboxylate (Intermediate 80) (34 mg, 0.0560 mmol) was dissolved in 4M HCl in dioxane (1 mL) and stirred at RT for 16 h. The reaction was concentrated in vacuo. The residue was taken up in DCM (1 mL) and DIPEA (40 uL, 0.230 mmol) then 12 M formaldehyde (10 uL, 0.120 mmol) was added. The reaction was stirred for 30 min then STAB (24 mg, 0.112 mmol) was added. The reaction was stirred for 1 h. The reaction was quenched with water and extracted with DCM. The organics were combined and concentrated in vacuo. The crude product was purified via flash chromatography (silica) eluting with 0-10% 10% ammonia in MeOH/DCM then lyophilised overnight to afford the title compound (13 mg, 46% yield) as a white solid. 1H NMR (500 MHz, DMSO-d6) δ 8.73-8.69 (m, 2H), 8.39 (dd, J=2.5, 0.6 Hz, 1H), 7.91 (dd, J=8.6, 2.4 Hz, 1H), 7.42-7.39 (m, 2H), 7.36-7.31 (m, 4H), 6.99 (dd, J=8.5, 0.6 Hz, 1H), 4.76 (s, 2H), 3.93 (s, 3H), 3.41-3.37 (m, 2H), 3.25-3.20 (m, 2H), 2.15-2.12 (m, 2H), 2.11 (s, 3H), 2.02-1.95 (m, 2H). LCMS (Analytical Method B) Rt=2.82 min, MS (ESIpos): m/z 503.4, 505.3 [M+H]+, Purity=100%.

### Synthesis of 2-[4-(4-chlorophenyl)-5-(pyridin-4-yl)-11H-imidazol-1-yl]-1-{3-[(1H-imidazol-1-yl)methyl]azetidin-1-yl}ethan-1-one/Compound 164 of Table 1

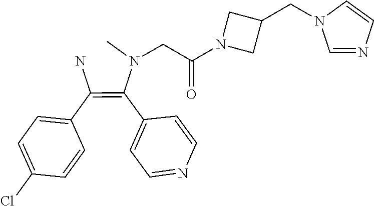 [SMILES: CN(CC(=O)N1CC(Cn2ccnc2)C1)/C(=C(\N)c1ccc(Cl)cc1)c1ccncc1]

T3P in EtOAc (50%, 270 uL, 0.454 mmol) was added to a stirred solution of 2-[4-(4-chlorophenyl)-5-(4-pyridyl)imidazol-1-yl]acetic acid; 2,2,2-trifluoroacetic acid (Intermediate 2a) (100 mg, 0.183 mmol) and DIPEA (160 uL, 0.916 mmol) in EtOAc (2 mL). After stirring for 5 min 1-(azetidin-3-ylmethyl)imidazole (Intermediate 81) (70% purity, 52 mg, 0.265 mmol) was incorporated to the reaction, and the mixture was stirred at RT for 19 h. 1 M aq. NaOH was added (3 mL) and the organic layer was separated. The aqueous layer was extracted with DCM (2×3 mL), and the organic layer were combined and dried using a hydrophobic Telos phase separator and evaporated under reduced pressure. The resulting residue was purified by preparative HPLC Method A1) to yield the title compound (15 mg, 18% yield) as a beige solid. 1H NMR (400 MHz, Chloroform-d) δ 8.76-8.70 (m, 2H), 7.65 (s, 1H), 7.47 (s, 1H), 7.39-7.33 (m, 2H), 7.28-7.27 (m, 2H), 7.24-7.19 (m, 2H), 7.09 (s, 1H), 6.87 (s, 1H), 4.36 (s, 2H), 4.20-4.05 (m, 3H), 4.00 (t, J=8.5 Hz, 1H), 3.74 (dd, 1H), 3.60 (dd, 1H), 3.12-3.00 (m, 1H). LCMS (Analytical Method B) Rt=2.30 min, MS (ESIpos): m/z 433.3, 435.2 [M+H]+, Purity=100%.

### Synthesis of 2-[4-(4-fluorophenyl)-5-(pyrimidin-4-yl)-1H-imidazol-1-yl]-1-(4-methylpiperazin-1-yl)ethan-1-one/Compound 111 of Table 1

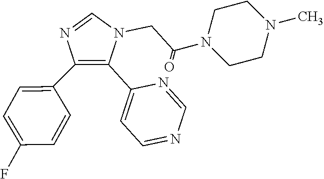 [SMILES: CN1CCN(C(=O)Cn2cnc(-c3ccc(F)cc3)c2-c2ccncn2)CC1]

2-[4-(4-Fluorophenyl)-5-pyrimidin-4-yl-imidazol-1-yl]-1-piperazin-1-yl-ethanone; hydrochloride (Intermediate 82) (70% purity, 42 mg, 0.0618 mmol) was suspended in DCM (1 mL) then DIPEA (35 uL, 0.200 mmol), 13 M formaldehyde (30 uL, 0.390 mmol) and STAB (50 mg, 0.236 mmol) were added. The mixture was stirred for 1 h. The reaction was retreated with DIPEA (35 uL, 0.200 mmol), 13 M formaldehyde (30 uL, 0.390 mmol) and STAB (50 mg, 0.236 mmol) and stirred at RT for 3 h. The reaction was quenched with water then concentrated in vacuo. The crude product was purified via preparative HPLC (Method A1) to afford the title compound (17 mg, 72% yield) as a white solid. 1H NMR (500 MHz, DMSO-d6) δ 9.18 (d, J=1.4 Hz, 1H), 8.62 (d, J=5.4 Hz, 1H), 7.88 (s, 1H), 7.50-7.45 (m, 2H), 7.24-7.17 (m, 3H), 5.33 (s, 2H), 3.51-3.45 (m, 2H), 2.37-2.32 (m, 2H), 2.19 (s, 3H), 2.18-2.14 (m, 2H). LCMS (Analytical Method B) Rt=2.00 min, MS (ESIpos): m/z 381.3 [M+H]+, Purity=100%.

### Synthesis of 2-[3-(4-fluorophenyl)-1-methyl-4-(pyridin-4-yl)-1H-pyrazol-5-yl]-1-(4-methylpiperazin-1-yl)ethan-1-one/Compound 117 of Table 1

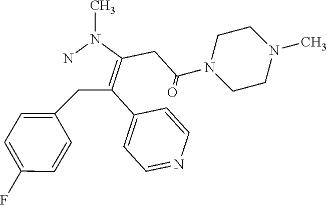 [SMILES: CN1CCN(C(=O)C/C(=C(/Cc2ccc(F)cc2)c2ccncc2)N(C)N)CC1]

To a stirred mixture of lithium(1+) 2-[3-(4-fluorophenyl)-1-methyl-4-(pyridin-4-yl)-1H-pyrazol-5-yl]acetate and lithium(1+) 2-[5-(4-fluorophenyl)-1-methyl-4-(pyridin-4-yl)-1H-pyrazol-3-yl]acetate (Intermediate 83) (78% purity, 200 mg, 0.492 mmol) in DIPEA (0.26 mL, 1.48 mmol) and EtOAc (4 mL) at RT was added T3P (50%, 0.59 mL, 0.983 mmol) followed by 1-methylpiperazine (82 uL, 0.738 mmol), and the resulting mixture was allowed to stir at 60° C. overnight. T3P (50%, 0.59 mL, 0.983 mmol), DIPEA (0.26 mL, 1.48 mmol), and 1-methylpiperazine (82 uL, 0.738 mmol) were added again and the mixture stirred at 60° C. overnight. The reaction was diluted with EtOAc (10 mL) and washed with sat. NaHCO₃(15 mL). The aq layer was extracted with EtOAc (2×15 mL). The combined organics were dried over MgSO₄, filtered, and evaporated under reduced pressure. The residue was purified by preparative HPLC (Instrument pump: Gilson 331 & 332; auto injector: Gilson GX281; UV detector: Gilson 159; collector: Gilson GX281; Column: Waters X-Bridge C18 19×100 mm, 5 μm; eluent A: water+0.2 vol % ammonium hydroxide, eluent B: acetonitrile+0.2 vol % ammonium hydroxide; gradient: 0-1.9 min 5% B, 1.9-2.0 min 5-20% B, 2.0-16.0 min 20-30% B, flow 20 mL/min; temperature: 25° C.; UV scan: 215 nm) to provide the desired regioisomer, and the undesired regioisomer. The desired regioisomer was dissolved in a small amount of MeCN, diluted with water and freeze dried overnight to provide the title compound (19 mg, 9.6% yield) 1H NMR (400 MHz, Chloroform-d) δ 8.57 (d, J=5.6 Hz, 2H), 7.38-7.30 (m, 2H), 7.11-7.04 (m, 2H), 7.01-6.92 (m, 2H), 3.93 (s, 3H), 3.70-3.62 (m, 4H), 3.42-3.34 (m, 2H), 2.44-2.36 (m, 2H), 2.33-2.26 (m, 5H). LCMS (Analytical Method B) Rt=2.27 min, MS (ESIpos): m/z 394.4 [M+H]+, Purity=98%.

### Synthesis of 2-[3-(4-fluorophenyl)-4-(pyridin-4-yl)-1H-pyrazol-5-yl]-1-(4-methylpiperazin-1-yl)ethan-1-one/Compound 118 of Table 1

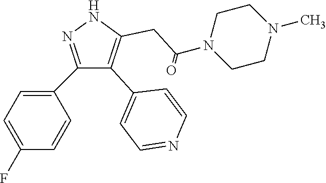 [SMILES: CN1CCN(C(=O)Cc2[nH]nc(-c3ccc(F)cc3)c2-c2ccncc2)CC1]

A solution of 2-[1-tert-butyl-5-(4-fluorophenyl)-4-(4-pyridyl)pyrazol-3-yl]-1-(4-methylpiperazin-1-yl)ethanone (Intermediate 84) (55 mg, 0.120 mmol) in formic acid (2.09 mL) was heated at 80° C. overnight. The reaction mixture was diluted with water (5 mL) and 1M aq. NaOH was added until basic pH (11) was achieved. The mixture was then extracted with DCM (2×15 mL) and the organic layers filtered through a Telos phase separator and evaporated in vacuo. The residue was purified by preparative HPLC (Method A1) and freeze dried overnight to yield the title compound (18 mg, 38% yield) as a white solid. 1H NMR (500 MHz, Chloroform-d) δ 8.61-8.56 (m, 2H), 7.37-7.29 (m, 2H), 7.15-7.10 (m, 2H), 7.00 (t, J=8.7 Hz, 2H), 3.75 (s, 2H), 3.70-3.65 (m, 2H), 3.48-3.41 (m, 2H), 2.42-2.37 (m, 2H), 2.37-2.32 (m, 2H), 2.30 (s, 3H). LCMS (Analytical Method B) Rt=2.05 min, MS (ESIpos): m/z 380.3 [M+H]+, Purity=96%.

### Synthesis of N-{4-[4-(4-fluorophenyl)-1-[2-(morpholin-4-yl)-2-oxoethyl]-1H-imidazol-5-yl]pyridin-2-yl}-2-methylpropanamide/Compound 123 of Table 1

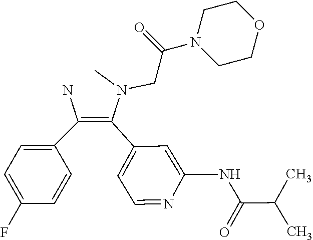

2-[5-(2-Amino-4-pyridyl)-4-(4-fluorophenyl)imidazol-1-yl]-1-morpholino-ethanone (Intermediate 85) (40 mg, 0.105 mmol) was dissolved in THF (1 mL) and DIPEA (46 uL, 0.262 mmol) then 2-methylpropanoyl chloride (23 uL, 0.220 mmol) was added. The mixture was stirred for 2 h. 2 M aq. NaOH (1 mL, 2.00 mmol) was added and the reaction was stirred for 1 h. The reaction was diluted with water and extracted with EtOAc. The organics were combined and concentrated in vacuo. The crude product was purified via preparative HPLC (Method A2) then lyophilised overnight to afford the title compound (22 mg, 46% yield). 1H NMR (400 MHz, DMSO-d6) δ 10.57 (s, 1H), 8.34-8.32 (m, 1H), 8.01-7.98 (m, 1H), 7.78 (s, 1H), 7.45-7.39 (m, 2H), 7.14-7.06 (m, 2H), 6.88 (dd, J=5.1, 1.5 Hz, 1H), 4.87 (s, 2H), 3.57-3.52 (m, 2H), 3.48-3.40 (m, 6H), 2.74 (d, J=6.9 Hz, 1H), 1.07 (d, J=6.8 Hz, 6H). LCMS (Analytical Method B) Rt=2.51 min, MS (ESIpos): m/z 452.3 [M+H]+, Purity=99%.

### Synthesis of 2-[4-(4-chlorophenyl)-5-(pyridin-4-yl)-1H-imidazol-1-yl]-1-[2-(oxetan-3-yl)-2,7-diazaspiro[3.5]nonan-7-yl]ethan-1-one/Compound 133 of Table 1

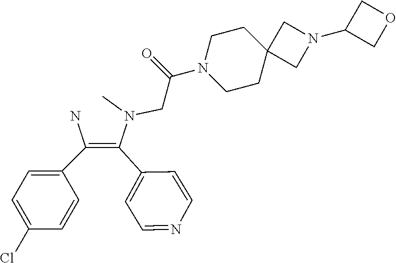 [SMILES: CN(CC(=O)N1CCC2(CC1)CN(C1COC1)C2)/C(=C(\N)c1ccc(Cl)cc1)c1ccncc1]

Oxetan-3-one (32 mg, 0.443 mmol) was added to a solution of 2-[4-(4-chlorophenyl)-5-(4-pyridyl)imidazol-1-yl]-1-(2,7-diazaspiro[3.5]nonan-7-yl)ethanone (compound 32 of table 1) (83% purity, 45 mg, 0.0885 mmol) in THF (2 mL) and the mixture was stirred at RT for 1 h. STAB (38 mg, 0.177 mmol) was then added and the reaction was stirred at RT/overnight. The reaction was concentrated in vacuo and the residue was partitioned between DCM (3 mL) and 1 M aq. NaOH (3 mL). The aqueous layer was extracted with DCM (2×3 mL). The combined organics were separated using a Telos phase separator. The filtrate was concentrated in vacuo and the residue was purified by preparative HPLC (Method A1) followed by flash chromatography (5 g, silica), eluting with 0-30% MeOH/DCM to yield the title compound (8.0 mg, 18% yield) as a white solid. 1H NMR (400 MHz, Methanol-d4) δ 8.62-8.57 (m, 2H), 7.85 (s, 1H), 7.39-7.30 (m, 4H), 7.30-7.22 (m, 2H), 4.99 (s, 2H), 4.73 (t, J=6.8 Hz, 2H), 4.48 (dd, J=6.8, 5.1 Hz, 2H), 3.88-3.78 (m, 1H), 3.48-3.40 (m, 2H), 3.40-3.34 (m, 2H), 3.19-3.09 (m, 4H), 1.67 (m, 4H). LCMS (Analytical Method B) Rt=2.32 min, MS (ESIpos): m/z 478.3, 480.3 [M+H]+, Purity=94%.

### Synthesis of 2-[4-(4-chlorophenyl)-5-(pyridin-4-yl)-1H-imidazol-1-yl]-N-methyl-N-{2-methyl-5-oxa-2-azaspiro[3.4]octan-7-yl}acetamide/Compound 146 of Table 1

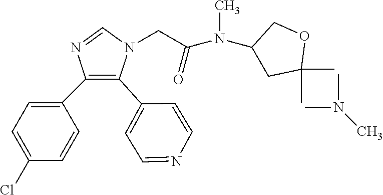

Formaldehyde (37% in water) (37%, 41 uL, 0.480 mmol) was added to a solution of 2-[4-(4-chlorophenyl)-5-(4-pyridyl)imidazol-1-yl]-N-methyl-N-(5-oxa-2-azaspiro[3.4]octan-7-yl)acetamide (Intermediate 86) (21 mg, 0.0480 mmol) in DCM (1 mL) and MeOH (0.2 mL). The reaction was stirred for 20 minutes then STAB (30 mg, 0.144 mmol) was added. The reaction was stirred for 1 hour then quenched into saturated NaHCO₃(aq). The aqueous layer was extracted into EtOAc (10 mL) three times, the combined organics washed with brine, dried over MgSO₄ and concentrated in vacuo. The residue was purified by preparative HPLC (Method A1). The relevant fractions were combined, the solvent volume reduced in vacuo and freeze dried to yield the title compound (16 mg, 74% yield) as a cream solid. 1H NMR (400 MHz, DMSO-d6) δ 8.64 (d, J=6.0 Hz, 2H), 7.77 (s, 1H), 7.38 (d, J=8.7 Hz, 2H), 7.30-7.24 (m, 4H), 4.85 (s, 2H), 3.67 (dd, J=9.5, 7.1 Hz, 1H), 3.53 (dd, J=9.4, 4.8 Hz, 1H), 3.34 (d, J=7.3 Hz, 1H), 3.22 (d, J=7.0 Hz, 1H), 2.95 (d, J=7.1 Hz, 1H), 2.70 (s, 3H), 2.28 (dd, J=13.5, 8.5 Hz, 1H), 2.23 (s, 3H), 1.92 (dd, J=13.5, 6.0 Hz, 1H). LCMS (Analytical Method B) Rt=2.59 min, MS (ESIpos): m/z 452.3, 454.2 [M+H]+, Purity=100%.

### Synthesis of 2-[2-chloro-4-(4-fluorophenyl)-5-(pyridin-4-yl)-1H-imidazol-1-yl]-1-{2-ethyl-2,7-diazaspiro[3.5]nonan-7-yl}ethan-1-one/Compound 151 of Table 1

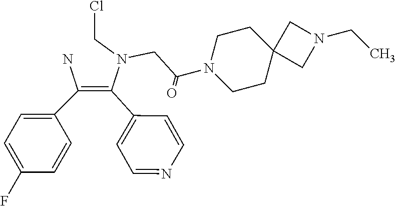 [SMILES: CCN1CC2(CCN(C(=O)CN(CCl)/C(=C(\N)c3ccc(F)cc3)c3ccncc3)CC2)C1]

Bromoethane (5.5 uL, 0.0747 mmol) was added to a solution of 2-[2-chloro-4-(4-fluorophenyl)-5-(4-pyridyl)imidazol-1-yl]-1-(2,7-diazaspiro[3.5]nonan-7-yl)ethanone (Intermediate 51) (31 mg, 0.0711 mmol) and N-ethyl-N-(propan-2-yl)propan-2-amine (25 uL, 0.142 mmol) in DMF-Anhydrous (1.5 mL). The reaction was stirred at 50° C. for 48 h then quenched into water. The aqueous layer was extracted into EtOAc (15 mL) three times, the combined organics washed with brine, dried over MgSO₄ and concentrated in vacuo. The residue was purified by preparative HPLC (Method A1) and freeze dried overnight to yield the title compound (8.1 mg, 24% yield) as a white solid. 1H NMR (400 MHz, Chloroform-d) δ 8.71-8.64 (m, 2H), 7.40-7.33 (m, 2H), 7.28-7.23 (m, 2H), 6.92 (t, J=8.8 Hz, 2H), 4.55 (s, 2H), 3.58-3.47 (m, 2H), 3.33-3.23 (m, 2H), 3.06 (d, J=7.3 Hz, 2H), 2.96 (d, J=7.1 Hz, 2H), 2.48 (q, J=7.1 Hz, 2H), 1.74-1.66 (m, 4H), 0.96 (t, J=7.2 Hz, 3H). LCMS (Analytical Method B) Rt=2.83 min, MS (ESIpos): m/z 468.3, 470.3 [M+H]+, Purity=97%.

### Synthesis of 2-[4-(4-chlorophenyl)-2-cyclopropyl-5-(pyridin-4-yl)-1H-imidazol-1-yl]-1-(4-methylpiperazin-1-yl)ethan-1-one/Compound 158 of Table 1

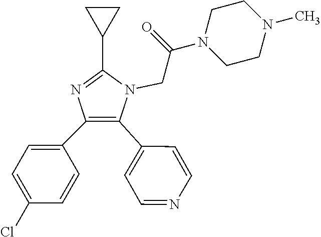 [SMILES: CN1CCN(C(=O)Cn2c(C3CC3)nc(-c3ccc(Cl)cc3)c2-c2ccncc2)CC1]

tert-Butyl 4-[2-[4-(4-chlorophenyl)-2-cyclopropyl-5-(4-pyridyl)imidazol-1-yl]acetyl]piperazine-1-carboxylate (Intermediate 87) (62% purity, 35 mg, 0.0416 mmol) was dissolved in 4M HCl in dioxane (1 mL) and stirred at RT for 2 h. The reaction mixture was concentrated in vacuo. The crude product was taken up in a solution of DCM (1 mL) and DIPEA (50 uL, 0.287 mmol), then 12 M formaldehyde (20 uL, 0.240 mmol) was added. The reaction was stirred for 20 min then STAB (20 mg, 0.0944 mmol) was added. The reaction was stirred for 1 h. The reaction was concentrated in vacuo and the crude product was purified via preparative HPLC (Method A1) and lyophilised overnight. Resulting residue was purified by preparative HPLC (instrument pump: Gilson 331 & 332; auto injector: Gilson GX281; UV detector: Gilson 159; collector: Gilson GX281; Column: Waters X-Bridge CSH 30×100 mm, 5 μm; eluent A: water+0.1 vol % formic acid, eluent B: acetonitrile+0.1 vol % formic acid; gradient: 0-2 min 5% B, 2-16 min 5-20% B, flow 20 mL/min; temperature: 25° C.; UV scan: 215 nm). The product was lyophilised overnight to give the title compound (7 mg, 39% yield) as a white solid. 1H NMR (500 MHz, DMSO-d6) δ 8.61-8.57 (m, 2H), 7.25-7.19 (m, 4H), 7.18-7.15 (m, 2H), 4.79 (s, 2H), 3.37-3.33 (m, 2H), 3.32-3.29 (m, 3H), 2.13-2.10 (m, 2H), 2.10-2.06 (m, 5H), 1.87-1.80 (m, 1H), 0.86-0.83 (m, 4H). LCMS (Analytical Method B) Rt=2.67 min, MS (ESIpos): m/z 436.4, 438.3 [M+H]+, Purity=100%.

### Synthesis of 2-[4-(4-chlorophenyl)-5-[2-(difluoromethyl)pyridin-4-yl]-2-hydroxy-1H-imidazol-1-yl]-1-{2-methyl-2,7-diazaspiro[3.5]nonan-7-yl}ethan-1-one/Compound 161 of Table 1

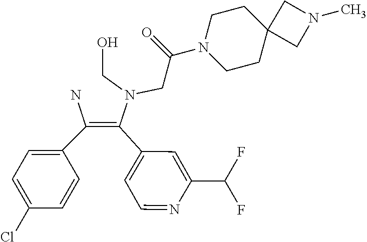 [SMILES: CN1CC2(CCN(C(=O)CN(CO)/C(=C(\N)c3ccc(Cl)cc3)c3ccnc(C(F)F)c3)CC2)C1]

12.7 M Formaldehyde (0.079 mL, 1.01 mmol) was added to a solution of 2-[4-(4-chlorophenyl)-5-[2-(difluoromethyl)-4-pyridyl]-2-hydroxy-imidazol-1-yl]-1-(2,7-diazaspiro[3.5]nonan-7-yl)ethanone (Intermediate 88) (14 mg, 0.0275 mmol) in THF (0.2 mL) and the mixture was stirred at RT for 2 h. STAB (12 mg, 0.0574 mmol) was then added and the reaction was stirred at RT for 5 h. The reaction was concentrated in vacuo and the residue was partitioned between DCM (3 mL) and 1 M aq. NaOH (3 mL). The aqueous layer was extracted with DCM (2×3 mL). The combined organics were separated using a Telos phase separator. The filtrate was concentrated in vacuo, purified by preparative HPLC (Method A1) and freeze dried overnight to yield the title compound (7.0 mg, 51% yield) as a pale-yellow solid. 1H NMR (400 MHz, Chloroform-d) δ 9.19 (s, 1H), 8.60 (d, J=5.0 Hz, 1H), 7.59 (s, 1H), 7.32 (d, J=5.1 Hz, 1H), 7.25-7.22 (m, 2H), 7.15-7.07 (m, 2H), 6.62 (t, J=55.3 Hz, 1H), 4.39 (s, 2H), 3.52-3.43 (m, 2H), 3.34-3.22 (m, 2H), 3.08 (d, J=7.1 Hz, 2H), 3.00 (d, J=7.1 Hz, 2H), 2.34 (s, 3H), 1.76-1.63 (m, 4H). LCMS (Analytical Method B) Rt=2.77 min, MS (ESIpos): m/z 502.4, 504.4 [M+H]+, Purity=100%.

### Synthesis of 2-[2-bromo-4-(4-chlorophenyl)-5-(pyridin-4-yl)-1H-imidazol-1-yl]-1-{2-methyl-2,7-diazaspiro[3.5]nonan-7-yl}ethan-1-one/Compound 165 of Table 1

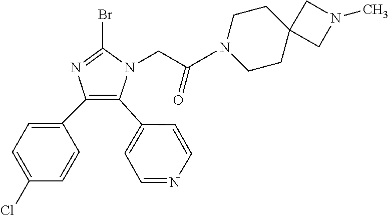

NBS (136 mg, 0.764 mmol) was added to a solution of 2-[4-(4-chlorophenyl)-5-(4-pyridyl)imidazol-1-yl]-1-(2-methyl-2,7-diazaspiro[3.5]nonan-7-yl)ethanone (compound 12 of Table 1) (222 mg, 0.509 mmol) in MeCN (5 mL) and the resulting mixture was allowed to stir at 60° C. for 5 h. The reaction was then retreated with NBS (136 mg, 0.764 mmol) and heated at 60° C. for 2 h. Reaction quenched with 1M aq. NaOH. Organic layer isolated using a Telos phase separator and evaporated under reduced pressure. The residue was purified by preparative HPLC (Method A1), and the product was freeze-dried overnight to yield the title compound (6.6 mg, 2.4% yield) as an off-white solid. 1H NMR (400 MHz, Methanol-d4) δ 8.65-8.61 (m, 2H), 7.39-7.34 (m, 2H), 7.34-7.29 (m, 2H), 7.29-7.24 (m, 2H), 4.87 (s, 2H), 3.51-3.45 (m, 2H), 3.41-3.36 (m, 2H), 3.22-3.11 (m, 4H), 2.40 (s, 3H), 1.71-1.63 (m, 4H). LCMS (Analytical Method A) Rt=1.67 min, MS (ESIpos): m/z 514.2, 516.2, 518.1 [M+H]+, Purity=94%.

### Synthesis of 2-[4-(4-chlorophenyl)-2-cyclopropyl-5-(pyridin-4-yl)-1H-imidazol-1-yl]-1-{2-methyl-2,7-diazaspiro[3.5]nonan-7-yl}ethan-1-one/Compound 167 of Table 1

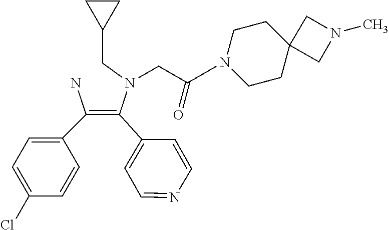

2-[2-Chloro-4-(4-chlorophenyl)-5-(4-pyridyl)imidazol-1-yl]-1-(2-methyl-2,7-diazaspiro[3.5]nonan-7-yl)ethanone (Intermediate 89) (24 mg, 0.0510 mmol), 2-cyclopropyl-4,4,5,5-tetramethyl-1,3,2-dioxaborolane (11 uL, 0.0604 mmol) and Tetrakis(triphenylphosphine)palladium(0) (6.0 mg, 5.19 μmol) were suspended in a solution of DME (0.5 mL) and water (0.15 mL). The mixture was degassed with nitrogen for 5 min then 2 M aq. Na₂CO₃ (57 uL, 0.114 mmol) was added. The reaction was stirred at 120° C. (microwave) for 5.5 hours. The reaction mixture was diluted with water (1 mL) and the organic layer extracted with EtOAc (1 mL). The aqueous was further extracted with EtOAc (2×1 mL). The organics were combined and concentrated in vacuo. The resulting residue was loaded onto a 5 g Biotage SCX-2 column. The column was washed with MeOH (5×20 mL) then the product was eluted with 3.5N ammonia MeOH (5×20 mL). The eluate was concentrated in vacuo. The resulting residue was purified by preparative HPLC (Instrument pump: Gilson 331 & 332; auto injector: Gilson GX281; UV detector: Gilson 159; collector: Gilson GX281; Column: Waters X-Bridge C18 19×100 mm, 5 μm; eluent A: water+0.1 vol % formic acid, eluent B: acetonitrile+0.1 vol % formic acid; 0-2 min 1% B, 2-16 min 1-55% B; flow 20 mL/min; temperature: 25° C.; UV scan: 215 nm) to afford the title compound (1.5 mg, 6.1% yield) as an off-white solid. 1H NMR (400 MHz, Chloroform-d) δ 8.65-8.60 (m, 2H), 7.35-7.30 (m, 2H), 7.23-7.20 (m, 2H), 7.18-7.14 (m, 2H), 4.62 (s, 2H), 3.52 (s, 2H), 3.27 (s, 2H), 3.08 (d, J=6.9 Hz, 2H), 3.00 (d, J=7.0 Hz, 2H), 2.34 (s, 3H), 1.74-1.65 (m, 5H), 1.15-1.09 (m, 2H), 0.99-0.92 (m, 2H). LCMS (Analytical Method B) Rt=2.88 min, MS (ESIpos): m/z 476.3 [M+H]+, Purity=98%.

### Example 2—Activity of Compounds of General Formula (I)

The DUX4 repression of compounds of general formula (I) was assayed following a known protocol (the protocol of Example 2 of WO2019/115711). Several compounds were incubated with primary FSHD cells for 72 hours. Results are shown in Table 4, showing DUX4 Count % inhibition. Table 5 shows additional results. Reference compounds 106, 107, and 108 from table 1 had DUX4 count % inhibition values of 17.3, 10.7, and 6.6, respectively.

TABLE 4
biological data for selected compounds of general formula (I)

| | DUX4 Count % inhibition | | |
|---|---|---|---|
| | Over | | |
| | 20%, | | |
| | below | Over 50%, | |
| | 50% | below 80% | Over 80% |
| Compound | 1, 54, | 2, 5, 10, 13, | 3, 4, 6, 7, 8, 9, 11, 12, 15, 17, |
| number in | 60, 69, | 14, 16, 18, 22, | 19, 20, 21, 23, 24, 25, 26, 27, |
| Table 1 | 104 | 28, 34, 40, 43, | 29, 30, 31, 32, 33, 35, 36, 37, |
| | | 45, 48, 49, 50, | 38, 39, 41, 42, 44, 46, 47, 52, |
| | | 51, 53, 55, 56, | 58, 59, 62, 65, 81, 82, 83, 84, |
| | | 57, 61, 63, 64, | 85, 86, 87, 88, 89, 91, 92, 93, |
| | | 90, 99 | 94, 95, 96, 97, 98, 100, 101, |
| | | | 102, 103, 105 |

TABLE 5
biological data for selected compounds ofcieneral formula (I)

| | DUX4 Count % inhibition | | |
|---|---|---|---|
| | Over 20%, below 50% | Over 50%, below 80% | Over 80% |
| Compound | 54, 60, 69, 99, 104, | 1, 2, 18, 21, 40, 43, | 3, 4, 5, 6, 7, 8, 9, 10, 11, 12, |
| number in | 168-R | 50, 51, 55, 56, 57, 61, | 13, 14, 15, 16, 17, 19, 20, 22, |
| Table 1 | | 63, 64, 65, 90, 143, | 23, 24, 25, 26, 27, 28, 29, 30, |
| | | 147, 148-R, 148-S, | 31, 32, 33, 34, 35, 36, 37, 38, |
| | | 149, 150, 151, 153, | 39, 41, 42, 44, 45, 46, 47, 48, |
| | | 154, 160, 161, 162, | 49, 52, 53, 58, 59, 62, 81, 82, |
| | | 164 | 83, 84, 85, 86, 87, 88, 89, 91, |
| | | | 92, 93, 94, 95, 96, 97, 98, |
| | | | 100, 101, 102, 103, 105, 137, |
| | | | 138-R, 138-S, 139, 140-R, |
| | | | 140-S, 141, 142-S, 142-R, |
| | | | 144, 145, 146, 152, 155, 156, |
| | | | 157-RR, 158, 159, 157-SS, |
| | | | 163, 165, 166-RR, 167 |

1. Compound of general formula (I): 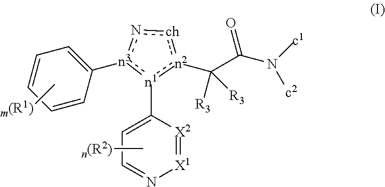
wherein
zero or one of n¹, n², and n³ are N, with the remainder of n¹, n², and n³ being C;
ch is CH, C(halogen), C(OH), C(—C₁₋₄alkyl), C(—C₁₋₄haloalkyl), C(—C₃₋₆cycloalkyl), C(—C₃₋₆ heterocycloalkyl), O, NH, N(—C₁₋₄alkyl), or N(—C₁₋₄haloalkyl);
R¹ is H, halogen, nitrile, —C₁₋₄alkyl, —C₁₋₃alkyl-nitrile, —C₁₋₄haloalkyl, —C₁₋₃haloalkyl-nitrile, —O—C₁₋₄alkyl, —O—C₁₋₃alkyl-nitrile, —O—C₁₋₄haloalkyl, —O—C₁₋₃haloalkyl-nitrile, —S—C₁₋₄alkyl, —S—C₁₋₃alkyl-nitrile, —S—C₁₋₄haloalkyl, or —S—C₁₋₃haloalkyl-nitrile;
m is 0, 1, 2, or 3;
R² is H, halogen, nitrile, —C₁₋₄alkyl, —C₁₋₃alkyl-nitrile, —C₁₋₄haloalkyl, —C₁₋₃haloalkyl-nitrile, —O—C₁₋₄alkyl, —O—C₁₋₃alkyl-nitrile, —O—C₁₋₄haloalkyl, —O—C₁₋₃haloalkyl-nitrile, —S—C₁₋₄alkyl, —S—C₁₋₃alkyl-nitrile, —S—C₁₋₄haloalkyl, —S—C₁₋₃haloalkyl-nitrile, or R² together with Q forms a bridging moiety;
n is 0, 1, or 2;
R³ is in each instance independently selected from H, halogen, or C₁₋₄alkyl;
X¹ is CH, C(R²), N, or C(Q);
X² is CH, C(R²), or N;
Q is H, halogen, C₁₋₆alkyl, —OH, —O—C₁₋₆alkyl, —O—C₁₋₆acyl, —NH₂, —NH—(C₁₋₆alkyl), —N(C₁₋₆alkyl)₂, —NH(C₁₋₈acyl), —N(C₁₋₈acyl)₂, —C₁₋₄alkyl-OH, —C₁₋₄alkyl-O—C₁₋₆alkyl, —C₁₋₄alkyl-O—C₁₋₆acyl, —C₁₋₄alkyl-NH₂, —C₁₋₄alkyl-NH—(C₁₋₆alkyl), —C₁₋₄alkyl-N(C₁₋₆alkyl)₂, —C₁₋₄alkyl-NH(C₁₋₈acyl), —C₁₋₄alkyl-N(C₁₋₈acyl)₂, —C₁₋₄alkyl-N—C(O)—NH—C₁₋₆alkyl, —C₁₋₄alkyl-N—C(O)—N(C₁₋₆alkyl)₂, —C₁₋₄alkyl-O—C(O)—NH—C₁₋₆alkyl, —C₁₋₄alkyl-O—C(O)—N(C₁₋₆alkyl)₂, —C₁₋₄alkyl-N—C(O)—O—C₁₋₆alkyl, or Q together with R² forms a bridging moiety selected from —NH—CH═CH—, —NH—(C₂₋₄alkyl)-, and —(C₁₋₃alkyl)-NH—(C₁₋₃alkyl)-;
c¹ is H, C₁₋₆alkyl, (C₁₋₂alkyl)₀₋₁C₃₋₆cycloalkyl, or (C₁₋₂alkyl)₀₋₁C₄₋₆heterocycloalkyl, preferably c¹ is H; and c² is C₄₋₈cycloalkyl, C₄₋₈heterocycloalkyl, C₄₋₈cycloalkyl-C₁₋₃alkyl, C₄₋₈heterocycloalkyl-C₁₋₃alkyl, C₁₋₃alkyl-C₄₋₈cycloalkyl, or C₁₋₃alkyl-C₄₋₈heterocycloalkyl; or c¹ and c² together form cyclic structure A;
A is a C₄₋₁₂cycloalkyl that can be cyclic, bicyclic, and tricyclic, and which is optionally unsaturated, and which is optionally substituted with halogen, C₁₋₆alkyl, C₃₋₆cycloalkyl, C₃₋₆ heterocycloalkyl, —O—C₁₋₄alkyl, hydroxyl, —NH₂, —NH(C₁₋₄alkyl), or —N(C₁₋₄alkyl)₂;
wherein each instance of acyl, alkyl, cycloalkyl, or heterocycloalkyl individually is optionally unsaturated, and optionally substituted with halogen, oxy, hydroxyl, methyl, ethyl, propyl, methoxy, ethoxy, trifluoromethyl, or optionally interrupted by one or more heteroatoms;
or a salt thereof.

2. Compound according to claim 1, wherein
n² is N and n¹ is C and n³ is C;
ch is CH, C(Cl), C(CH₃), C(isopropyl), C(CF₃), O, NH, or N(CH₃);
R¹ is H, fluorine, chlorine, —CH₃, —CF₃, —O—CH₃, or nitrile;
m is 0 or 1;
R² is H, fluorine, chlorine, or forms a bridging moiety;
n is 0;
R³ is H or —CH₃;
X¹ is C(Q);
X² is CH;
Q is H, F, —CH₃, —CH₂F, —CHF₂, —CF₃, —OCH₃, —OCH₂F, —OCHF₂, —OCF₃, —NH—C(O)—CH₃, —NH—C(O)-cyclopropyl, —NH—C(O)-phenyl, —NH—C(O)-halophenyl, —NH—C(O)-piperidinyl, —NH—C(O)-pyridinyl, —NH—C(O)-morpholinyl, —NH—C(O)-oxanyl, —NH₂, —NH(CH₃), —NH(cyclopentyl), —CH₂—NH—C(O)—CH₃, —CH₂—N(CH₃)₂, —CH₂—NH₂, —CH₂—NH—(CH₃), —CH₂—NH-(cyclopentyl), or together with R² forms —NH—CH═CH—; and/or wherein
c¹ is H and c² is pyridyl, —CH₂-pyridyl, piperidinyl, N-methylpiperidinyl, —CH₂-piperidinyl, —CH₂-(N-methylpiperidinyl), cyclopentyl, hydroxycyclopentyl, —CH₂-cyclopentyl, —CH₂-hydroxycyclopentyl, pyrrolidinyl, N-methylpyrrolidinyl, —CH₂-pyrrolidinyl, —CH₂-(N-methylpyrrolidinyl), or c¹ and c² together form cyclic structure A.

3. Compound according to claim 1, wherein Q is H, F, —NH—C(O)—CH₃, —NH—C(O)-cyclopropyl, —NH—C(O)-phenyl, —NH—C(O)-halophenyl, —NH₂, —NH(CH₃), —NH(cyclopentyl), —CH₂—NH—C(O)—CH₃, —CH₂—N(CH₃)₂, —CH₂—NH₂, —CH₂—NH—(CH₃), —CH₂—NH-(cyclopentyl), or together with R² forms —NH—CH═CH—; and/or wherein R³ is H; and/or wherein R¹ is H, fluorine, chlorine, —CH₃, —CF₃, or —O—CH₃.

4. Compound according to claim 1, wherein A is optionally substituted and optionally unsaturated azetidinyl, pyrrolidinyl, imidazolidinyl, oxazolidinyl, piperidinyl, piperazinyl, morpholinyl, azacycloheptyl, diazacycloheptyl, or oxoazacycloheptyl;
wherein each optional substitution can be a substitution with halogen, C₁₋₆alkyl, C₃₋₆ cycloalkyl, C₃₋₆heterocycloalkyl, —O—C₁₋₄alkyl, hydroxyl, —NH₂, —NH(C₁₋₄alkyl), or —N(C₁₋₄alkyl)₂; preferably each optional substitution is independently selected from methyl, dimethylamine, methoxyl, propyl, hydroxyl, a bridging C₁₋₃alkyl moiety, spiro azetidinyl, spiro N-methylazetidinyl, spiro oxetanyl, oxetanyl, spiro piperidinyl, difluoropiperidinyl, spiro N-methylpiperidinyl, spiro cyclopropyl, fused pyrrolidinyl, or fused N-methylpyrrolidinyl.

5. Compound according to claim 1, wherein it is of general formula (I-A):

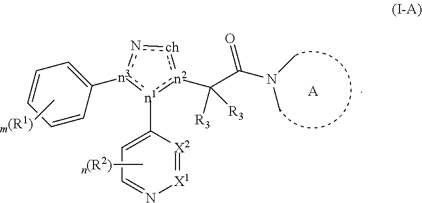

6. Compound according to claim 1, wherein it is of general formula (II) or (II-A):

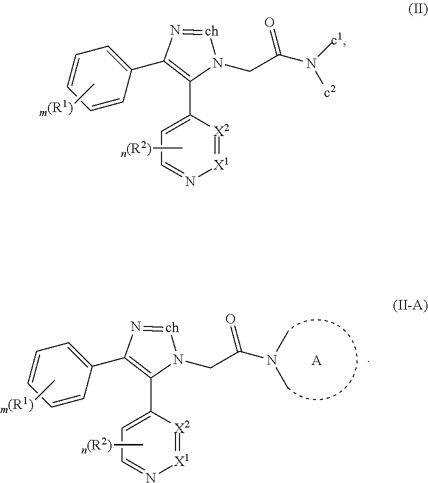

7. Compound according claim 1, wherein it is of general formula (III) or (III-A)

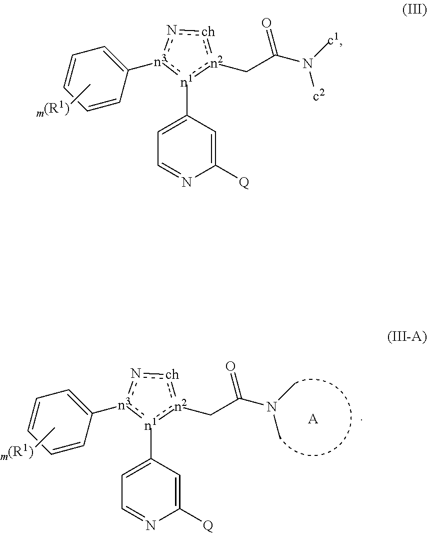

8. Compound according to claim 1, wherein A comprises an amine, more preferably wherein A is selected from A1, A2, A4, A5, A7, A8, A10-A13, A16-A38, and A41.

9. Compound according to claim 1, wherein m is 1, and wherein R¹ is para to the central ring, preferably wherein R¹ is halogen, more preferably fluorine.

10. Compound of general formula (I) wherein the compound is selected from compounds 1-105 and 109-168 as listed in table 1, preferably from compounds 1-105.

11. Compound of general formula (I), wherein the compound is selected from compounds 2, 5, 10, 13, 14, 16, 18, 22, 28, 34, 40, 43, 45, 48, 49, 50, 51, 53, 55, 56, 57, 61, 63, 64, 90, 99, 3, 4, 6, 7, 8, 9, 11, 12, 15, 17, 19, 20, 21, 23, 24, 25, 26, 27, 29, 30, 31, 32, 33, 35, 36, 37, 38, 39, 41, 42, 44, 46, 47, 52, 58, 59, 62, 65, 81, 82, 83, 84, 85, 86, 87, 88, 89, 91, 92, 93, 94, 95, 96, 97, 98, 100, 101, 102, 103, and 105 as listed in table 1.

12.-14. (canceled)

15. A method for reducing DUX4 expression in a subject in need thereof, the method comprising the step of administering an effective amount of a compound of general formula (I) as defined in claim 1.

16. The method according to claim 15, wherein the method is for the treatment of a disease or condition associated with DUX4 expression, and wherein the compound of general formula (I) reduces DUX4 expression.

17. The method according to claim 16, wherein the disease or condition associated with DUX4 expression is a muscular dystrophy.

18. The method according to claim 16, wherein the disease or condition associated with DUX4 expression facioscapulohumeral muscular dystrophy (FSHD).
